(19) United States
(12) Patent Application Publication
(10) Pub. No.: US 2018/0282410 A1
(43) Pub. Date: Oct. 4, 2018

(54) ANTI-CD3XROB04 BISPECIFIC T CELL ACTIVATING ANTIGEN BINDING MOLECULES

(71) Applicant: Hoffmann-La Roche Inc., Little Falls, NJ (US)

(72) Inventors: Oliver AST, Bassersdorf (CH); Marina BACAC, Zürich (CH); Sabine BAUER, Wolfratshausen (DE); Sabine IMHOF-JUNG, Planegg (DE); Christian KLEIN, Bonstetten (CH); Stefan KLOSTERMANN, Neuried (DE); Michael MOLHOJ, München (DE); Samuel MOSER, Rotkreuz (CH); Christiane NEUMANN, Niederweningen (CH); Joerg Thomas REGULA, München (DE); Wolfgang SCHAEFER, Mannheim (DE); Pablo UMAÑA, Wollerau (CH); Tina WEINZIERL, Schlieren (CH)

(73) Assignee: Hoffmann-La Roche Inc., Little Falls, NJ (US)

(21) Appl. No.: 15/764,939

(22) PCT Filed: Sep. 29, 2016
(86) PCT No.: PCT/EP2016/073178

§ 371 Date: Mar. 30, 2018

(30) Foreign Application Priority Data
Oct. 2, 2015 (EP) 15188266.9

(51) Int. Cl. C07K 16/28; A61P 35/00; C07K 16/46

(52) U.S. Cl. CPC C07K 16/283; A61P 35/00; C07K 16/468; C07K 16/2809; C07K 2317/31; C07K 2317/33; C07K 2319/00; C07K 2317/41; C07K 2317/526; C07K 2317/55; C07K 2317/66; C07K 2317/732; C07K 2317/92; C07K 2317/35

(57) ABSTRACT

The present invention generally relates to bispecific antigen binding molecules for activating T cells, more particularly bispecific antigen binding molecules for activating T cells targeting the Robo 4 receptor. In addition, the present invention relates to polynucleotides encoding such bispecific antigen binding molecules, and vectors and host cells comprising such polynucleotides. The invention further relates to methods for producing the bispecific antigen binding molecules of the invention, and to methods of using these bispecific antigen binding molecules in the treatment of disease. In addition, the invention also relates to antibodies that specifically bind to Robo 4.

### FIELD OF THE INVENTION

The present invention generally relates to bispecific antigen binding molecules for activating T cells, more particularly bispecific antigen binding molecules for activating T cells targeting the Robo 4 receptor. In addition, the present invention relates to polynucleotides encoding such bispecific antigen binding molecules, and vectors and host cells comprising such polynucleotides. The invention further relates to methods for producing the bispecific antigen binding molecules of the invention, and to methods of using these bispecific antigen binding molecules in the treatment of disease. In addition, the invention also relates to antibodies that specifically bind to Robo 4.

### BACKGROUND

The selective elimination of an individual cell or a specific cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. CTLs constitute the most potent effector cells of the immune system, however they cannot be activated by the effector mechanism mediated by the Fc domain of conventional therapeutic antibodies.

In this regard, bispecific antibodies designed to bind with one “arm” to a surface antigen on target cells, and with the second “arm” to an activating, invariant component of the T cell receptor (TCR) complex, have become of interest in the recent years. The simultaneous binding of such an antibody to both of its targets will force a temporary interaction (“crosslinking”) between a target cell and a T cell, causing activation of T cells and subsequent lysis of the target cell. Hence, the immune response is re-directed to the target cells and is independent of peptide antigen presentation by the target cell or the specificity of the T cell as would be relevant for normal MHC-restricted activation of CTLs. In this context it is crucial that CTLs are only activated when a target cell is presenting the bispecific antibody to them, i.e. the immunological synapse is mimicked. Particularly desirable are bispecific antibodies that do not require lymphocyte preconditioning or co-stimulation in order to elicit efficient lysis of target cells.

Previous approaches have focused on the direct destruction of tumor cells, by targeting an antigen expressed on the tumor cell surface. In contrast thereto, the present inventors have developed bispecific T cell activating antigen binding molecules directed to a target antigen on the tumor vasculature, enabling the destruction of vascular endothelial cells in the tumor and consequently reduction of tumor progression by abolishing the supply of nutrients and oxygen through the tumor vasculature.

Known pharmacologic approaches for inhibition of pathologic and tumor angiogenesis developed in the past were designed to target the VEGFR2/VEGF signaling pathway on endothelial cells. These classical antiangiogenic agents function through neutralization of the VEGF or VEGFR-2 pathway, immunization against VEGFR-2, coupling of VEGF to toxins or disruption of VEGFR genes. However, despite the multitude of approaches their effects are transient, resulting in cytostatic rather than cytotoxic activity, mostly because of the redundancy of angiogenic pathways activated within tumors. For that reason alternative approaches engaging immune effector cells against tumor vasculature have been developed. Chinnasamy et al. (Chinnasamy et al., J Clin Invest 120, 3953-3968 (2010)) used genetically engineered autologous T cells expressing a chimeric antigen receptor (CAR) targeting VEGFR-2 and demonstrated that a single dose of VEGFR-2 CAR T cells and exogenous IL-2 significantly inhibited the growth of five different established, vascularized syngeneic tumors and prolonged mice survival. In addition, immunohistochemical analysis of tumors treated with VEGFR2 CAR-transduced T cells showed their co-localization with tumor endothelial cells and increased infiltration within tumor compared to the empty vector-transduced T cells, suggesting that endothelial cell destruction renders the tumor vessels more permissive for extravasation and infiltration of adoptively transferred T cells into the tumor. As some human tumor cells have been reported to express VEGFR-2 on their surface, this may further enhance the antitumor effects during treatment of cancer patients. However, the main drawback of using engineered T cells is the need of engineering and ex vivo expansion of autologous T cells from a patient to be treated. In addition, exogenous IL-2 is required for effective tumor treatment.

To overcome the limitations associated with the engineered T cell approach the inventors of the present invention developed an antibody bispecific platform engaging T cells and redirecting them against the tumor neovasculature by targeting Robo 4. Robo 4 (also known as Magic Roundabout) is a tumor-specific vascular target, exclusively expressed at sites of active neo-angiogenesis. Robo 4 is a member of the Roundabout family of receptors, which further includes Robo 1, 2 and 3. It is specifically expressed on endothelial cells of tumor vessels in a vast panel of malignancies, but was not detectable in normal tissues in vivo, making it an attractive target for cancer therapy (Legg et al., Angiogenesis 11, 13-21 (2008)). Recent studies pointed out that Robo 4 stabilizes the vascular network by inhibiting VEGF-induced pathologic angiogenesis and endothelial hyperpermeability (Jones et al., Nat Med 14, 448-453 (2008)). Koch and colleagues elucidated that Robo 4 maintains vessel integrity and inhibits angiogenesis by interacting with UNC5B and proposed that Robo 4-UNC5B signaling maintains vascular integrity by counteracting VEGF signaling in endothelial cells (Koch et al., Dev Cell 20, 33-46 (2011)).

Redirecting T cells to Robo 4-expressing tumor neo-vasculature with the T cell bispecific antibodies of the present invention has multiple advantages. Firstly, vascular targets and effector cells circulating in the blood stream are directly accessible to the bispecific antibodies, without the need of T cell extravasation and migration into deeper tumor sites for activity. Therefore, the immune cell-mediated vasculature targeting approach offers an attractive alternative to overcome the limitations associated with classical antiangiogenic therapy. A further advantage of this approach as compared to direct targeting of tumor cells is a decreased likelihood of development of resistance by genetically more stable endothelial cells as compared to tumor cells. Further, the vascular-disruptive activity of the T cell bispecific antibodies disclosed herein is achieved by engaging a large number of circulating effector T cells. This vascular-disruptive activity does not require and is not limited by T cell extravasation. Next, the T cell bispecific antibodies provide constant access to fresh circulating T cells, which are not exposed to tumor immunosuppressive environment, thereby preserving higher cytotoxic activity. In addition, through the T cell bispecific antibodies, a robust cytotoxic effect rather than a cytostatic effect is achieved as long as the vascular target remains expressed. Bispecific T cell activating antigen binding molecules targeting the vasculature could also be valuable in combination therapies.

Several bispecific antibody formats have been developed and their suitability for T cell mediated immunotherapy investigated. Out of these, the so-called BiTE (bispecific T cell engager) molecules have been very well characterized and already shown some promise in the clinic (reviewed in Nagorsen and Bauerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). A more recent development are the so-called DART (dual affinity retargeting) molecules, which are based on the diabody format but feature a C-terminal disulfide bridge for additional stabilization (Moore et al., Blood 117, 4542-51 (2011)). The so-called triomabs, which are whole hybrid mouse/rat IgG molecules and also currently being evaluated in clinical trials, represent a larger sized format (reviewed in Seimetz et al., Cancer Treat Rev 36, 458-467 (2010)).

The variety of formats that are being developed shows the great potential attributed to T cell re-direction and activation in immunotherapy. The task of generating bispecific antibodies suitable therefor is, however, by no means trivial, but involves a number of challenges that have to be met related to efficacy, toxicity, applicability and produceability of the antibodies.

Small constructs such as, for example, BiTE molecules—while being able to efficiently crosslink effector and target cells—have a very short serum half life requiring them to be administered to patients by continuous infusion. IgG-like formats on the other hand—while having the great benefit of a long half life—suffer from toxicity associated with the native effector functions inherent to IgG molecules. Their immunogenic potential constitutes another unfavorable feature of IgG-like bispecific antibodies, especially non-human formats, for successful therapeutic development. Finally, a major challenge in the general development of bispecific antibodies has been the production of bispecific antibody constructs at a clinically sufficient quantity and purity, due to the mispairing of antibody heavy and light chains of different specificities upon co-expression, which decreases the yield of the correctly assembled construct and results in a number of non-functional side products from which the desired bispecific antibody may be difficult to separate.

Different approaches have been taken to overcome the chain association issue in bispecific antibodies (see e.g. Klein et al., mAbs 6, 653-663 (2012)). For example, the ‘knobs-into-holes’ strategy aims at forcing the pairing of two different antibody heavy chains by introducing mutations into the CH3 domains to modify the contact interface. On one chain bulky amino acids are replaced by amino acids with short side chains to create a ‘hole’. Conversely, amino acids with large side chains are introduced into the other CH3 domain, to create a ‘knob’. By coexpressing these two heavy chains (and two identical light chains, which have to be appropriate for both heavy chains), high yields of heterodimer (‘knob-hole’) versus homodimer (‘hole-hole’ or ‘knob-knob’) are observed (Ridgway, J. B., et al., Protein Eng. 9 (1996) 617-621; and WO 96/027011). The percentage of heterodimer could be further increased by remodeling the interaction surfaces of the two CH3 domains using a phage display approach and the introduction of a disulfide bridge to stabilize the heterodimers (Merchant, A. M., et al., Nature Biotech. 16 (1998) 677-681; Atwell, S., et al., J. Mol. Biol. 270 (1997) 26-35). New approaches for the knobs-into-holes technology are described in e.g. in EP 1870459 A1.

The ‘knobs-into-holes’ strategy does, however, not solve the problem of heavy chain-light chain mispairing, which occurs in bispecific antibodies comprising different light chains for binding to the different target antigens.

A strategy to prevent heavy chain-light chain mispairing is to exchange domains between the heavy and light chains of one of the binding arms of a bispecific antibody (see WO 2009/080251, WO 2009/080252, WO 2009/080253, WO 2009/080254 and Schaefer, W. et al, PNAS, 108 (2011) 11187-11191, which relate to bispecific IgG antibodies with a domain crossover).

Exchanging the heavy and light chain variable domains VH and VL in one of the binding arms of the bispecific antibody (WO2009/080252, see also Schaefer, W. et al, PNAS, 108 (2011) 11187-11191) clearly reduces the side products caused by the mispairing of a light chain against a first antigen with the wrong heavy chain against the second antigen (compared to approaches without such domain exchange). Nevertheless, these antibody preparations are not completely free of side products. The main side product is based on a Bence Jones-type interaction (Schaefer, W. et al, PNAS, 108 (2011) 11187-11191; in Fig. S1I of the Supplement). A further reduction of such side products is thus desirable to improve e.g. the yield of such bispecific antibodies.

The present invention provides novel bispecific antigen binding molecules designed for T cell activation and re-direction, targeting Robo 4 and an activating T cell antigen such as CD3, that combine good efficacy and produceability with low toxicity and favorable pharmacokinetic properties.

### SUMMARY OF THE INVENTION

In a first aspect the present invention provides a T cell activating bispecific antigen binding molecule comprising

(a) a first antigen binding moiety which specifically binds to a first antigen;
(b) a second antigen binding moiety which specifically binds to a second antigen;
wherein the first antigen is an activating T cell antigen and the second antigen is Robo 4, or the first antigen is Robo 4 and the second antigen is an activating T cell antigen.

In particular embodiments, the first and/or the second antigen binding moiety is a Fab molecule. In a particular embodiment, the second antigen binding moiety is a Fab molecule which specifically binds to a second antigen, and wherein the variable domains VL and VH or the constant domains CL and CH1 of the Fab light chain and the Fab heavy chain are replaced by each other (i.e. according to such embodiment, the second Fab molecule is a crossover Fab molecule wherein the variable or constant domains of the Fab light chain and the Fab heavy chain are exchanged).

In particular embodiments, the first (and the third, if any) Fab molecule is a conventional Fab molecule. In a further particular embodiment, not more than one Fab molecule capable of specific binding to an activating T cell antigen is present in the T cell activating bispecific antigen binding molecule (i.e. the T cell activating bispecific antigen binding molecule provides monovalent binding to the activating T cell antigen).

In one embodiment, the first antigen is Robo 4 and the second antigen is an activating T cell antigen. In some embodiments, the activating T cell antigen is CD3, particularly CD3 epsilon. In a particular embodiment, the T cell activating bispecific antigen binding molecule of the invention comprises

(a) a first Fab molecule which specifically binds to a first antigen;
(b) a second Fab molecule which specifically binds a second antigen, and wherein the variable domains VL and VH or the constant domains CL and CH1 of the Fab light chain and the Fab heavy chain are replaced by each other;
wherein the first antigen is Robo 4 and the second antigen is an activating T cell antigen. According to a further aspect of the invention, the ratio of a desired bispecific antibody compared to undesired side products, in particular Bence Jones-type side products occurring in bispecific antibodies with a VH/VL domain exchange in one of their binding arms, can be improved by the introduction of charged amino acids with opposite charges at specific amino acid positions in the CH1 and CL domains (sometimes referred to herein as “charge modifications”).

Thus, in some embodiments the first antigen binding moiety under (a) is a first Fab molecule which specifically binds to a first antigen, the second antigen binding moiety under (b) is a second Fab molecule which specifically binds to a second antigen wherein the variable domains VL and VH of the Fab light chain and the Fab heavy chain are replaced by each other; and
i) in the constant domain CL of the first Fab molecule under a) the amino acid at position 124 is substituted independently by lysine (K), arginine (R) or histidine (H) (numbering according to Kabat), and wherein in the constant domain CH1 of the first Fab molecule under a) the amino acid at position 147 or the amino acid at position 213 is substituted independently by glutamic acid (E), or aspartic acid (D) (numbering according to Kabat EU index); or
ii) in the constant domain CL of the second Fab molecule under b) the amino acid at position 124 is substituted independently by lysine (K), arginine (R) or histidine (H) (numbering according to Kabat), and wherein in the constant domain CH1 of the second Fab molecule under b) the amino acid at position 147 or the amino acid at position 213 is substituted independently by glutamic acid (E), or aspartic acid (D) (numbering according to Kabat EU index).

In one such embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In yet another embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a particular embodiment, in the constant domain CL of the first Fab molecule under a) the amino acid at position 124 is substituted by lysine (K) (numbering according to Kabat) and the amino acid at position 123 is substituted by lysine (K) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In another particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an alternative embodiment, in the constant domain CL of the second Fab molecule under b) the amino acid at position 124 is substituted independently by lysine (K), arginine (R) or histidine (H) (numbering according to Kabat) (in one preferred embodiment independently by lysine (K) or arginine (R)), and in the constant domain CH1 of the second Fab molecule under b) the amino acid at position 147 or the amino acid at position 213 is substituted independently by glutamic acid (E), or aspartic acid (D) (numbering according to Kabat EU index).

In a further embodiment, in the constant domain CL of the second Fab molecule under b) the amino acid at position 124 is substituted independently by lysine (K), arginine (R) or histidine (H) (numbering according to Kabat), and in the constant domain CH1 of the second Fab molecule under b) the amino acid at position 147 is substituted independently by glutamic acid (E), or aspartic acid (D) (numbering according to Kabat EU index).

In still another embodiment, in the constant domain CL of the second Fab molecule under b) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the second Fab molecule under b) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one embodiment, in the constant domain CL of the second Fab molecule under b) the amino acid at position 124 is substituted by lysine (K) (numbering according to Kabat) and the amino acid at position 123 is substituted by lysine (K) (numbering according to Kabat), and in the constant domain CH1 of the second Fab molecule under b) the amino acid at position 147 is substituted by glutamic acid (E) (numbering according to Kabat EU index) and the amino acid at position 213 is substituted by glutamic acid (E) (numbering according to Kabat EU index).

In another embodiment, in the constant domain CL of the second Fab molecule under b) the amino acid at position 124 is substituted by lysine (K) (numbering according to Kabat) and the amino acid at position 123 is substituted by arginine (R) (numbering according to Kabat), and in the constant domain CH1 of the second Fab molecule under b) the amino acid at position 147 is substituted by glutamic acid (E) (numbering according to Kabat EU index) and the amino acid at position 213 is substituted by glutamic acid (E) (numbering according to Kabat EU index).

In some embodiments, the antigen binding moiety, particularly Fab molecule, which specifically binds to Robo 4 specifically binds to an epitope in the Ig-like domain 1 (position 20-119 of SEQ ID NO: 15) and/or the Ig-like domain 2 (position 20-107 of SEQ ID NO: 17) of the extracellular domain of Robo 4.

In a specific embodiment, the antigen binding moiety, particularly Fab molecule, which specifically binds to Robo 4 comprises a heavy chain variable region comprising the heavy chain complementarity determining region (HCDR) 1 of SEQ ID NO: 91, the HCDR 2 of SEQ ID NO: 92 and the HCDR 3 of SEQ ID NO: 93, and a light chain variable region comprising the light chain complementarity determining region (LCDR) 1 of SEQ ID NO: 94, the LCDR 2 of SEQ ID NO: 95 and the LCDR 3 of SEQ ID NO: 96. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 19 and a light chain variable region comprising an amino acid sequence that is at least about 95%, 96%, 97%, 98%, 99% or 100% identical to the amino acid sequence of SEQ ID NO: 21.

In another specific embodiment, the antigen binding moiety, particularly Fab molecule, which specifically binds to Robo 4 comprises a heavy chain variable region comprising the heavy chain complementarity determining region (HCDR) 1 of SEQ ID NO: 103, the HCDR 2 of SEQ ID NO: 104 and the HCDR 3 of SEQ ID NO: 105, and a light chain variable region comprising the light chain complementarity determining region (LCDR) 1 of SEQ ID NO: 106, the LCDR 2 of SEQ ID NO: 107 and the LCDR 3 of SEQ ID NO: 108. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 29.

In yet another specific embodiment, the antigen binding moiety, particularly Fab molecule, which specifically binds to Robo 4 comprises a heavy chain variable region comprising the heavy chain complementarity determining region (HCDR) 1 of SEQ ID NO: 109, the HCDR 2 of SEQ ID NO: 110 and the HCDR 3 of SEQ ID NO: 111, and a light chain variable region comprising the light chain complementarity determining region (LCDR) 1 of SEQ ID NO: 112, the LCDR 2 of SEQ ID NO: 113 and the LCDR 3 of SEQ ID NO: 114. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 31 and a light chain variable region comprising an amino acid sequence that is at least about 95%, 96%, 97%, 98%, 99% or 100% identical to the amino acid sequence of SEQ ID NO: 33. In other embodiments, the antigen binding moiety, particularly Fab molecule, which specifically binds to Robo 4 specifically binds to an epitope in the fibronectin-like domain 1 (position 20-108 of SEQ ID NO: 11) and/or the fibronectin-like domain 2 (position 20-111 of SEQ ID NO: 11) of the extracellular domain of Robo 4.

In a specific embodiment, the antigen binding moiety, particularly Fab molecule, which specifically binds to Robo 4 comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 23 and a light chain variable region comprising an amino acid sequence that is at least about 95%, 96%, 97%, 98%, 99% or 100% identical to the amino acid sequence of SEQ ID NO: 25.

In a particular embodiment, the T cell activating bispecific antigen binding molecule of the invention comprises

(a) a first Fab molecule which specifically binds to a first antigen;
(b) a second Fab molecule which specifically binds to a second antigen, and wherein the variable domains VL and VH of the Fab light chain and the Fab heavy chain are replaced by each other;
wherein the first antigen is Robo 4 and the second antigen is an activating T cell antigen;

wherein the first Fab molecule under (a) comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102; and

wherein in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In some embodiments, the T cell activating bispecific antigen binding molecule according to the invention further comprises a third antigen binding moiety which specifically binds to the first antigen. In particular embodiments, the third antigen binding moiety is identical to the first antigen binding moiety. In one embodiment, the third antigen binding moiety is a Fab molecule.

In particular embodiments, the third and the first antigen binding moiety are each a Fab molecule and the third Fab molecule is identical to the first Fab molecule. In these embodiments, the third Fab molecule thus comprises the same amino acid substitutions, if any, as the first Fab molecule. Like the first Fab molecule, the third Fab molecule particularly is a conventional Fab molecule.

If a third antigen binding moiety is present, in a particular embodiment the first and the third antigen moiety specifically bind to Robo 4, and the second antigen binding moiety specifically binds to an activating T cell antigen, particularly CD3, more particularly CD3 epsilon.

In some embodiments of the T cell activating bispecific antigen binding molecule according to the invention the first antigen binding moiety under a) and the second antigen binding moiety under b) are fused to each other, optionally via a peptide linker. In particular embodiments, the first and the second antigen binding moiety are each a Fab molecule. In a specific such embodiment, the second Fab molecule is fused at the C-terminus of the Fab heavy chain to the N-terminus of the Fab heavy chain of the first Fab molecule. In an alternative such embodiment, the first Fab molecule is fused at the C-terminus of the Fab heavy chain to the N-terminus of the Fab heavy chain of the second Fab molecule. In embodiments wherein either (i) the second Fab molecule is fused at the C-terminus of the Fab heavy chain to the N-terminus of the Fab heavy chain of the first Fab molecule or (ii) the first Fab molecule is fused at the C-terminus of the Fab heavy chain to the N-terminus of the Fab heavy chain of the second Fab molecule, additionally the Fab light chain of the Fab molecule and the Fab light chain of the second Fab molecule may be fused to each other, optionally via a peptide linker.

In particular embodiments, the T cell activating bispecific antigen binding molecule according to the invention additionally comprises an Fc domain composed of a first and a second subunit capable of stable association.

The T cell activating bispecific antigen binding molecule according to the invention can have different configurations, i.e. the first, second (and optionally third) antigen binding moiety may be fused to each other and to the Fc domain in different ways. The components may be fused to each other directly or, preferably, via one or more suitable peptide linkers. Where fusion of a Fab molecule is to the N-terminus of a subunit of the Fc domain, it is typically via an immunoglobulin hinge region.

In one embodiment, the first and the second antigen binding moiety are each a Fab molecule and the second antigen binding moiety is fused at the C-terminus of the Fab heavy chain to the N-terminus of the first or the second subunit of the Fc domain. In such embodiment, the first antigen binding moiety may be fused at the C-terminus of the Fab heavy chain to the N-terminus of the Fab heavy chain of the second antigen binding moiety or to the N-terminus of the other one of the subunits of the Fc domain.

In one embodiment, the first and the second antigen binding moiety are each a Fab molecule and the first and the second antigen binding moiety are each fused at the C-terminus of the Fab heavy chain to the N-terminus of one of the subunits of the Fc domain. In this embodiment, the T cell activating bispecific antigen binding molecule essentially comprises an immunoglobulin molecule, wherein in one of the Fab arms the heavy and light chain variable regions VH and VL (or the constant regions CH1 and CL in embodiments wherein no charge modifications as described herein are introduced in CH1 and CL domains) are exchanged/replaced by each other (see FIG. 29A, D).

In alternative embodiments, a third antigen binding moiety, particularly a third Fab molecule, is fused at the C-terminus of the Fab heavy chain to the N-terminus of the first or second subunit of the Fc domain. In a particular such embodiment, the second and the third antigen binding moiety are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second Fab molecule. In this embodiment, the T cell activating bispecific antigen binding molecule essentially comprises an immunoglobulin molecule, wherein in one of the Fab arms the heavy and light chain variable regions VH and VL (or the constant regions CH1 and CL in embodiments wherein no charge modifications as described herein are introduced in CH1 and CL domains) are exchanged/replaced by each other, and wherein an additional (conventional) Fab molecule is N-terminally fused to said Fab arm (see FIG. 29B, E). In another such embodiment, the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. In this embodiment, the T cell activating bispecific antigen binding molecule essentially comprises an immunoglobulin molecule with an additional Fab molecule N-terminally fused to one of the immunoglobulin Fab arms, wherein in said additional Fab molecule the heavy and light chain variable regions VH and VL (or the constant regions CH1 and CL in embodiments wherein no charge modifications as described herein are introduced in CH1 and CL domains) are exchanged/replaced by each other (see FIG. 29C, F).

In a particular embodiment, the immunoglobulin molecule comprised in the T cell activating bispecific antigen binding molecule according to the invention is an IgG class immunoglobulin.

In an even more particular embodiment the immunoglobulin is an IgG₁ subclass immunoglobulin. In another embodiment, the immunoglobulin is an IgG₄ subclass immunoglobulin.

In a particular embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH or the constant domains CL and CH1 of the Fab light chain and the Fab heavy chain are replaced by each other;
c) a third Fab molecule which specifically binds to the first antigen; and
d) an Fc domain composed of a first and a second subunit capable of stable association;
wherein the first antigen is Robo 4 and the second antigen is an activating T cell antigen, particularly CD3, more particularly CD3 epsilon;
wherein the third Fab molecule under c) is identical to the first Fab molecule under a);
wherein
(i) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d), or
(ii) the second Fab molecule under b) is fused at the C-terminus of the Fab heavy chain to the N-terminus of the Fab heavy chain of the first Fab molecule under a), and the first Fab molecule under a) and the third Fab molecule under c) are each fused at the C-terminus of the Fab heavy chain to the N-terminus of one of the subunits of the Fc domain under d); and
wherein the first Fab molecule under a) and the third Fab molecule under c) comprise a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102.

In another embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH or the constant domains CL and CH1 of the Fab light chain and the Fab heavy chain are replaced by each other;
c) an Fc domain composed of a first and a second subunit capable of stable association; wherein the first antigen is Robo 4 and the second antigen is an activating T cell antigen, particularly CD3, more particularly CD3 epsilon;
wherein
(i) the first Fab molecule under a) is fused at the C-terminus of the Fab heavy chain to the N-terminus of the Fab heavy chain of the second Fab molecule under b), and the second Fab molecule under b) is fused at the C-terminus of the Fab heavy chain to the N-terminus of one of the subunits of the Fc domain under c), or
(ii) the second Fab molecule under b) is fused at the C-terminus of the Fab heavy chain to the N-terminus of the Fab heavy chain of the first Fab molecule under a), and the first Fab molecule under a) is fused at the C-terminus of the Fab heavy chain to the N-terminus of one of the subunits of the Fc domain under c); and
wherein the first Fab molecule under a) comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102.

In a further embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH or the constant domains CL and CH1 of the Fab light chain and the Fab heavy chain are replaced by each other; and
c) an Fc domain composed of a first and a second subunit capable of stable association; wherein
(i) the first antigen is Robo 4 and the second antigen is an activating T cell antigen, particularly CD3, more particularly CD3 epsilon; or
(ii) the second antigen is Robo 4 and the first antigen is an activating T cell antigen, particularly CD3, more particularly CD3 epsilon;
wherein the first Fab molecule under a) and the second Fab molecule under b) are each fused at the C-terminus of the Fab heavy chain to the N-terminus of one of the subunits of the Fc domain under c); and
wherein the Fab molecule which specifically binds to Robo 4 comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region, particularly a humanized light chain variable region, comprising the light chain complementarity determining region (LCDR) 1 of SEQ ID NO: 100, the LCDR 2 of SEQ ID NO: 101 and the LCDR 3 of SEQ ID NO: 102.

In all of the different configurations of the T cell activating bispecific antigen binding molecule according to the invention, the amino acid substitutions described herein, if present, may either be in the CH1 and CL domains of the first and (if present) the third Fab molecule, or in the CH1 and CL domains of the second Fab molecule. Preferably, they are in the CH1 and CL domains of the first and (if present) the third Fab molecule. In accordance with the concept of the invention, if amino acid substitutions as described herein are made in the first (and, if present, the third) Fab molecule, no such amino acid substitutions are made in the second Fab molecule. Conversely, if amino acid substitutions as described herein are made in the second Fab molecule, no such amino acid substitutions are made in the first (and, if present, the third) Fab molecule. No amino acid substitutions are made in T cell activating bispecific antigen binding molecules comprising a Fab molecule wherein the constant domains CL and CH1 of the Fab light chain and the Fab heavy chain are replaced by each other.

In particular embodiments of the T cell activating bispecific antigen binding molecule according to the invention, particularly wherein amino acid substitutions as described herein are made in the first (and, if present, the third) Fab molecule, the constant domain CL of the first (and, if present, the third) Fab molecule is of kappa isotype. In other embodiments of the T cell activating bispecific antigen binding molecule according to the invention, particularly wherein amino acid substitutions as described herein are made in the second Fab molecule, the constant domain CL of the second Fab molecule is of kappa isotype. In some embodiments, the constant domain CL of the first (and, if present, the third) Fab molecule and the constant domain CL of the second Fab molecule are of kappa isotype.

In a particular embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH of the Fab light chain and the Fab heavy chain are replaced by each other;
c) a third Fab molecule which specifically binds to the first antigen; and
d) an Fc domain composed of a first and a second subunit capable of stable association; wherein the first antigen is Robo 4 and the second antigen is an activating T cell antigen, particularly CD3, more particularly CD3 epsilon;
wherein the third Fab molecule under c) is identical to the first Fab molecule under a); wherein in the constant domain CL of the first Fab molecule under a) and the third Fab molecule under c) the amino acid at position 124 is substituted by lysine (K) (numbering according to Kabat) and the amino acid at position 123 is substituted by lysine (K) or arginine (R) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index);
wherein
(i) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d), or
(ii) the second Fab molecule under b) is fused at the C-terminus of the Fab heavy chain to the N-terminus of the Fab heavy chain of the first Fab molecule under a), and the first Fab molecule under a) and the third Fab molecule under c) are each fused at the C-terminus of the Fab heavy chain to the N-terminus of one of the subunits of the Fc domain under d); and
wherein the first Fab molecule under a) and the third Fab molecule under c) comprise a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region, particularly a humanized light chain variable region, comprising the light chain complementarity determining region (LCDR) 1 of SEQ ID NO: 100, the LCDR 2 of SEQ ID NO: 101 and the LCDR 3 of SEQ ID NO: 102.

In an even more particular embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH of the Fab light chain and the Fab heavy chain are replaced by each other;
c) a third Fab molecule which specifically binds to the first antigen; and
d) an Fc domain composed of a first and a second subunit capable of stable association; wherein the first antigen is Robo 4 and the second antigen is an activating T cell antigen, particularly CD3, more particularly CD3 epsilon;
wherein the third Fab molecule under c) is identical to the first Fab molecule under a);
wherein in the constant domain CL of the first Fab molecule under a) and the third Fab molecule under c) the amino acid at position 124 is substituted by lysine (K) (numbering according to Kabat) and the amino acid at position 123 is substituted by lysine (K) (numbering according to Kabat), and wherein in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index);
wherein the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d); and wherein the first Fab molecule under a) and the third Fab molecule under c) comprise a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102.

In another embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH of the Fab light chain and the Fab heavy chain are replaced by each other;
c) an Fc domain composed of a first and a second subunit capable of stable association; wherein the first antigen is Robo 4 and the second antigen is an activating T cell antigen, particularly CD3, more particularly CD3 epsilon;
wherein in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index); wherein
(i) the first Fab molecule under a) is fused at the C-terminus of the Fab heavy chain to the N-terminus of the Fab heavy chain of the second Fab molecule under b), and the second Fab molecule under b) is fused at the C-terminus of the Fab heavy chain to the N-terminus of one of the subunits of the Fc domain under c), or
(ii) the second Fab molecule under b) is fused at the C-terminus of the Fab heavy chain to the N-terminus of the Fab heavy chain of the first Fab molecule under a), and the first Fab molecule under a) is fused at the C-terminus of the Fab heavy chain to the N-terminus of one of the subunits of the Fc domain under c); and
wherein the first Fab molecule under a) comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102.

In a further embodiment, the invention provides a T cell activating bispecific antigen binding molecule comprising

a) a first Fab molecule which specifically binds to a first antigen;
b) a second Fab molecule which specifically binds to a second antigen, and wherein the variable domains VL and VH of the Fab light chain and the Fab heavy chain are replaced by each other; and
c) an Fc domain composed of a first and a second subunit capable of stable association;
wherein
(i) the first antigen is Robo 4 and the second antigen is an activating T cell antigen, particularly CD3, more particularly CD3 epsilon; or
(ii) the second antigen is Robo 4 and the first antigen is an activating T cell antigen, particularly CD3, more particularly CD3 epsilon;
wherein in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index); wherein the first Fab molecule under a) and the second Fab molecule under b) are each fused at the C-terminus of the Fab heavy chain to the N-terminus of one of the subunits of the Fc domain under c); and
wherein the Fab molecule which specifically binds to Robo 4 comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102.

In particular embodiments of the T cell activating bispecific antigen binding molecule, the Fc domain is an IgG Fc domain. In a specific embodiment, the Fc domain is an IgG₁ Fc domain. In another specific embodiment, the Fc domain is an IgG₄ Fc domain. In an even more specific embodiment, the Fc domain is an IgG₄ Fc domain comprising the amino acid substitution S228P (Kabat numbering). In particular embodiments the Fc domain is a human Fc domain.

In particular embodiments, the Fc domain comprises a modification promoting the association of the first and the second Fc domain subunit. In a specific such embodiment, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable.

In a particular embodiment the Fc domain exhibits reduced binding affinity to an Fc receptor and/or reduced effector function, as compared to a native IgG₁ Fc domain. In certain embodiments the Fc domain is engineered to have reduced binding affinity to an Fc receptor and/or reduced effector function, as compared to a non-engineered Fc domain. In one embodiment, the Fc domain comprises one or more amino acid substitution that reduces binding to an Fc receptor and/or effector function. In one embodiment, the one or more amino acid substitution in the Fc domain that reduces binding to an Fc receptor and/or effector function is at one or more position selected from the group of L234, L235, and P329 (Kabat EU index numbering). In particular embodiments, each subunit of the Fc domain comprises three amino acid substitutions that reduce binding to an Fc receptor and/or effector function wherein said amino acid substitutions are L234A, L235A and P329G (Kabat EU index numbering). In one such embodiment, the Fc domain is an IgG₁ Fc domain, particularly a human IgG₁ Fc domain. In other embodiments, each subunit of the Fc domain comprises two amino acid substitutions that reduce binding to an Fc receptor and/or effector function wherein said amino acid substitutions are L235E and P329G (Kabat EU index numbering). In one such embodiment, the Fc domain is an IgG₄ Fc domain, particularly a human IgG₄ Fc domain. In one embodiment, the Fc domain of the T cell activating bispecific antigen binding molecule is an IgG₄ Fc domain and comprises the amino acid substitutions L235E and S228P (SPLE) (Kabat EU index numbering).

In one embodiment the Fc receptor is an Fcγ receptor. In one embodiment the Fc receptor is a human Fc receptor. In one embodiment, the Fc receptor is an activating Fc receptor. In a specific embodiment, the Fc receptor is human FcγRIIa, FcγRI, and/or FcγRIIIa. In one embodiment, the effector function is antibody-dependent cell-mediated cytotoxicity (ADCC).

In a specific embodiment of the T cell activating bispecific antigen binding molecule according to the invention, the antigen binding moiety which specifically binds to an activating T cell antigen, particularly CD3, more particularly CD3 epsilon, comprises a heavy chain variable region comprising the heavy chain complementarity determining region (HCDR) 1 of SEQ ID NO: 141, the HCDR 2 of SEQ ID NO: 142, the HCDR 3 of SEQ ID NO: 143, and a light chain variable region comprising the light chain complementarity determining region (LCDR) 1 of SEQ ID NO: 145, the LCDR 2 of SEQ ID NO: 146 and the LCDR 3 of SEQ ID NO: 147. In an even more specific embodiment, the antigen binding moiety which specifically binds to an activating T cell antigen, particularly CD3, more particularly CD3 epsilon, comprises a heavy chain variable region comprising an amino acid sequence that is at least about 95%, 96%, 97%, 98%, 99% or 100% identical to the amino acid sequence of SEQ ID NO: 140 and a light chain variable region comprising an amino acid sequence that is at least about 95%, 96%, 97%, 98%, 99% or 100% identical to the amino acid sequence of SEQ ID NO: 144. In some embodiments, the antigen binding moiety which specifically binds to an activating T cell antigen is a Fab molecule. In one specific embodiment, the second antigen binding moiety, particularly Fab molecule, comprised in the T cell activating bispecific antigen binding molecule according to the invention specifically binds to CD3, more particularly CD3 epsilon, and comprises the heavy chain complementarity determining region (CDR) 1 of SEQ ID NO: 141, the heavy chain CDR 2 of SEQ ID NO: 142, the heavy chain CDR 3 of SEQ ID NO: 143, the light chain CDR 1 of SEQ ID NO: 145, the light chain CDR 2 of SEQ ID NO: 146 and the light chain CDR 3 of SEQ ID NO: 147. In an even more specific embodiment, said second antigen binding moiety, particularly Fab molecule, comprises a heavy chain variable region comprising the amino acid sequence of SEQ ID NO: 140 and a light chain variable region comprising the amino acid sequence of SEQ ID NO: 144.

In a further specific embodiment of the T cell activating bispecific antigen binding molecule according to the invention, the antigen binding moiety, particularly Fab molecule, which specifically binds to Robo 4 comprises the heavy chain complementarity determining region (CDR) 1 of SEQ ID NO: 97, the heavy chain CDR 2 of SEQ ID NO: 98, the heavy chain CDR 3 of SEQ ID NO: 99, the light chain CDR 1 of SEQ ID NO: 100, the light chain CDR 2 of SEQ ID NO: 101 and the light chain CDR 3 of SEQ ID NO: 102. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 23 and a light chain variable region comprising an amino acid sequence that is at least about 95%, 96%, 97%, 98%, 99% or 100% identical to the amino acid sequence of SEQ ID NO: 25. In one specific embodiment, the first (and, if present, the third) antigen binding moiety, particularly Fab molecule, comprised in the T cell activating bispecific antigen binding molecule according to the invention specifically binds to Robo 4, and comprises the heavy chain complementarity determining region (CDR) 1 of SEQ ID NO: 97, the heavy chain CDR 2 of SEQ ID NO: 98, the heavy chain CDR 3 of SEQ ID NO: 99, the light chain CDR 1 of SEQ ID NO: 100, the light chain CDR 2 of SEQ ID NO: 101 and the light chain CDR 3 of SEQ ID NO: 102. In an even more specific embodiment, said first (and, if present, said third) antigen binding moiety, particularly Fab molecule, comprises a heavy chain variable region comprising the amino acid sequence of SEQ ID NO: 23 and a light chain variable region comprising the amino acid sequence of SEQ ID NO: 25.

In a further specific embodiment of the T cell activating bispecific antigen binding molecule according to the invention, the antigen binding moiety, particularly Fab molecule, which specifically binds to Robo 4 comprises the heavy chain complementarity determining region (CDR) 1 of SEQ ID NO: 91, the heavy chain CDR 2 of SEQ ID NO: 92, the heavy chain CDR 3 of SEQ ID NO: 93, the light chain CDR 1 of SEQ ID NO: 94, the light chain CDR 2 of SEQ ID NO: 95 and the light chain CDR 3 of SEQ ID NO: 96. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 19 and a light chain variable region comprising an amino acid sequence that is at least about 95%, 96%, 97%, 98%, 99% or 100% identical to the amino acid sequence of SEQ ID NO: 21. In one specific embodiment, the first (and, if present, the third) antigen binding moiety, particularly Fab molecule, comprised in the T cell activating bispecific antigen binding molecule according to the invention specifically binds to Robo 4, and comprises the heavy chain complementarity determining region (CDR) 1 of SEQ ID NO: 91, the heavy chain CDR 2 of SEQ ID NO: 92, the heavy chain CDR 3 of SEQ ID NO: 93, the light chain CDR 1 of SEQ ID NO: 94, the light chain CDR 2 of SEQ ID NO: 95 and the light chain CDR 3 of SEQ ID NO: 96. In an even more specific embodiment, said first (and, if present, said third) antigen binding moiety, particularly Fab molecule, comprises a heavy chain variable region comprising the amino acid sequence of SEQ ID NO: 19 and a light chain variable region comprising the amino acid sequence of SEQ ID NO: 21.

In a further specific embodiment of the T cell activating bispecific antigen binding molecule according to the invention, the antigen binding moiety, particularly Fab molecule, which specifically binds to Robo 4 comprises the heavy chain complementarity determining region (CDR) 1 of SEQ ID NO: 103, the heavy chain CDR 2 of SEQ ID NO: 104, the heavy chain CDR 3 of SEQ ID NO: 105, the light chain CDR 1 of SEQ ID NO: 106, the light chain CDR 2 of SEQ ID NO: 107 and the light chain CDR 3 of SEQ ID NO: 108. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 29. In one specific embodiment, the first (and, if present, the third) antigen binding moiety, particularly Fab molecule, comprised in the T cell activating bispecific antigen binding molecule according to the invention specifically binds to Robo 4, and comprises the heavy chain complementarity determining region (CDR) 1 of SEQ ID NO: 103, the heavy chain CDR 2 of SEQ ID NO: 104, the heavy chain CDR 3 of SEQ ID NO: 105, the light chain CDR 1 of SEQ ID NO: 106, the light chain CDR 2 of SEQ ID NO: 107 and the light chain CDR 3 of SEQ ID NO: 108. In an even more specific embodiment, said first (and, if present, said third) antigen binding moiety, particularly Fab molecule, comprises a heavy chain variable region comprising the amino acid sequence of SEQ ID NO: 27 and a light chain variable region comprising the amino acid sequence of SEQ ID NO: 29.

In a further specific embodiment of the T cell activating bispecific antigen binding molecule according to the invention, the antigen binding moiety, particularly Fab molecule, which specifically binds to Robo 4 comprises the heavy chain complementarity determining region (CDR) 1 of SEQ ID NO: 109, the heavy chain CDR 2 of SEQ ID NO: 110, the heavy chain CDR 3 of SEQ ID NO: 111, the light chain CDR 1 of SEQ ID NO: 112, the light chain CDR 2 of SEQ ID NO: 113 and the light chain CDR 3 of SEQ ID NO: 114. In an even more specific embodiment, the antigen binding moiety, particularly Fab molecule, which specifically binds to Robo 4 comprises a heavy chain variable region comprising an amino acid sequence that is at least about 95%, 96%, 97%, 98%, 99% or 100% identical to the amino acid sequence of SEQ ID NO: 31 and a light chain variable region comprising an amino acid sequence that is at least about 95%, 96%, 97%, 98%, 99% or 100% identical to the amino acid sequence of SEQ ID NO: 33. In one specific embodiment, the first (and, if present, the third) antigen binding moiety, particularly Fab molecule, comprised in the T cell activating bispecific antigen binding molecule according to the invention specifically binds to Robo 4, and comprises the heavy chain complementarity determining region (CDR) 1 of SEQ ID NO: 109, the heavy chain CDR 2 of SEQ ID NO: 110, the heavy chain CDR 3 of SEQ ID NO: 111, the light chain CDR 1 of SEQ ID NO: 112, the light chain CDR 2 of SEQ ID NO: 113 and the light chain CDR 3 of SEQ ID NO: 114. In an even more specific embodiment, said first (and, if present, said third) antigen binding moiety, particularly Fab molecule, comprises a heavy chain variable region comprising the amino acid sequence of SEQ ID NO: 31 and a light chain variable region comprising the amino acid sequence of SEQ ID NO: 33.

In a particular aspect, the invention provides a T cell activating bispecific antigen binding molecule comprising

a) a first Fab molecule which specifically binds to a first antigen;
b) a second Fab molecule which specifically binds to a second antigen, and wherein the variable domains VL and VH or the constant domains CL and CH1 of the Fab light chain and the Fab heavy chain are replaced by each other;
c) a third Fab molecule which specifically binds to the first antigen; and
d) an Fc domain composed of a first and a second subunit capable of stable association;
wherein
(i) the first antigen is Robo 4 and the second antigen is CD3, particularly CD3 epsilon;
(ii) the first Fab molecule under a) and the third Fab molecule under c) each comprise the heavy chain complementarity determining region (CDR) 1 of SEQ ID NO: 97, the heavy chain CDR 2 of SEQ ID NO: 98, the heavy chain CDR 3 of SEQ ID NO: 99, the light chain CDR 1 of SEQ ID NO: 100, the light chain CDR 2 of SEQ ID NO: 101 and the light chain CDR 3 of SEQ ID NO: 102, and the second Fab molecule under b) comprises the heavy chain CDR 1 of SEQ ID NO: 141, the heavy chain CDR 2 of SEQ ID NO: 142, the heavy chain CDR 3 of SEQ ID NO: 143, the light chain CDR 1 of SEQ ID NO: 145, the light chain CDR 2 of SEQ ID NO: 146 and the light chain CDR 3 of SEQ ID NO: 147; and
(iii) the first Fab molecule under a) is fused at the C-terminus of the Fab heavy chain to the N-terminus of the Fab heavy chain of the second Fab molecule under b), and the second Fab molecule under b) and the third Fab molecule under c) are each fused at the C-terminus of the Fab heavy chain to the N-terminus of one of the subunits of the Fc domain under d).

In one embodiment, in the second Fab molecule under b) the variable domains VL and VH are replaced by each other and further (iv) in the constant domain CL of the first Fab molecule under a) and the third Fab molecule under c) the amino acid at position 124 is substituted by lysine (K) (numbering according to Kabat) and the amino acid at position 123 is substituted by lysine (K) or arginine (R), particularly by lysine (K) (numbering according to Kabat), and in the constant domain CH1 of the first Fab molecule under a) and the third Fab molecule under c) the amino acid at position 147 is substituted by glutamic acid (E) (numbering according to Kabat EU index) and the amino acid at position 213 is substituted by glutamic acid (E) (numbering according to Kabat EU index).

According to another aspect of the invention there is provided one or more isolated polynucleotide(s) encoding a T cell activating bispecific antigen binding molecule of the invention. The invention further provides one or more expression vector(s) comprising the isolated polynucleotide(s) of the invention, and a host cell comprising the isolated polynucleotide(s) or the expression vector(s) of the invention. In some embodiments the host cell is a eukaryotic cell, particularly a mammalian cell.

In another aspect is provided a method of producing the T cell activating bispecific antigen binding molecule of the invention, comprising the steps of a) culturing the host cell of the invention under conditions suitable for the expression of the T cell activating bispecific antigen binding molecule and b) recovering the T cell activating bispecific antigen binding molecule. The invention also encompasses a T cell activating bispecific antigen binding molecule produced by the method of the invention.

The invention further provides a pharmaceutical composition comprising the T cell activating bispecific antigen binding molecule of the invention and a pharmaceutically acceptable carrier. Also encompassed by the invention are methods of using the T cell activating bispecific antigen binding molecule and pharmaceutical composition of the invention. In one aspect the invention provides a T cell activating bispecific antigen binding molecule or a pharmaceutical composition of the invention for use as a medicament. In one aspect is provided a T cell activating bispecific antigen binding molecule or a pharmaceutical composition according to the invention for use in the treatment of a disease in an individual in need thereof. In a specific embodiment the disease is cancer.

Also provided is the use of a T cell activating bispecific antigen binding molecule of the invention for the manufacture of a medicament for the treatment of a disease in an individual in need thereof; as well as a method of treating a disease in an individual, comprising administering to said individual a therapeutically effective amount of a composition comprising the T cell activating bispecific antigen binding molecule according to the invention in a pharmaceutically acceptable form. In a specific embodiment the disease is cancer. In any of the above embodiments the individual preferably is a mammal, particularly a human.

The invention also provides a method for inducing lysis of a target cell, particularly a cell expressing Robo 4, comprising contacting a target cell with a T cell activating bispecific antigen binding molecule of the invention in the presence of a T cell, particularly a cytotoxic T cell.

In a further aspect the invention provides an antibody that specifically binds to Robo 4, wherein said antibody specifically binds to an epitope in the Ig-like domain 1 (position 20-119 of SEQ ID NO: 15) and/or the Ig-like domain 2 (position 20-107 of SEQ ID NO: 17) of the extracellular domain of Robo 4.

The invention further provides an antibody that specifically binds to Robo 4, wherein said antibody comprises a heavy chain variable region comprising the heavy chain complementarity determining region (HCDR) 1 of SEQ ID NO: 91, the HCDR 2 of SEQ ID NO: 92 and the HCDR 3 of SEQ ID NO: 93, and a light chain variable region comprising the light chain complementarity determining region (LCDR) 1 of SEQ ID NO: 94, the LCDR 2 of SEQ ID NO: 95 and the LCDR 3 of SEQ ID NO: 96. In a more specific embodiment, said antibody comprises a heavy chain variable region comprising an amino acid sequence that is at least about 95%, 96%, 97%, 98%, 99% or 100% identical to the amino acid sequence of SEQ ID NO: 19 and a light chain variable region comprising an amino acid sequence that is at least about 95%, 96%, 97%, 98%, 99% or 100% identical to the amino acid sequence of SEQ ID NO: 21.

The invention further provides an antibody that specifically binds to Robo 4, wherein said antibody comprises a heavy chain variable region comprising the heavy chain complementarity determining region (HCDR) 1 of SEQ ID NO: 103, the HCDR 2 of SEQ ID NO: 104 and the HCDR 3 of SEQ ID NO: 105, and a light chain variable region comprising the light chain complementarity determining region (LCDR) 1 of SEQ ID NO: 106, the LCDR 2 of SEQ ID NO: 107 and the LCDR 3 of SEQ ID NO: 108. In a more specific embodiment, said antibody comprises a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 29.

The invention further provides an antibody that specifically binds to Robo 4, wherein said antibody comprises a heavy chain variable region comprising the heavy chain complementarity determining region (HCDR) 1 of SEQ ID NO: 109, the HCDR 2 of SEQ ID NO: 110 and the HCDR 3 of SEQ ID NO: 111, and a light chain variable region comprising the light chain complementarity determining region (LCDR) 1 of SEQ ID NO: 112, the LCDR 2 of SEQ ID NO: 113 and the LCDR 3 of SEQ ID NO: 114. In a more specific embodiment, said antibody comprises a heavy chain variable region comprising an amino acid sequence that is at least about 95%, 96%, 97%, 98%, 99% or 100% identical to the amino acid sequence of SEQ ID NO: 31 and a light chain variable region comprising an amino acid sequence that is at least about 95%, 96%, 97%, 98%, 99% or 100% identical to the amino acid sequence of SEQ ID NO: 33.

In a further aspect the invention provides an antibody that specifically binds to Robo 4, wherein said antibody specifically binds to an epitope in the fibronectin-like domain 1 (position 20-108 of SEQ ID NO: 11) and/or the fibronectin-like domain 2 (position 20-111 of SEQ ID NO: 11) of the extracellular domain of Robo 4.

The invention further provides an antibody that specifically binds to Robo 4, wherein said antibody comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102. In a more specific embodiment, said antibody comprises a heavy chain variable region comprising an amino acid sequence that is at least about 95%, 96%, 97%, 98%, 99% or 100% identical to the amino acid sequence of SEQ ID NO: 23 and a light chain variable region comprising an amino acid sequence that is at least about 95%, 96%, 97%, 98%, 99% or 100% identical to the amino acid sequence of SEQ ID NO: 25.

### BRIEF DESCRIPTION OF THE DRAWINGS

FIG. 1. Analysis of purified Robo 4 antigens. (A, B) SDS PAGE of human (A) and murine (B) Robo 4 antigens (4-12% Bis/Tris (NuPage, Invitrogen); Coomassie stained; reduced). (C, D) Analytical size exclusion chromatography of human (C) and murine (D) Robo 4 antigens (Superdex 200 10/300 GL (GE Healthcare); 2 mM MOPS pH 7.3, 150 mM NaCl, 0.02% (w/v) NaN₃; 50 μg sample injected).

FIG. 2. Robo 4 antibody titers in blood of immunized hamsters as determined by ELISA after three (A) or four (B) immunizations.

FIG. 3. SDS PAGE analysis of purified anti-Robo 4 IgGs (4-12% Bis/Tris (NuPage, Invitrogen); Coomassie stained). (A) 7G2 IgG (reduced). (B) 7G2 IgG (non-reduced). (C) 01E06 IgG (reduced). (D) 01E06 IgG (non-reduced). (E) 01F05 IgG (reduced). (F) 01F05 IgG (non-reduced). (G) 01F09 IgG (reduced). (H) 01F09 IgG (non-reduced).

FIG. 4. Analysis of purified human Robo 1 antigen. (A) SDS PAGE (4-12% Bis/Tris (NuPage, Invitrogen); Coomassie stained; reduced). (B) Analytical size exclusion chromatography (Superdex 200 10/300 GL (GE Healthcare); 2 mM MOPS pH 7.3, 150 mM NaCl, 0.02% (w/v) NaN₃; 50 μg sample injected).

FIG. 5. Analysis of purified cynomolgus Robo 4 antigen. (A, B) SDS PAGE (4-12% Bis/Tris (NuPage, Invitrogen); Coomassie stained) in the absence (A) or presence (B) of a reducing agent. (C) Analytical size exclusion chromatography (TSKgel G3000 SW XL (Tosoh); 25 mM K₂HPO₄, 125 mM NaCl, 200 mM L-arginine monohydrochloride, 0.02% (w/v) NaN₃, pH 6.7; 20 μg sample injected).

FIG. 6. SDS PAGE analysis of purified human Robo 4 domain-Fc fusion proteins (4-12% Bis/Tris (NuPage, Invitrogen); Coomassie stained). (A) FN-like domain 1-Fc (reduced). (B) FN-like domain 1-Fc (non-reduced). (C) FN-like domain 2-Fc (reduced). (D) Ig-like domain 1-Fc (reduced). (E) Ig-like domain 1-Fc (non-reduced). (F) Ig-like domain 2-Fc (reduced).

FIG. 7. Analytical size exclusion chromatography of purified human Robo 4 domain-Fc fusion proteins (TSKgel G3000 SW XL (Tosoh); 25 mM K₂HPO₄, 125 mM NaCl, 200 mM L-arginine monohydrochloride, 0.02% (w/v) NaN₃, pH 6.7; 20 μg sample injected). (A) FN-like domain 1-Fc. (B) FN-like domain 2-Fc. (C) Ig-like domain 1-Fc, (D) Ig-like domain 2-Fc.

FIG. 8. Schematic illustration of the 1+1 Crossfab-IgG (A), the 2+1 CrossFab-IgG (B), the Fab-CrossFab (C) and the Fab-Fab-CrossFab (D) molecules.

FIG. 9. SDS PAGE analysis of purified anti-Robo 4/anti-CD3 1+1 CrossFab-IgG constructs (4-12% Bis/Tris (NuPage, Invitrogen); Coomassie stained). (A) Molecule A (01F09/V9), reduced. (B) Molecule A (01F09/V9), non-reduced. (C) Molecule B (01F05/V9), reduced. (D) Molecule B (01F05/V9), non-reduced. (E) Molecule C (01E06/V9), reduced. (F) Molecule C (01E06/V9), non-reduced. (G) Molecule D (7G2/V9), reduced. (H) Molecule D (7G2/V9), non-reduced. (I) Molecule E (01F05/2C11), lane 1: non-reduced, lane 2: reduced.

FIG. 10. Analytical size exclusion chromatography of purified anti-Robo 4/anti-CD3 1+1 CrossFab-IgG constructs (A-D: Superdex 200 10/300 GL (GE Healthcare); 2 mM MOPS pH 7.3, 150 mM NaCl, 0.02% (w/v) NaCl; 50 μg sample injected. E: TSKgel G3000 SW XL (Tosoh); 25 mM K₂HPO₄, 125 mM NaCl, 200 mM L-arginine monohydrochloride, 0.02% (w/v) NaN₃, pH 6.7; 20 μg sample injected). (A) Molecule A (01F09/V9). (B) Molecule B (01F05/V9). (C) Molecule C (01E06/V9). (D) Molecule D (7G2/V9). (E) Molecule E (01F05/2C11).

FIG. 11. CE-SDS analysis of purified anti-Robo 4/anti-CD3 2+1 CrossFab-IgG construct shown as SDS-PAGE. Electropherogram of molecule F (01F05/V9), non-reduced (A) and reduced (B).

FIG. 12. Analytical size exclusion chromatography of purified anti-Robo 4/anti-CD3 2+1 CrossFab-IgG construct (TSKgel G3000 SW XL (Tosoh); 25 mM K₂HPO₄, 125 mM NaCl, 200 mM L-arginine monohydrochloride, 0.02% (w/v) NaN₃, pH 6.7; 20 μg sample molecule F (01F05/V9) injected.

FIG. 13. SDS PAGE analysis of purified anti-Robo 4/anti-CD3 Fab-CrossFab and Fab-Fab-CrossFab constructs (4-12% Bis/Tris (NuPage, Invitrogen); Coomassie stained). (A) lane 1: Molecule G (01E06/V9 Fab-CrossFab), reduced; lane 2: Molecule H (7G2/V9 Fab-CrossFab), reduced; lane 3: Molecule I (01F09/V9 Fab-CrossFab), reduced; lane 4: Molecule J (01F05/V9 Fab-CrossFab), reduced. (B) lane 1: Molecule G (01E06/V9 Fab-CrossFab), non-reduced; lane 2: Molecule H (7G2/V9 Fab-CrossFab), non-reduced; lane 3: Molecule I (01F09/V9 Fab-CrossFab), non-reduced; lane 4: Molecule J (01F05/V9 Fab-CrossFab), non-reduced. (C) lane 1: Molecule K (01F05/2C11 Fab-CrossFab), non-reduced; lane 2: Molecule K (01F05/2C11 Fab-CrossFab), reduced. (D) Molecule L (01F05/V9 Fab-Fab-CrossFab), reduced. (E) Molecule L (01F05/V9 Fab-Fab-CrossFab), non-reduced.

FIG. 14. Analytical size exclusion chromatography of purified anti-Robo 4/anti-CD3 Fab-CrossFab and Fab-Fab-CrossFab constructs (A-D: Superdex 200 10/300 GL (GE Healthcare); 2 mM MOPS pH 7.3, 150 mM NaCl, 0.02% (w/v) NaCl; 50 μg sample injected. E-F: TSKgel G3000 SW XL (Tosoh); 25 mM K₂HPO₄, 125 mM NaCl, 200 mM L-arginine monohydrochloride, 0.02% (w/v) NaN₃, pH 6.7; 20 μg sample injected). (A) Molecule G (01E06/V9 Fab-CrossFab). (B) Molecule H (7G2/V9 Fab-CrossFab). (C) Molecule I (01F09/V9 Fab-CrossFab). (D) Molecule J (01F05/V9 Fab-CrossFab). (E) Molecule K (01F05/2C11 Fab-CrossFab). (F) Molecule L (01F05/V9 Fab-Fab-CrossFab).

FIG. 15. Binding of anti-Robo 4 IgGs derived from phage display (7G2) and hamster immunization (01F05, 01E06, 01F09) to CHO-Robo 4 cells.

FIG. 16. Antibody-dependent cell-mediated cytotoxicity (ADCC) induced by anti-Robo 4 IgGs. (A) Killing of HUVECs by human PBMCs as measured by LDH release (E:T=25:1, incubation time 4 h) induced by wildtype (wt; 7G2, 01F05) and glycoengineered (g2; 7G2, 01F05, 01F09) anti-Robo 4 IgGs. (B) Killing of HUVECs by human PBMCs as measured by LDH release (E:T=25:1, incubation time 4 h) induced by wildtype (wt) 01E06 anti-Robo 4 IgG and glycoengineered (g2), one-armed (OA) 01E06 anti-Robo 4 IgG.

FIG. 17. T-cell mediated killing of human endothelial cells (HUVECs) induced by anti-Robo 4/anti-CD3 bispecific antibodies in the Fab-CrossFab (A) and the 1+1 CrossFab-IgG (B) format (E:T=5:1, incubation time 22 h).

FIG. 18. CD25 upregulation on human CD4+(A) and CD8+(B) T cells after T cell-mediated killing of human endothelial cells (E:T=5:1, 17 h incubation) induced by anti-Robo 4/anti-CD3 bispecific antibodies in the Fab-CrossFab format (referred to as “B”) or the 1+1 CrossFab-IgG format (referred to as “C”).

FIG. 19. T-cell mediated killing of human endothelial cells (HUVECs) induced by anti-Robo 4 (01F05)/anti-CD3 (V9) bispecific antibodies in the Fab-CrossFab, the Fab-Fab-CrossFab, the 1+1 CrossFab-IgG and the 2+1 CrossFab-IgG format (E:T=10:1, incubation time 24 h (A) or 45 h (B)). A 2+1 CrossFab-IgG construct comprising non-binding IgG was used as control.

FIG. 20. Upregulation of CD25 (A, C) and CD69 (B, D) on human CD4+(A, B) and CD8+(C, D) T cells after T cell-mediated killing of human endothelial cells (E:T=10:1, 24 h incubation) induced by anti-Robo 4 (01F05)/anti-CD3 (V9) bispecific antibodies in the Fab-CrossFab, the Fab-Fab-CrossFab, the 1+1 CrossFab-IgG and the 2+1 CrossFab-IgG format. A 2+1 CrossFab-IgG construct comprising non-binding IgG was used as control.

FIG. 21. Secretion of Granzyme B (A), interferon-γ (B), TNFα (C), IL-2 (D), IL-4 (E) and IL-10 (F) by human PBMCs after T cell mediated killing of human endothelial cells (HUVECs) induced by anti-Robo 4 (01F05)/anti-CD3 (V9) bispecific antibodies in the Fab-CrossFab, the Fab-Fab-CrossFab, the 1+1 CrossFab-IgG and the 2+1 CrossFab-IgG format. A 2+1 CrossFab-IgG construct comprising non-binding IgG was used as control.

FIG. 22. Proliferation of CD4⁺ (A) and CD8⁺ (B) T cells after T cell mediated killing of human endothelial cells (HUVECs) induced by different concentrations of anti-Robo 4 (01F05)/anti-CD3 (V9) bispecific antibodies in the Fab-CrossFab (molecule J), the Fab-Fab-CrossFab (molecule L), the 1+1 CrossFab-IgG (molecule B) and the 2+1 CrossFab-IgG format (molecule F). A 2+1 CrossFab-IgG construct comprising non-binding IgG was used as control (untarg.).

FIG. 23. T-cell mediated killing of mouse endothelial cells (MS-1) by human T cells, induced by anti-Robo 4/anti-CD3 bispecific antibodies in the Fab-CrossFab (A) and the 1+1 CrossFab-IgG (B) format (E:T=5:1, incubation time 17 h).

FIG. 24. CD25 upregulation on human CD4+(A) and CD8+(B) T cells after T cell-mediated killing of murine endothelial cells (E:T=5:1, 17 h incubation) induced by anti-Robo 4/anti-CD3 bispecific antibodies in the Fab-CrossFab format (referred to as “B”) or the 1+1 CrossFab-IgG format (referred to as “C”).

FIG. 25. T-cell mediated killing of mouse endothelial cells (MS-1) by murine splenocytes, induced by anti-Robo 4/anti-CD3 (01F05/2C11) bispecific Fab-CrossFab antibody (molecule K) (E:T=10:1, incubation time 48 and 72 h).

FIG. 26. In vivo anti-tumor efficacy of anti-Robo4/anti-mouse or human CD3 (01F05/C11 (molecule K) or 01F05/V9 (molecule J), respectively) bispecific Fab-CrossFab antibodies in N-Ras melanoma-bearing mice. Treatment from day 8 to 20 after tumor cell inoculation, n=10 mice per treatment group.

FIG. 27. Ex vivo FACS analysis of peripheral T cell in N-Ras melanoma-bearing mice treated with anti-Robo4/anti-mouse or human CD3 (01F05/C11 (molecule K) or 01F05/V9 (molecule J), respectively) bispecific Fab-CrossFab antibodies. PBMCs were harvested after 11 days of treatment and analysed for T cell surface markers CD4 and CD8, as well as proliferation marker Ki67.

FIG. 28. Number of CD3 positive cells detected by immunohistochemistry (IHC) in tumor tissue sections from N-Ras melanoma-bearing mice treated with anti-Robo4/anti-mouse or human CD3 (01F05/C11 (molecule K) or 01F05/V9 (molecule J), respectively) bispecific Fab-CrossFab antibodies.

FIG. 29. Exemplary configurations of the T cell activating bispecific antigen binding molecules (TCBs) of the invention. (A, D) Illustration of the “1+1 CrossMab” molecule. (B, E) Illustration of the “2+1 CrossFab-IgG” molecule with alternative order of Crossfab and Fab components (“inverted”). (C, F) Illustration of the “2+1 CrossFab-IgG” molecule. (G, K) Illustration of the “1+1 CrossFab-IgG” molecule with alternative order of Crossfab and Fab components (“inverted”). (H, L) Illustration of the “1+1 CrossFab-IgG” molecule. (I, M) Illustration of the “2+1 CrossFab-IgG” molecule with two CrossFabs. (J, N) Illustration of the “2+1 CrossFab-IgG” molecule with two CrossFabs and alternative order of Crossfab and Fab components (“inverted”). (0, S) Illustration of the “Fab-CrossFab” molecule. (P, T) Illustration of the “CrossFab-Fab” molecule. (Q, U) Illustration of the “(Fab)₂-CrossFab” molecule. (R, V) Illustration of the “CrossFab-(Fab)₂” molecule. (W, Y) Illustration of the “Fab-(CrossFab)₂” molecule. (X, Z) Illustration of the “(CrossFab)₂-Fab” molecule. Black dot: optional modification in the Fc domain promoting heterodimerization. ++, −−: amino acids of opposite charges optionally introduced in the CH1 and CL domains. Crossfab molecules are depicted as comprising an exchange of VH and VL regions, but may—in embodiments wherein no charge modifications are introduced in CH1 and CL domains—alternatively comprise an exchange of the CH1 and CL domains.

FIG. 30. Illustration of the anti-Robo 4/anti-CD3 bispecific antibody prepared in Example 25 (Molecule M): “2+1 CrossFab-IgG, inverted” with charge modifications (VH/VL exchange in CD3 binder, charge modification in Robo 4 binders, EE=147E, 213E; RK=123R, 124K).

FIG. 31. CE-SDS analysis of the anti-Robo 4/anti-CD3 bispecific antibody prepared in Example 25, molecule M (final purified preparations, electropherogram, lane A=non-reduced, lane B=reduced).

FIG. 32. SDS-PAGE analysis (4-12% Bis-Tris, Coomassie stained, non reduced) of the anti-Robo 4/anti-CD3 bispecific antibody prepared in Example 25 (molecule M) after the first purification step (Protein A affinity chromatography). Lane 1=marker (HiMark, Invitrogen); lane 4-12=fractions from Protein A affinity chromatography of molecule A.

FIG. 33. T-cell killing of human endothelial cells (HUVEC) induced by anti-Robo 4/anti-CD3 bispecific antibodies of different formats after 24 h (A) or 48 h (B).

FIG. 34. T-cell killing of mouse endothelial cells (MS-1) induced by anti-Robo 4/anti-CD3 bispecific antibodies of different formats after 24 h (A) or 48 h (B).

FIG. 35. Upregulation of CD25 (A, C) and CD69 (B, D) on CD8+(A, B) and CD4+(C, D) T cells after T cell-mediated killing of human endothelial cells (HUVEC) induced by anti-Robo 4/anti-CD3 bispecific antibodies for 48 h.

FIG. 36. Upregulation of CD25 (A, C) and CD69 (B, D) on CD8+(A, B) and CD4+(C, D) T cells after T cell-mediated killing of mouse endothelial cells (MS-1) induced by anti-Robo 4/anti-CD3 bispecific antibodies for 48 h.

FIG. 37. Secretion of Granzyme B (A), interferon-γ (B), IL-2 (C), TNFα (D) and IL-10 (E) by human effector cells (PBMCs) after T cell-mediated killing of human endothelial cells (HUVEC) induced by anti-Robo 4/anti-CD3 bispecific antibodies.

FIG. 38. CD3 activation on Jurkat-NFAT reporter cells induced by anti-Robo 4/anti-CD3 bispecific antibodies in the presence of human (HUVEC, panel A) or mouse (MS-1, panel B) endothelial cells, or in the absence of target cells (panel C).

FIG. 39. Pharmacokinetic parameters of a 0.5 mg/kg and of a 2.5 mg/kg iv bolus administration of anti-Robo 4/anti-CD3 bispecific antibody “molecule M” from sparse sampling data in NOG mice.

### DETAILED DESCRIPTION OF THE INVENTION

### Definitions

Terms are used herein as generally used in the art, unless otherwise defined in the following.

As used herein, the term “antigen binding molecule” refers in its broadest sense to a molecule that specifically binds an antigenic determinant. Examples of antigen binding molecules are immunoglobulins and derivatives, e.g. fragments, thereof.

As used herein, the term “antigen binding molecule” refers in its broadest sense to a molecule that specifically binds an antigen. Examples of antigen binding molecules are immunoglobulins and derivatives, e.g. fragments, thereof.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. Robo 4, CD3) can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, the T cell activating bispecific antigen binding molecule of the invention is capable of specific binding to human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1 or SEQ ID NO: 136. The amino acid sequence of cynomolgus [Macaca fascicularis] CD3ε is shown in NCBI GenBank no. BAB71849.1 or SEQ ID NO: 137.

“Robo 4” or “Roundabout homolog 4”, refers to any native Robo 4 from any vertebrate source, including mammals such as primates (e.g. humans), non-human primates (e.g. cynomolgus monkeys) and rodents (e.g. mice and rats), unless otherwise indicated. The term encompasses “full-length,” unprocessed Robo 4 as well as any form of Robo 4 that results from processing in the cell. The term also encompasses naturally occurring variants of Robo 4, e.g., splice variants or allelic variants. In one embodiment, the T cell activating bispecific antigen binding molecule of the invention is capable of specific binding to human Robo 4, particularly the extracellular domain of human Robo 4.

The amino acid sequence of human Robo 4 (also known as Magic roundabout) is shown in UniProt (www.uniprot.org) accession no. Q8WZ75 (version 92), or NCBI (www.ncbi.nlm.nih.gov/) RefSeq NP_061928.4. The extracellular domain (ECD) of human Robo 4 (isoform 1) extends from amino acid position 28 to around position 468. The nucleotide and amino acid sequences of a human Robo 4 ECD (isoform 1) fused to a PreScission protease recognition site, an Avi- and a 6×His-tag is shown in SEQ ID NOs 2 and 1, respectively. The Robo 4 ECD comprises the Ig-like domain 1, which extends from amino acid position 32 of the full sequence to around amino acid position 131 (SEQ ID NOs 16 and 15 show nucleotide and amino acid sequences of a human Robo 4 Ig-like domain 1 fused to a human Fc region), the Ig-like domain 2, which extends from around amino acid position 137 of the full sequence to around amino acid position 224 (SEQ ID NOs 18 and 17 show nucleotide and amino acid sequences of a human Robo 4 Ig-like domain 2 fused to a human Fc region), the Fibronectin (FN)-like domain 1, which extends from around amino acid position 252 of the full sequence to around amino acid position 340 (SEQ ID NOs 12 and 11 show nucleotide and amino acid sequences of a human Robo 4 FN-like domain 1 fused to a human Fc region), and the FN-like domain 2, which extends from around amino acid position 347 of the full sequence to around amino acid position 438 (SEQ ID NOs 14 and 13 show nucleotide and amino acid sequences of a human Robo 4 FN-like domain 2 fused to a human Fc region).

In one embodiment, the T cell activating bispecific antigen binding molecule is also capable of binding to mouse Robo 4, particularly the extracellular domain of mouse Robo 4. The sequence of mouse Robo 4 is shown in UniProt (www.uniprot.org) accession no. Q8C310 (version 84), or NCBI (www.ncbi.nlm.nih.gov/) RefSeq NP_083059.2. SEQ ID NOs 4 and 3 show the nucleotide and amino acid sequences, respectively, of a mouse Robo 4 ECD fused to a PreScission protease recognition site, an Avi- and a 6×His-tag. In yet another embodiment, the T cell activating bispecific antigen binding molecule is also capable of binding to cynomolgus Robo 4, particularly the extracellular domain of cynomolgus Robo 4. SEQ ID NOs 10 and 9 show the nucleotide and amino acid sequences, respectively, of a cynomolgus Robo 4 ECD fused to a AcTEV protease recognition site, an Avi- and a 6×His-tag.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is Robo 4, particularly human Robo 4.

As used herein, the terms “first”, “second” or “third” with respect to Fab molecules etc., are used for convenience of distinguishing when there is more than one of each type of moiety. Use of these terms is not intended to confer a specific order or orientation of the T cell activating bispecific antigen binding molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g. a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

By a “crossover” Fab molecule (also termed “CrossFab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (lc) and lambda (λ), based on the amino acid sequence of its constant domain. An immunoglobulin essentially consists of two Fab molecules and an Fc domain, linked via the immunoglobulin hinge region.

The term “antibody” herein is used in the broadest sense and encompasses various antibody structures, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161;

Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., Sequences of Proteins of Immunological Interest, 5th Ed. Public Health Service, National Institutes of Health, Bethesda, Md. (1991) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table A as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE A
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein in connection with variable region seqeunces, “Kabat numbering” refers to the numbering system set forth by Kabat et al., Sequences of Proteins of Immunological Interest, 5th Ed. Public Health Service, National Institutes of Health, Bethesda, Md. (1991). Unless otherwise specified, references to the numbering of specific amino acid residue positions in an antibody variable region are according to the Kabat numbering system. As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, Md. (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The polypeptide sequences of the sequence listing are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. Such variable domains are referred to herein as “humanized variable region”. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprised in a T cell activating bispecific antigen binding molecule according to the invention, comprises an additional C-terminal glycine-lysine dipeptide (G446 and K447, numbering according to EU index of Kabat). In one embodiment of the invention, a heavy chain including a subunit of an Fc domain as specified herein, comprised in a T cell activating bispecific antigen binding molecule according to the invention, comprises an additional C-terminal glycine residue (G446, numbering according to EU index of Kabat). Compositions of the invention, such as the pharmaceutical compositions described herein, comprise a population of T cell activating bispecific antigen binding molecules of the invention. The population of T cell activating bispecific antigen binding molecule may comprise molecules having a full-length heavy chain and molecules having a cleaved variant heavy chain. The population of T cell activating bispecific antigen binding molecules may consist of a mixture of molecules having a full-length heavy chain and molecules having a cleaved variant heavy chain, wherein at least 50%, at least 60%, at least 70%, at least 80% or at least 90% of the T cell activating bispecific antigen binding molecules have a cleaved variant heavy chain. In one embodiment of the invention a composition comprising a population of T cell activating bispecific antigen binding molecules of the invention comprises an T cell activating bispecific antigen binding molecule comprising a heavy chain including a subunit of an Fc domain as specified herein with an additional C-terminal glycine-lysine dipeptide (G446 and K447, numbering according to EU index of Kabat). In one embodiment of the invention a composition comprising a population of T cell activating bispecific antigen binding molecules of the invention comprises an T cell activating bispecific antigen binding molecule comprising a heavy chain including a subunit of an Fc domain as specified herein with an additional C-terminal glycine residue (G446, numbering according to EU index of Kabat). In one embodiment of the invention such a composition comprises a population of T cell activating bispecific antigen binding molecules comprised of molecules comprising a heavy chain including a subunit of an Fc domain as specified herein; molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program. The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, T cell activating bispecific antigen binding molecules of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

### DETAILED DESCRIPTION OF THE EMBODIMENTS

### Charge Modifications

The T cell activating bispecific antigen binding molecules of the invention may comprise amino acid substitutions in Fab molecules comprised therein which are particularly efficient in reducing mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT application no. PCT/EP2015/057165, particularly the examples therein, incorporated herein by reference in its entirety).

Accordingly, in particular embodiments, the T cell activating bispecific antigen binding molecule of the invention comprises

(a) a first Fab molecule which specifically binds to a first antigen
(b) a second Fab molecule which specifically binds to a second antigen, and wherein the variable domains VL and VH of the Fab light chain and the Fab heavy chain are replaced by each other, wherein the first antigen is an activating T cell antigen and the second antigen is Robo 4, or the first antigen is Robo 4 and the second antigen is an activating T cell antigen; and wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).

The T cell activating bispecific antigen binding molecule does not comprise both modifications mentioned under i) and ii). The constant domains CL and CH1 of the second Fab molecule are not replaced by each other (i.e. remain unexchanged).

In one embodiment of the T cell activating bispecific antigen binding molecule according to the invention, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by lysine (K) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In particular embodiments, the constant domain CL of the first Fab molecule under a) is of kappa isotype.

Alternatively, the amino acid substitutions according to the above embodiments may be made in the constant domain CL and the constant domain CH1 of the second Fab molecule under b) instead of in the constant domain CL and the constant domain CH1 of the first Fab molecule under a). In particular such embodiments, the constant domain CL of the second Fab molecule under b) is of kappa isotype.

The T cell activating bispecific antigen binding molecule according to the invention may further comprise a third Fab molecule which specifically binds to the first antigen. In particular embodiments, said third Fab molecule is identical to the first Fab molecule under a). In these embodiments, the amino acid substitutions according to the above embodiments will be made in the constant domain CL and the constant domain CH1 of each of the first Fab molecule and the third Fab molecule. Alternatively, the amino acid substitutions according to the above embodiments may be made in the constant domain CL and the constant domain CH1 of the second Fab molecule under b), but not in the constant domain CL and the constant domain CH1 of the first Fab molecule and the third Fab molecule.

In particular embodiments, the T cell activating bispecific antigen binding molecule according to the invention further comprises an Fc domain composed of a first and a second subunit capable of stable association.

T Cell Activating Bispecific Antigen Binding Molecule Formats

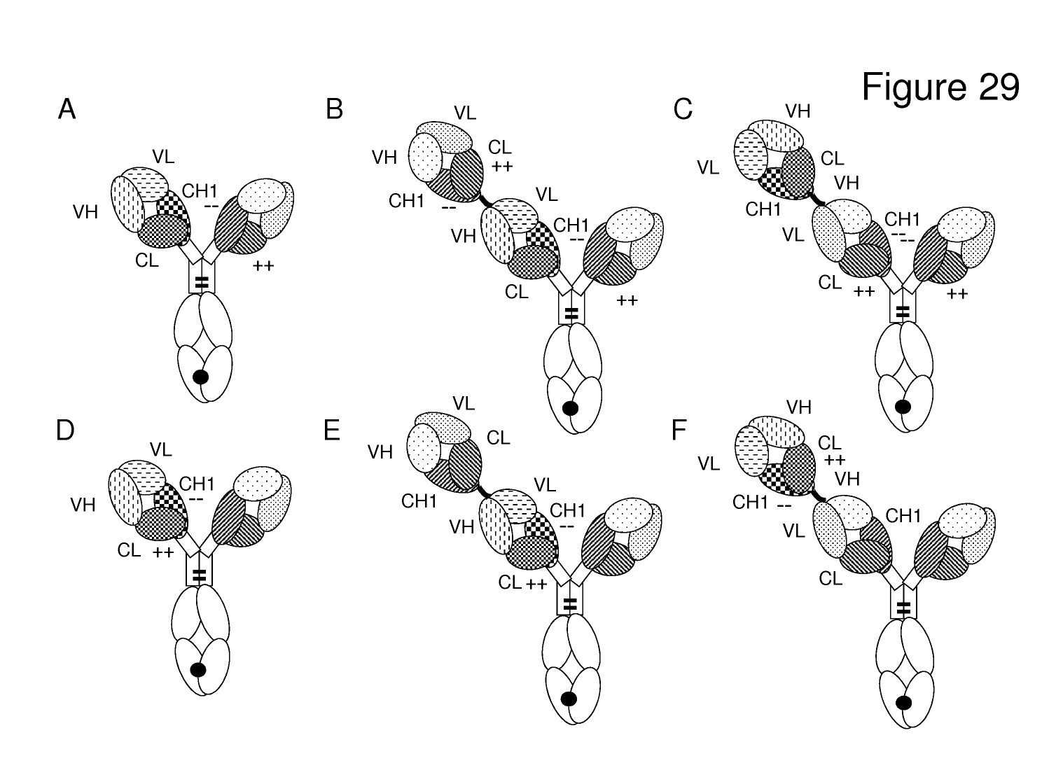

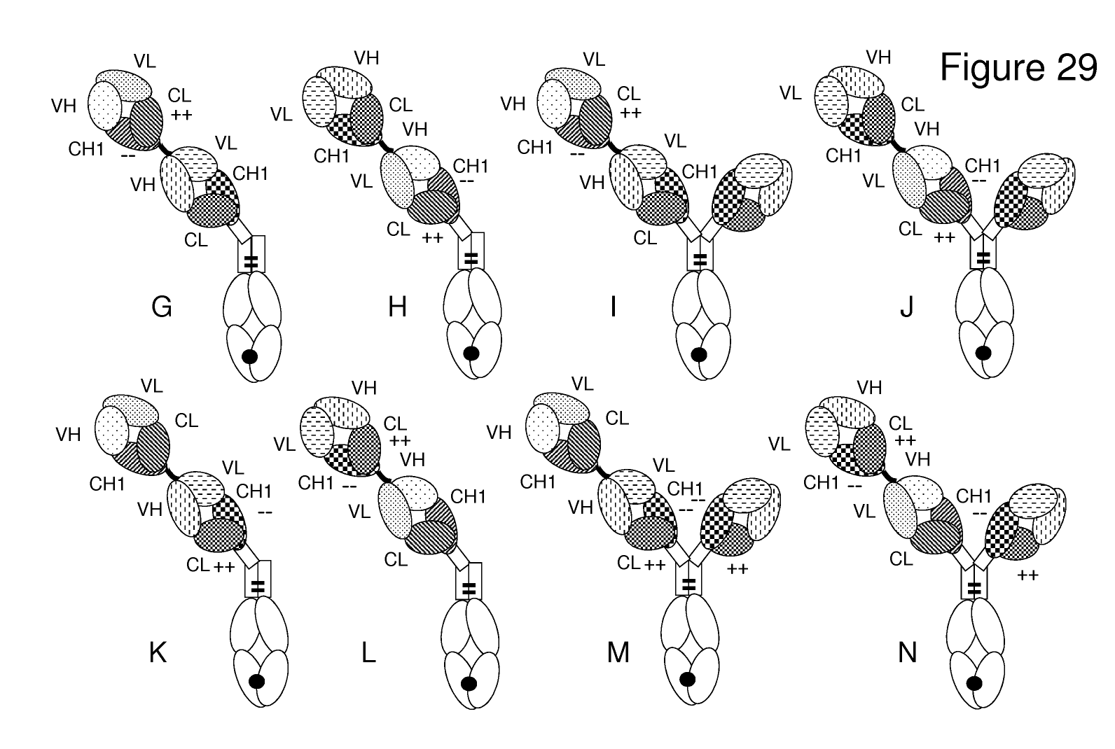

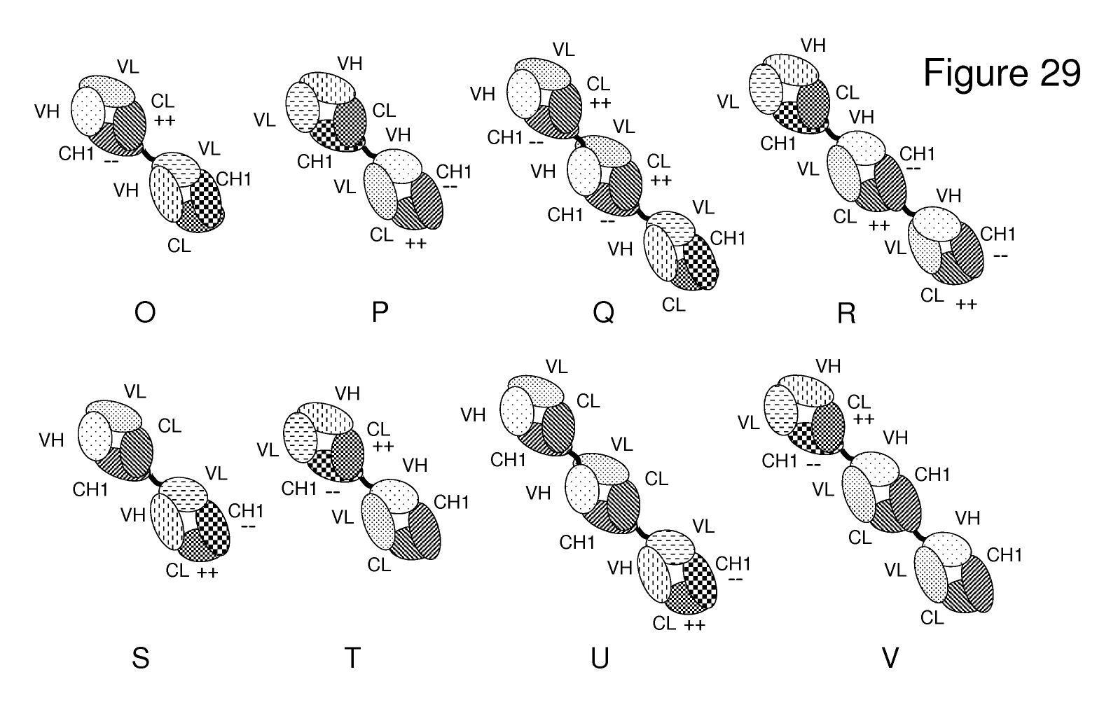

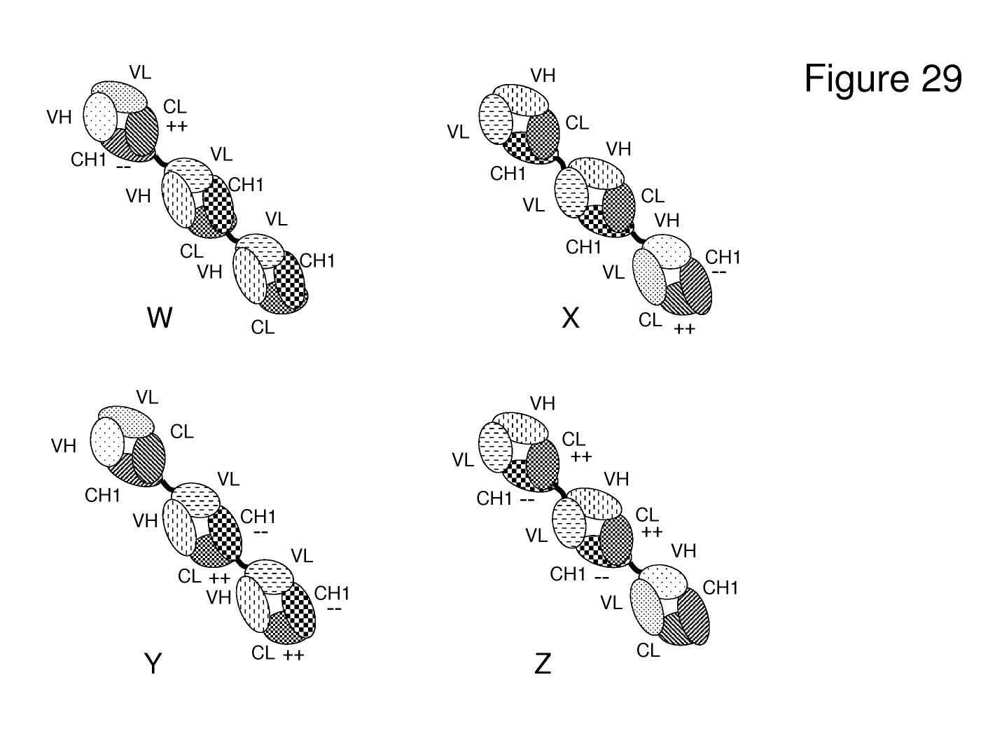

The components of the T cell activating bispecific antigen binding molecule can be fused to each other in a variety of configurations. Exemplary configurations are depicted in FIG. 29. In particular embodiments, the antigen binding moieties comprised in the T cell activating bispecific antigen binding molecule are Fab molecules. In such embodiments, the first, second, third etc. antigen binding moiety may be referred to herein as first, second, third etc. Fab molecule, respectively. Furthermore, in particular embodiments, the T cell activating bispecific antigen binding molecule comprises an Fc domain composed of a first and a second subunit capable of stable association.

In some embodiments, the second Fab molecule is fused at the C-terminus of the Fab heavy chain to the N-terminus of the first or the second subunit of the Fc domain.

In one such embodiment, the first Fab molecule is fused at the C-terminus of the Fab heavy chain to the N-terminus of the Fab heavy chain of the second Fab molecule. In a specific such embodiment, the T cell activating bispecific antigen binding molecule essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first or the second subunit of the Fc domain. Such a configuration is schematically depicted in FIGS. 29G and 29K. Optionally, the Fab light chain of the first Fab molecule and the Fab light chain of the second Fab molecule may additionally be fused to each other.

In another such embodiment, the first Fab molecule is fused at the C-terminus of the Fab heavy chain to the N-terminus of the first or second subunit of the Fc domain. In a specific such embodiment, the T cell activating bispecific antigen binding molecule essentially consists of the first and the second Fab molecule, the Fc domain composed of a first and a second subunit, and optionally one or more peptide linkers, wherein the first and the second Fab molecule are each fused at the C-terminus of the Fab heavy chain to the N-terminus of one of the subunits of the Fc domain. Such a configuration is schematically depicted in FIGS. 29A and 29D. The first and the second Fab molecule may be fused to the Fc domain directly or through a peptide linker. In a particular embodiment the first and the second Fab molecule are each fused to the Fc domain through an immunoglobulin hinge region. In a specific embodiment, the immunoglobulin hinge region is a human IgG₁ hinge region, particularly where the Fc domain is an IgG₁ Fc domain.

In other embodiments, the first Fab molecule is fused at the C-terminus of the Fab heavy chain to the N-terminus of the first or second subunit of the Fc domain.

In one such embodiment, the second Fab molecule is fused at the C-terminus of the Fab heavy chain to the N-terminus of the Fab heavy chain of the first Fab molecule. In a specific such embodiment, the T cell activating bispecific antigen binding molecule essentially consists of the first and the second Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first or the second subunit of the Fc domain. Such a configuration is schematically depicted in FIGS. 29H and 29L. Optionally, the Fab light chain of the first Fab molecule and the Fab light chain of the second Fab molecule may additionally be fused to each other.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GxS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=2. In one embodiment said peptide linker is (G₄S)₂. A particularly suitable peptide linker for fusing the Fab light chains of the first and the second Fab molecule to each other is (G₄S)₂. An exemplary peptide linker suitable for connecting the Fab heavy chains of the first and the second Fab fragments comprises the sequence (D)-(G₄S)₂ (SEQ ID NOs 148 and 149). Another suitable such linker comprises the sequence (G₄S)₄. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

A T cell activating bispecific antigen binding molecule with a single antigen binding moiety (such as a Fab molecule) capable of specific binding to a target cell antigen such as Robo 4 (for example as shown in FIG. 29A, D, G, H, K, L) is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availablity.

In many other cases, however, it will be advantageous to have a T cell activating bispecific antigen binding molecule comprising two or more antigen binding moieties (such as Fab moelcules) specific for a target cell antigen such as Robo 4 (see examples shown in FIG. 29B, 29C, 29E, 29F, 29I, 29J. 29M or 29N), for example to optimize targeting to the target site, to allow crosslinking of target cell antigens, or to enhance binding avidity.

Accordingly, in particular embodiments, the T cell activating bispecific antigen binding molecule of the invention further comprises a third Fab molecule which specifically binds to the first antigen. The first antigen preferably is Robo 4. In one embodiment, the third Fab molecule is a conventional Fab molecule. In one embodiment, the third Fab molecule is identical to the first Fab molecule (i.e. the first and the third Fab molecule comprise the same heavy and light chain amino acid sequences and have the same arrangement of domains (i.e. conventional or crossover)). In a particular embodiment, the second Fab molecule specifically binds to an activating T cell antigen, particularly CD3, and the first and third Fab molecule specifically bind to Robo 4.

In alternative embodiments, the T cell activating bispecific antigen binding molecule of the invention further comprises a third Fab molecule which specifically binds to the second antigen. In these embodiments, the second antigen preferably is Robo 4. In one such embodiment, the third Fab molecule is a crossover Fab molecule (a Fab molecule wherein the variable domains VH and VL or the constant domains CL and CH1 of the Fab heavy and light chains are exchanged/replaced by each other). In one such embodiment, the third Fab molecule is identical to the second Fab molecule (i.e. the second and the third Fab molecule comprise the same heavy and light chain amino acid sequences and have the same arrangement of domains (i.e. conventional or crossover)). In one such embodiment, the first Fab molecule specifically binds to an activating T cell antigen, particularly CD3, and the second and third Fab molecule specifically bind to Robo 4.

In one embodiment, the third Fab molecule is fused at the C-terminus of the Fab heavy chain to the N-terminus of the first or second subunit of the Fc domain.

In a particular embodiment, the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule. In a specific such embodiment, the T cell activating bispecific antigen binding molecule essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIGS. 29B and 29E (particular embodiments, wherein the third Fab molecule is a conventional Fab molecule and preferably identical to the first Fab molecule), and FIGS. 29I and 29M (alternative embodiments, wherein the third Fab molecule is a crossover Fab molecule and preferably identical to the second Fab molecule). The second and the third Fab molecule may be fused to the Fc domain directly or through a peptide linker. In a particular embodiment the second and the third Fab molecule are each fused to the Fc domain through an immunoglobulin hinge region. In a specific embodiment, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In another embodiment, the first and the third Fab molecule are each fused at the C-terminus of the Fab heavy chain to the N-terminus of one of the subunits of the Fc domain, and the second Fab molecule is fused at the C-terminus of the Fab heavy chain to the N-terminus of the Fab heavy chain of the first Fab molecule. In a specific such embodiment, the T cell activating bispecific antigen binding molecule essentially consists of the first, the second and the third Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIGS. 29C and 29F (particular embodiments, wherein the third Fab molecule is a conventional Fab molecule and preferably identical to the first Fab molecule) and in FIGS. 29J and 29N (alternative embodiments, wherein the third Fab molecule is a crossover Fab molecule and preferably identical to the second Fab molecule). The first and the third Fab molecule may be fused to the Fc domain directly or through a peptide linker. In a particular embodiment the first and the third Fab molecule are each fused to the Fc domain through an immunoglobulin hinge region. In a specific embodiment, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In configurations of the T cell activating bispecific antigen binding molecule wherein a Fab molecule is fused at the C-terminus of the Fab heavy chain to the N-terminus of each of the subunits of the Fc domain through an immunoglobulin hinge regions, the two Fab molecules, the hinge regions and the Fc domain essentially form an immunoglobulin molecule. In a particular embodiment the immunoglobulin molecule is an IgG class immunoglobulin. In an even more particular embodiment the immunoglobulin is an IgG₁ subclass immunoglobulin. In another embodiment the immunoglobulin is an IgG₄ subclass immunoglobulin. In a further particular embodiment the immunoglobulin is a human immunoglobulin. In other embodiments the immunoglobulin is a chimeric immunoglobulin or a humanized immunoglobulin.

In some of the T cell activating bispecific antigen binding molecule of the invention, the Fab light chain of the first Fab molecule and the Fab light chain of the second Fab molecule are fused to each other, optionally via a peptide linker. Depending on the configuration of the first and the second Fab molecule, the Fab light chain of the first Fab molecule may be fused at its C-terminus to the N-terminus of the Fab light chain of the second Fab molecule, or the Fab light chain of the second Fab molecule may be fused at its C-terminus to the N-terminus of the Fab light chain of the first Fab molecule. Fusion of the Fab light chains of the first and the second Fab molecule further reduces mispairing of unmatched Fab heavy and light chains, and also reduces the number of plasmids needed for expression of some of the T cell activating bispecific antigen binding molecules of the invention.

In certain embodiments the T cell activating bispecific antigen binding molecule according to the invention comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with an Fc domain subunit (VL₍₂₎-CH1₍₂₎-CH2-CH3(-CH4)), and a polypeptide wherein the Fab heavy chain of the first Fab molecule shares a carboxy-terminal peptide bond with an Fc domain subunit (VH₍₁₎-CH1₍₁₎-CH2-CH3(-CH4)). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In certain embodiments the polypeptides are covalently linked, e.g., by a disulfide bond. In certain embodiments the T cell activating bispecific antigen binding molecule according to the invention comprises a polypeptide wherein the Fab heavy chain variable region of the second Fab molecule shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with an Fc domain subunit (VH₍₂₎-CL₍₂₎-CH2-CH3(-CH4)), and a polypeptide wherein the Fab heavy chain of the first Fab molecule shares a carboxy-terminal peptide bond with an Fc domain subunit (VH₍₁₎-CH1₍₁₎-CH2-CH3(-CH4)). In some embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In certain embodiments the polypeptides are covalently linked, e.g., by a disulfide bond. In some embodiments, the T cell activating bispecific antigen binding molecule comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule, which in turn shares a carboxy-terminal peptide bond with an Fc domain subunit (VL₍₂₎-CH1₍₂₎-VH₍₁₎-CH1₍₁₎-CH2-CH3(-CH4)). In other embodiments, the T cell activating bispecific antigen binding molecule comprises a polypeptide wherein the Fab heavy chain of the first Fab molecule shares a carboxy-terminal peptide bond with the Fab light chain variable region of the second Fab molecule which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with an Fc domain subunit (VH₍₁₎-CH1₍₁₎-VL₍₂₎-CH1₍₂₎-CH2-CH3 (-CH4)).

In some of these embodiments the T cell activating bispecific antigen binding molecule further comprises a crossover Fab light chain polypeptide of the second Fab molecule, wherein the Fab heavy chain variable region of the second Fab molecule shares a carboxy-terminal peptide bond with the Fab light chain constant region of the second Fab molecule (VH₍₂₎-CL₍₂₎), and the Fab light chain polypeptide of the first Fab molecule (VL₍₁₎-CL₍₁₎). In others of these embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule which in turn shares a carboxy-terminal peptide bond with the Fab light chain polypeptide of the first Fab molecule (VH₍₂₎-CL₍₂₎-VL₍₁₎-CL₍₁₎), or a polypeptide wherein the Fab light chain polypeptide of the first Fab molecule shares a carboxy-terminal peptide bond with the Fab heavy chain variable region of the second Fab molecule which in turn shares a carboxy-terminal peptide bond with the Fab light chain constant region of the second Fab molecule (VL₍₁₎-CL₍₁₎-VH₍₂₎-CL₍₂₎), as appropriate.

The T cell activating bispecific antigen binding molecule according to these embodiments may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain embodiments the polypeptides are covalently linked, e.g., by a disulfide bond.

In some embodiments, the T cell activating bispecific antigen binding molecule comprises a polypeptide wherein the Fab heavy chain variable region of the second Fab molecule shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain of the first Fab molecule, which in turn shares a carboxy-terminal peptide bond with an Fc domain subunit (VH₍₂₎-CL₍₂₎-VH₍₁₎-CH1₍₁₎-CH2-CH3(-CH4)). In other embodiments, the T cell activating bispecific antigen binding molecule comprises a polypeptide wherein the Fab heavy chain of the first Fab molecule shares a carboxy-terminal peptide bond with the Fab heavy chain variable region of the second Fab molecule which in turn shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with an Fc domain subunit (VH₍₁₎-CH1₍₁₎-VH₍₂₎-CL₍₂₎-CH2-CH3 (-CH4)).

In some of these embodiments the T cell activating bispecific antigen binding molecule further comprises a crossover Fab light chain polypeptide of the second Fab molecule, wherein the Fab light chain variable region of the second Fab molecule shares a carboxy-terminal peptide bond with the Fab heavy chain constant region of the second Fab molecule (VL₍₂₎-CH1₍₂₎), and the Fab light chain polypeptide of the first Fab molecule (VL₍₁₎-CL₍₁₎). In others of these embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule which in turn shares a carboxy-terminal peptide bond with the Fab light chain polypeptide of the first Fab molecule (VL₍₂₎-CH1₍₂₎-VL₍₁₎-CL₍₁₎), or a polypeptide wherein the Fab light chain polypeptide of the first Fab molecule shares a carboxy-terminal peptide bond with the Fab heavy chain variable region of the second Fab molecule which in turn shares a carboxy-terminal peptide bond with the Fab light chain constant region of the second Fab molecule (VL₍₁₎-CL₍₁₎-VH₍₂₎-CL₍₂₎), as appropriate. The T cell activating bispecific antigen binding molecule according to these embodiments may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain embodiments the polypeptides are covalently linked, e.g., by a disulfide bond.

In some embodiments, the first Fab molecule is fused at the C-terminus of the Fab heavy chain to the N-terminus of the Fab heavy chain of the second Fab molecule. In certain such embodiments, the T cell activating bispecific antigen binding molecule does not comprise an Fc domain. In certain embodiments, the T cell activating bispecific antigen binding molecule essentially consists of the first and the second Fab molecule, and optionally one or more peptide linkers, wherein the first Fab molecule is fused at the C-terminus of the Fab heavy chain to the N-terminus of the Fab heavy chain of the second Fab molecule. Such a configuration is schematically depicted in FIGS. 29O and 29S.

In other embodiments, the second Fab molecule is fused at the C-terminus of the Fab heavy chain to the N-terminus of the Fab heavy chain of the first Fab molecule. In certain such embodiments, the T cell activating bispecific antigen binding molecule does not comprise an Fc domain. In certain embodiments, the T cell activating bispecific antigen binding molecule essentially consists of the first and the second Fab molecule, and optionally one or more peptide linkers, wherein the second Fab molecule is fused at the C-terminus of the Fab heavy chain to the N-terminus of the Fab heavy chain of the first Fab molecule. Such a configuration is schematically depicted in FIGS. 29P and 29T.

In some embodiments, the first Fab molecule is fused at the C-terminus of the Fab heavy chain to the N-terminus of the Fab heavy chain of the second Fab molecule, and the T cell activating bispecific antigen binding molecule further comprises a third Fab molecule, wherein said third Fab molecule is fused at the C-terminus of the Fab heavy chain to the N-terminus of the Fab heavy chain of the first Fab molecule. In particular such embodiments, said third Fab molecule is a conventional Fab molecule. In other such embodiments, said third Fab molecule is a crossover Fab molecule as described herein, i.e. a Fab molecule wherein the variable domains VH and VL or the constant domains CL and CH1 of the Fab heavy and light chains are exchanged/replaced by each other. In certain such embodiments, the T cell activating bispecific antigen binding molecule essentially consists of the first, the second and the third Fab molecule, and optionally one or more peptide linkers, wherein the first Fab molecule is fused at the C-terminus of the Fab heavy chain to the N-terminus of the Fab heavy chain of the second Fab molecule, and the third Fab molecule is fused at the C-terminus of the Fab heavy chain to the N-terminus of the Fab heavy chain of the first Fab molecule. Such a configuration is schematically depicted in FIGS. 29Q and 29U (particular embodiments, wherein the third Fab molecule is a conventional Fab molecule and preferably identical to the first Fab molecule).

In some embodiments, the first Fab molecule is fused at the C-terminus of the Fab heavy chain to the N-terminus of the Fab heavy chain of the second Fab molecule, and the T cell activating bispecific antigen binding molecule further comprises a third Fab molecule, wherein said third Fab molecule is fused at the N-terminus of the Fab heavy chain to the C-terminus of the Fab heavy chain of the second Fab molecule. In particular such embodiments, said third Fab molecule is a crossover Fab molecule as described herein, i.e. a Fab molecule wherein the variable domains VH and VL or the constant domains CH1 and CL of the Fab heavy and light chains are exchanged/replaced by each other. In other such embodiments, said third Fab molecule is a conventional Fab molecule. In certain such embodiments, the T cell activating bispecific antigen binding molecule essentially consists of the first, the second and the third Fab molecule, and optionally one or more peptide linkers, wherein the first Fab molecule is fused at the C-terminus of the Fab heavy chain to the N-terminus of the Fab heavy chain of the second Fab molecule, and the third Fab molecule is fused at the N-terminus of the Fab heavy chain to the C-terminus of the Fab heavy chain of the second Fab molecule. Such a configuration is schematically depicted in FIGS. 29W and 29Y (particular embodiments, wherein the third Fab molecule is a crossover Fab molecule and preferably identical to the second Fab molecule).

In some embodiments, the second Fab molecule is fused at the C-terminus of the Fab heavy chain to the N-terminus of the Fab heavy chain of the first Fab molecule, and the T cell activating bispecific antigen binding molecule further comprises a third Fab molecule, wherein said third Fab molecule is fused at the N-terminus of the Fab heavy chain to the C-terminus of the Fab heavy chain of the first Fab molecule. In particular such embodiments, said third Fab molecule is a conventional Fab molecule. In other such embodiments, said third Fab molecule is a crossover Fab molecule as described herein, i.e. a Fab molecule wherein the variable domains VH and VL or the constant domains CH1 and CL of the Fab heavy and light chains are exchanged/replaced by each other. In certain such embodiments, the T cell activating bispecific antigen binding molecule essentially consists of the first, the second and the third Fab molecule, and optionally one or more peptide linkers, wherein the second Fab molecule is fused at the C-terminus of the Fab heavy chain to the N-terminus of the Fab heavy chain of the first Fab molecule, and the third Fab molecule is fused at the N-terminus of the Fab heavy chain to the C-terminus of the Fab heavy chain of the first Fab molecule. Such a configuration is schematically depicted in FIGS. 29R and 29V (particular embodiments, wherein the third Fab molecule is a conventional Fab molecule and preferably identical to the first Fab molecule).

In some embodiments, the second Fab molecule is fused at the C-terminus of the Fab heavy chain to the N-terminus of the Fab heavy chain of the first Fab molecule, and the T cell activating bispecific antigen binding molecule further comprises a third Fab molecule, wherein said third Fab molecule is fused at the C-terminus of the Fab heavy chain to the N-terminus of the Fab heavy chain of the second Fab molecule. In particular such embodiments, said third Fab molecule is a crossover Fab molecule as described herein, i.e. a Fab molecule wherein the variable domains VH and VL or the constant domains CH1 and CL of the Fab heavy and light chains are exchanged/replaced by each other. In other such embodiments, said third Fab molecule is a conventional Fab molecule. In certain such embodiments, the T cell activating bispecific antigen binding molecule essentially consists of the first, the second and the third Fab molecule, and optionally one or more peptide linkers, wherein the second Fab molecule is fused at the C-terminus of the Fab heavy chain to the N-terminus of the Fab heavy chain of the first Fab molecule, and the third Fab molecule is fused at the C-terminus of the Fab heavy chain to the N-terminus of the Fab heavy chain of the second Fab molecule. Such a configuration is schematically depicted in FIGS. 29X and 29Z (particular embodiments, wherein the third Fab molecule is a crossover Fab molecule and preferably identical to the first Fab molecule).

In certain embodiments the T cell activating bispecific antigen binding molecule according to the invention comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region) (VH₍₁₎-CH1₍₁₎-VL₍₂₎-CH1₍₂₎). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments the T cell activating bispecific antigen binding molecule according to the invention comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₂₎-CH1₍₂₎-VH₍₁₎-CH1₍₁₎). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments the T cell activating bispecific antigen binding molecule according to the invention comprises a polypeptide wherein the Fab heavy chain variable region of the second Fab molecule shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain of the first Fab molecule (VH₍₂₎-CL₍₂₎-VH₍₁₎-CH1₍₁₎). In some embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎).

In certain embodiments the T cell activating bispecific antigen binding molecule according to the invention comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region) (VH₍₃₎-CH1₍₃₎-VH₍₁₎-CH1₍₁₎-VL₍₂₎-CH1₍₂₎). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the T cell activating bispecific antigen binding molecule further comprises the Fab light chain polypeptide of a third Fab molecule (VL₍₃₎-CL₍₃₎).

In certain embodiments the T cell activating bispecific antigen binding molecule according to the invention comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab heavy chain variable region of the second Fab molecule, which in turn shares a carboxy-terminal peptide bond with the Fab light chain constant region of the second Fab molecule (i.e. the second Fab molecule comprises a crossover Fab heavy chain, wherein the heavy chain constant region is replaced by a light chain constant region) (VH₍₃₎-CH1₍₃₎-VH₍₁₎-CH1₍₁₎-VH₍₂₎-CL₍₂₎). In some embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments the T cell activating bispecific antigen binding molecule further comprises the Fab light chain polypeptide of a third Fab molecule (VL₍₃₎-CL₍₃₎).

In certain embodiments the T cell activating bispecific antigen binding molecule according to the invention comprises a polypeptide wherein the Fab light chain variable region of the second Fab molecule shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule, which in turn shares a carboxy-terminal peptide bond with the Fab heavy chain of a third Fab molecule (VL₍₂₎-CH1₍₂₎-VH₍₁₎-CH1₍₁₎-VH₍₃₎-CH1₍₃₎). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the T cell activating bispecific antigen binding molecule further comprises the Fab light chain polypeptide of a third Fab molecule (VL₍₃₎-CL₍₃₎).

In certain embodiments the T cell activating bispecific antigen binding molecule according to the invention comprises a polypeptide wherein the Fab heavy chain variable region of the second Fab molecule shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain of the first Fab molecule, which in turn shares a carboxy-terminal peptide bond with the Fab heavy chain of a third Fab molecule (VH₍₂₎-CL₍₂₎-VH₍₁₎-CH1₍₁₎-VH₍₃₎-CH1₍₃₎). In some embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments the T cell activating bispecific antigen binding molecule further comprises the Fab light chain polypeptide of a third Fab molecule (VL₍₃₎-CL₍₃₎).

In certain embodiments the T cell activating bispecific antigen binding molecule according to the invention comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab light chain variable region of a third Fab molecule, which in turn shares a carboxy-terminal peptide bond with the Fab heavy chain constant region of a third Fab molecule (i.e. the third Fab molecule comprises a crossover Fab heavy chain, wherein the heavy chain variable region is replaced by a light chain variable region) (VH₍₁₎-CH1₍₁₎-VL₍₂₎-CH1₍₂₎-VL₍₃₎-CH1₍₃₎). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the T cell activating bispecific antigen binding molecule further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments the T cell activating bispecific antigen binding molecule according to the invention comprises a polypeptide wherein the Fab heavy chain of the first Fab molecule shares a carboxy-terminal peptide bond with the Fab heavy chain variable region of the second Fab molecule, which in turn shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain variable region of a third Fab molecule, which in turn shares a carboxy-terminal peptide bond with the Fab light chain constant region of a third Fab molecule (i.e. the third Fab molecule comprises a crossover Fab heavy chain, wherein the heavy chain constant region is replaced by a light chain constant region) (VH₍₁₎-CH1₍₁₎-VH₍₂₎-CL₍₂₎-VH₍₃₎-CL₍₃₎). In some embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments the T cell activating bispecific antigen binding molecule further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

In certain embodiments the T cell activating bispecific antigen binding molecule according to the invention comprises a polypeptide wherein the Fab light chain variable region of a third Fab molecule shares a carboxy-terminal peptide bond with the Fab heavy chain constant region of a third Fab molecule (i.e. the third Fab molecule comprises a crossover Fab heavy chain, wherein the heavy chain variable region is replaced by a light chain variable region), which in turn shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₃₎-CH1₍₃₎-VL₍₂₎-CH1₍₂₎-VH₍₁₎-CH1₍₁₎). In some embodiments the T cell activating bispecific antigen binding molecule further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments the T cell activating bispecific antigen binding molecule further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments the T cell activating bispecific antigen binding molecule according to the invention comprises a polypeptide wherein the Fab heavy chain variable region of a third Fab molecule shares a carboxy-terminal peptide bond with the Fab light chain constant region of a third Fab molecule (i.e. the third Fab molecule comprises a crossover Fab heavy chain, wherein the heavy chain constant region is replaced by a light chain constant region), which in turn shares a carboxy-terminal peptide bond with the Fab heavy chain variable region of the second Fab molecule, which in turn shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain of the first Fab molecule (VH₍₃₎-CL₍₃₎-VH₍₂₎-CL₍₂₎-VH₍₁₎-CH1₍₁₎). In some embodiments the T cell activating bispecific antigen binding molecule further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments the T cell activating bispecific antigen binding molecule further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

According to any of the above embodiments, components of the T cell activating bispecific antigen binding molecule (e.g. Fab molecules, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally an integer from 1 to 10, typically from 2 to 4.

### Fc Domain

The Fc domain of the T cell activating bispecific antigen binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the T cell activating bispecific antigen binding molecule of the invention comprises not more than one Fc domain.

In one embodiment according the invention the Fc domain of the T cell activating bispecific antigen binding molecule is an IgG Fc domain. In a particular embodiment the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is human. An exemplary sequence of a human IgG₁ Fc region is given in SEQ ID NO: 150.

Fc Domain Modifications Promoting Heterodimerization

T cell activating bispecific antigen binding molecules according to the invention comprise different Fab molecules, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of T cell activating bispecific antigen binding molecules in recombinant production, it will thus be advantageous to introduce in the Fc domain of the T cell activating bispecific antigen binding molecule a modification promoting the association of the desired polypeptides.

Accordingly, in particular embodiments the Fc domain of the T cell activating bispecific antigen binding molecule according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain. There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homdimers between the two first or the two second CH3 domains are formed). These different approaches for improved heavy chain heterodimerization are contemplated as different alternatives in combination with the heavy-light chain modifications (VH and VL exchange/replacement in one binding arm and the introduction of substitutions of charged amino acids with opposite charges in the CH1/CL interface) in the T cell activating bispecific antigen binding molecule according to the invention which reduce light chain mispairing and Bence Jones-type side products.

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. No. 5,731,168; U.S. Pat. No. 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the T cell activating bispecific antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain (the “knobs” subunit) the threonine residue at position 366 is replaced with a tryptophan residue (T366W), and in the CH3 domain of the second subunit of the Fc domain (the “hole” subunit) the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the Fc domain comprises amino acid substitutions S354C and T366W, and the second subunit of the Fc domain comprises amino acid substitutions Y349C, T366S, L368A and Y407V (numbering according to Kabat EU index).

In a particular embodiment the Fab molecule which specifically binds an activating T cell antigen is fused (optionally via a Fab molecule which specifically binds to Robo 4) to the first subunit of the Fc domain (comprising the “knob” modification). Without wishing to be bound by theory, fusion of the Fab molecule which specifically binds an activating T cell antigen to the knob-containing subunit of the Fc domain will (further) minimize the generation of antigen binding molecules comprising two Fab molecules which bind to an activating T cell antigen (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment the heterodimerization approach described in EP 1870459 A1, is used alternatively. This approach is based on the introduction of charged amino acids with opposite charges at specific amino acid positions in the CH3/CH3 domain interface between the two subunits of the Fc domain. One preferred embodiment for the T cell activating bispecific antigen binding molecule of the invention are amino acid mutations R409D; K370E in one of the two CH3 domains (of the Fc domain) and amino acid mutations D399K; E357K in the other one of the CH3 domains of the Fc domain (numbering according to Kabat EU index).

In another embodiment the T cell activating bispecific antigen binding molecule of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another embodiment T cell activating bispecific antigen binding molecule of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said T cell activating bispecific antigen binding molecule comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one embodiment the heterodimerization approach described in WO 2013/157953 is used alternatively. In one embodiment a first CH3 domain comprises amino acid mutation T366K and a second CH3 domain comprises amino acid mutation L351D (numberings according to Kabat EU index). In a further embodiment the first CH3 domain comprises further amino acid mutation L351K. In a further embodiment the second CH3 domain comprises further an amino acid mutation selected from Y349E, Y349D and L368E (preferably L368E) (numberings according to Kabat EU index).

In one embodiment the heterodimerization approach described in WO 2012/058768 is used alternatively. In one embodiment a first CH3 domain comprises amino acid mutations L351Y, Y407A and a second CH3 domain comprises amino acid mutations T366A, K409F. In a further embodiment the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F4051, F405M, F405T, F405S, F405V or F405W, e) N390R, N390K or N390D, f) K392V, K392M, K392R, K392L, K392F or K392E (numberings according to Kabat EU index). In a further embodiment a first CH3 domain comprises amino acid mutations L351Y, Y407A and a second CH3 domain comprises amino acid mutations T366V, K409F. In a further embodiment a first CH3 domain comprises amino acid mutation Y407A and a second CH3 domain comprises amino acid mutations T366A, K409F. In a further embodiment the second CH3 domain further comprises amino acid mutations K392E, T411E, D399R and S400R (numberings according to Kabat EU index).

In one embodiment the heterodimerization approach described in WO 2011/143545 is used alternatively, e.g. with the amino acid modification at a position selected from the group consisting of 368 and 409 (numbering according to Kabat EU index).

In one embodiment the heterodimerization approach described in WO 2011/090762, which also uses the knobs-into-holes technology described above, is used alternatively. In one embodiment a first CH3 domain comprises amino acid mutation T366W and a second CH3 domain comprises amino acid mutation Y407A. In one embodiment a first CH3 domain comprises amino acid mutation T366Y and a second CH3 domain comprises amino acid mutation Y407T (numberings according to Kabat EU index).

In one embodiment the T cell activating bispecific antigen binding molecule or its Fc domain is of IgG₂ subclass and the heterodimerization approach described in WO 2010/129304 is used alternatively.

In an alternative embodiment a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment a first CH3 domain comprises amino acid substitution of K392 or N392 with a negatively charged amino acid (e.g. glutamic acid (E), or aspartic acid (D), preferably K392D or N392D) and a second CH3 domain comprises amino acid substitution of D399, E356, D356, or E357 with a positively charged amino acid (e.g. lysine (K) or arginine (R), preferably D399K, E356K, D356K, or E357K, and more preferably D399K and E356K). In a further embodiment the first CH3 domain further comprises amino acid substitution of K409 or R409 with a negatively charged amino acid (e.g. glutamic acid (E), or aspartic acid (D), preferably K409D or R409D). In a further embodiment the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further embodiment the heterodimerization approach described in WO 2007/147901 is used alternatively. In one embodiment a first CH3 domain comprises amino acid mutations K253E, D282K, and K322D and a second CH3 domain comprises amino acid mutations D239K, E240K, and K292D (numberings according to Kabat EU index).

In still another embodiment the heterodimerization approach described in WO 2007/110205 can be used alternatively.

In one embodiment, the first subunit of the Fc domain comprises amino acid substitutions K392D and K409D, and the second subunit of the Fc domain comprises amino acid substitutions D356K and D399K (numbering according to Kabat EU index).

Fc Domain Modifications Reducing Fc Receptor Binding and/or Effector Function

The Fc domain confers to the T cell activating bispecific antigen binding molecule favorable pharmacokinetic properties, including a long serum half-life which contributes to good accumulation in the target tissue and a favorable tissue-blood distribution ratio. At the same time it may, however, lead to undesirable targeting of the T cell activating bispecific antigen binding molecule to cells expressing Fc receptors rather than to the preferred antigen-bearing cells. Moreover, the co-activation of Fc receptor signaling pathways may lead to cytokine release which, in combination with the T cell activating properties and the long half-life of the antigen binding molecule, results in excessive activation of cytokine receptors and severe side effects upon systemic administration. Activation of (Fc receptor-bearing) immune cells other than T cells may even reduce efficacy of the T cell activating bispecific antigen binding molecule due to the potential destruction of T cells e.g. by NK cells.

Accordingly, in particular embodiments, the Fc domain of the T cell activating bispecific antigen binding molecules according to the invention exhibits reduced binding affinity to an Fc receptor and/or reduced effector function, as compared to a native IgG₁ Fc domain. In one such embodiment the Fc domain (or the T cell activating bispecific antigen binding molecule comprising said Fc domain) exhibits less than 50%, preferably less than 20%, more preferably less than 10% and most preferably less than 5% of the binding affinity to an Fc receptor, as compared to a native IgG₁ Fc domain (or a T cell activating bispecific antigen binding molecule comprising a native IgG₁ Fc domain), and/or less than 50%, preferably less than 20%, more preferably less than 10% and most preferably less than 5% of the effector function, as compared to a native IgG₁ Fc domain domain (or a T cell activating bispecific antigen binding molecule comprising a native IgG₁ Fc domain). In one embodiment, the Fc domain domain (or the T cell activating bispecific antigen binding molecule comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or the T cell activating bispecific antigen binding molecule comprising said Fc domain) exhibits greater than about 70%, particularly greater than about 80%, more particularly greater than about 90% of the binding affinity of a native IgG₁ Fc domain (or the T cell activating bispecific antigen binding molecule comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the T cell activating bispecific antigen binding molecule comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the T cell activating bispecific antigen binding molecule comprising an engineered Fc domain exhibits less than 20%, particularly less than 10%, more particularly less than 5% of the binding affinity to an Fc receptor as compared to a T cell activating bispecific antigen binding molecule comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the T cell activating bispecific antigen binding molecule comprising said Fc domain) exhibits greater than about 70% of the binding affinity of a non-engineered form of the Fc domain (or the T cell activating bispecific antigen binding molecule comprising said non-engineered form of the Fc domain) to FcRn. The Fc domain, or T cell activating bispecific antigen binding molecules of the invention comprising said Fc domain, may exhibit greater than about 80% and even greater than about 90% of such affinity. In certain embodiments the Fc domain of the T cell activating bispecific antigen binding molecule is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a T cell activating bispecific antigen binding molecule comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329 (numberings according to Kabat EU index). In a more specific embodiment the Fc domain comprises an amino acid substitution at a position selected from the group of L234, L235 and P329 (numberings according to Kabat EU index). In some embodiments the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G (numberings according to Kabat EU index). In one embodiment the Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the Fc domain comprises amino acid substitutions at positions P329, L234 and L235(numberings according to Kabat EU index). In more particular embodiments the Fc domain comprises the amino acid mutations L234A, L235A and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the T cell activating bispecific antigen binding molecules of the invention is an IgG₄ Fc domain, particularly a human IgG₄ Fc domain. In one embodiment the IgG₄ Fc domain comprises amino acid substitutions at position S228, specifically the amino acid substitution S228P (numberings according to Kabat EU index). To further reduce its binding affinity to an Fc receptor and/or its effector function, in one embodiment the IgG₄ Fc domain comprises an amino acid substitution at position L235, specifically the amino acid substitution L235E (numberings according to Kabat EU index). In another embodiment, the IgG₄ Fc domain comprises an amino acid substitution at position P329, specifically the amino acid substitution P329G (numberings according to Kabat EU index). In a particular embodiment, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In a particular embodiment the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain embodiments N-glycosylation of the Fc domain has been eliminated. In one such embodiment the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or a T cell activating bispecific antigen binding molecule comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTIrm non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. Clq binding assays may be carried out to determine whether the T cell activating bispecific antigen binding molecule is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Antigen Binding Moieties

The antigen binding molecule of the invention is bispecific, i.e. it comprises at least two antigen binding moieties capable of specific binding to two distinct antigens. According to particular embodiments of the invention, the antigen binding moieties are Fab molecules (i.e. antigen binding domains composed of a heavy and a light chain, each comprising a variable and a constant region). In one embodiment said Fab molecules are human. In another embodiment said Fab molecules are humanized. In yet another embodiment said Fab molecules comprise human heavy and light chain constant regions.

Preferably, at least one of the antigen binding moieties is a crossover Fab molecule. Such modification reduces mispairing of heavy and light chains from different Fab molecules, thereby improving the yield and purity of the T cell activating bispecific antigen binding molecule of the invention in recombinant production. In a particular crossover Fab molecule useful for the T cell activating bispecific antigen binding molecule of the invention, the variable domains of the Fab light chain and the Fab heavy chain (VL and VH, respectively) are exchanged. Even with this domain exchange, however, the preparation of the T cell activating bispecific antigen binding molecule may comprise certain side products due to a so-called Bence Jones-type interaction between mispaired heavy and light chains (see Schaefer et al, PNAS, 108 (2011) 11187-11191). To further reduce mispairing of heavy and light chains from different Fab molecules and thus increase the purity and yield of the desired T cell activating bispecific antigen binding molecule, according to the present invention charged amino acids with opposite charges may be introduced at specific amino acid positions in the CH1 and CL domains of either the Fab molecule(s) specifically binding to a target cell antigen, or the Fab molecule specifically binding to an activating T cell antigen. Charge modifications are made either in the conventional Fab molecule(s) comprised in the T cell activating bispecific antigen binding molecule (such as shown e.g. in FIGS. 29 A-C, G-J), or in the VH/VL crossover Fab molecule(s) comprised in the T cell activating bispecific antigen binding molecule (such as shown e.g. in FIG. 29 D-F, K—N) (but not in both). In particular embodiments, the charge modifications are made in the conventional Fab molecule(s) comprised in the T cell activating bispecific antigen binding molecule (which in particular embodiments specifically bind(s) to the target cell antigen).

In a particular embodiment according to the invention, the T cell activating bispecific antigen binding molecule is capable of simultaneous binding to Robo 4 and an activating T cell antigen, particularly CD3. In one embodiment, the T cell activating bispecific antigen binding molecule is capable of crosslinking a T cell and a Robo 4 expressing target cell by simultaneous binding to Robo 4 and an activating T cell antigen. In an even more particular embodiment, such simultaneous binding results in lysis of the target cell, particularly an endothelial cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the T cell activating bispecific antigen binding molecule to the activating T cell antigen without simultaneous binding to Robo 4 does not result in T cell activation.

In one embodiment, the T cell activating bispecific antigen binding molecule is capable of redirecting cytotoxic activity of a T cell to a Robo 4 expressing target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

Activating T Cell Antigen Binding Moiety

The T cell activating bispecific antigen binding molecule of the invention comprises at least one antigen binding moiety, particularly a Fab molecule, which specifically binds to an activating T cell antigen (also referred to herein as an “activating T cell antigen binding moiety, or activating T cell antigen binding Fab molecule”). In a particular embodiment, the T cell activating bispecific antigen binding molecule comprises not more than one antigen binding moiety capable of specific binding to an activating T cell antigen. In one embodiment the T cell activating bispecific antigen binding molecule provides monovalent binding to the activating T cell antigen. In particular embodiments, the antigen binding moiety which specifically binds an activating T cell antigen is a crossover Fab molecule as described herein, i.e. a Fab molecule wherein the variable domains VH and VL or the constant domains CH1 and CL of the Fab heavy and light chains are exchanged/replaced by each other. In such embodiments, the antigen binding moiety(ies) which specifically binds a target cell antigen is preferably a conventional Fab molecule. In embodiments where there is more than one antigen binding moiety, particularly Fab molecule, which specifically binds to a target cell antigen comprised in the T cell activating bispecific antigen binding molecule, the antigen binding moiety which specifically binds to an activating T cell antigen preferably is a crossover Fab molecule and the antigen binding moieties which specifically bind to a target cell antigen are conventional Fab molecules.

In alternative embodiments, the antigen binding moiety which specifically binds an activating T cell antigen is a conventional Fab molecule. In such embodiments, the antigen binding moiety(ies) which specifically binds a target cell antigen is a crossover Fab molecule as described herein, i.e. a Fab molecule wherein the variable domains VH and VL or the constant domains CH1 and CL of the Fab heavy and light chains are exchanged/replaced by each other. In a particular embodiment the activating T cell antigen is CD3, particularly human CD3 or cynomolgus CD3, most particularly human CD3. In a particular embodiment the activating T cell antigen binding moiety is cross-reactive for (i.e. specifically binds to) human and cynomolgus CD3. In some embodiments, the activating T cell antigen is the epsilon subunit of CD3 (CD3ε), particulary human CD3ε(SEQ ID NO: 136) or cynomolgus CD3ε(SEQ ID NO: 137), most particularly human CD3ε.

In some embodiments, the activating T cell antigen binding moiety specifically binds to CD3, particularly CD3 epsilon, and comprises at least one heavy chain complementarity determining region (CDR) selected from the group consisting of SEQ ID NO: 141, SEQ ID NO: 142 and SEQ ID NO: 143 and at least one light chain CDR selected from the group of SEQ ID NO: 145, SEQ ID NO: 146, SEQ ID NO: 147.

In one embodiment the CD3 binding antigen binding moiety, particularly Fab molecule, comprises a heavy chain variable region comprising the heavy chain CDR1 of SEQ ID NO: 141, the heavy chain CDR2 of SEQ ID NO: 142, the heavy chain CDR3 of SEQ ID NO: 143, and a light chain variable region comprising the light chain CDR1 of SEQ ID NO: 145, the light chain CDR2 of SEQ ID NO: 146, and the light chain CDR3 of SEQ ID NO: 147.

In one embodiment the CD3 binding antigen binding moiety, particularly Fab molecule, comprises a heavy chain variable region sequence that is at least about 95%, 96%, 97%, 98%, 99% or 100% identical to SEQ ID NO: 140 and a light chain variable region sequence that is at least about 95%, 96%, 97%, 98%, 99% or 100% identical to SEQ ID NO: 144.

In one embodiment the CD3 binding antigen binding moiety, particularly Fab molecule, comprises a heavy chain variable region comprising the amino acid sequence of SEQ ID NO: 140 and a light chain variable region comprising the amino acid sequence of SEQ ID NO: 144. In one embodiment the CD3 binding antigen binding moiety, particularly Fab molecule, comprises the heavy chain variable region sequence of SEQ ID NO: 140 and the light chain variable region sequence of SEQ ID NO: 144.

In one embodiment, the activating T cell antigen binding moiety can compete with monoclonal antibody H2C (described in PCT publication no. WO 2008/119567) for binding an epitope of CD3. In another embodiment, the activating T cell antigen binding moiety can compete with monoclonal antibody V9 (described in Rodrigues et al., Int J Cancer Suppl 7, 45-50 (1992) and U.S. Pat. No. 6,054,297) for binding an epitope of CD3. In yet another embodiment, the activating T cell antigen binding moiety can compete with monoclonal antibody FN18 (described in Nooij et al., Eur J Immunol 19, 981-984 (1986)) for binding an epitope of CD3. In a particular embodiment, the activating T cell antigen binding moiety can compete with monoclonal antibody SP34 (described in Pessano et al., EMBO J 4, 337-340 (1985)) for binding an epitope of CD3. In one embodiment, the activating T cell antigen binding moiety binds to the same epitope of CD3 as monoclonal antibody SP34. In one embodiment, the activating T cell antigen binding moiety comprises the heavy chain CDR1 of SEQ ID NO: 122, the heavy chain CDR2 of SEQ ID NO: 123, the heavy chain CDR3 of SEQ ID NO: 124, the light chain CDR1 of SEQ ID NO: 125, the light chain CDR2 of SEQ ID NO: 126, and the light chain CDR3 of SEQ ID NO: 127. In a further embodiment, the activating T cell antigen binding moiety comprises a heavy chain variable region sequence that is at least about 80%, 85%, 90%, 95%, 96%, 97%, 98%, 99% or 100% identical to SEQ ID NO: 85 and a light chain variable region sequence that is at least about 80%, 85%, 90%, 95%, 96%, 97%, 98%, 99% or 100% identical to SEQ ID NO: 87, or variants thereof that retain functionality.

In one embodiment, the activating T cell antigen binding moiety comprises the heavy chain CDR1 of SEQ ID NO: 128, the heavy chain CDR2 of SEQ ID NO: 129, the heavy chain CDR3 of SEQ ID NO: 130, the light chain CDR1 of SEQ ID NO: 131, the light chain CDR2 of SEQ ID NO: 132, and the light chain CDR3 of SEQ ID NO: 133. In one embodiment, the activating T cell antigen binding moiety can compete for binding an epitope of CD3 with an antigen binding moiety comprising the heavy chain CDR1 of SEQ ID NO: 128, the heavy chain CDR2 of SEQ ID NO: 129, the heavy chain CDR3 of SEQ ID NO: 130, the light chain CDR1 of SEQ ID NO: 131, the light chain CDR2 of SEQ ID NO: 132, and the light chain CDR3 of SEQ ID NO: 133. In one embodiment, the activating T cell antigen binding moiety binds to the same epitope of CD3 as an antigen binding moiety comprising the heavy chain CDR1 of SEQ ID NO: 128, the heavy chain CDR2 of SEQ ID NO: 129, the heavy chain CDR3 of SEQ ID NO: 130, the light chain CDR1 of SEQ ID NO: 131, the light chain CDR2 of SEQ ID NO: 132, and the light chain CDR3 of SEQ ID NO: 133. In a further embodiment, the activating T cell antigen binding moiety comprises a heavy chain variable region sequence that is at least about 80%, 85%, 90%, 95%, 96%, 97%, 98%, 99% or 100% identical to SEQ ID NO: 134 and a light chain variable region sequence that is at least about 80%, 85%, 90%, 95%, 96%, 97%, 98%, 99% or 100% identical to SEQ ID NO: 135, or variants thereof that retain functionality. In one embodiment, the activating T cell antigen binding moiety can compete for binding an epitope of CD3 with an antigen binding moiety comprising the heavy chain variable region sequence of SEQ ID NO: 134 and the light chain variable region sequence of SEQ ID NO: 135. In one embodiment, the activating T cell antigen binding moiety binds to the same epitope of CD3 as an antigen binding moiety comprising the heavy chain variable region sequence of SEQ ID NO: 134 and the light chain variable region sequence of SEQ ID NO: 135. In another embodiment, the activating T cell antigen binding moiety comprises a humanized version of the heavy chain variable region sequence of SEQ ID NO: 134 and a humanized version of the light chain variable region sequence of SEQ ID NO: 135. In one embodiment, the activating T cell antigen binding moiety comprises the heavy chain CDR1 of SEQ ID NO: 128, the heavy chain CDR2 of SEQ ID NO: 129, the heavy chain CDR3 of SEQ ID NO: 130, the light chain CDR1 of SEQ ID NO: 131, the light chain CDR2 of SEQ ID NO: 132, the light chain CDR3 of SEQ ID NO: 133, and human heavy and light chain variable region framework sequences.

Robo 4 Antigen Binding Moiety

The T cell activating bispecific antigen binding molecule of the invention comprises at least one antigen binding moiety, particularly a Fab molecule, which specifically binds to Robo 4 (also referred to herein as a “Robo 4 antigen binding moiety”). In certain embodiments, the T cell activating bispecific antigen binding molecule comprises more than one, particularly two, antigen binding moieties, particularly Fab molecules, which specifically bind to Robo 4. In such embodiments the T cell activating bispecific antigen binding molecule provides multivalent, particularly bivalent, binding to Robo 4. In a particular such embodiment, each of these antigen binding moieties specifically binds to the same antigenic determinant. In an even more particular embodiment, all of these antigen binding moieties are identical, i.e. they comprise the same amino acid sequences including the same amino acid substitutions in the CH1 and CL domain as described herein (if any). In one embodiment, the T cell activating bispecific antigen binding molecule comprises an immunoglobulin molecule which specifically binds to Robo 4. In one embodiment the T cell activating bispecific antigen binding molecule comprises not more than two antigen binding moieties, particularly Fab molecules, which specifically bind to Robo 4. In particular embodiments, the antigen binding moiety(ies) which specifically bind to Robo 4 is/are a conventional Fab molecule. In such embodiments, the antigen binding moiety(ies) which specifically binds an activating T cell antigen is a crossover Fab molecule as described herein, i.e. a Fab molecule wherein the variable domains VH and VL or the constant domains CH1 and CL of the Fab heavy and light chains are exchanged/replaced by each other.

In alternative embodiments, the antigen binding moiety(ies) which specifically bind to Robo 4 is/are a crossover Fab molecule as described herein, i.e. a Fab molecule wherein the variable domains VH and VL or the constant domains CH1 and CL of the Fab heavy and light chains are exchanged/replaced by each other. In such embodiments, the antigen binding moiety(ies) which specifically binds an activating T cell antigen is a conventional Fab molecule.

The Robo 4 binding moiety is able to direct the T cell activating bispecific antigen binding molecule to a target site, for example to a specific type of cell that expresses Robo 4 (such as a tumor endothelial cell).

In a particular embodiment, the Robo 4 is human Robo 4 (SEQ ID NO: 138). In another embodiment, the Robo 4 is cynomolgus monkey (Macaca fascicularis) Robo 4. In yet another embodiment, the Robo 4 is mouse Robo 4 (SEQ ID NO: 139). In some embodiments the Robo 4 antigen binding moiety is cross-reactive for (i.e. specifically binds to) (i) human and cynomolgus Robo 4, (ii) human and mouse Robo 4, or (iii) human, cynomolgus and mouse Robo 4. In a particular embodiment, the Robo 4 antigen binding moiety binds to the extracellular domain (ECD) of Robo 4.

As shown in the Examples, anti-Robo 4 monoclonal antibody clones “01E06” (shown in SEQ ID NO: 19 (VH) and SEQ ID NO: 21 (VL)), “01F09” (shown in SEQ ID NO: 27 (VH) and SEQ ID NO: 29 (VL)) and “7G2” (shown in SEQ ID NO: 31 (VH) and SEQ ID NO: 33 (VL)) bind to the Ig-like domain 1 and/or 2 of Robo 4. Accordingly, in some embodiments, the Robo 4 antigen binding moiety specifically binds to an epitope in the Ig-like domain 1 (position 20-119 of SEQ ID NO: 15) and/or the Ig-like domain 2 (position 20-107 of SEQ ID NO: 17) of the extracellular domain of Robo 4. In one such embodiment, the Robo 4 antigen binding moiety can compete with monoclonal antibody 01E06 for binding an epitope of Robo 4. In another embodiment, the Robo 4 antigen binding moiety can compete with monoclonal antibody 01F09 for binding an epitope of Robo 4. In yet another embodiment, the Robo 4 antigen binding moiety can compete with monoclonal antibody 7G2 for binding an epitope of Robo 4.

In a specific embodiment, the Robo 4 antigen binding moiety comprises the heavy chain CDR1 of SEQ ID NO: 91, the heavy chain CDR2 of SEQ ID NO: 92, the heavy chain CDR3 of SEQ ID NO: 93, the light chain CDR1 of SEQ ID NO: 94, the light chain CDR2 of SEQ ID NO: 95, and the light chain CDR3 of SEQ ID NO: 96. In a further specific embodiment, the Robo 4 antigen binding moiety comprises a heavy chain variable region sequence that is at least about 80%, 85%, 90%, 95%, 96%, 97%, 98%, 99% or 100% identical to SEQ ID NO: 19 and a light chain variable region sequence that is at least about 80%, 85%, 90%, 95%, 96%, 97%, 98%, 99% or 100% identical to SEQ ID NO: 21, or variants thereof that retain functionality. In one embodiment, the Robo 4 antigen binding moiety comprises the heavy chain variable region sequence of SEQ ID NO: 19, and the light chain variable region sequence of SEQ ID NO: 21. In another embodiment, the Robo 4 antigen binding moiety comprises a humanized version of the heavy chain variable region sequence of SEQ ID NO: 19 and a humanized version of the light chain variable region sequence of SEQ ID NO: 21. In one embodiment, the Robo 4 antigen binding moiety comprises the heavy chain CDR1 of SEQ ID NO: 91, the heavy chain CDR2 of SEQ ID NO: 92, the heavy chain CDR3 of SEQ ID NO: 93, the light chain CDR1 of SEQ ID NO: 94, the light chain CDR2 of SEQ ID NO: 95, the light chain CDR3 of SEQ ID NO: 96, and human heavy and light chain variable region framework sequences.

In another specific embodiment, the Robo 4 antigen binding moiety comprises the heavy chain CDR1 of SEQ ID NO: 103, the heavy chain CDR2 of SEQ ID NO: 104, the heavy chain CDR3 of SEQ ID NO: 105, the light chain CDR1 of SEQ ID NO: 106, the light chain CDR2 of SEQ ID NO: 107, and the light chain CDR3 of SEQ ID NO: 108. In a further specific embodiment, the Robo 4 antigen binding moiety comprises a heavy chain variable region sequence that is at least about 80%, 85%, 90%, 95%, 96%, 97%, 98%, 99% or 100% identical to SEQ ID NO: 27 and a light chain variable region sequence that is at least about 80%, 85%, 90%, 95%, 96%, 97%, 98%, 99% or 100% identical to SEQ ID NO: 29, or variants thereof that retain functionality. In one embodiment, the Robo 4 antigen binding moiety comprises the heavy chain variable region sequence of SEQ ID NO: 27, and the light chain variable region sequence of SEQ ID NO: 29. In another embodiment, the Robo 4 antigen binding moiety comprises a humanized version of the heavy chain variable region sequence of SEQ ID NO: 27 and a humanized version of the light chain variable region sequence of SEQ ID NO: 29. In one embodiment, the Robo 4 antigen binding moiety comprises the heavy chain CDR1 of SEQ ID NO: 103, the heavy chain CDR2 of SEQ ID NO: 104, the heavy chain CDR3 of SEQ ID NO: 105, the light chain CDR1 of SEQ ID NO: 106, the light chain CDR2 of SEQ ID NO: 107, the light chain CDR3 of SEQ ID NO: 108, and human heavy and light chain variable region framework sequences.

In yet a further specific embodiment, the Robo 4 antigen binding moiety comprises the heavy chain CDR1 of SEQ ID NO: 109, the heavy chain CDR2 of SEQ ID NO: 110, the heavy chain CDR3 of SEQ ID NO: 111, the light chain CDR1 of SEQ ID NO: 112, the light chain CDR2 of SEQ ID NO: 113, and the light chain CDR3 of SEQ ID NO: 114. In a further specific embodiment, the Robo 4antigen binding moiety comprises a heavy chain variable region sequence that is at least about 80%, 85%, 90%, 95%, 96%, 97%, 98%, 99% or 100% identical to SEQ ID NO: 31 and a light chain variable region sequence that is at least about 80%, 85%, 90%, 95%, 96%, 97%, 98%, 99% or 100% identical to SEQ ID NO: 33, or variants thereof that retain functionality. In one embodiment, the Robo 4 antigen binding moiety comprises the heavy chain variable region sequence of SEQ ID NO: 31, and the light chain variable region sequence of SEQ ID NO: 33.

As shown in the Examples, anti-Robo 4 monoclonal antibody clone “01F05” (shown in SEQ ID NO: 23 (VH) and SEQ ID NO: 25 (VL)), binds to the fibronectin (FN)-like domain 2 of Robo 4. Hence, in some embodiments, the Robo 4 antigen binding moiety specifically binds to an epitope in the fibronectin-like domain 1 (position 20-108 of SEQ ID NO: 11) and/or the fibronectin-like domain 2 (position 20-111 of SEQ ID NO: 11) of the extracellular domain of Robo 4. In one such embodiment, the Robo 4 antigen binding moiety can compete with monoclonal antibody 01F05 for binding an epitope of Robo 4.

In a particular embodiment, the Robo 4 antigen binding moiety comprises the heavy chain CDR1 of SEQ ID NO: 97, the heavy chain CDR2 of SEQ ID NO: 98, the heavy chain CDR3 of SEQ ID NO: 99, the light chain CDR1 of SEQ ID NO: 100, the light chain CDR2 of SEQ ID NO: 101, and the light chain CDR3 of SEQ ID NO: 102. In a further specific embodiment, the Robo 4 antigen binding moiety comprises a heavy chain variable region sequence that is at least about 80%, 85%, 90%, 95%, 96%, 97%, 98%, 99% or 100% identical to SEQ ID NO: 23 and a light chain variable region sequence that is at least about 80%, 85%, 90%, 95%, 96%, 97%, 98%, 99% or 100% identical to SEQ ID NO: 25, or variants thereof that retain functionality. In one embodiment, the Robo 4 antigen binding moiety comprises the heavy chain variable region sequence of SEQ ID NO: 23, and the light chain variable region sequence of SEQ ID NO: 25. In another embodiment, the Robo 4 antigen binding moiety comprises a humanized version of the heavy chain variable region sequence of SEQ ID NO: 23 and a humanized version of the light chain variable region sequence of SEQ ID NO: 25. In one embodiment, the Robo 4 antigen binding moiety comprises the heavy chain CDR1 of SEQ ID NO: 97, the heavy chain CDR2 of SEQ ID NO: 98, the heavy chain CDR3 of SEQ ID NO: 99, the light chain CDR1 of SEQ ID NO: 100, the light chain CDR2 of SEQ ID NO: 101, the light chain CDR3 of SEQ ID NO: 102, and human heavy and light chain variable region framework sequences.

In a particular embodiment, the T cell activating bispecific antigen binding molecule comprises a polypeptide that is at least 95%, 96%, 97%, 98%, or 99% identical to the sequence of SEQ ID NO: 151, a polypeptide that is at least 95%, 96%, 97%, 98%, or 99% identical to the sequence of SEQ ID NO: 152, a polypeptide that is at least 95%, 96%, 97%, 98%, or 99% identical to the sequence of SEQ ID NO: 153, and a polypeptide that is at least 95%, 96%, 97%, 98%, or 99% identical to the sequence of SEQ ID NO: 154. In a further particular embodiment, the T cell activating bispecific antigen binding molecule comprises a polypeptide sequence of SEQ ID NO: 151, a polypeptide sequence of SEQ ID NO: 152, a polypeptide sequence of SEQ ID NO: 153 and a polypeptide sequence of SEQ ID NO: 154.

Robo 4 Antibodies

The invention also provides antibodies which specifically bind to Robo 4 (also referred to herein as “Robo 4 antibody”).

As shown in the Examples, anti-Robo 4 monoclonal antibody clones “01E06” (shown in SEQ ID NO: 19 (VH) and SEQ ID NO: 21 (VL)), “01F09” (shown in SEQ ID NO: 27 (VH) and SEQ ID NO: 29 (VL)) and “7G2” (shown in SEQ ID NO: 31 (VH) and SEQ ID NO: 33 (VL)) bind to the Ig-like domain 1 and/or 2 of Robo 4. Accordingly, in some embodiments, the Robo 4 antibody specifically binds to an epitope in the Ig-like domain 1 (position 20-119 of SEQ ID NO: 15) and/or the Ig-like domain 2 (position 20-107 of SEQ ID NO: 17) of the extracellular domain of Robo 4. In one such embodiment, the Robo 4 antibody can compete with monoclonal antibody 01E06 for binding an epitope of Robo 4. In another embodiment, the Robo 4 antibody can compete with monoclonal antibody 01F09 for binding an epitope of Robo 4. In yet another embodiment, the Robo 4 antibody can compete with monoclonal antibody 7G2 for binding an epitope of Robo 4.

In a specific embodiment, the Robo 4 antibody comprises the heavy chain CDR1 of SEQ ID NO: 91, the heavy chain CDR2 of SEQ ID NO: 92, the heavy chain CDR3 of SEQ ID NO: 93, the light chain CDR1 of SEQ ID NO: 94, the light chain CDR2 of SEQ ID NO: 95, and the light chain CDR3 of SEQ ID NO: 96. In a further specific embodiment, the Robo 4 antibody comprises a heavy chain variable region sequence that is at least about 80%, 85%, 90%, 95%, 96%, 97%, 98%, 99% or 100% identical to SEQ ID NO: 19 and a light chain variable region sequence that is at least about 80%, 85%, 90%, 95%, 96%, 97%, 98%, 99% or 100% identical to SEQ ID NO: 21, or variants thereof that retain functionality. In one embodiment, the Robo 4 antibody comprises the heavy chain variable region sequence of SEQ ID NO: 19, and the light chain variable region sequence of SEQ ID NO: 21. In another embodiment, the Robo 4 antibody comprises a humanized version of the heavy chain variable region sequence of SEQ ID NO: 19 and a humanized version of the light chain variable region sequence of SEQ ID NO: 21. In one embodiment, the Robo 4 antibody comprises the heavy chain CDR1 of SEQ ID NO: 91, the heavy chain CDR2 of SEQ ID NO: 92, the heavy chain CDR3 of SEQ ID NO: 93, the light chain CDR1 of SEQ ID NO: 94, the light chain CDR2 of SEQ ID NO: 95, the light chain CDR3 of SEQ ID NO: 96, and human heavy and light chain variable region framework sequences.

In another specific embodiment, the Robo 4 antibody comprises the heavy chain CDR1 of SEQ ID NO: 103, the heavy chain CDR2 of SEQ ID NO: 104, the heavy chain CDR3 of SEQ ID NO: 105, the light chain CDR1 of SEQ ID NO: 106, the light chain CDR2 of SEQ ID NO: 107, and the light chain CDR3 of SEQ ID NO: 108. In a further specific embodiment, the Robo 4 antibody comprises a heavy chain variable region sequence that is at least about 80%, 85%, 90%, 95%, 96%, 97%, 98%, 99% or 100% identical to SEQ ID NO: 27 and a light chain variable region sequence that is at least about 80%, 85%, 90%, 95%, 96%, 97%, 98%, 99% or 100% identical to SEQ ID NO: 29, or variants thereof that retain functionality. In one embodiment, the Robo 4 antibody comprises the heavy chain variable region sequence of SEQ ID NO: 27, and the light chain variable region sequence of SEQ ID NO: 29. In another embodiment, the Robo 4 antibody comprises a humanized version of the heavy chain variable region sequence of SEQ ID NO: 27 and a humanized version of the light chain variable region sequence of SEQ ID NO: 29. In one embodiment, the Robo 4 antibody comprises the heavy chain CDR1 of SEQ ID NO: 103, the heavy chain CDR2 of SEQ ID NO: 104, the heavy chain CDR3 of SEQ ID NO: 105, the light chain CDR1 of SEQ ID NO: 106, the light chain CDR2 of SEQ ID NO: 107, the light chain CDR3 of SEQ ID NO: 108, and human heavy and light chain variable region framework sequences.

In yet a further specific embodiment, the Robo 4 antibody comprises the heavy chain CDR1 of SEQ ID NO: 109, the heavy chain CDR2 of SEQ ID NO: 110, the heavy chain CDR3 of SEQ ID NO: 111, the light chain CDR1 of SEQ ID NO: 112, the light chain CDR2 of SEQ ID NO: 113, and the light chain CDR3 of SEQ ID NO: 114. In a further specific embodiment, the Robo 4antibody comprises a heavy chain variable region sequence that is at least about 80%, 85%, 90%, 95%, 96%, 97%, 98%, 99% or 100% identical to SEQ ID NO: 31 and a light chain variable region sequence that is at least about 80%, 85%, 90%, 95%, 96%, 97%, 98%, 99% or 100% identical to SEQ ID NO: 33, or variants thereof that retain functionality. In one embodiment, the Robo 4 antibody comprises the heavy chain variable region sequence of SEQ ID NO: 31, and the light chain variable region sequence of SEQ ID NO: 33.

As shown in the Examples, anti-Robo 4 monoclonal antibody clone “01F05” (shown in SEQ ID NO: 23 (VH) and SEQ ID NO: 25 (VL)), binds to the fibronectin (FN)-like domain 2 of Robo 4. Hence, in some embodiments, the Robo 4 antibody specifically binds to an epitope in the fibronectin-like domain 1 (position 20-108 of SEQ ID NO: 11) and/or the fibronectin-like domain 2 (position 20-111 of SEQ ID NO: 11) of the extracellular domain of Robo 4. In one such embodiment, the Robo 4 antibody can compete with monoclonal antibody 01F05 for binding an epitope of Robo 4.

In a particular embodiment, the Robo 4 antibody comprises the heavy chain CDR1 of SEQ ID NO: 97, the heavy chain CDR2 of SEQ ID NO: 98, the heavy chain CDR3 of SEQ ID NO: 99, the light chain CDR1 of SEQ ID NO: 100, the light chain CDR2 of SEQ ID NO: 101, and the light chain CDR3 of SEQ ID NO: 102. In a further specific embodiment, the Robo 4 antibody comprises a heavy chain variable region sequence that is at least about 80%, 85%, 90%, 95%, 96%, 97%, 98%, 99% or 100% identical to SEQ ID NO: 23 and a light chain variable region sequence that is at least about 80%, 85%, 90%, 95%, 96%, 97%, 98%, 99% or 100% identical to SEQ ID NO: 25, or variants thereof that retain functionality. In one embodiment, the Robo 4 antibody comprises the heavy chain variable region sequence of SEQ ID NO: 23, and the light chain variable region sequence of SEQ ID NO: 25. In another embodiment, the Robo 4 antibody comprises a humanized version of the heavy chain variable region sequence of SEQ ID NO: 23 and a humanized version of the light chain variable region sequence of SEQ ID NO: 25. In one embodiment, the Robo 4 antibody comprises the heavy chain CDR1 of SEQ ID NO: 97, the heavy chain CDR2 of SEQ ID NO: 98, the heavy chain CDR3 of SEQ ID NO: 99, the light chain CDR1 of SEQ ID NO: 100, the light chain CDR2 of SEQ ID NO: 101, the light chain CDR3 of SEQ ID NO: 102, and human heavy and light chain variable region framework sequences. In one embodiment the Robo 4 antibody is a full-length antibody. In one embodiment, the Robo 4 antibody is an antibody fragment, such as a Fab molecule, a scFv molecule or the like. In one embodiment the Robo 4 antibody is an IgG molecule, particularly an IgG1 molecule. The IgG molecule may incorporate any of the features described herein in relation to IgG molecules. In one embodiment, the Robo 4 antibody comprises an Fc domain. The Fc domain may incorporate any of the features described herein in relation to Fc domains. In one embodiment the Robo 4 antibody is a multispecific antibody, particularly a bispecific antibody.

Polynucleotides

The invention further provides isolated polynucleotides encoding a T cell activating bispecific antigen binding molecule as described herein or a fragment thereof. In some embodiments, said fragment is an antigen binding fragment.

Polynucleotides of the invention include those that are at least about 80%, 85%, 90%, 95%, 96%, 97%, 98%, 99%, or 100% identical to the sequences set forth in SEQ ID NOs 20, 22, 24, 26, 28, 30, 32, 34, 36, 38, 40, 42, 44, 46, 48, 50, 52, 54, 56, 58, 60, 62, 64, 66, 68, 70, 80, 82 and 84, including functional fragments or variants thereof.

The polynucleotides encoding T cell activating bispecific antigen binding molecules of the invention may be expressed as a single polynucleotide that encodes the entire T cell activating bispecific antigen binding molecule or as multiple (e.g., two or more) polynucleotides that are co-expressed. Polypeptides encoded by polynucleotides that are co-expressed may associate through, e.g., disulfide bonds or other means to form a functional T cell activating bispecific antigen binding molecule. For example, the light chain portion of a Fab molecule may be encoded by a separate polynucleotide from the portion of the T cell activating bispecific antigen binding molecule comprising the heavy chain portion of the Fab molecule, an Fc domain subunit and optionally (part of) another Fab molecule. When co-expressed, the heavy chain polypeptides will associate with the light chain polypeptides to form the Fab molecule. In another example, the portion of the T cell activating bispecific antigen binding molecule comprising one of the two Fc domain subunits and optionally (part of) one or more Fab molecules could be encoded by a separate polynucleotide from the portion of the T cell activating bispecific antigen binding molecule comprising the the other of the two Fc domain subunits and optionally (part of) a Fab molecule. When co-expressed, the Fc domain subunits will associate to form the Fc domain. In some embodiments, the isolated polynucleotide encodes the entire T cell activating bispecific antigen binding molecule according to the invention as described herein. In other embodiments, the isolated polynucleotide encodes a polypeptides comprised in the T cell activating bispecific antigen binding molecule according to the invention as described herein.

In another embodiment, the present invention is directed to an isolated polynucleotide encoding a T cell activating bispecific antigen binding molecule of the invention or a fragment thereof, wherein the polynucleotide comprises a sequence that encodes a variable region sequence as shown in SEQ ID NOs 19, 21, 23, 25, 27, 29, 31, 33, 140 and 144. In another embodiment, the present invention is directed to an isolated polynucleotide encoding a T cell activating bispecific antigen binding molecule or fragment thereof, wherein the polynucleotide comprises a sequence that encodes a polypeptide sequence as shown in SEQ ID NOs 35, 37, 39, 41, 43, 45, 47, 49, 51, 53, 55, 57, 59, 61, 63, 65, 67, 69, 79, 81 and 83, 151-154. In another embodiment, the invention is further directed to an isolated polynucleotide encoding a T cell activating bispecific antigen binding molecule of the invention or a fragment thereof, wherein the polynucleotide comprises a sequence that is at least about 80%, 85%, 90%, 95%, 96%, 97%, 98%, or 99% identical to a nucleotide sequence shown in SEQ ID NOs 20, 22, 24, 26, 28, 30, 32, 34, 36, 38, 40, 42, 44, 46, 48, 50, 52, 54, 56, 58, 60, 62, 64, 66, 68, 70, 80, 82 or 84, 157-162. In another embodiment, the invention is directed to an isolated polynucleotide encoding a T cell activating bispecific antigen binding molecule of the invention or a fragment thereof, wherein the polynucleotide comprises a nucleic acid sequence shown in SEQ ID NOs 20, 22, 24, 26, 28, 30, 32, 34, 36, 38, 40, 42, 44, 46, 48, 50, 52, 54, 56, 58, 60, 62, 64, 66, 68, 70, 80, 82 or 84, 157-162. In another embodiment, the invention is directed to an isolated polynucleotide encoding a T cell activating bispecific antigen binding molecule of the invention or a fragment thereof, wherein the polynucleotide comprises a sequence that encodes a variable region sequence that is at least about 80%, 85%, 90%, 95%, 96%, 97%, 98%, or 99% identical to an amino acid sequence in SEQ ID NOs 19, 21, 23, 25, 27, 29, 31, 33, 140 and 144. In another embodiment, the invention is directed to an isolated polynucleotide encoding a T cell activating bispecific antigen binding molecule or fragment thereof, wherein the polynucleotide comprises a sequence that encodes a polypeptide sequence that is at least 80%, 85%, 90%, 95%, 96%, 97%, 98%, or 99% identical to an amino acid sequence in SEQ ID NOs 35, 37, 39, 41, 43, 45, 47, 49, 51, 53, 55, 57, 59, 61, 63, 65, 67, 69, 79, 81, 83, 151, 152, 153 or 154. The invention encompasses an isolated polynucleotide encoding a T cell activating bispecific antigen binding molecule of the invention or a fragment thereof, wherein the polynucleotide comprises a sequence that encodes the variable region sequence of SEQ ID NOs 19, 21, 23, 25, 27, 29, 31, 33, 140 or 144 with conservative amino acid substitutions. The invention also encompasses an isolated polynucleotide encoding a T cell activating bispecific antigen binding molecule of the invention or fragment thereof, wherein the polynucleotide comprises a sequence that encodes the polypeptide sequence of SEQ ID NOs 35, 37, 39, 41, 43, 45, 47, 49, 51, 53, 55, 57, 59, 61, 63, 65, 67, 69, 79, 81, 83, 151, 152, 153 or 154 with conservative amino acid substitutions.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

T cell activating bispecific antigen binding molecules of the invention may be obtained, for example, by solid-state peptide synthesis (e.g. Merrifield solid phase synthesis) or recombinant production. For recombinant production one or more polynucleotide encoding the T cell activating bispecific antigen binding molecule (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a T cell activating bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the T cell activating bispecific antigen binding molecule (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the T cell activating bispecific antigen binding molecule (fragment) of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the T cell activating bispecific antigen binding molecule is desired, DNA encoding a signal sequence may be placed upstream of the nucleic acid encoding a T cell activating bispecific antigen binding molecule of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the T cell activating bispecific antigen binding molecule may be included within or at the ends of the T cell activating bispecific antigen binding molecule (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) a vector comprising a polynucleotide that encodes (part of) a T cell activating bispecific antigen binding molecule of the invention. As used herein, the term “host cell” refers to any kind of cellular system which can be engineered to generate the T cell activating bispecific antigen binding molecules of the invention or fragments thereof. Host cells suitable for replicating and for supporting expression of T cell activating bispecific antigen binding molecules are well known in the art. Such cells may be transfected or transduced as appropriate with the particular expression vector and large quantities of vector containing cells can be grown for seeding large scale fermenters to obtain sufficient quantities of the T cell activating bispecific antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhff CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NSO, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a T cell activating bispecific antigen binding molecule according to the invention is provided, wherein the method comprises culturing a host cell comprising a polynucleotide encoding the T cell activating bispecific antigen binding molecule, as provided herein, under conditions suitable for expression of the T cell activating bispecific antigen binding molecule, and recovering the T cell activating bispecific antigen binding molecule from the host cell (or host cell culture medium).

The components of the T cell activating bispecific antigen binding molecule may be genetically fused to each other. T cell activating bispecific antigen binding molecule can be designed such that its components are fused directly to each other or indirectly through a linker sequence. The composition and length of the linker may be determined in accordance with methods well known in the art and may be tested for efficacy. Examples of linker sequences between different components of T cell activating bispecific antigen binding molecules are found in the sequences provided herein. Additional sequences may also be included to incorporate a cleavage site to separate the individual components of the fusion if desired, for example an endopeptidase recognition sequence.

In certain embodiments the one or more antigen binding moieties of the T cell activating bispecific antigen binding molecules comprise at least an antibody variable region capable of binding an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of antibody, antibody fragment, antigen binding domain or variable region can be used in the T cell activating bispecific antigen binding molecules of the invention. Non-limiting antibodies, antibody fragments, antigen binding domains or variable regions useful in the present invention can be of murine, primate, or human origin. If the T cell activating bispecific antigen binding molecule is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the T cell activating bispecific antigen binding molecule of the invention to bind to a specific antigen can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g. an antibody that competes with the V9 antibody for binding to CD3. In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.). In an exemplary competition assay, immobilized antigen (e.g. CD3) is incubated in a solution comprising a first labeled antibody that binds to the antigen (e.g. V9 antibody) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch. 14 (Cold Spring Harbor Laboratory, Cold Spring Harbor, N.Y.).

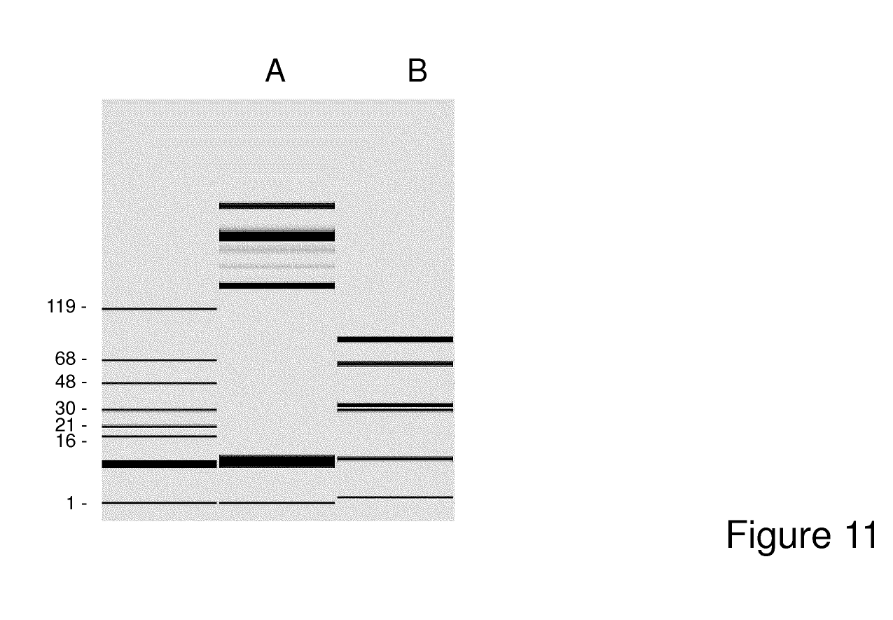

T cell activating bispecific antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the T cell activating bispecific antigen binding molecule binds. For example, for affinity chromatography purification of T cell activating bispecific antigen binding molecules of the invention, a matrix with protein A or protein G may be used. Sequential Protein A or G affinity chromatography and size exclusion chromatography can be used to isolate a T cell activating bispecific antigen binding molecule essentially as described in the Examples. The purity of the T cell activating bispecific antigen binding molecule can be determined by any of a variety of well-known analytical methods including gel electrophoresis, high pressure liquid chromatography, and the like. For example, the heavy chain fusion proteins expressed as described in the Examples were shown to be intact and properly assembled as demonstrated by reducing SDS-PAGE (see e.g. FIG. 11B). Three bands were resolved at approximately Mr 25,000, Mr 50,000 and Mr 75,000, corresponding to the predicted molecular weights of the T cell activating bispecific antigen binding molecule light chain, heavy chain and heavy chain/light chain fusion protein.

Assays

T cell activating bispecific antigen binding molecules provided herein may be identified, screened for, or characterized for their physical/chemical properties and/or biological activities by various assays known in the art.

Affinity Assays

The affinity of the T cell activating bispecific antigen binding molecule for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of T cell activating bispecific antigen binding molecules for different receptors or target antigens may be evaluated using cell lines expressing the particular receptor or target antigen, for example by flow cytometry (FACS). A specific illustrative and exemplary embodiment for measuring binding affinity is described in the following and in the Examples below.

According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 μg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25° C. at a flow rate of 30 μl/min for 120 s.

To determine the affinity to the target antigen, antigen binding molecules are captured by an anti human Fab specific antibody (GE Healthcare) that is immobilized on an activated CM5-sensor chip surface as described for the anti Penta-His antibody. The final amount of coupled protein is approximately 12500 RU. The antigen binding molecules are captured for 60 s at 50 nM. The target antigens are passed through the flow cells for 90 s at a concentration range from approximately 0.5 to 1000 nM with a flowrate of 30 μl/min. The dissociation is monitored for 120 s.

Bulk refractive index differences are corrected for by subtracting the response obtained on reference flow cell. The steady state response is used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

Activity Assays

Biological activity of the T cell activating bispecific antigen binding molecules of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as Robo 4 expressing (endothelial) cells, and the induction of tumor regression and/or the improvement of survival.

Compositions, Formulations, and Routes of Administration

In a further aspect, the invention provides pharmaceutical compositions comprising any of the T cell activating bispecific antigen binding molecules provided herein, e.g., for use in any of the below therapeutic methods. In one embodiment, a pharmaceutical composition comprises any of the T cell activating bispecific antigen binding molecules provided herein and a pharmaceutically acceptable carrier. In another embodiment, a pharmaceutical composition comprises any of the T cell activating bispecific antigen binding molecules provided herein and at least one additional therapeutic agent, e.g., as described below.

Further provided is a method of producing a T cell activating bispecific antigen binding molecule of the invention in a form suitable for administration in vivo, the method comprising (a) obtaining a T cell activating bispecific antigen binding molecule according to the invention, and (b) formulating the T cell activating bispecific antigen binding molecule with at least one pharmaceutically acceptable carrier, whereby a preparation of T cell activating bispecific antigen binding molecule is formulated for administration in vivo.

Pharmaceutical compositions of the present invention comprise a therapeutically effective amount of one or more T cell activating bispecific antigen binding molecule dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one T cell activating bispecific antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. T cell activating bispecific antigen binding molecules of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the T cell activating bispecific antigen binding molecules of the invention.

Parenteral compositions include those designed for administration by injection, e.g. subcutaneous, intradermal, intralesional, intravenous, intraarterial intramuscular, intrathecal or intraperitoneal injection. For injection, the T cell activating bispecific antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the T cell activating bispecific antigen binding molecules may be in powder form for constitution with a suitable vehicle, e.g., sterile pyrogen-free water, before use. Sterile injectable solutions are prepared by incorporating the T cell activating bispecific antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the T cell activating bispecific antigen binding molecules may also be formulated as a depot preparation. Such long acting formulations may be administered by implantation (for example subcutaneously or intramuscularly) or by intramuscular injection. Thus, for example, the T cell activating bispecific antigen binding molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the T cell activating bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The T cell activating bispecific antigen binding molecules may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

Therapeutic Methods and Compositions

Any of the T cell activating bispecific antigen binding molecules provided herein may be used in therapeutic methods. T cell activating bispecific antigen binding molecules of the invention can be used as immunotherapeutic agents, for example in the treatment of cancers.

For use in therapeutic methods, T cell activating bispecific antigen binding molecules of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, T cell activating bispecific antigen binding molecules of the invention for use as a medicament are provided. In further aspects, T cell activating bispecific antigen binding molecules of the invention for use in treating a disease are provided. In certain embodiments, T cell activating bispecific antigen binding molecules of the invention for use in a method of treatment are provided. In one embodiment, the invention provides a T cell activating bispecific antigen binding molecule as described herein for use in the treatment of a disease in an individual in need thereof. In certain embodiments, the invention provides a T cell activating bispecific antigen binding molecule for use in a method of treating an individual having a disease comprising administering to the individual a therapeutically effective amount of the T cell activating bispecific antigen binding molecule. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a T cell activating bispecific antigen binding molecule as described herein for use in inducing lysis of a target cell, particularly a Robo 4 expressing cell, more particularly a Robo 4 expressing endothelial cell. In certain embodiments, the invention provides a T cell activating bispecific antigen binding molecule for use in a method of inducing lysis of a target cell, particularly a Robo 4 expressing cell, more particularly a Robo 4 expressing endothelial cell, in an individual comprising administering to the individual an effective amount of the T cell activating bispecific antigen binding molecule to induce lysis of a target cell. An “individual” according to any of the above embodiments is a mammal, preferably a human.

In a further aspect, the invention provides for the use of a T cell activating bispecific antigen binding molecule of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a Robo 4 expressing cell, more particularly a Robo 4 expressing endothelial cell. In still a further embodiment, the medicament is for use in a method of inducing lysis of a target cell, particularly a Robo 4 expressing cell, more particularly a Robo 4 expressing endothelial cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a T cell activating bispecific antigen binding molecule of the invention. In one embodiment a composition is administered to said invididual, comprising the T cell activating bispecific antigen binding molecule of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human.

In a further aspect, the invention provides a method for inducing lysis of a target cell, particularly a Robo 4 expressing cell, more particularly a Robo 4 expressing endothelial cell. In one embodiment the method comprises contacting a target cell with a T cell activating bispecific antigen binding molecule of the invention in the presence of a T cell, particularly a cytotoxic T cell. In a further aspect, a method for inducing lysis of a target cell, particularly a Robo 4 expressing cell, more particularly a Robo 4 expressing endothelial cell, in an individual is provided. In one such embodiment, the method comprises administering to the individual an effective amount of a T cell activating bispecific antigen binding molecule to induce lysis of a target cell. In one embodiment, an “individual” is a human.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a T cell activating bispecific antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the T cell activating bispecific antigen binding molecule may not provide a cure but may only provide partial benefit. In some embodiments, a physiological change having some benefit is also considered therapeutically beneficial. Thus, in some embodiments, an amount of T cell activating bispecific antigen binding molecule that provides a physiological change is considered an “effective amount” or a “therapeutically effective amount”. The subject, patient, or individual in need of treatment is typically a mammal, more specifically a human.

In some embodiments, an effective amount of a T cell activating bispecific antigen binding molecule of the invention is administered to a cell. In other embodiments, a therapeutically effective amount of a T cell activating bispecific antigen binding molecule of the invention is administered to an individual for the treatment of disease.

For the prevention or treatment of disease, the appropriate dosage of a T cell activating bispecific antigen binding molecule of the invention (when used alone or in combination with one or more other additional therapeutic agents) will depend on the type of disease to be treated, the route of administration, the body weight of the patient, the type of T cell activating bispecific antigen binding molecule, the severity and course of the disease, whether the T cell activating bispecific antigen binding molecule is administered for preventive or therapeutic purposes, previous or concurrent therapeutic interventions, the patient's clinical history and response to the T cell activating bispecific antigen binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The T cell activating bispecific antigen binding molecule is suitably administered to the patient at one time or over a series of treatments. Depending on the type and severity of the disease, about 1 μg/kg to 15 mg/kg (e.g. 0.1 mg/kg-10 mg/kg) of T cell activating bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the T cell activating bispecific antigen binding molecule would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the T cell activating bispecific antigen binding molecule). An initial higher loading dose, followed by one or more lower doses may be administered. However, other dosage regimens may be useful. The progress of this therapy is easily monitored by conventional techniques and assays.

The T cell activating bispecific antigen binding molecules of the invention will generally be used in an amount effective to achieve the intended purpose. For use to treat or prevent a disease condition, the T cell activating bispecific antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the T cell activating bispecific antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the T cell activating bispecific antigen binding molecules may not be related to plasma concentration. One having skill in the art will be able to optimize therapeutically effective local dosages without undue experimentation.

A therapeutically effective dose of the T cell activating bispecific antigen binding molecules described herein will generally provide therapeutic benefit without causing substantial toxicity. Toxicity and therapeutic efficacy of a T cell activating bispecific antigen binding molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. T cell activating bispecific antigen binding molecules that exhibit large therapeutic indices are preferred. In one embodiment, the T cell activating bispecific antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with T cell activating bispecific antigen binding molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The T cell activating bispecific antigen binding molecules of the invention may be administered in combination with one or more other agents in therapy. For instance, a T cell activating bispecific antigen binding molecule of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of T cell activating bispecific antigen binding molecule used, the type of disorder or treatment, and other factors discussed above. The T cell activating bispecific antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the T cell activating bispecific antigen binding molecule of the invention can occur prior to, simultaneously, and/or following, administration of the additional therapeutic agent and/or adjuvant. T cell activating bispecific antigen binding molecules of the invention can also be used in combination with radiation therapy.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a T cell activating bispecific antigen binding molecule of the invention. The label or package insert indicates that the composition is used for treating the condition of choice. Moreover, the article of manufacture may comprise (a) a first container with a composition contained therein, wherein the composition comprises a T cell activating bispecific antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### Example 1

Preparation of Recombinant Human and Murine Robo 4 for Hamster Immunization and Phage Display

The molecules were produced by transfecting HEK293 EBNA cells with a mammalian expression vector encoding the human or murine Robo 4 extracellular domain (ECD) where the ECD encoding fragment is separated from a downstream Avi-tag (Avi) and His-tag (His) encoding sequence. The transfection was performed by using the 293Fectin transfection reagent (Invitrogen). Sequences of human and murine Robo 4 antigens are shown in SEQ ID NOs 1 and 3, respectively.

HEK293 EBNA cells were cultivated in suspension in serum free conditions in FreeStyle 293 expression medium (Invitrogen). For the production in 100 ml shake flasks, 1.5 million HEK293 EBNA cells were seeded per flask. Expression vectors were mixed in 32.9 ml Opti-MEM medium (Invitrogen) to a final amount of 600 μg DNA. 293Fectin solution was prepared by adding 2 ml 293Fectin to 31.2 ml Opti-MEM, and incubated for 5 minutes before addition to the DNA solution. The mixture was subsequently incubated for 20 minutes at room temperature. 6.64 ml of the DNA/293Fectin solution was added per 100 ml shake flask and cells were incubated at 135 rpm, 37° C. and 5% CO₂. After 7 days cultivation, supernatant was collected for purification by centrifugation for 15 min at 210×g, the solution was sterile filtered (0.22 μm filter), sodium azide in a final concentration of 0.01% w/v was added, and the solution was kept at 4° C.

The secreted proteins were purified from cell culture supernatants by metal chelating affinity chromatography, followed by a size exclusion chromatographic step. To avoid leakage of Ni-ions coupled to the affinity chromatography matrix, supernatants had to be diafiltrated prior to the first purification step. Therefore supernatants were first concentrated to 210 ml using a crossflow equipped with a Hydrosart membrane (MWCO 30 kDa, Sartorius) and equilibrated with 20 mM sodium phosphate, 500 mM sodium chloride pH 7.4 (equilibration buffer). Concentrated supernatant was diluted up to 1 L with equilibration buffer and again concentrated to 210 ml. This procedure was repeated three times to ensure a complete buffer exchange of the supernatant. Final volume of the concentrate was 210 ml.

For affinity chromatography, the concentrate was loaded on a HisTrap FF column (CV=5 mL, GE Healthcare) equilibrated with 25 ml 20 mM sodium phosphate, 500 mM sodium chloride pH 7.4. Unbound protein was removed by washing with 16 column volumes 20 mM sodium phosphate, 500 mM sodium chloride pH 7.4. Subsequently, target protein was eluted in a linear gradient to 45% (v/v) 20 mM sodium phosphate, 500 mM sodium chloride, 500 mM imidazole, pH 7.4 over 100 ml. Remaining protein was removed by washing the column with a gradient from 45-100% 20 mM sodium phosphate, 500 mM sodium chloride, 500 mM imidazole, pH 7.4 over 10 ml, and an additional wash with 20 mM sodium phosphate, 500 mM sodium chloride, 500 mM imidazole, pH 7.4 over 20 ml.

EDTA was added to the eluted protein to a final concentration of 5 mM. Fractions from metal chelate chromatography were concentrated using spin concentrator Amicon (Millipore; MWCO 30 kDa).

Target protein was subsequently loaded on a HiLoad Superdex 200 column (GE Healthcare) equilibrated with 2 mM MOPS, 150 mM sodium chloride pH 7.4.

The protein concentration of purified protein samples was determined by measuring the optical density (OD) at 280 nm, using the molar extinction coefficient calculated on the basis of the amino acid sequence. Purity and molecular weight of the antigens was analyzed by SDS PAGE in the presence of a reducing agent (5 mM 1,4-dithiotreitol) and staining with Coomassie

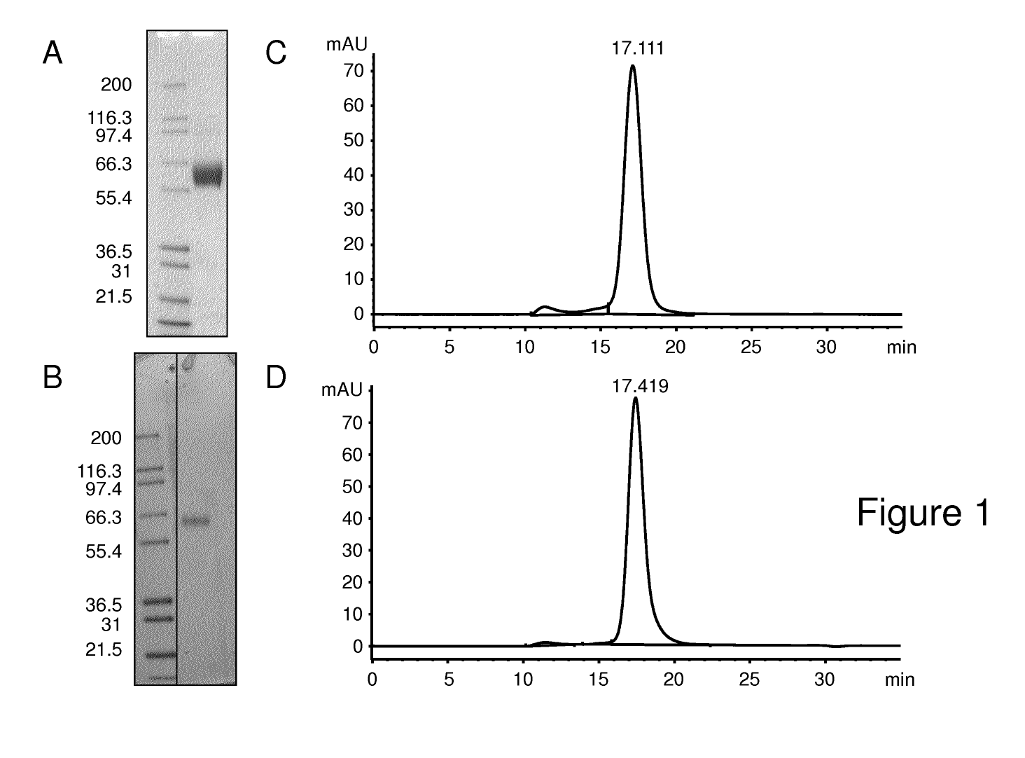

(SimpleBlue™ SafeStain from Invitrogen) (FIGS. 1, A and B). The NuPAGE® Pre-Cast gel system (Invitrogen) was used according to the manufacturer's instructions (4-12% Bis-Tris gel). The aggregate content of recombinant proteins was analyzed using a Superdex 200 10/300GL analytical size exclusion column (GE Healthcare) in 2 mM MOPS, 150 mM NaCl, 0.02% (w/v) NaN₃, pH 7.3 running buffer at 25° C. (FIGS. 1, C and D).

### Example 2

Generation of Robo 4 Binders 01E06, 01F05 and 01F09 by Immunization

The Robo 4 binders 01E06, 01F05 and 01F09 were generated by immunizing five Armenian hamsters with human (hu) Robo 4 extracellular domain (ECD)-precision site (PreS)-Avi-tag (Avi)-6× histidine (His) (SEQ ID NO: 1) and murine (mu) Robo 4 ECD-PreS-Avi-His (SEQ ID NO: 3). Subsequently spleens were removed, dissolved into single cells, and fused with a mouse myeloma cell line. The fusions were plated into 96-well plates for selection of primary wells and, after selection, seeded by FACS for single cell cloning. The resulting clones were assayed for hamster IgG secretion, human Robo 4 binding, as well as mouse Robo 4 binding. The best clones were banked and supernatant as well as cell pellets were prepared for further analysis.

Immunization of Animals and Detection of Robo 4 Specific Antibodies

Five Armenian hamsters were immunized with human Robo 4 and murine Robo 4. At day 0, the hamsters were immunized with 100 μg human Robo 4 emulsified with complete Freund's adjuvant (CFA), injected intraperitoneally (i.p.). The second immunization was performed 4 weeks later using 100 μg human Robo 4 emulsified with incomplete Freund's adjuvant (IFA) i.p. The third immunization was performed 8 weeks after the initial immunization with 100 μg murine Robo 4 emulsified with IFA i.p. The last immunization was performed in week 12 using 100 μg huRobo 4 emulsified with IFA i.p. Three days after the third and the forth immunization blood from the tail vein was taken and analyzed for Robo 4 specific antibody titers. Three days after the fourth immunization the animals were sacrificed and the spleens removed.

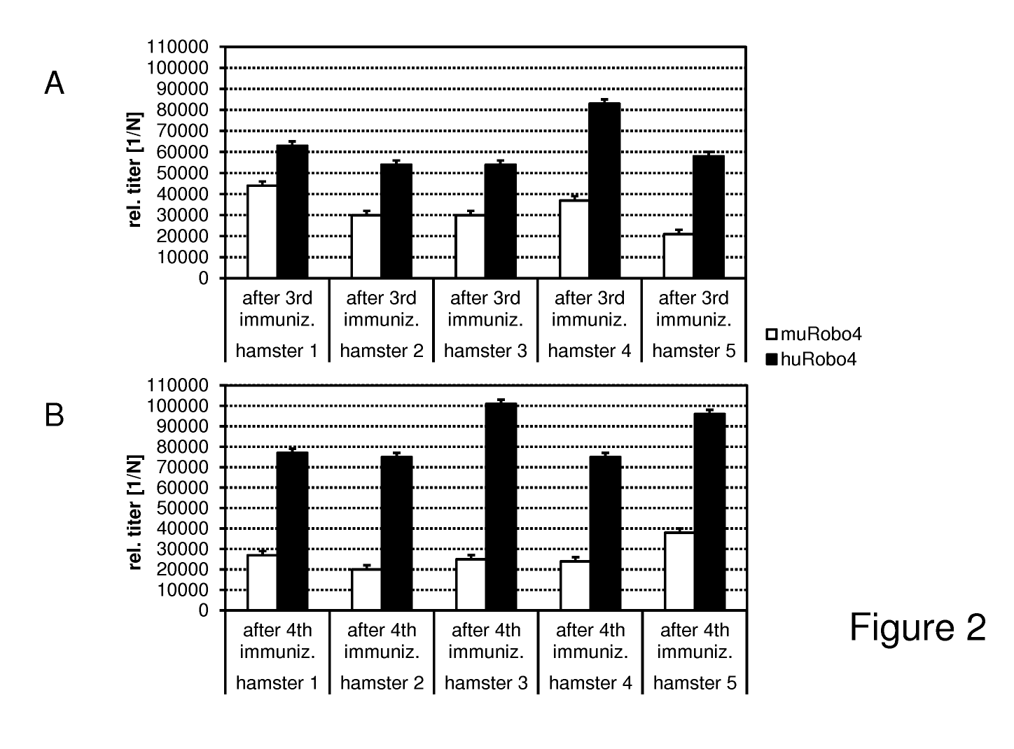

The titer analysis for Robo 4 specific antibodies was performed using enzyme linked immunosorbent assay (ELISA). For the human Robo 4 specific ELISA, a 96-well plate was coated with 100 μl/well of human Robo 4 at a concentration of 0.078 μg/ml in carbonate buffer for 1 h at 37° C. For the murine Robo 4 specific ELISA, a 96-well plate was coated with 100 μl/well of murine Robo 4 at a concentration of 0.3125 μg/ml in carbonate buffer for 1h at 37° C. Subsequently, the plates were washed three times with PBS containing 0.05% Tween 20. After washing, unspecific binding was blocked using 200 μl/well of 1% Crotein C in PBS for 1 h at 37° C. Excess protein was washed away using the previously mentioned washing protocol. 100 μl/well serum samples in different dilutions in sample buffer were added and incubated for 1 h at 37° C. (for human Robo 4) or overnight at 4° C. (for murine Robo 4), before washing the plates again. For detection, 100 μl/well peroxidase-conjugated affinity purified goat-anti Armenian hamster IgG (Dianova, #127-035-160) was added in a dilution of 1:20000 for 1h at 37° C., before washing again. For the colorimetric read out, 50 μl/well BM Blue POD substrate was added for 2 min at room temperature, and the reaction was stopped using 50 μl/well 0.5 M H₂SO₄. Adsorption was measured using a photometer at 450/690 nm. The results are shown in FIG. 2.

Fusion and Selection of Hybridoma

P3x63-Ag8.653 cells were cultivated in exponential phase for at least 10 days in RPMI 1640 medium (Life Technologies) supplemented with 10% ultra-low IgG fetal bovine serum (FBS) (PAN Biotech), 2 mM L-glutamine (Life Technologies), 1 mM sodium pyruvate (Life Technologies), and 1× non-essential amino acids (NEAA) (Life Technologies). For the last three days prior to utilizing the cells as fusion partners the medium was supplemented with 8-azaguanin.

After removal of the spleens from the immunized hamsters, the spleens were washed in RPMI 1640 medium supplemented with 1× penicillin/streptomycin (P/S) solution (Roche Applied Sciences), punctured, and cut. The spleens were washed with medium to remove the cells. The cell suspension was resuspended and passed through a 40 μm sieve into a 50 ml falcon tube and the volume was adjusted to 40 ml using RPMI 1640 supplemented with P/S soltion. The falcon tube was centrifuged for 10 min at 300×g and the supernatant discarded. The cell pellet was washed twice with fresh medium and finally resuspended in 5 ml medium. An aliquot was taken for determination of cell number and viability using a Vi-cell XR (Beckman Coulter).

Splenocytes and P3x63-Ag8.653 cells were mixed at ratios 1:1 and 1:2 in RPMI1640, centrifuged, and the supernatant was discarded. After gentle disruption of the dry cell pellet 1 ml of poly ethylene glycol (PEG) was added slowly followed by the slow addition of first 2 ml of RPMI 1640, second 5 ml of RPMI 1640, third 10 ml of RPMI 1640, and finally of 7 ml of RPMI 1640 supplemented with 10% FBS, 2 mM L-glutamine, 1 mM Na-pyruvate, 1×NEAA and P/S solution. All additions were made while the tube containing the cell suspension was slowly swirled. The final cell suspension was incubated overnight at 37° C. After the incubation period the cell suspension was centrifuged at 300×g for 10 minutes. The supernatant was discarded and the cell pellet resuspended in hybridoma growth medium consisting of 50 ml RPMI 1640 supplemented with 10% ultra-low IgG FBS, 2 mM L-glutamine, 1 mM sodium pyruvate, 1×NEAA, and 1×Nutridoma-CS (Roche Applied Sciences), murine IL-6 and 1× azaserine hypoxanthine (Sigma #A9666).

Selection and Analysis of Primary Wells

The cell suspension was diluted with hybridoma growth medium and seeded in 96-well plates.

The plates were incubated at 37° C., 5% CO₂ for several days. Growing clones were transferred into 24-well plates and the supernatants were assayed by ELISA for the expression of hamster IgG, as well as binding to human Robo 4, murine Robo 4 and human Robol (for protocol see details above).

Nine primary wells showing best binding to human and murine Robo 4 in ELISA, good binding to human Robo 4 on cells, and no binding to human Robol were selected for cloning. The cells from the primary wells were expanded in T75 flasks in hybridoma growth medium before seeding as single cells into 96-well plates using FACS.

Subcloning of Primary Wells

From each cloned primary well, several clones were propagated from 96-well to 24-well plates. The supernatant from the 24-well plates was assayed for human Robo 4 binding by ELISA and FACS on human Robo 4 expressing CHO cell lines.

Clones showing best binding to human and murine Robo 4 in ELISA, good binding to human Robo 4 on cells, and no binding to human Robol were selected for expansion and sequencing. Positive tested single clones (named 01E06, 01F05 and 01F09) were expanded in hybridoma growth media and cryopreserved for future studies. DNA was prepared to allow sequencing. The heavy and light chain variable region sequences of antibody clones 01E06, 01F05 and 01F09 are shown in SEQ ID NOs 19 and 20, SEQ ID NOs 23 and 25, and SEQ ID NOs 27 and 29, respectively.

### Example 3

Generation of Robo 4 Binder 7G2 by Phage Display

The antibody 7G2 with specificity for human and cynomolgus Robo 4 was selected from a generic phage-displayed antibody library in the Fab format (DP47-3). This library was constructed on the basis of human germline genes using the V-domain pairing Vk3_20 (kappa light chain) and VH3_23 (heavy chain), comprising randomized sequence space in CDR3 of the light chain (L3) and CDR3 of the heavy chain (H3). Library generation was performed by assembly of three PCR-amplified fragments applying splicing by overlapping extension (SOE) PCR. Fragment 1 comprises the 5′ end of the antibody gene including randomized L3, fragment 2 is a central constant fragment spanning from L3 to H3 whereas fragment 3 comprises randomized H3 and the 3′ portion of the antibody gene (SEQ ID NO 115). The following primer combinations were used to generate these library fragments for the DP47-3 library: fragment 1 (LMB3 (SEQ ID NO: 116)—LibL1b_new (SEQ ID NO: 117)), fragment 2 (MS63 (SEQ ID NO: 118)—MS64 (SEQ ID NO: 119)) and fragment 3 (Lib2H (SEQ ID NO: 120)—fdseqlong (SEQ ID NO: 121)). PCR parameters for generation of library fragments were 5 min initial denaturation at 94° C., 25 cycles of 1 min 94° C., 1 min 58° C. and 1 min 72° C., and terminal elongation for 10 min at 72° C. For assembly PCR, using equimolar ratios of the three fragments as template, parameters were 3 min initial denaturation at 94° C. and 5 cycles of 30 s 94° C., 1 min 58° C. and 2 min 72° C. At this stage, outer primers were added and additional 20 cycles performed prior to a terminal elongation for 10 min at 72° C. After assembly of sufficient amounts of full-length randomized Fab constructs, they were digested using NcoI and NotI restriction enzymes alongside with similarly treated acceptor phagemid vector. 22.8 μg of Fab library were ligated with 16.2 μg of phagemid vector. Purified ligations were used for 68 transformations to obtain a final library size of 4.2×10¹⁰. Phagemid particles displaying the Fab library were rescued and purified by PEG/NaCl purification to be used for selections.

Antigens for the phage display selections were transiently expressed in HEK EBNA cells (see above) and in vivo biotinylated via co-expression of BirA. Selections were carried out against the biotinylated ectodomain of human Robo 4 with a C-terminal AcTEV protease site, followed by an Avi-tag for enzymatic site-specific biotinylation and an 6×His-tag for purification (see SEQ ID NO: 5). Panning rounds were performed in solution according to the following pattern: 1) Incubation of 10¹² phagemid particles with 100 nM biotinylated human Robo 4 as well as 100 nM non-biotinylated CH3-avi-tag-H6-tag (in order to competitively avoid tag-binders) for 0.5 h in a total volume of 1 ml. 2) Capture of biotinylated human Robo 4 and attached specifically binding phage by addition of 5.4×10⁷ streptavidin-coated magnetic beads for 10 min (round 1 and 3). 3) Washing of beads using 5×1 ml PBS/Tween 20 and 5×1 ml PBS. 4) Elution of phage particles by addition of 1 ml 100 mM triethylamine (TEA) for 10 min and neutralization by addition of 500 μl 1M Tris/HCl pH 7.4. 5) Re-infection of log-phase E. coli TG1 cells with the eluted phage particles, infection with helperphage VCSM13 and subsequent PEG/NaCl precipitation of phagemid particles to be used in subsequent selection rounds. Selections were carried out over three rounds using constant antigen concentrations of 100 nM, however, in round 3, murine Robo 4 was used to potentially enable selection of species cross-reactive phage antibodies. In round 2, in order to avoid binders against streptavidin, capture of antigen-phage complexes was performed by use of neutravidin-coated plates. Specific binders were identified by ELISA as follows: 100 μl of 100 nM and 50 nM biotinylated human Robo 4, murine Robo 4 and CH3 were coated on neutravidin plates. Fab-containing bacterial supernatants were added and binding Fabs were detected via their Flag-tags using an anti-Flag/HRP secondary antibody. Clones exhibiting signals either on only human or human and murine Robo 4 but not on CH3 were short-listed for further analyses. They were bacterially expressed in a 0.5 L culture volume, affinity purified and further characterized by SPR-analysis using BioRad's ProteOn XPR36 biosensor. This way, amongst others, clone 7G2 was identified. It is cross-reactive for human and cynomolgus Robo 4 (14.9 nM and 20.5 nM monovalent affinities, respectively) but does not recognize murine Robo 4. The heavy and light chain variable region sequences of antibody clone 7G2 are shown in SEQ ID NOs 31 and 33, respectively.

### Example 4

Preparation of Anti-Robo 4 IgG Antibodies

The DNA fragments comprising the heavy and light chain variable domains were inserted in frame into either the human IgG₁ constant heavy chain or the human constant light chain containing recipient mammalian expression vector, respectively. The antibody expression was driven by an MPSV promoter and transcription terminated by a synthetic polyA signal sequence located downstream of the CDS. In addition to the expression cassette each vector contained an EBV oriP sequence.

The molecules were produced by co-transfecting HEK293 EBNA cells with the appropriate mammalian expression vectors in a 1:1 ratio using calcium-phosphate transfection.

For transfection, cells were grown as adherent monolayer cultures in T-flasks using DMEM culture medium supplemented with 10% (v/v) fetal calf serum (FCS), and transfected when they were between 50 and 80% confluent. For the transfection of a T150 flask, 15 million cells were seeded 24 hours before transfection in 25 ml DMEM culture medium supplemented with 10% FCS (v/v), and incubated at 37° C., 5% CO₂ overnight. For each T150 flask to be transfected, a solution of DNA, CaCl₂ and water was prepared by mixing 94 μg total plasmid vector DNA (1:1 ratio of the corresponding vectors), water to a final volume of 469 μl, and 469 μl of a 1 M CaCl₂ solution. To this mixture, 938 μl of a 50 mM HEPES, 280 mM NaCl, 1.5 mM Na₂HPO₄ solution at pH 7.05 was added, mixed immediately for 10 s and left to stand at room temperature for 20 s. The suspension was diluted with 10 ml of DMEM supplemented with 2% (v/v) FCS, and added to the cells in place of the existing medium. Subsequently, additional 13 ml of transfection medium were added. The cells were incubated at 37° C., 5% CO₂ for about 17 to 20 hours before the medium was replaced with 25 ml DMEM, 10% FCS. The conditioned culture medium was harvested approx. 7 days post-media exchange by centrifugation for 15 min at 210×g, the solution was sterile filtered (0.22 μm filter) and sodium azide in a final concentration of 0.01% (w/v) was added. The solutions were kept at 4° C.

The secreted proteins were purified from the cell culture supernatants by Protein A affinity chromatography, followed by a size exclusion chromatographic step.

For affinity chromatography supernatant was loaded on a HiTrap Protein A HP column (CV=5 mL, GE Healthcare), equilibrated with 25 ml 20 mM sodium phosphate, 20 mM sodium citrate, pH 7.5. Unbound protein was removed by washing with at least 10 column volumes 20 mM sodium phosphate, 20 mM sodium citrate, 0.5 M sodium chloride, pH 7.5, followed by an additional wash step using 6 column volumes 10 mM sodium phosphate, 20 mM sodium citrate, 0.5 M sodium chloride, pH 5.45. The column was washed subsequently with 20 ml 10 mM MES, 100 mM sodium chloride, pH 5.0 and target protein eluted in 6 column volumes 20 mM sodium citrate, 100 mM sodium chloride, 100 mM glycine, pH 3.0. The protein solution was neutralized by adding 1/10 of 0.5M sodium phosphate. Target protein was concentrated and filtrated before loading on a HiLoad Superdex 200 column (GE Healthcare) equilibrated with 20 mM histidine, 150 mM NaCl, pH6.0.

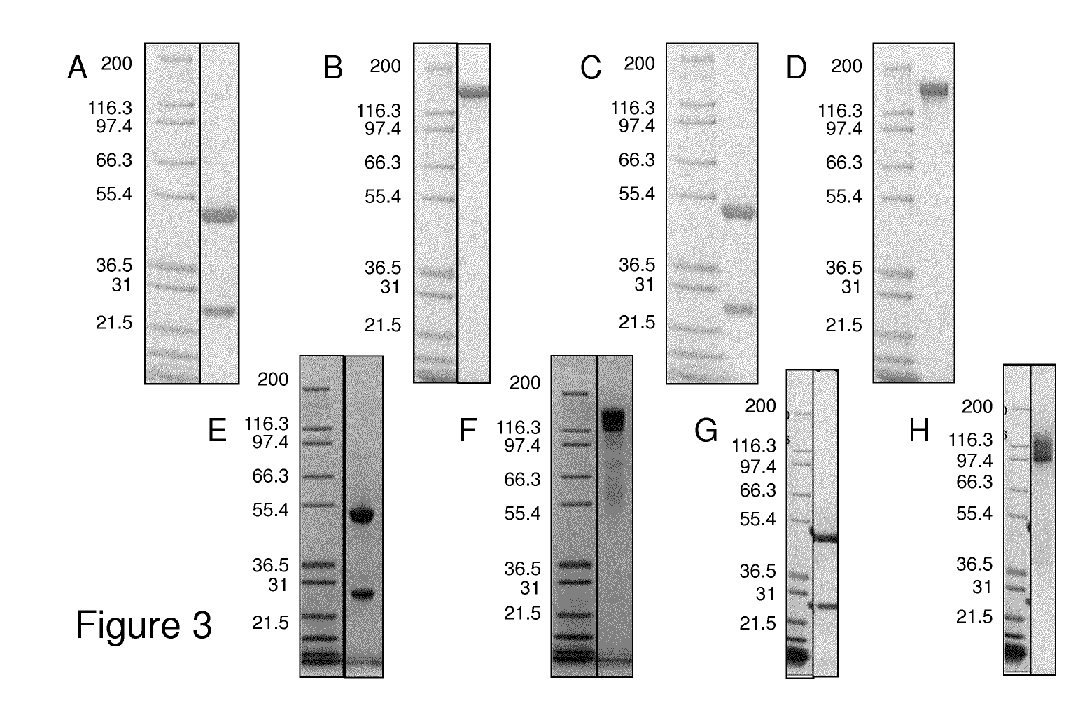

The protein concentration of purified protein samples was determined by measuring the optical density (OD) at 280 nm, using the molar extinction coefficient calculated on the basis of the amino acid sequence. Purity and molecular weight of antibodies were analyzed by SDS PAGE in the presence and absence of a reducing agent (5 mM 1,4-dithiotreitol) and staining with Coomassie (SimpleBlue™ SafeStain, Invitrogen) (FIG. 3). The NuPAGE® Pre-Cast gel system (Invitrogen) was used according to the manufacturer's instructions (4-12% Bis-Tris gels).

### Example 5

Preparation of Recombinant Human Robol for Characterization of Anti-Robo 4 IgGs

The molecule was produced by transfecting HEK293-EBNA cells with the corresponding mammalian expression vector using calcium phosphate-transfection as described above for the anti-Robo 4 IgGs. The sequence of the human Robol antigen is shown in SEQ ID NO: 7. The secreted protein was purified from cell culture supernatants by metal chelating affinity chromatography, followed by a size exclusion chromatographic step, essentially as described above for the human and murine Robo 4 antigens.

For affinity chromatography the protein was loaded on a HisTrap FF column (CV=5 mL, GE Healthcare) equilibrated with 40 ml 20 mM sodium phosphate, 500 mM sodium chloride, pH 7.4. Unbound protein was removed by washing with 10 column volumes 20 mM sodium phosphate, 500 mM sodium chloride, pH 7.4. For elution, the column was first washed with 5 column volumes of 5% (v/v) elution buffer (20 mM sodium phosphate, 500 mM sodium chloride, 500 mM imidazole, pH 7.4). Subsequently, the target protein was eluted in a linear gradient to 45% (v/v) elution buffer over 50 ml. Remaining protein was removed by washing the column with 10 ml 20 mM sodium phosphate, 500 mM sodium chloride, 500 mM imidazole, pH 7.4.

EDTA was added to the eluted protein to a final concentration of 5 mM. Fractions from metal chelate chromatography were concentrated using spin concentrator Amicon (Millipore; MWCO 30 kDa).

Subsequently, the protein was loaded on a HiLoad Superdex 200 column (GE Healthcare) equilibrated with 2 mM MOPS, 150 mM sodium chloride solution of pH 7.4.

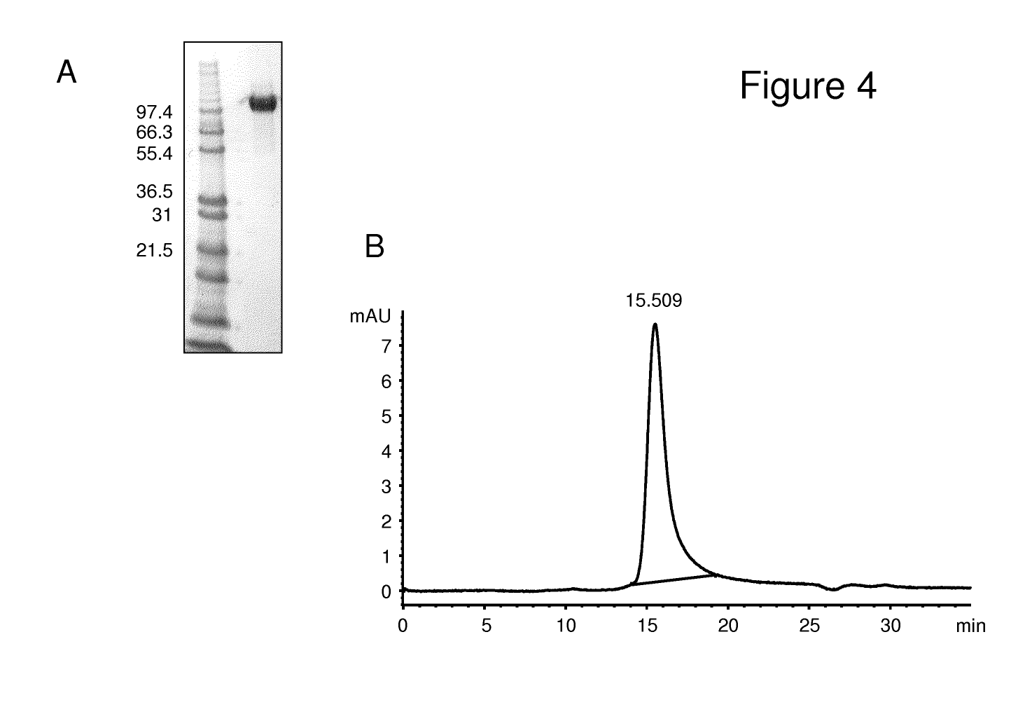

Concentration of the purified protein was determined and the protein analysed by SDS PAGE and analytical size exclusion chromatography as described above for the human and murine Robo 4 antigens (FIG. 4).

### Example 6

Preparation of Recombinant Cynomolgus Robo 4 for Characterization of Anti-Robo 4 IgGs

The molecule was produced by transfecting HEK293 EBNA cells with the corresponding mammalian expression vector using polyethylenimine (PEI). The sequence of the antigen is shown in SEQ ID NO: 9.

HEK293 EBNA cells were cultivated in suspension in serum free CD CHO culture medium. For the production in 500 ml shake flask 400 million HEK293 EBNA cells are seeded 24 hours before transfection. For transfection, cells were centrifuged for 5 min by 210×g, and supernatant was replaced by 20 ml pre-warmed CD CHO medium. Expression vectors were mixed in 20 ml CD CHO medium to a final amount of 200 μg DNA. After addition of 540 μl PEI solution, the mixture was vortexed for 15 s and subsequently incubated for 10 min at room temperature. Afterwards cells were mixed with the DNA/PEI solution, transferred to a 500 ml shake flask and incubated for 3 hours at 37° C., 5% CO₂. After the incubation, 160 ml F17 medium was added and cells were cultivated for 24 hours. One day after the transfection, 1 mM valproic acid and 7% Feed 1 was added. After 7 days cultivation, supernatant was collected for purification by centrifugation for 15 min at 210×g, the solution was sterile filtered (0.22 μm filter) and sodium azide in a final concentration of 0.01% (w/v) was added. The solution was kept at 4° C.

The secreted protein was purified from cell culture supernatants by affinity chromatography using metal chelating affinity chromatography, followed by a size exclusion chromatographic step essentially as described above for the human and murine Robo 4 antigens.

For affinity chromatography the protein was loaded on a HisTrap FF column (CV=5 mL, GE Healthcare) equilibrated with 25 ml 20 mM sodium phosphate, 500 mM sodium chloride pH7.4. Unbound protein is removed by washing with 10 column volumes 20 mM sodium phosphate, 500 mM sodium chloride, pH 7.4. For elution, the column was first washed with 12 column volumes of 5% (v/v) elution buffer (20 mM sodium phosphate, 500 mM sodium chloride, 500 mM imidazole, pH 7.4), before target protein was eluted in a linear gradient to 45% (v/v) elution buffer over 60 ml. Remaining protein was removed by washing the column with 15 ml 20 mM sodium phosphate, 500 mM sodium chloride, 500 mM imidazole, pH 7.4.

EDTA is added to the eluted protein to a final concentration of 5 mM. Fractions from metal chelate chromatography are concentrated using spin concentrator Amicon (Millipore; MWCO 30 kDa).

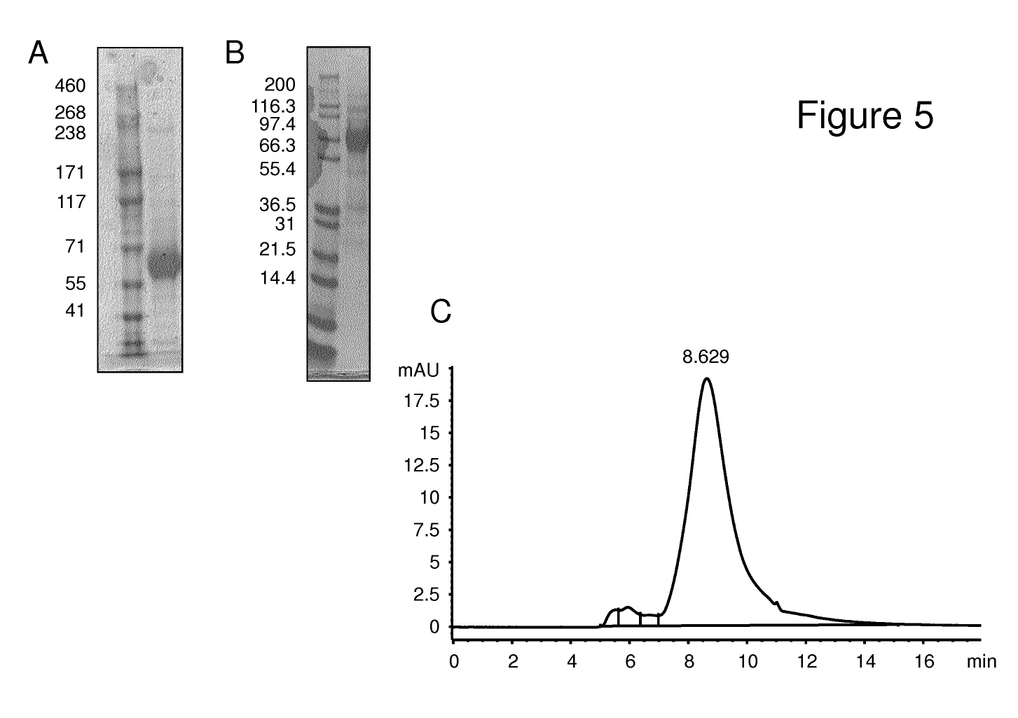

Purity and molecular weight were analyzed by SDS PAGE in the presence and absence of a reducing agent (5 mM 1,4-dithiotreitol) and staining with Coomassie (SimpleBlue™ SafeStain, Invitrogen) (FIGS. 5, A and B). The NuPAGE® Pre-Cast gel system (Invitrogen) is used according to the manufacturer's instructions (4-12% Tris-Acetate or 4-12% Bis-Tris gels). Aggregate content was analyzed using a TSKgel G3000 SW XL analytical size-exclusion column (Tosoh) equilibrated in 25 mM K₂HPO₄, 125 mM NaCl, 200 mM L-arginine monohydrocloride, 0.02% (w/v) NaN₃, pH 6.7 running buffer at 25° C. (FIG. 5C).

### Example 7

Preparation of Recombinant Human Robo 4 Fibronectin (FN)-Like Domain 1, FN-Like Domain 2, IgG-Like Domain 1 and Ig-Like Domain 2 for Characterization of Anti-Robo 4 IgGs

The DNA fragments comprising the sequence of the respective human Robo 4 ECD domains were inserted in frame into a generic mammalian expression vector encoding the human Fc knob followed by an Avi-tag. The co-expression of a corresponding Fc hole domain (SEQ ID NO: 89) leads to the formation of a monomeric Fc containing antigen domain. The sequences of the antigens are shown in SEQ ID NOs 11, 13, 15 and 17.

The molecules were produced by co-transfecting HEK293-EBNA cells with the corresponding mammalian expression vectors using polyethylenimine as described above for the cynomolgus Robo 4 antigen. The cells were transfected with the corresponding expression vectors in a 1:8 ratio (“vector Fc(hole)”: “vector antigen-Fc(knob)”).

The secreted proteins were purified from cell culture supernatants by Protein A affinity chromatography followed by a size exclusion chromatographic step.

For affinity chromatography supernatant was loaded on a HiTrap ProteinA HP column (CV=5 mL, GE Healthcare) equilibrated with 40 ml 20 mM sodium phosphate, 20 mM sodium citrate, 500 mM NaCl, 0.01% (v/v) Tween 20, pH 7.5. Unbound protein was removed by washing with at least 10 column volumes equilibration buffer. Target protein was eluted in a linear pH-gradient over 20 column volumes to 20 mM sodium citrate, 500 mM sodium chloride, 0.01% (v/v) Tween 20, pH 3.0. The column was washed subsequently with 10 column volumes 20 mM sodium citrate, 500 mM sodium chloride, 0.01% (v/v) Tween 20, pH 3.0. The protein solution was neutralized by adding 1/10 of 0.5 M sodium phosphate, and concentrated before loading on a HiLoad Superdex 200 column (GE Healthcare) equilibrated with 2 mM MOPS, 150 mM sodium chloride, pH 7.4.

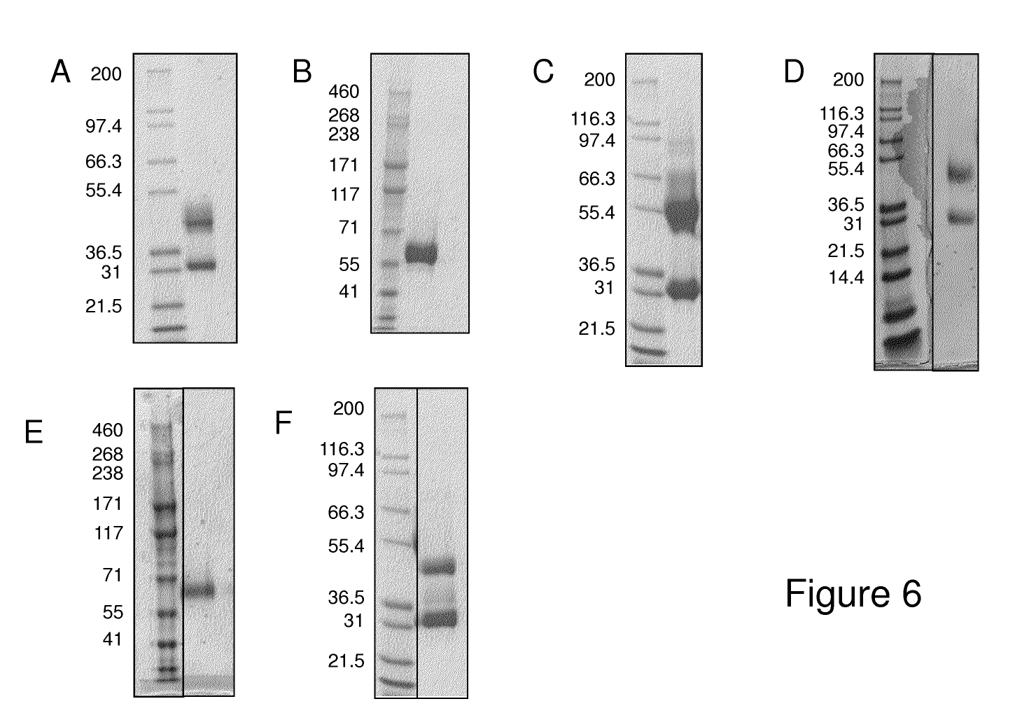

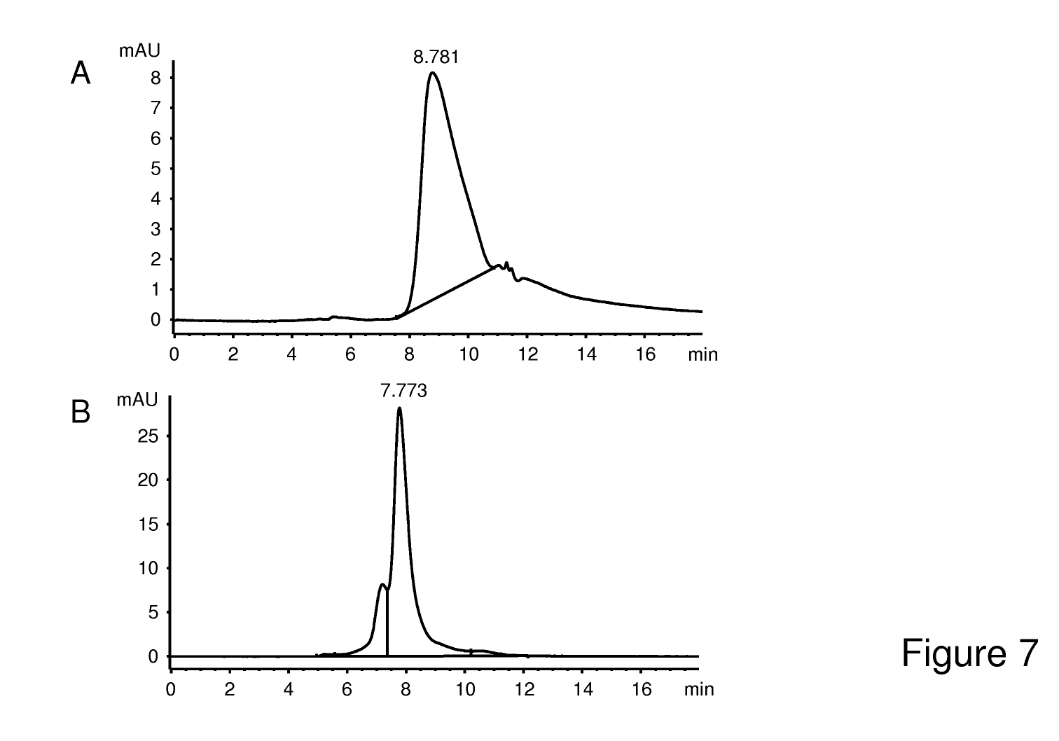

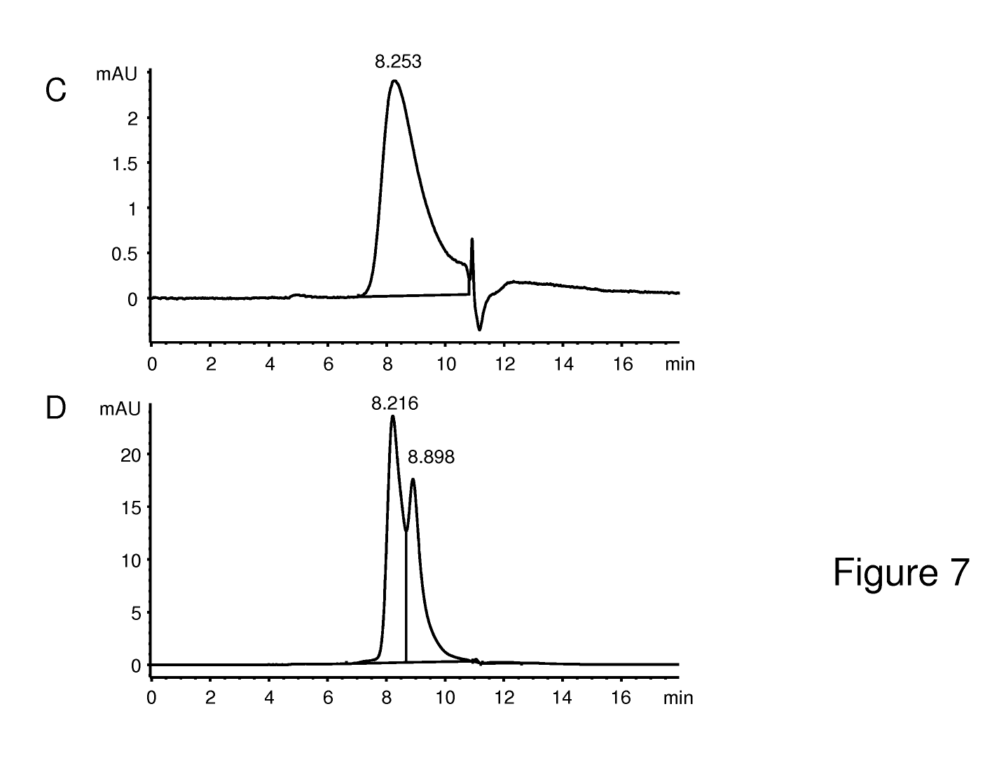

The protein was analysed as described above for the cynomolgus Robo 4 antigen (FIGS. 6 and 7).

### Example 8

Surface Plasmon Resonance (SPR) for Characterization of Anti Robo 4 IgGs

All surface plasmon resonance (SPR) experiments are performed on a Biacore T100 at 25° C. with HBS-EP as running buffer (0.01 M HEPES pH 7.4, 0.15 M NaCl, 3 mM EDTA, 0.005% Surfactant P20 (Biacore)).

For determination of kinetic values of interaction between anti-Robo 4 antibodies and recombinant human, murine and cynomolgus Robo 4, direct coupling of around 12500 resonance units (RU) of anti-human Fab-specific antibody (GE Healthcare) was performed on a CM5 chip at pH 5.0 using the standard amine coupling kit (Biacore). Anti Robo 4 antibodies were captured for 60 s at 50 nM. Recombinant human, murine and cynomolgus Robo 4 were passed at a concentration range from 0.46-1000 nM with a flow of 30 μl/min through the flow cells over 90 s. The dissociation was monitored for 120 s. Bulk refractive index differences were corrected for by subtracting the response obtained on a reference flow cell. Here, the antigens were flown over a surface with immobilized anti-human Fab specific antibody on which HBS-EP has been injected rather than the antibodies.

Determination of avidity was done by direct immobilization of biotinylated recombinant human, murine and cynomolgus Robo 4 on a Streptavidin sensor chip. Immobilization level ranged from 300 to 1000 RU. Anti-Robo 4 antibodies were passed through the flow cells for 220 s at 30 μl/min in a concentration range from 0.78-50 nM. Dissociation was monitored for 220 s. For the blank and the 25 nM injection dissociation was monitored for 600 s.

Kinetic constants were derived using the Biacore T100 Evaluation Software (vAA, Biacore), to fit rate equations for 1:1 Langmuir binding by numerical integration. Kinetic values are shown in Tables 1 and 2.

For determination of the epitope of the four analyzed anti-Robo 4 antibodies, domain variants of human Robo 4 (FN-like domain 1, FN-like domain 2, Ig-like domain 1 and Ig-like domain 2) were used. Anti-Robo 4 antibodies were captured for 60 s at 50 nM on a sensorchip surface with immobilized anti-human Fab specific antibody (GE Healthcare). Domain variants of human Robo 4 were passed at a concentration range of 0.46-1000 nM with a flow of 30 μl/min through the flow cells over 90 s. The dissociation was monitored for 120 s. Bulk refractive index differences were corrected for by subtracting the response obtained on reference flow cell as described above. Results are summarized in Table 3.

Antibody clones 7G2, 01E06 and 01F09 bind to human Robo 4 Ig-like domain 2. 7G2 shows also a weaker binding to human Robo 4 Ig-like domain 1, indicating that the epitope of this antibody clone might be within Ig-like domain 1 and 2. 01F05 binds an epitope located in the human Robo 4 FN-like domain 2.

TABLE 1
Affinity rate constants of anti Robo 4 antibodies to different Robo 4 antigens.

| | Robo 4 | | | | | | | | |
|---|---|---|---|---|---|---|---|---|---|
| | human | | | murine | | | cynomolgus | | |
| | kon | koff | KD | kon | koff | KD | kon | koff | KD |
| | (×10⁴ M⁻¹s⁻¹) | (×10⁻³ s⁻¹) | [nM] | (×10⁴ M⁻¹s⁻¹) | (×10⁻³ s⁻¹) | [nM] | (×10⁴ M⁻¹s⁻¹) | (×10⁻³ s⁻¹) | [nM] |
| 7G2 | 4.38 | 1.02 | 23.4 | nb | nb | nb | 1.81 | 1.16 | 64.1 |
| 01E06 | 47.9 | 0.33 | 0.69 | 7.71 | 12.8 | 166 | 8.04 | 0.08 | 0.96 |
| 01F05 | 5.96 | 0.91 | 15.3 | 3.08 | 0.31 | 10.1 | 1.33 | 1.91 | 144 |
| 01F09 | 24.1 | 0.63 | 2.63 | 12.0 | 30.7 | 256 | 6.66 | 31.5 | 474 |

nb: no binding

TABLE 2
Avidity of anti-Robo 4 antibodies to different Robo 4 antigens.

| | Robo 4 | | | | | | | | |
|---|---|---|---|---|---|---|---|---|---|
| | human | | | murine | | | cynomolgus | | |
| | kon | koff | KD | kon | koff | KD | kon | koff | KD |
| | (×10⁴ M⁻¹s⁻¹) | (×10⁻³ s⁻¹) | [nM] | (×10⁴ M⁻¹s⁻¹) | (×10⁻³ s⁻¹) | [nM] | (×10⁴ M⁻¹s⁻¹) | (×10⁻³ s⁻¹) | [nM] |
| 7G2 | 36.1 | 15.2 | 0.421 | nb | nb | nb | 31.0 | 29.1 | 0.94 |
| 01E06 | 176 | 1250 | 0.0007 | 75.0 | 80.5 | 1.07 | 113 | 15500 | 0.0001 |
| 01F05 | 65.7 | 613 | 0.093 | 37.3 | 732 | 0.19 | 82.2 | 47.5 | 0.58 |
| 01F09 | 210 | 378 | 0.018 | 105 | 1.15 | 1.09 | 222 | 81.7 | 0.37 |

nb: no binding

TABLE 3
Affinity of anti-Robo 4 antibodies to
different domains of human Robo 4.

| | FN-like | FN-like | IgG-like | IgG-like |
|---|---|---|---|---|
| | domain 1 | domain 2 | domain 1 | domain 2 |
| | KD [nM] | KD [nM] | KD [nM] | KD [nM] |
| 7G2 | nb | nb | 151 | 66.7 |
| 01E06 | nb | nb | nb | 4.9 |
| 01F05 | nb | 30.6 | nb | nb |
| 01F09 | nb | nb | nb | 55.5 |

nb: no binding

### Example 9

Preparation of Anti-Robo 4/Anti-CD3 1+1 and 2+1 CrossFab-IgG Bispecific Antibodies

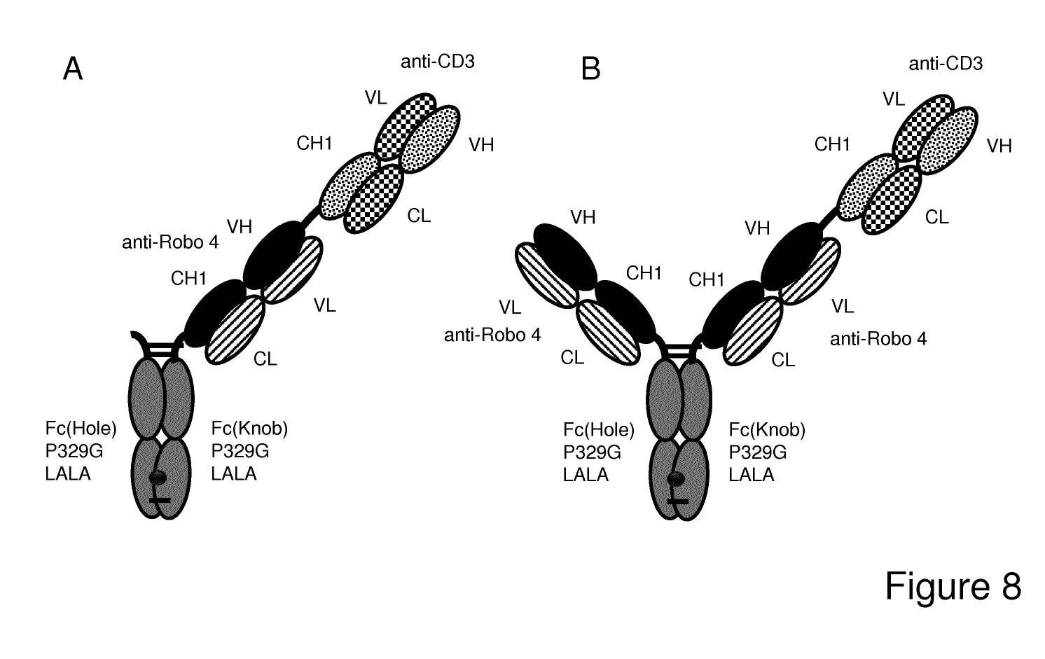

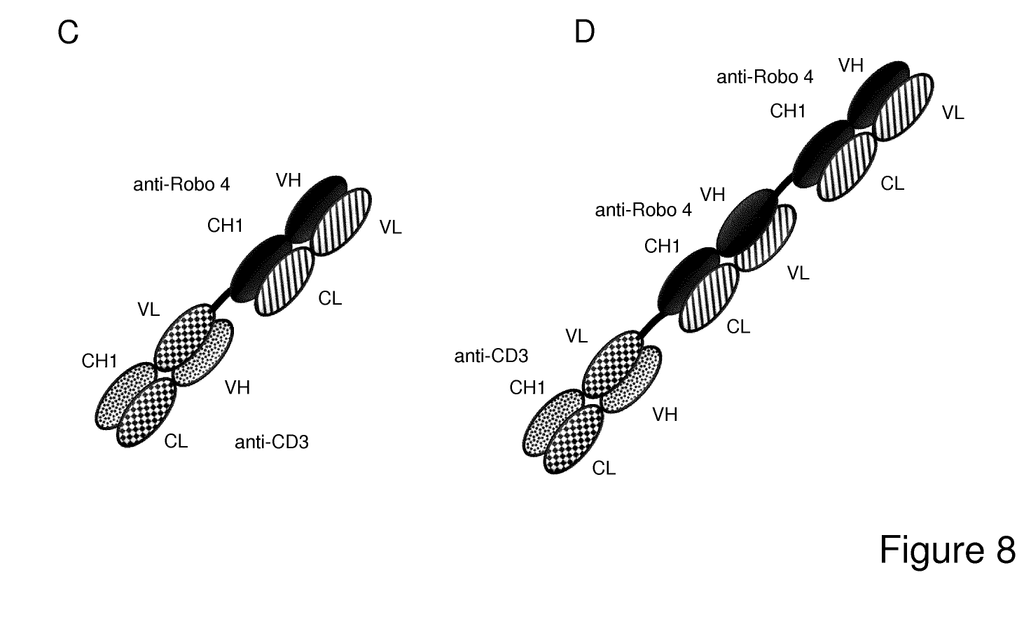

The IgG-based molecules are bispecific, meaning that the molecules comprise an antigen binding moiety capable of specific binding to CD3 and at least one antigen binding moiety capable of specific binding to Robo 4. The antigen binding moieties are Fab fragments composed of a heavy and a light chain, each comprising a variable and a constant region. At least one of the Fab fragments is a “CrossFab” fragment, wherein the variable domains of the Fab heavy and light chain are exchanged. The exchange of heavy and light chain variable domains within Fab fragments assures that Fab fragments of different specificity do not have identical domain arrangement and consequently do not “interchange” light chains. The bispecific molecule can be monovalent for both antigens (1+1, see FIG. 8A) or monovalent for CD3 and bivalent for Robo 4 (2+1, see FIG. 8B).

The following molecules were prepared in this example; a schematic illustration thereof is shown in FIG. 8:
A. “1+1 CrossFab-IgG” (VH/VL exchange in CD3 binder, CD3 binder V9, Robo 4 binder 01F09) (FIG. 8A, SEQ ID NOs 55, 59, 79, 83).
B. “1+1 CrossFab-IgG” (VH/VL exchange in CD3 binder, CD3 binder V9, Robo 4 binder 01F05) (FIG. 8A, SEQ ID NOs 41, 53, 79, 83).
C. “1+1 CrossFab-IgG” (VH/VL exchange in CD3 binder, CD3 binder V9, Robo 4 binder 01E06) (FIG. 8A, SEQ ID NOs 35, 39, 79, 83).
D. “1+1 CrossFab-IgG” (VH/VL exchange in CD3 binder, CD3 binder V9, Robo 4 binder 7G2) (FIG. 8A, SEQ ID NOs 61, 65, 79, 83).
E. “1+1 CrossFab-IgG” (VH/VL exchange in CD3 binder, CD3 binder 2C11, Robo 4 binder 01F05) (FIG. 8A, SEQ ID NOs 43, 53, 81, 83).
F. “2+1 CrossFab-IgG” (VH/VL exchange in CD3 binder, CD3 binder V9, Robo 4 binders 01F05) (FIG. 8B, SEQ ID NOs 41, 45, 53, 79).

The molecules were produced by co-transfecting HEK293 EBNA cells growing in suspension with the mammalian expression vectors using polyethylenimine (PEI) as described above for the cynomolgus Robo 4 antigen. For preparation of 1+1 CrossFab-IgG constructs, cells were transfected with the corresponding expression vectors in a 1:1:1:1 ratio (“vector Fc(knob)”: “vector light chain”: “vector light chain CrossFab”: “vector heavy chain-CrossFab”). For preparation of 2+1 CrossFab-IgG constructs, cells were transfected with the corresponding expression vectors in a 1:2:1:1 ratio (“vector Fc(knob)”: “vector light chain”: “vector light chain CrossFab”: “vector heavy chain-CrossFab”).

The secreted proteins were purified from cell culture supernatants by Protein A affinity chromatography, followed by a size exclusion chromatographic step.

For affinity chromatography, supernatant was loaded on a HiTrap ProteinA HP column (CV=5 mL, GE Healthcare) equilibrated with 25 ml 20 mM sodium phosphate, 20 mM sodium citrate, 500 mM NaCl, pH 7.5. Unbound protein was removed by washing with at least 10 column volumes 20 mM sodium phosphate, 20 mM sodium citrate, 0.5 M sodium chloride, pH 7.5. Target protein is eluted in a linear pH gradient over 20 column volumes to 20 mM sodium citrate, 500 mM sodium chloride, pH 3.0. The column was subsequently washed with 10 column volumes 20 mM sodium citrate, 500 mM sodium chloride, pH 3.0. The protein solution was neutralized by adding 1/10 of 0.5 M sodium phosphate, concentrated and filtrated, before loading on a HiLoad Superdex 200 column (GE Healthcare) equilibrated with 20 mM histidine, 140 mM sodium chloride, pH 6.0.

Concentrations of the purified protein samples were determined by measuring the optical density (OD) at 280 nm, using the molar extinction coefficient calculated on the basis of the amino acid sequence. Purity and molecular weight of antibodies were analyzed by SDS PAGE in the presence and absence of a reducing agent (5 mM 1,4-dithiotreitol) and staining with Coomassie (SimpleBlue™ SafeStain, Invitrogen). The NuPAGE® Pre-Cast gel system (Invitrogen, USA) was used according to the manufacturer's instructions (4-12% Tris-Acetate or 4-12% Bis-Tris gels). Alternatively, purity and molecular weight were analysed by CE-SDS analyses in the presence and absence of a reducing agent. The Caliper LabChip GXII system (Caliper Lifescience) was used according to the manufacturer's instructions, with 2 μg samples. The aggregate content of antibody samples was analyzed using either a Superdex 200 10/300GL analytical size-exclusion column (GE Healthcare) equilibrated in 2 mM MOPS, 150 mM NaCl, 0.02% (w/v) NaN₃, pH 7.3, or a TSKgel G3000 SW XL analytical size-exclusion column (Tosoh) equilibrated in 25 mM K₂HPO₄, 125 mM NaCl, 200 mM L-arginine monohydrocloride, 0.02% (w/v) NaN₃, pH 6.7 running buffer at 25° C.

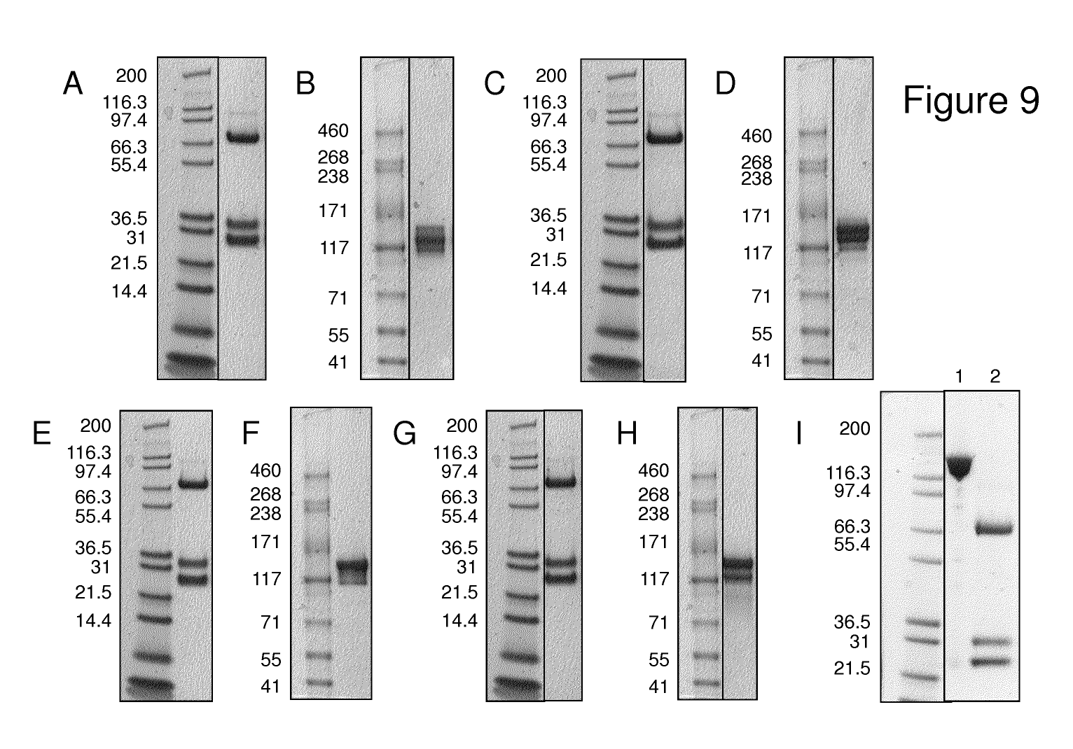

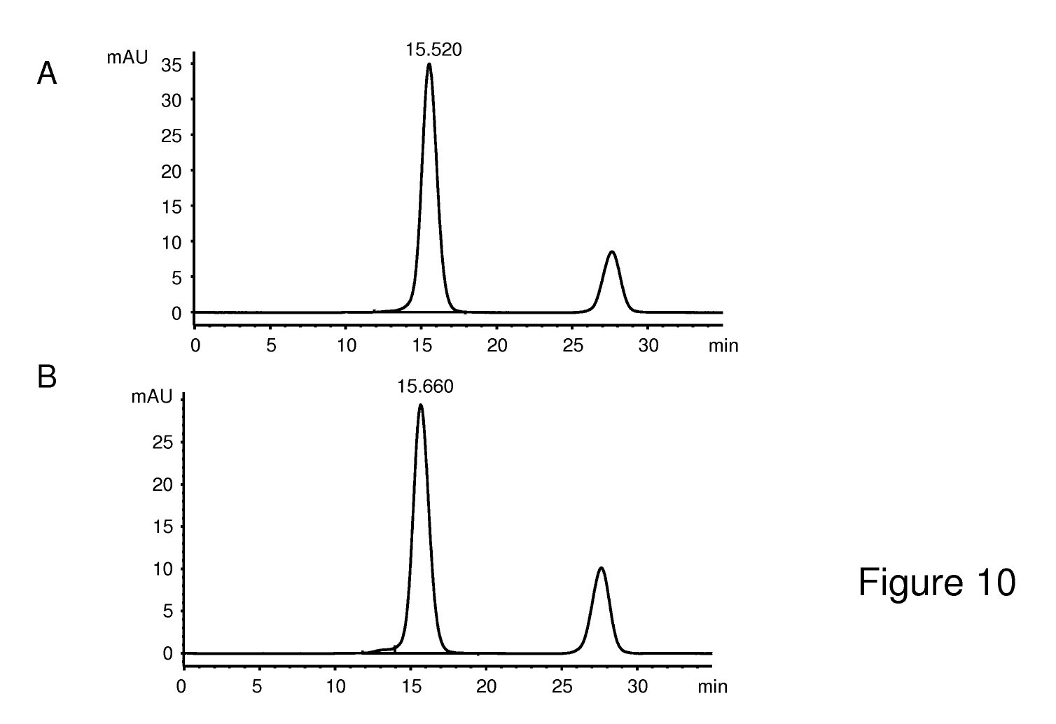

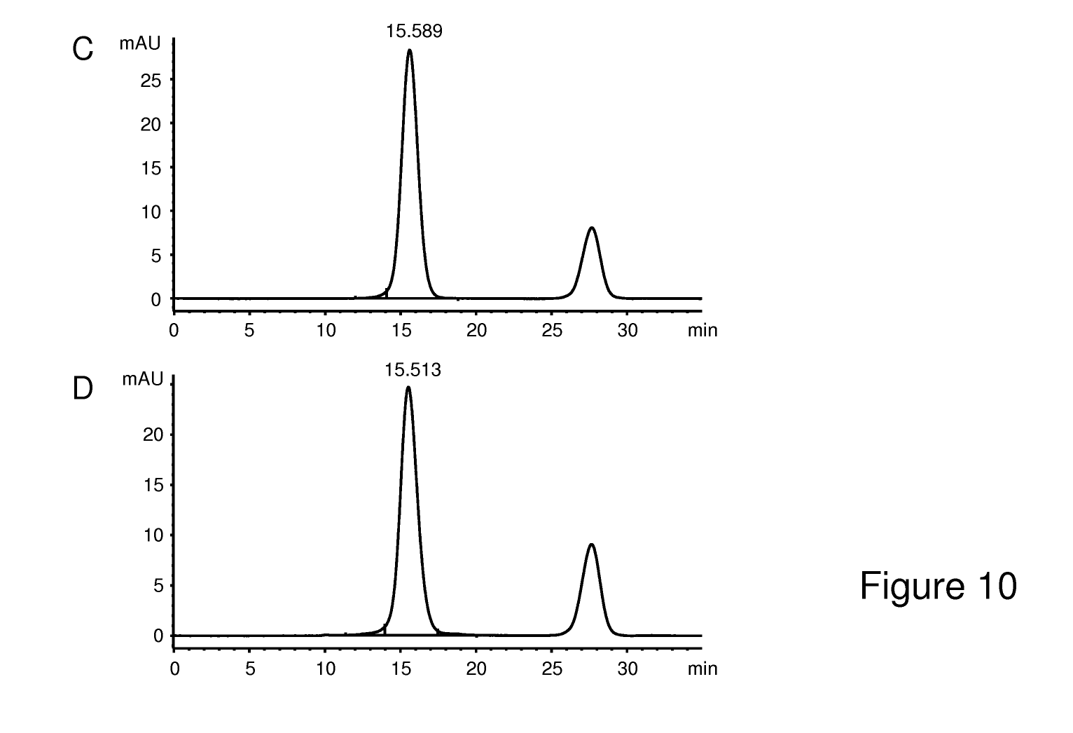

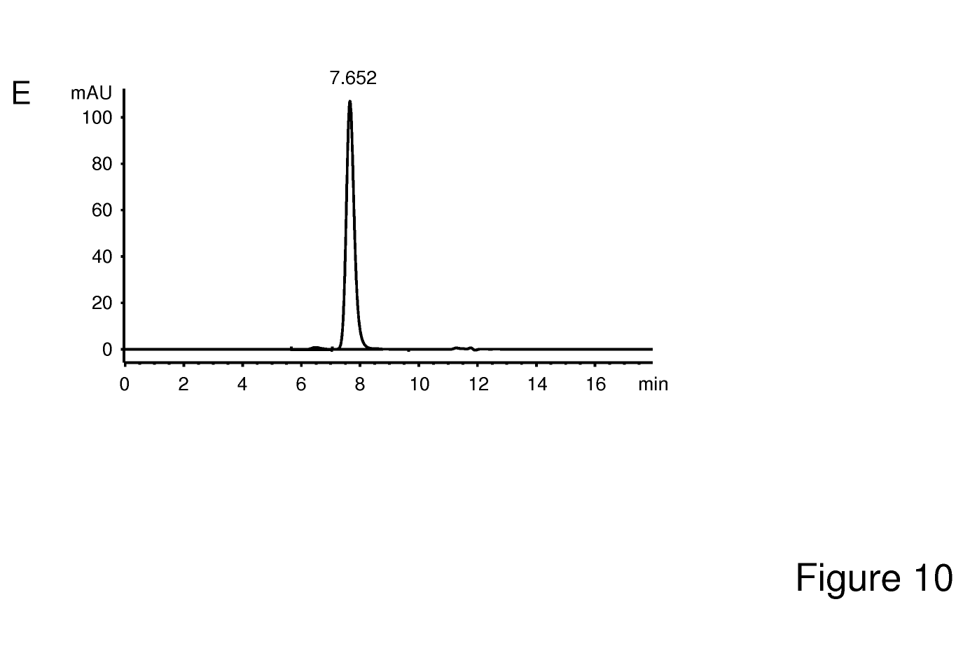

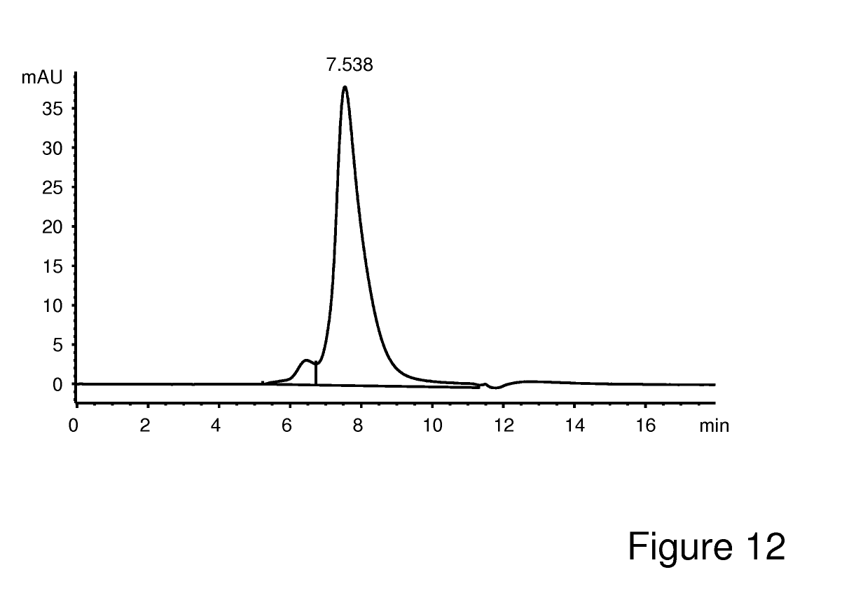

Results for the 1+1 CrossFab-IgG constructs are shown in FIGS. 9 and 10, and Table 4, results for the 2+1 CrossFab-IgG construct in FIGS. 11 and 12 and Table 5.

TABLE 4
Yield and aggregate content of 1 + 1 CrossFab-IgG preparations.

| | Yield | HMW | LMW | Monomer |
|---|---|---|---|---|
| Construct | [mg/l] | [%] | [%] | [%] |
| A | 3.14 | 0.5 | 0 | 99.5 |
| B | 11.8 | 3.9 | 0 | 96.1 |
| C | 13.7 | 0.5 | 0 | 99.5 |
| D | 12.7 | 0.6 | 0 | 99.4 |
| E | 49.2 | 1.1 | 0 | 98.9 |

TABLE 5
Yield and aggregate content of 2 + 1 CrossFab-IgG preparation.

| | Yield | HMW | LMW | Monomer |
|---|---|---|---|---|
| Construct | [mg/l] | [%] | [%] | [%] |
| F | 2.25 | 5.2 | 0 | 94.8 |

### Example 10

Preparation of Anti-Robo 4/Anti-CD3 Fab-CrossFab and Fab-Fab-CrossFab Bispecific Antibodies

The non-IgG-based molecules are bispecific, meaning that the molecules comprise an antigen binding moiety capable of specific binding to CD3 and at least one antigen binding moiety capable of specific binding to Robo 4. The antigen binding moieties are Fab fragments composed of a heavy and a light chain, each comprising a variable and a constant region. At least one of the Fab fragments is a “CrossFab” fragment, wherein the variable domains of the Fab heavy and light chain are exchanged. The exchange of heavy and light chain variable domains within Fab fragments assures that Fab fragments of different specificity do not have identical domain arrangement and consequently do not “interchange” light chains. The bispecific molecule can be monovalent for both antigens (1+1, see FIG. 8C) or monovalent for CD3 and bivalent for Robo 4 (2+1, see FIG. 8D).

The following molecules were prepared in this example; a schematic illustration thereof is shown in FIG. 8:
G. “1+1 Fab-CrossFab” (VH/VL exchange in CD3 binder, CD3 binder V9, Robo 4 binder 01E06) (FIG. 8C, SEQ ID NOs 37, 39, 79).
H. “1+1 Fab-CrossFab” (VH/VL exchange in CD3 binder, CD3 binder V9, Robo 4 binder 7G2) (FIG. 8C, SEQ ID NOs 63, 65, 79).
I. “1+1 Fab-CrossFab” (VH/VL exchange in CD3 binder, CD3 binder V9, Robo 4 binder 01F09) (FIG. 8C, SEQ ID NOs 57, 59, 79).
J. “1+1 Fab-CrossFab” (VH/VL exchange in CD3 binder, CD3 binder V9, Robo 4 binder 01F05) (FIG. 8C, SEQ ID NOs 47, 53, 79).
K. “1+1 Fab-CrossFab” (VH/VL exchange in CD3 binder, CD3 binder 2C11, Robo 4 binder 01F05) (FIG. 8C, SEQ ID NOs 49, 53, 81).
L. “2+1 Fab-Fab-CrossFab” (VH/VL exchange in CD3 binder, CD3 binder V9, Robo 4 binders 01F05) (FIG. 8D, SEQ ID NOs 51, 53, 79).

The molecules were produced by co-transfecting HEK293-EBNA cells with the mammalian expression vectors using polyethylenimine (PEI) as described above. For preparation of 1+1 Fab-CrossFab constructs, cells were transfected with the corresponding expression vectors in a 1:1:1 ratio (“vector CH1-VH-CL-VH”: “vector light chain VL-CL”: “vector light chain CH1-VL”). For preparation of 2+1 Fab-Fab-CrossFab constructs, cells were transfected with the corresponding expression vectors in a 1:1:1 ratio (“vector CH1-VH-CH1-VH-CL-VH”: “vector light chain VL-CL”: “vector light chain CH1-VL”).

The secreted proteins were purified from cell culture supernatants by Protein A and Protein G affinity chromatography, followed by a size exclusion chromatographic step.

For affinity chromatography supernatant was loaded on a HiTrap Protein A HP column (CV=5 mL, GE Healthcare) coupled to a HiTrap Protein G HP column (CV=5 mL, GE Healthcare), each column equilibrated with 30 ml 20 mM sodium phosphate, 20 mM sodium citrate, pH 7.5. Unbound protein was removed by washing both columns with 6 column volumes 20 mM sodium phosphate, 20 mM sodium citrate, pH 7.5. Subsequently, an additional wash step was necessary to wash only the HiTrap Protein G HP column, using at least 8 column volumes 20 mM sodium phosphate, 20 mM sodium citrate, pH 7.5. The target protein was eluted from the HiTrap Protein G HP column using a step gradient with 7 column volumes 8.8 mM formic acid, pH 3.0. The protein solution was neutralized by adding 1/10 of 0.5 M sodium phosphate, pH 8.0, concentrated and filtrated before loading on a HiLoad Superdex 200 column (GE Healthcare) equilibrated with 25 mM potassium phosphate, 125 mM sodium chloride, 100 mM glycine, pH 6.7.

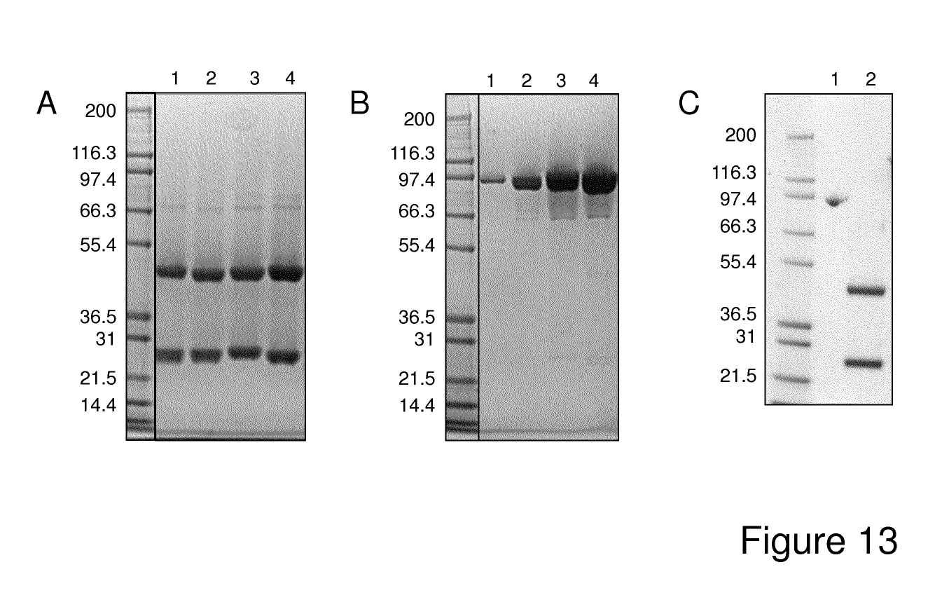

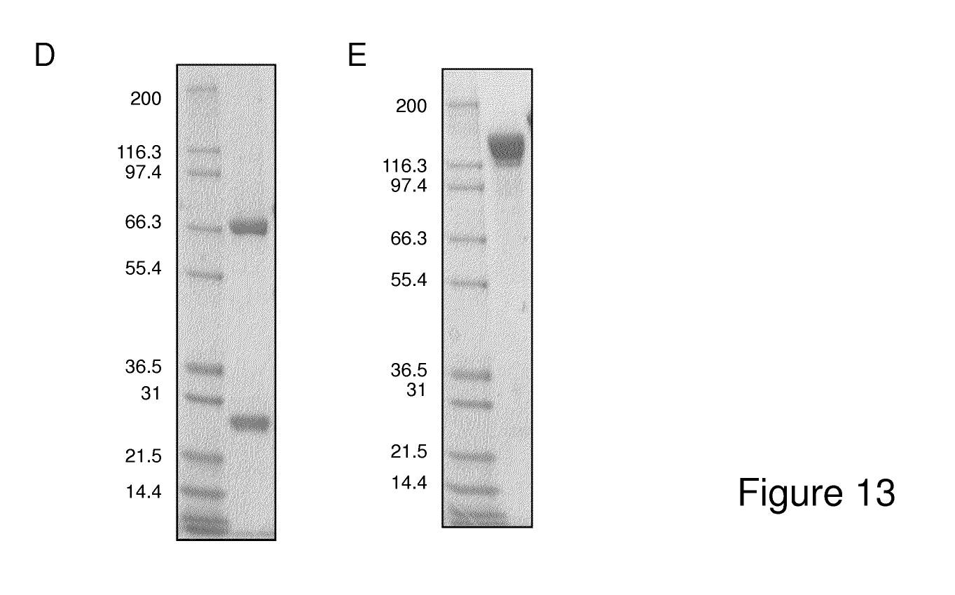

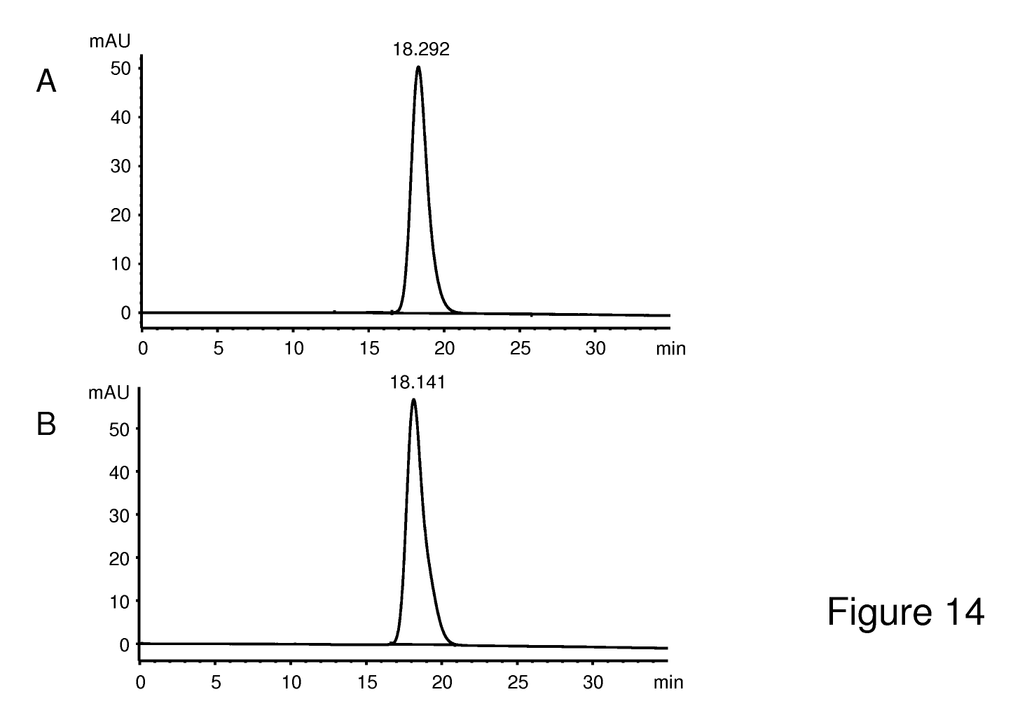

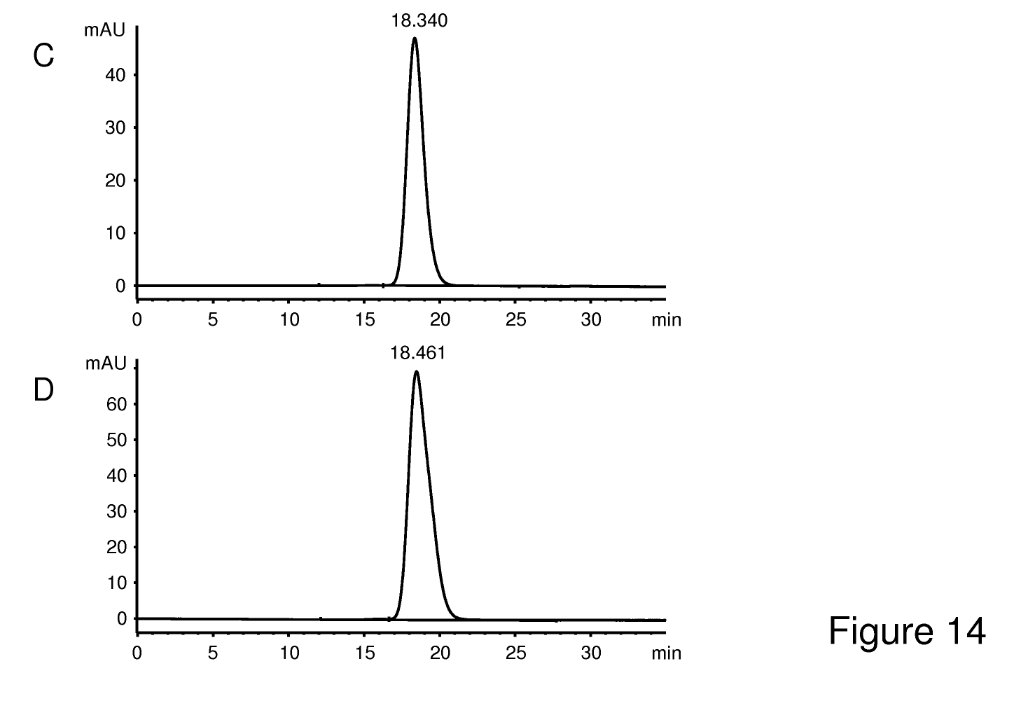

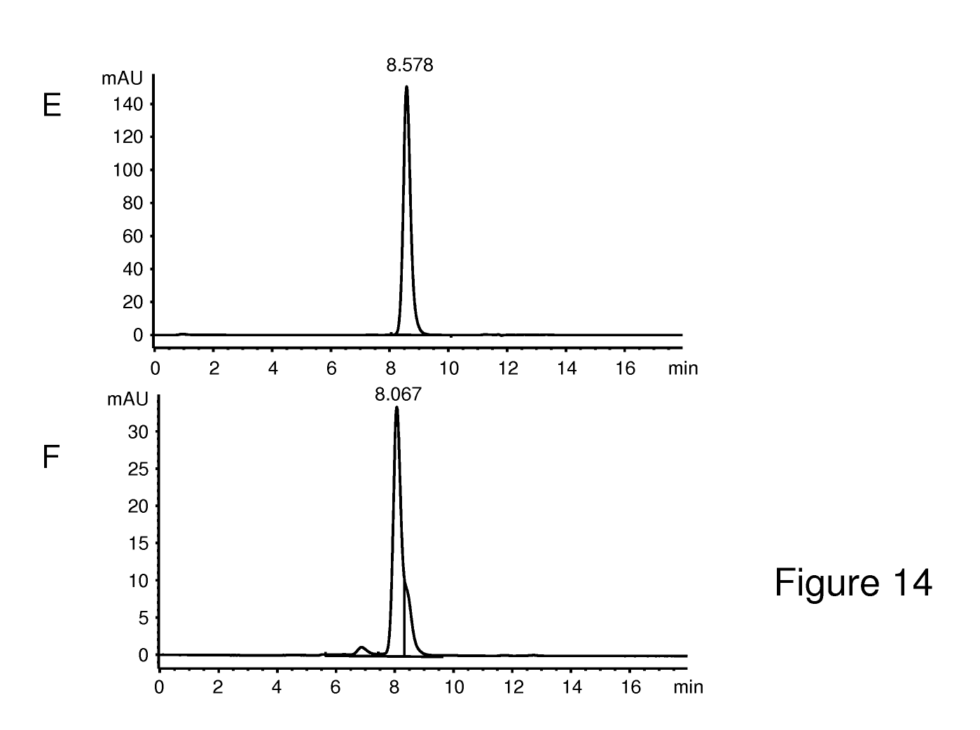

The purified proteins were analyzed by SDS PAGE and analytical size exclusion chromatography as described above for the CrossFab-IgG constructs. Results are shown in FIGS. 13 and 14, and Table 6 and 7.

TABLE 6
Yield and aggregate content of 1 + 1 Fab-CrossFab preparations.

| | Yield | HMW | LMW | Monomer |
|---|---|---|---|---|
| Construct | [mg/l] | [%] | [%] | [%] |
| G | 7.78 | 0.4 | 0 | 99.6 |
| H | 3.44 | 0 | 0 | 100 |
| I | 7.78 | 0.1 | 0 | 99.9 |
| J | 23.15 | 0.5 | 0 | 99.5 |
| K | 10.5 | 0 | 0 | 100 |

TABLE 7
Yield and aggregate content of 2 +
1 Fab-Fab-CrossFab preparations.

| | Yield | HMW | LMW | Monomer |
|---|---|---|---|---|
| Construct | [mg/l] | [%] | [%] | [%] |
| L | 6.75 | 5.2 | 20 | 75 |

### Example 11

Binding of Anti-Robo 4 IgGs to CHO-Robo 4 Cells

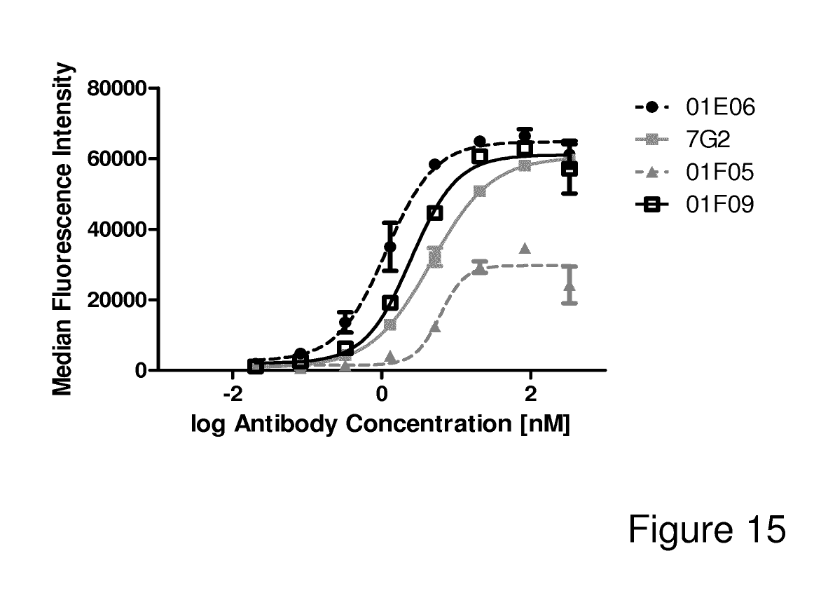

Binding of anti-Robo 4 IgGs was tested on CHO cells stably expressing full-length human Robo 4 (CHO-Robo 4). Briefly, cells were harvested, counted and checked for viability. 200 000 cells/well in 100 ml PBS 0.1% BSA were incubated in a round-bottom 96-well plate for 30 min at 4° C. with increasing concentrations of the anti-Robo 4 IgGs (333 nM-0.02 nM) or corresponding isotype controls, washed twice with cold PBS containing 0.1% BSA, re-incubated for further 30 min at 4° C. with the PE-conjugated AffiniPure F(ab′)2 Fragment goat anti-human IgG Fcg Fragment Specific (Jackson Immuno Research Lab PE #109-116-170) secondary antibody, washed twice with cold PBS/0.1% BSA and immediately analyzed by FACS using a FACSCantoII (Software FACS Diva) by gating live, DAPI-negative, cells. Binding curves and EC50 values for 7G2 (4.6 nM), 01F05 (6.1 nM), 01E06 (1.1 nM) and 01F09 (2.5 nM) were obtained using GraphPadPrism5 (FIG. 15).

### Example 12

Antibody-Dependent Cell-Mediated Cytotoxicity (ADCC) Using Wildtype and Glycoengineered Anti-Robo 4 IgGs

The potential of different anti-Robo 4 IgGs to induce ADCC was assessed. In one experiment, wildtype (clones 7G2, 01F05) and glycoengineered (having an increased proportion of non-fucosylated oligosaccharide residues in the Fc region; clones 7G2, 01F05, 01F09) anti-Robo 4 IgGs were used. In a second experiment, a wildtype anti-Robo 4 IgG (clone 01E06) was compared to a corresponding glycoengineered anti-Robo 4 IgG wherein one binding arm has been deleted (one-armed (OA), monovalent binder).

HUVEC cells were harvested with Cell Dissociation Buffer, washed, and plated at a density of 30 000 cells/well using flat-bottom 96-well plates. Cells were left to adhere overnight. Human peripheral blood mononuclear cells (PBMCs) were prepared by Histopaque density centrifugation from enriched lymphocyte preparations (buffy coats) obtained from local blood banks or from fresh blood from healthy human donors. Briefly, blood was diluted with sterile PBS and carefully layered over a Histopaque gradient (Sigma, #H8889). After centrifugation (450×g, 30 minutes, room temperature, no brake), part of the plasma above the PBMC-containing interphase was discarded. The PBMCs were transferred in a new 50 ml falcon tube subsequently filled up with PBS to a final volume of 50 ml. The mixture was centrifuged at room temperature (400×g, 10 minutes), the supernatant discarded and the PBMC pellet washed twice with sterile PBS (centrifugation steps for 10 minutes at 350×g). The resulting PBMC population was counted automatically (ViCell) and stored in RPMI1640 medium containing 10% FCS and 1% L-alanyl-L-glutamine (Biochrom, K0302) at 37° C., 5% CO₂ in cell the incubator until further use.

PBMCs were added to target cells (medium exchanged to AIM-V) at an effector to target cell ratio (E:T, PBMCs:HUVEC) of 25:1. The respective anti-Robo 4 IgGs (1 pg/ml-10 mg/ml) were added (in triplicate) to the PBMCs:HUVEC co-cultures and incubated for 4 h at 37° C., 5% CO₂. Target cell killing was assessed by measuring LDH release using a commercially available kit (LDH detection kit, Roche Applied Science, #11 644 793 001) according the to manufacturer's instructions. ADCC was calculated using the following formula:

Percentage ADCC=([sample release−spontaneous release]/[maximal release−spontaneous release])×100

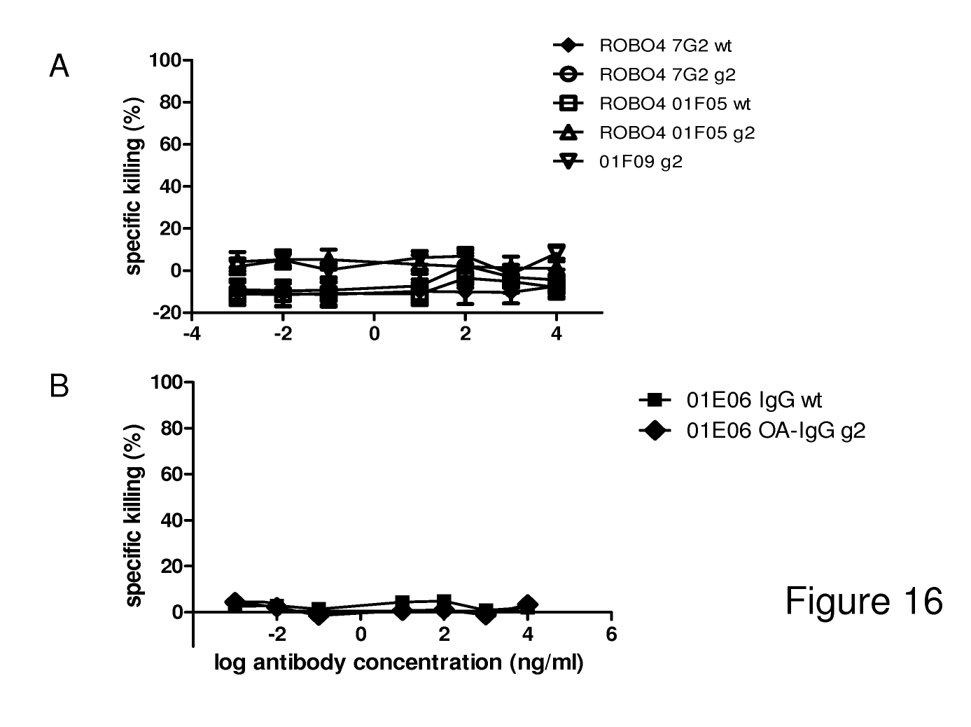

No target cell killing (HUVEC) was detected with any of the wildtype or glycoengineered mono- or bivalent anti-Robo 4 IgGs (FIG. 16), showing that anti-Robo 4 antibodies are unable to induce ADCC irrespective of glycosylation or binding valency.

### Example 13

T-Cell Killing Induced by Anti-Robo 4/Anti-CD3 Bispecific Antibodies

T-cell mediated killing of human endothelial cells (HUVECs) induced by anti-Robo 4/anti-CD3 bispecific antibodies in the Fab-CrossFab and the 1+1 CrossFab-IgG format was assessed. Four different anti-Robo 4 antibody clones (01F05, 01E06, 01F09, 7G2) were compared in the two formats. All constructs contained the anti-human CD3 antibody V9 (described in Rodrigues et al., Int J Cancer Suppl 7, 45-50 (1992) and U.S. Pat. No. 6,054,297; see SEQ ID NOs 85 (VH) and 87 (VL)).

Briefly, HUVEC cells were harvested with Cell Dissociation Buffer, washed, and plated at a density of 30 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from local blood banks or of fresh blood from healthy human donors as described above. T cell enrichment from PBMCs was performed using the Pan T Cell Isolation Kit II (Miltenyi Biotec #130-091-156), according to the manufacturer's instructions. Briefly, the cell pellet was diluted in 40 μl cold buffer per 10 million cells (PBS with 0.5% BSA, 2 mM EDTA, sterile filtered) and incubated with 10 μl Biotin-Antibody Cocktail per 10 million cells for 10 min at 4° C. 30 μl cold buffer and 20 μl Anti-Biotin magnetic beads per 10 million cells were added, and the mixture incubated for another 15 min at 4° C. Cells were washed by adding 10-20× the volume of the antibody incubation mix described above and a subsequent centrifugation step at 300×g for 10 min. Up to 100 million cells were resuspended in 500 μl buffer. Magnetic separation of unlabeled human pan T cells was performed using LS columns (Miltenyi Biotec #130-042-401) according to the manufacturer's instructions. The resulting T cell population was counted automatically (ViCell) and stored in AIM-V medium at 37° C., 5% CO₂ in the incubator until further use (not longer than 24 h).

For the killing assay, the respective antibody dilutions were added at the indicated concentrations (concentration range of 0.5 pM-50 nM; in triplicate). Human isolated pan T cells were added to HUVECs at a final E:T ratio of 5:1. Target cell killing was assessed after 22 h incubation at 37° C., 5% CO₂ by quantification of LDH released into cell supernatants by apoptotic/necrotic cells (LDH detection kit, Roche Applied Science, #11 644 793 001), according to the manufacturer's instructions.

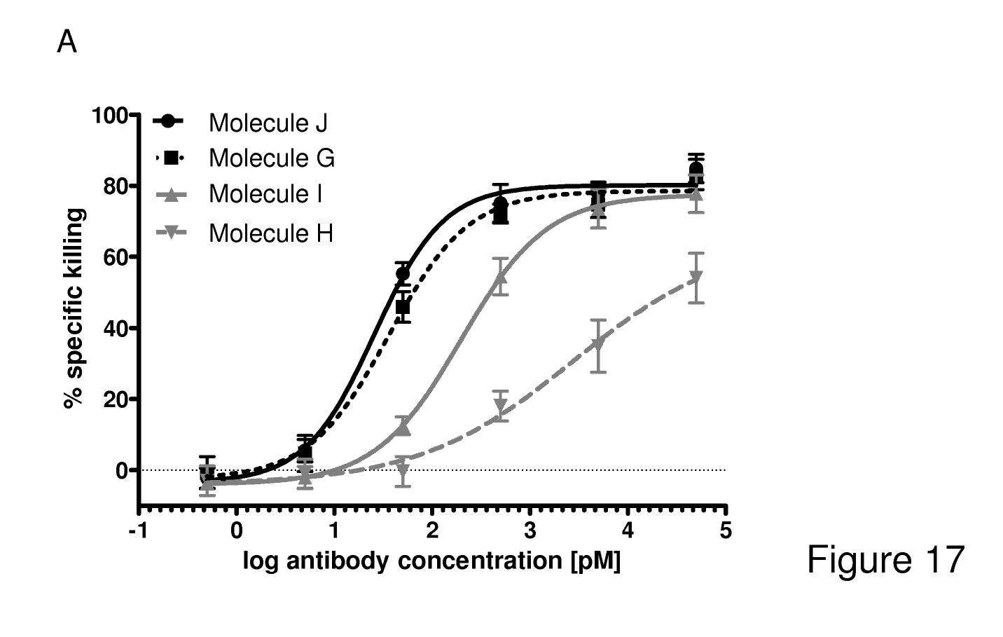

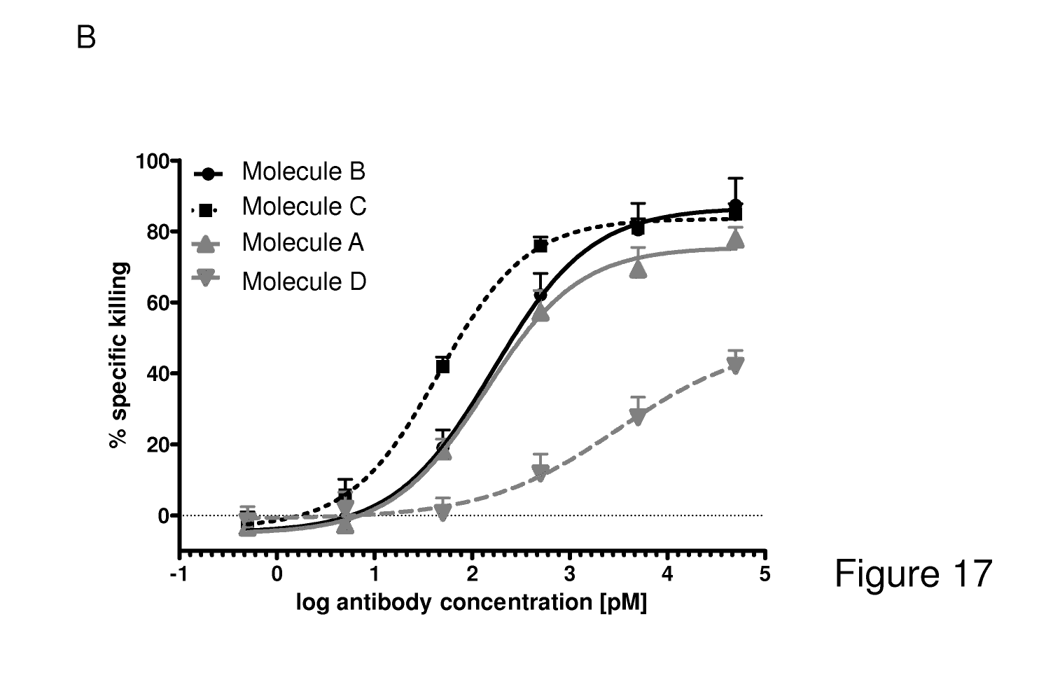

The results of the experiment are shown in FIG. 17. Maximal lysis of the target cells (=100%) was achieved by incubation of target cells with 1% Triton X-100. Minimal lysis (=0%) refers to target cells co-incubated with effector cells without bispecific construct or control IgG. EC50 values related to killing assays, calculated using GraphPadPrism5, are given in Table 8.

TABLE 8
EC50 values (pM) for T-cell mediated killing
of human endothelial cells (HUVECs) induced by
anti-Robo 4/anti-CD3 bispecific antibodies.

| | | Molecule | |
|---|---|---|---|
| Molecule | EC50 | (1 + 1 | EC50 |
| (Fab-CrossFab) | [pM] | CrossFab-IgG) | [pM] |
| J (01F05/V9) | 26 | B (01F05/V9) | 164 |
| G (01E06/V9) | 36 | C (01E06/V9) | 46 |
| I (01F09/V9) | 198 | A (01F09/V9) | 137 |
| H (7G2/V9) | 2763 | D (7G2/V9) | 3143 |

### Example 14

CD25 Upregulation on Human Effector Cells after T Cell-Mediated Killing of Human Endothelial Cells Induced by Anti-Robo 4/Anti-CD3 Bispecific Antibodies

Activation of CD4⁺ and CD8⁺ T cells after T-cell mediated killing of HUVECs induced by the anti-Robo 4/anti-CD3 bispecific antibodies in the Fab-CrossFab and the 1+1 CrossFab-IgG format was assessed by FACS analysis using antibodies recognizing the T cell activation marker CD25.

The same antibodies were used and the killing assay was performed essentially as described above (Example 13), using an E:T ratio of 5:1 and an incubation time of 17 h. The bispecific constructs and the different IgG controls were adjusted to the same molarity (concentration range of 0.5 pM-50 nM; in triplicate). PHA-M 1-10 μg/ml (Sigma #L8902), a mixture of isolectins isolated from Phaseolus vulgaris, was used as a mitogenic stimulus to induce human T cell activation.

After the incubation, PBMCs were transferred to a round-bottom 96-well plate, centrifuged at 350×g for 5 min and washed twice with PBS containing 0.1% BSA. Surface staining for CD8 (BD #555634), CD4 (Biolegend #344612) and CD25 (BD #555434) was performed according to the suppliers' indications. Cells were washed twice with 150 μl/well PBS containing 0.1% BSA and fixed for 15 min at 4° C. using 100 μl/well fixation buffer (BD #554655). After centrifugation, the samples were resuspended in 200 μl/well PBS 0.1% BSA and analyzed at FACS CantoII (Software FACS Diva).

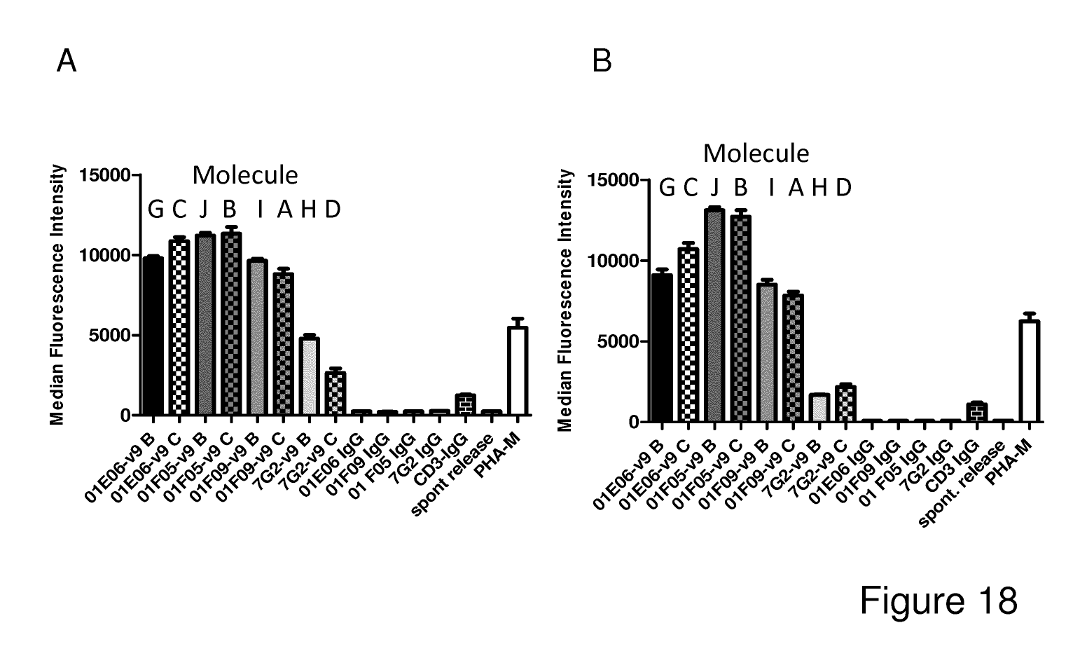

The results are shown in FIG. 18.

### Example 15

T-Cell Killing Induced by Anti-Robo 4/Anti-CD3 Bispecific Antibodies of Different Formats

T-cell mediated killing of human endothelial cells (HUVECs) induced by anti-Robo 4/anti-CD3 bispecific antibodies of different bispecific antibody formats was compared: the Fab-CrossFab format, the Fab-Fab-CrossFab format, the 1+1 CrossFab-IgG format and the 2+1 CrossFab-IgG format—all comprising the anti-Robo 4 binder 01F05 and the anti-human CD3 antibody V9 (molecule J (SEQ ID NOs 47, 53 and 79), molecule L (SEQ ID NOs 51, 53 and 79), molecule B (SEQ ID NOs 41, 53, 79 and 83), and molecule F (SEQ ID NOs 41, 45, 53 and 79), respectively). A 2+1 CrossFab-IgG construct comprising the V9 antibody (CrossFab fragment) and a non-binding IgG was used as control (see SEQ ID NOs 67, 71, 77 and 79).

The killing assay was performed essentially as described above, using freshly isolated human PBMCs. Briefly, HUVEC cells were harvested with Cell Dissociation Buffer, washed, and plated at a density of 30 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from local blood banks or of fresh blood from healthy human donors as described above. For the killing assay, the respective antibody dilutions were added at the indicated concentrations (3 pM-50 nM, in triplicate). Human PBMCs were added at a final E:T ratio of 10:1. Target cell killing was assessed after 24 and 45 h incubation at 37° C., 5% CO₂ by quantification of LDH released in cell supernatants by apoptotic/necrotic cells (LDH detection kit, Roche Applied Science, #11 644 793 001), according to the manufacturer's instructions.

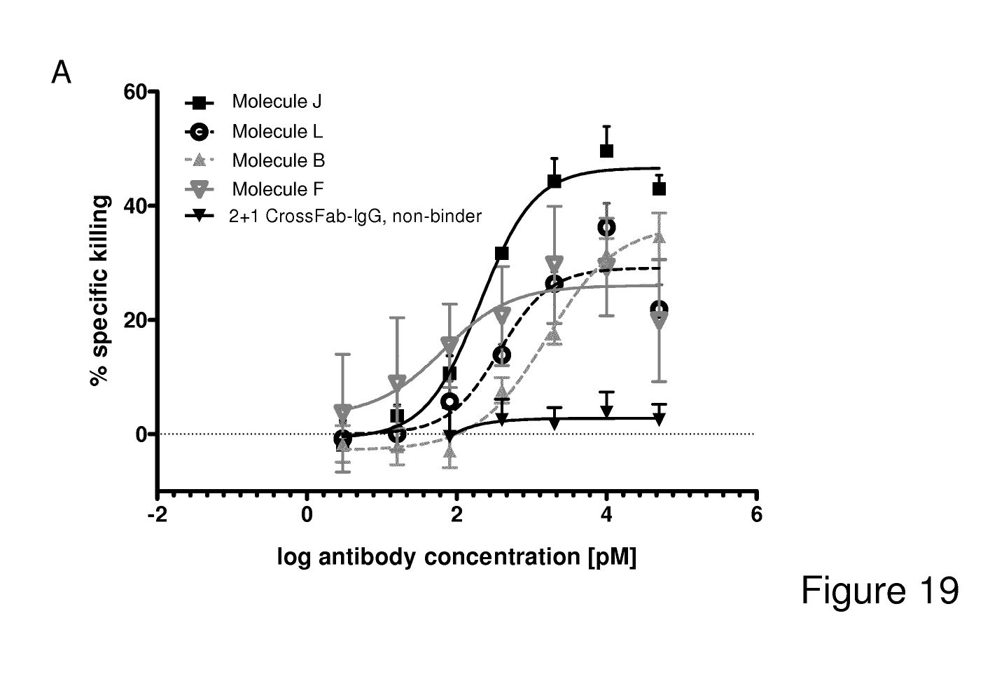

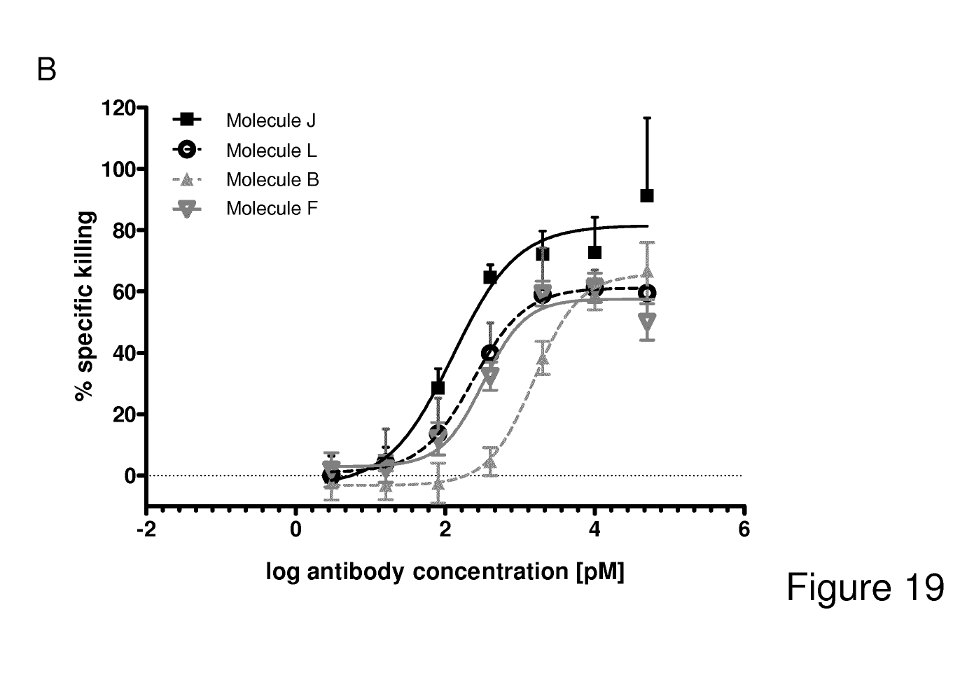

The results of the experiment are shown in FIG. 19. Maximal lysis of the target cells (=100%) was achieved by incubation of target cells with 1% Triton X-100. Minimal lysis (=0%) refers to target cells co-incubated with effector cells without bispecific construct or control IgG. EC50 values related to killing assays, calculated using GraphPadPrism5, are given in Table 9.

TABLE 9
EC50 values (pM) for T-cell mediated killing
of human endothelial cells (HUVECs) induced by
anti-Robo 4/anti-CD3 bispecific antibodies.

| | EC50 [pM] | EC50 [pM] |
|---|---|---|
| Molecule | 24 h | 48 h |
| J (Fab-CrossFab) | 204 | 127 |
| L (Fab-Fab-CrossFab) | 372 | 236 |
| B (1 + 1 CrossFab-IgG) | 1606 | 1548 |
| F (2 + 1 CrossFab-IgG) | 65 | 322 |
| untargeted (2 + 1 CrossFab-IgG) | not calc. | — |

### Example 16

CD25 and CD69 Upregulation on Human Effector Cells after T Cell-Mediated Killing of Human Endothelial Cells Induced by Anti-Robo 4/Anti-CD3 Bispecific Antibodies

Activation of CD4⁺ and CD8⁺ T cells after T-cell mediated killing of HUVECs induced by the anti-Robo 4/anti-CD3 bispecific antibodies in the Fab-CrossFab, the Fab-Fab-CrossFab, the 1+1 CrossFab-IgG and the 2+1 CrossFab-IgG format was assessed by FACS analysis using antibodies recognizing the T cell activation markers CD25 (late activation marker) and CD69 (early activation marker).

The same antibodies were used (molecule J, L, B and F) and the killing assay was performed essentially as described above (Example 15), using an E:T ratio of 10:1 and an incubation time of 24 h.

After the incubation, PBMCs were transferred to a round-bottom 96-well plate, centrifuged at 350×g for 5 min and washed twice with PBS containing 0.1% BSA. Surface staining for CD8 (BD #555634), CD4 (Biolegend #344612), CD69 (Biolegend #310906) and CD25 (BD #555434) was performed according to the suppliers' indications. Cells were washed twice with 150 μl/well PBS containing 0.1% BSA and fixed for 15 min at 4° C. using 100 μl/well fixation buffer (BD #554655). After centrifugation, the samples were resuspended in 200 μl/well PBS 0.1% BSA and analyzed at FACS CantoII (Software FACS Diva).

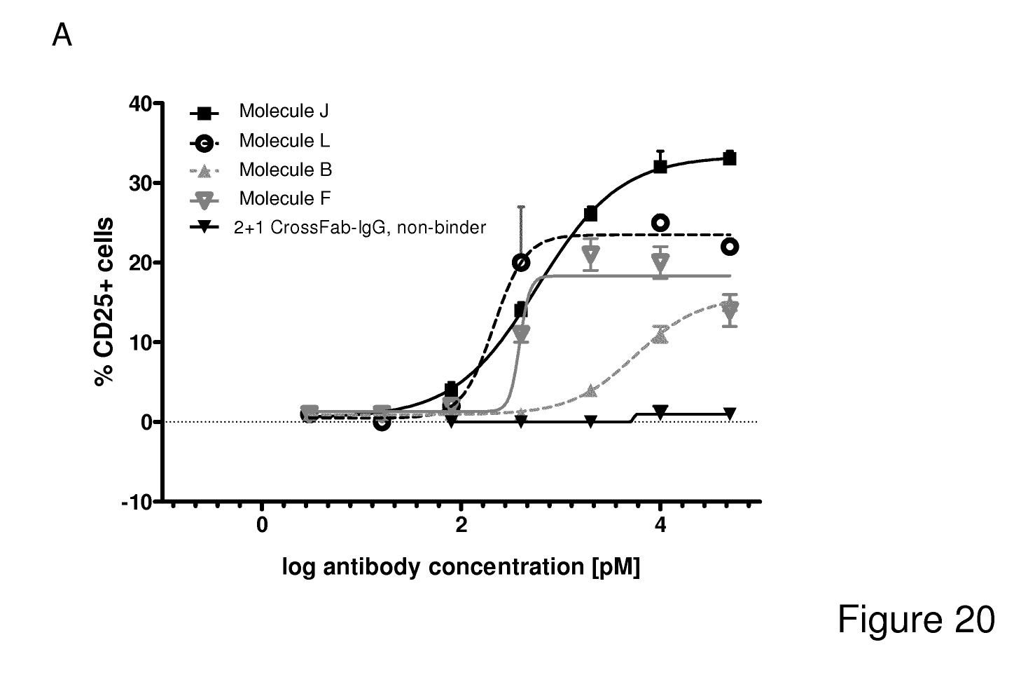

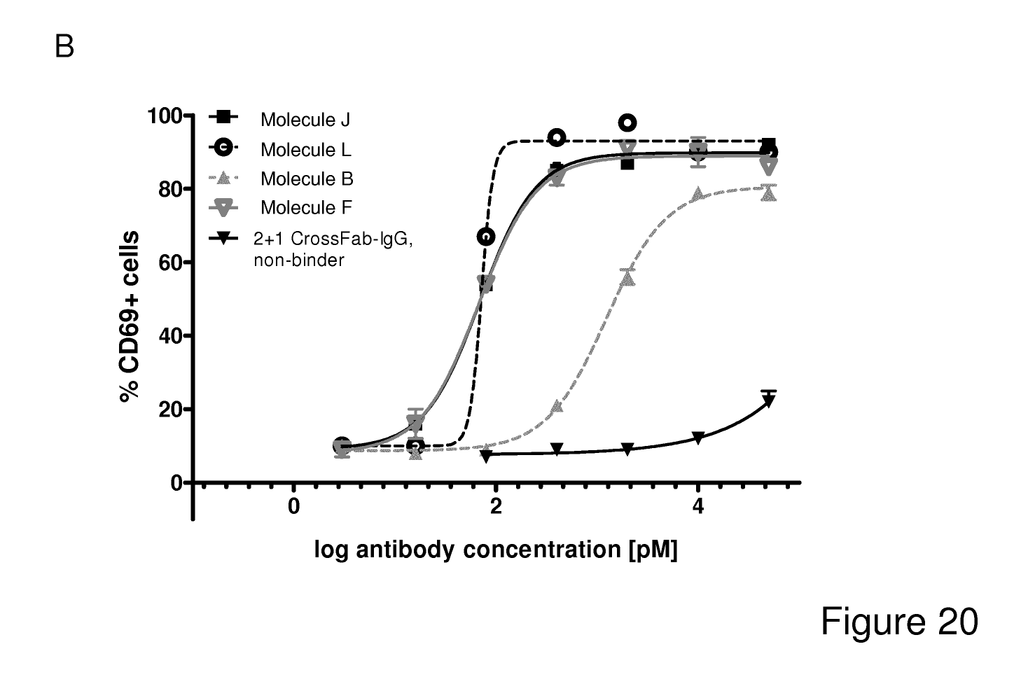

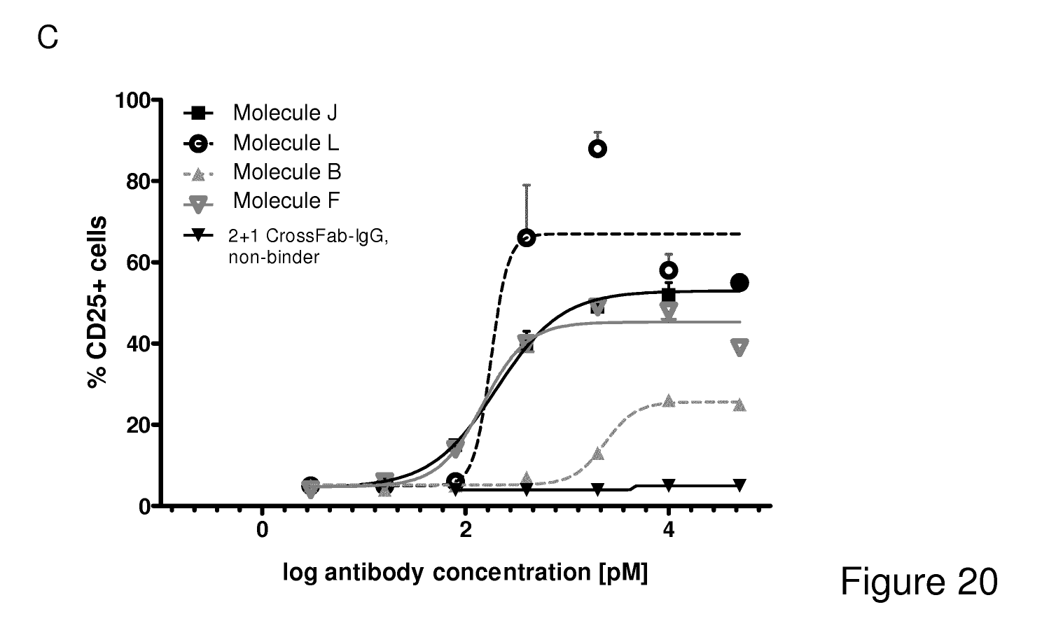

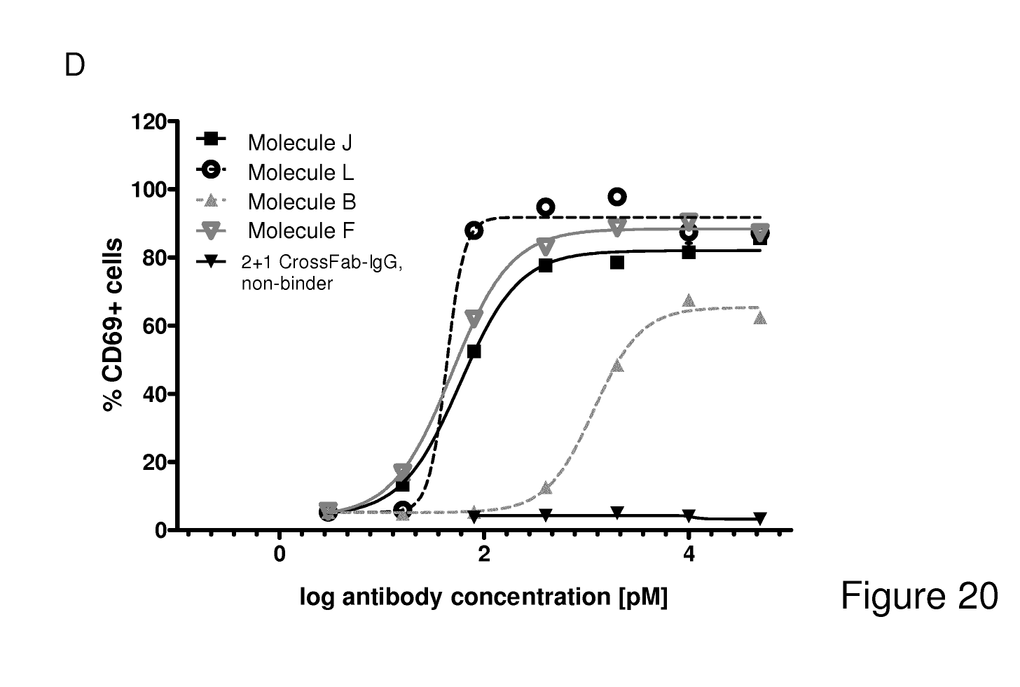

The results are shown in FIG. 20. As for the killing activity (see FIG. 19) molecule B (1+1 CrossFab-IgG format) was less active in inducing T cell activation markers as compared to antibodies in the other formats. The non-binding control molecule was inactive.

### Example 17

Cytokine Secretion by Human Effector Cells after T Cell-Mediated Killing of Human Endothelial Cells Induced by Anti-Robo 4/Anti-CD3 Bispecific Antibodies

Cytokine secretion by human PBMCs after T-cell mediated killing of HUVECs induced by the anti-Robo 4/anti-CD3 bispecific antibodies in the Fab-CrossFab, the Fab-Fab-CrossFab, the 1+1 CrossFab-IgG and the 2+1 CrossFab-IgG format was assessed by FACS analysis of cell supernatants after the killing assay.

The same antibodies were used (molecule J, L, B and F) and the killing assay was performed essentially as described above (Example 15 and 16), using an E:T ratio of 10:1 and an incubation time of 24 h.

At the end of the incubation time, the plate was centrifuged for 5 min at 350×g, the supernatant transferred in a new 96-well plate and stored at −20° C. until subsequent analysis. Granzyme B, TNFα, interferon-γ, IL-2, IL-4 and IL-10 secreted into in cell supernatants were detected using the BD CBA Human Soluble Protein Flex Set, according to manufacturer's instructions on a FACS Cantoll. The following kits were used: BD CBA human Granzyme B Flex Set #BD 560304; BD CBA human TNF Flex Set #BD 558273; BD CBA human IFN-γ Flex Set #BD 558269; BD CBA human IL-2 Flex Set #BD 558270; BD CBA human IL-4 Flex Set #BD 558272; BD CBA human IL-10 Flex Set #BD 558274.

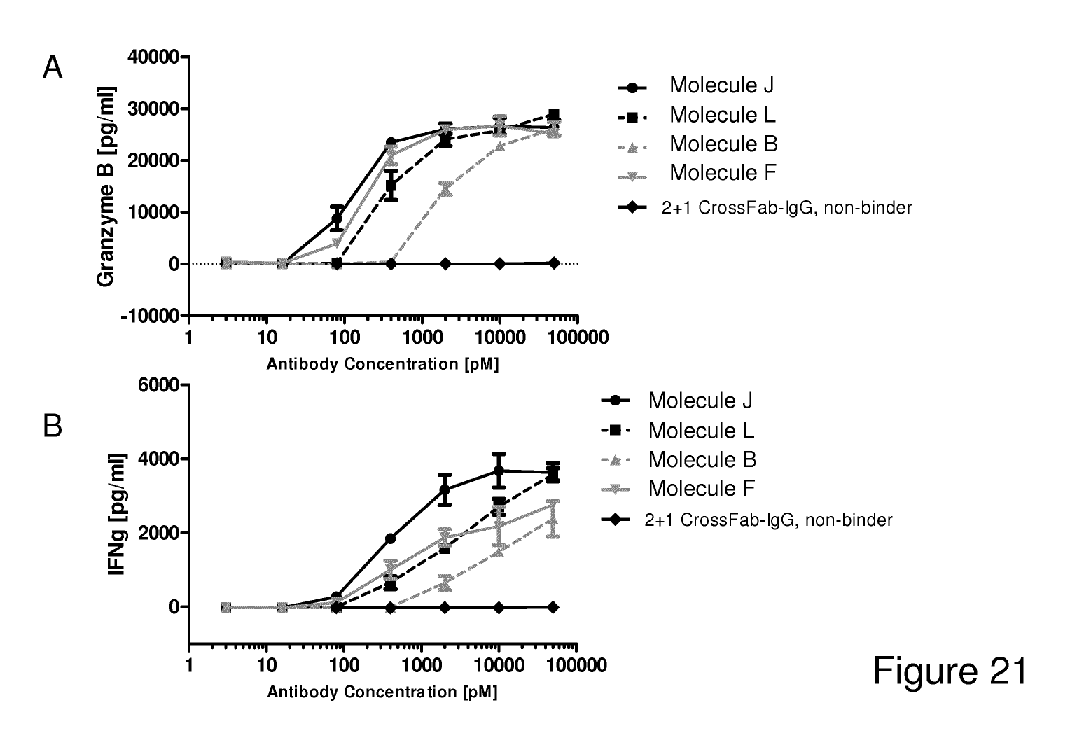

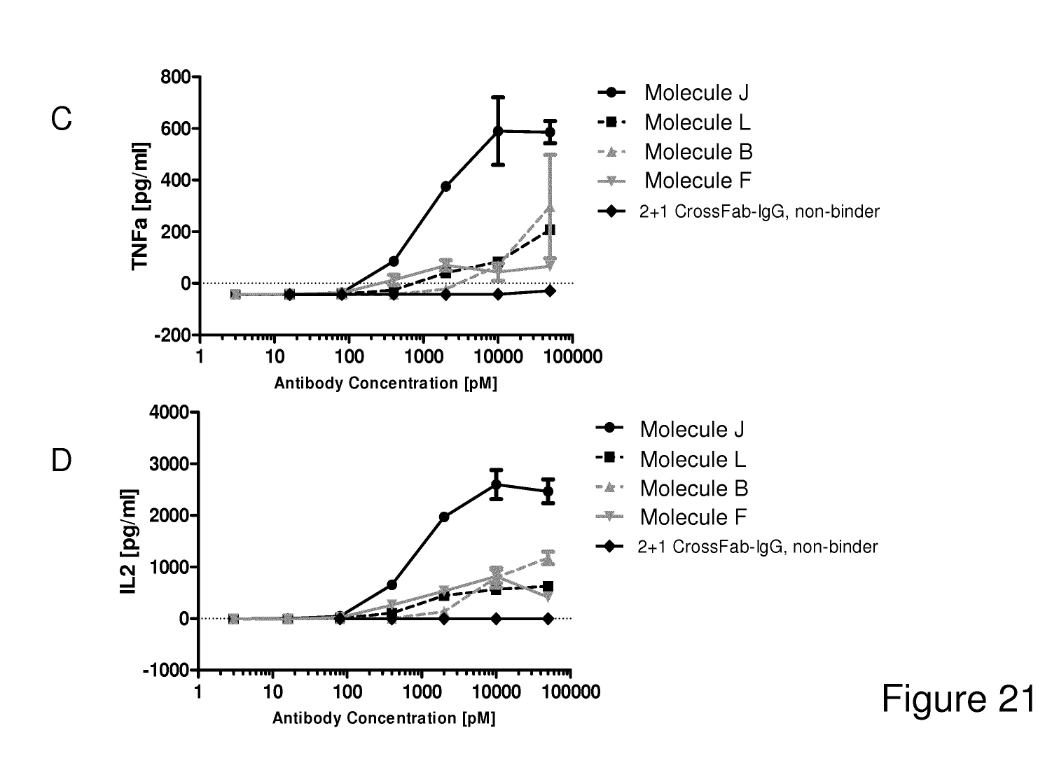

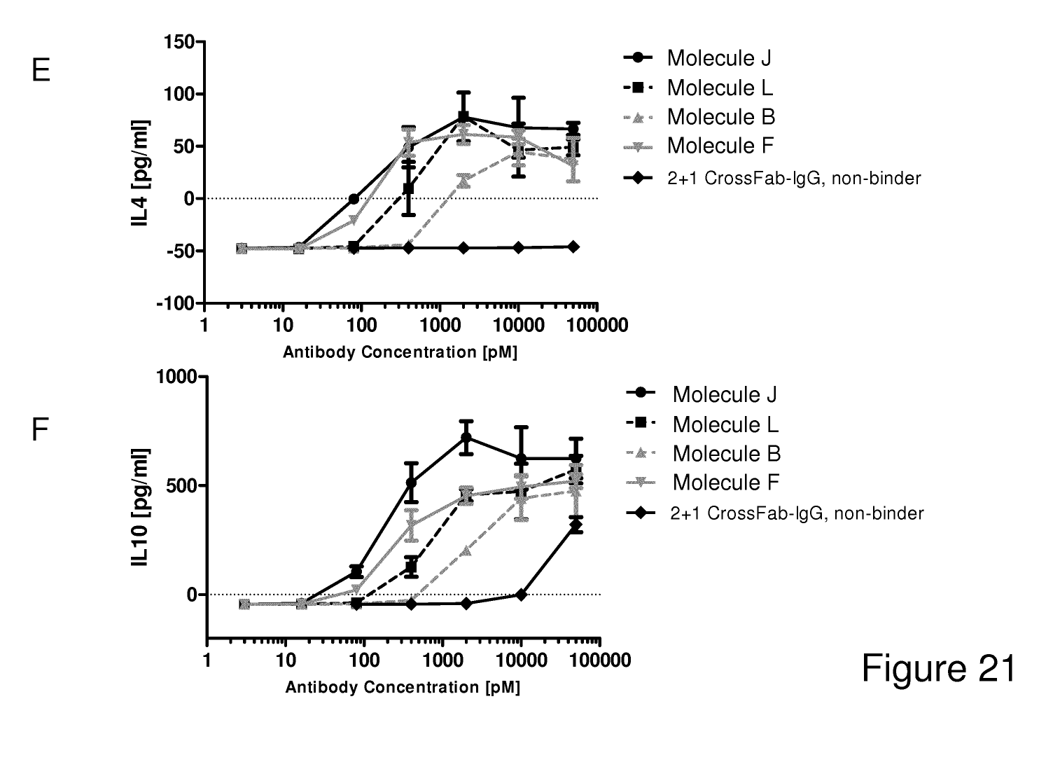

The results are shown in FIG. 21. All bispecific antibodies (except the non-binding control) induced dose dependent Granzyme B, IFNγ, TNFα, IL-2, IL-4 and IL-10 secretion. In line with the T cell killing data, all constructs were comparable in inducing Granzyme B, IFNγ, IL-4 and IL-10 secretion with molecule B (1+1 CrossFab-IgG) being the least efficacious one. Of note, molecule J (Fab-CrossFab) was the most efficacious in inducing IL-2 and TNFα secretion.

### Example 18

Proliferation of T Cells after T Cell-Mediated Killing of Human Endothelial Cells Induced by Anti-Robo 4/Anti-CD3 Bispecific Antibodies

Proliferation of CD4⁺ and CD8⁺ T cells was assessed seven days after T-cell mediated killing of human endothelial cells (HUVECs) by freshly isolated human PBMCs, induced by the anti-Robo 4/anti-CD3 bispecific antibodies in the Fab-CrossFab, the Fab-Fab-CrossFab, the 1+1 CrossFab-IgG and the 2+1 CrossFab-IgG format.

The same antibodies were used (molecule J, L, B and F) and the killing assay was performed essentially as described above (Example 15-17), using eFluor-670 labeled PBMCs at an E:T ratio of 10:1 and an incubation time of 24 h. Antibodies were tested at the concentration of 5 pM, 500 pM and 50 nM.

Freshly isolated PBMCs (20 million/ml) were stained with 5 μM eFluor® 670 (eBioscience #65-0840-85, diluted in PBS pre-warmed to room temperature) for 10 minutes at 37° C., 5% CO₂, in the dark. The labeling was stopped by adding 4-5 volumes of cold complete media (containing ≥10% serum) and incubating on ice for 5 minutes. Subsequently, cells were washed 3× with cold PBS and finally resuspended in RPMI+2% FCS+1% Glutamax. 0.03 million/well HUVEC target cells were plated 24 h before in a round-bottom 96-well plate and the different bispecific constructs added at the indicated concentrations (in triplicate). Finally, eFluor-stained PBMCs were added to a final E:T of 10:1 and the plate was incubated for seven days at 37° C., 5% CO₂. To ensure that T-cell killing occurred efficiently, target cell killing was assessed after 21 h incubation at 37° C., 5% CO₂ by quantification of LDH released in cell supernatants (LDH detection kit, Roche Applied Science, #11 644 793 001), according to manufacturer's instructions. CD4⁺ and CD8⁺ T cell proliferation of was quantified after seven days of incubation by assessing the eFluor dye dilution in antibody-treated samples when compared to untreated controls. Cells were analyzed by FACS using a FACS CantoII.

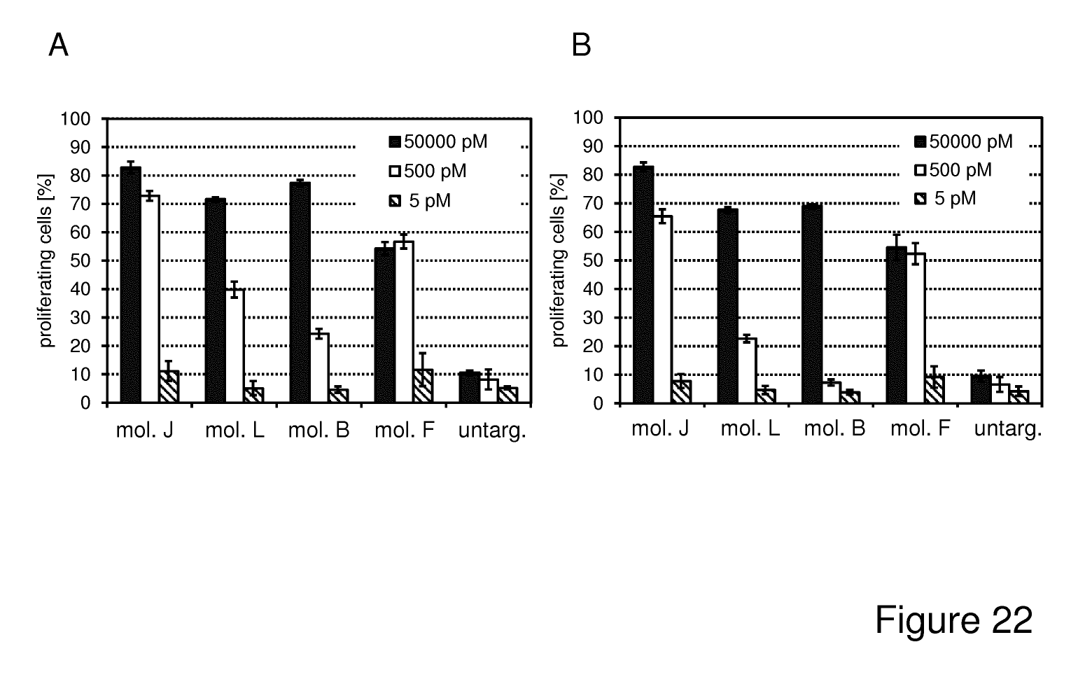

The results of this experiment are shown in FIG. 22. All constructs except the non-binding control induced a dose-dependent proliferation of CD4⁺ and CD8⁺ T cells. Molecule J and molecule F (Fab-CrossFab and 2+1 CrossFab-IgG, respectively) were the most efficacious in inducing T cell proliferation already at 500 pM. At 50 nM the proliferation induction was comparable for all constructs. No proliferation was induced with any of the constructs when these were used at 5 pM.

### Example 19

T Cell Mediated Killing of Murine Endothelial Cells (MS-1) by Human T Cells Induced by Anti-Robo 4/Anti-CD3 Bispecific Antibodies

T cell mediated killing of MS-1 mouse endothelial cells by freshly isolated human T cells, induced by anti-Robo 4/anti-CD3 bispecific antibodies was assessed.

Three different, human/mouse crossreactive anti-Robo 4 clones (01F05, 01E06, 01F09) were compared in the Fab-CrossFab format (molecule J (SEQ ID NOs 47, 53 and 79), molecule G (SEQ ID NOs 37, 39 and 79), and molecule I (SEQ ID NOs 57, 59 and 79), respectively) and the 1+1 CrossFab-IgG format (molecule B (SEQ ID NOs 41, 53, 79 and 83), molecule C (SEQ ID NOs 35, 39, 79 and 83), and molecule A (SEQ ID NOs 55, 59, 79 and 83), respectively). All constructs contained the anti-human CD3 antibody (V9).

Briefly, MS-1 cells were harvested with Cell Dissociation Buffer, washed, and plated at a density of 30 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from local blood banks or of fresh blood from healthy human donors as described above. T cell enrichment from PBMCs was performed using the Pan T Cell Isolation Kit II (Miltenyi Biotec #130-091-156), as described above. For the killing assay, the respective antibody dilutions were added at the indicated concentrations (concentration range of 5 pM -500 nM; in triplicate). Human isolated pan T cells were added at a final E:T ratio of 5:1. Target cell killing was assessed after 17 h incubation at 37° C., 5% CO₂ by quantification of LDH released in cell supernatants by apoptotic/necrotic cells (LDH detection kit, Roche Applied Science, #11 644 793 001), according to the manufacturer's instructions.

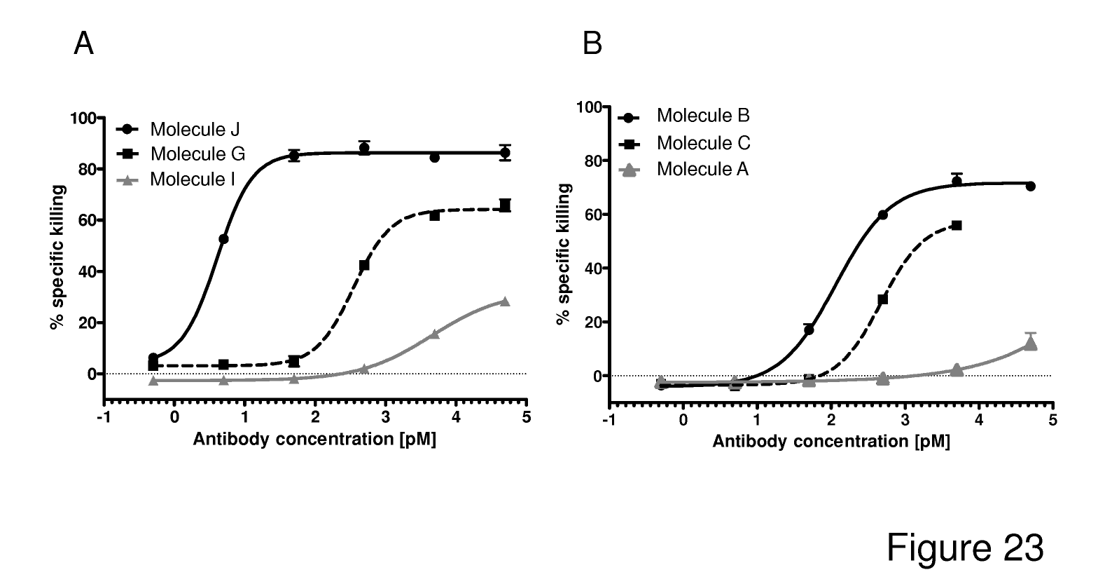

The results of the experiment are shown in FIG. 23. Maximal lysis of the target cells (=100%) was achieved by incubation of target cells with 1% Triton X-100. Minimal lysis (=0%) refers to target cells co-incubated with effector cells without bispecific construct or control IgG. EC50 values related to killing assays, calculated using GraphPadPrism5, are given in Table 10. In this experiment, anti-Robo 4 antibody clone 01F05 shows superior activity when compared to clones 01E06 and 01F09 in both formats.

TABLE 10
EC50 values (pM) for T-cell mediated killing of murine endothelial
cells (MS-1) induced by anti-Robo 4/anti-CD3 bispecific antibodies.

| | | Molecule | |
|---|---|---|---|
| Molecule | EC50 | (1 + 1 | EC50 |
| (Fab-CrossFab) | [pM] | CrossFab-IgG) | [pM] |
| J (01F05/V9) | 4 | B (01F05/V9) | 115 |
| G (01E06/V9) | 352 | C (01E06/V9) | 472 |
| I (01F09/V9) | 4558 | A (01F09/V9) | n.d. |

### Example 20

CD25 Upregulation on Human Effector Cells after T Cell-Mediated Killing of Mouse Endothelial Cells Induced by Anti-Robo 4/Anti-CD3 Bispecific Antibodies

Activation of CD4⁺ and CD8⁺ T cells after T-cell mediated killing of MS-1 cells induced by the anti-Robo 4/anti-CD3 bispecific antibodies in the Fab-CrossFab and the 1+1 CrossFab-IgG format was assessed by FACS analysis using antibodies recognizing the T cell activation marker CD25.

The same antibodies were used (molecules J, G, I, B, C and A, concentration 50 nM) and the killing assay was performed essentially as described above (Example 19), using an E:T ratio of 5:1 and an incubation time of 17 h. The bispecific constructs and the corresponding human/mouse crossreactive anti-Robo 4 IgG controls were adjusted to the same molarity. PHA-M 1-10 μg/ml (Sigma #L8902) was used as a mitogenic stimulus to induce human T cell activation.

After the incubation, T-cells were transferred to a round-bottom 96-well plate, centrifuged at 350×g for 5 min and washed twice with PBS containing 0.1% BSA. Surface staining for CD8 (BD #555634), CD4 (Biolegend #344612) and CD25 (BD #555434) was performed according to the suppliers' indications. Cells were washed twice with 150 μl/well PBS containing 0.1% BSA and fixed for 15 min at 4° C. using 100 μl/well fixation buffer (BD #554655). After centrifugation, the samples were resuspended in 200 μl/well PBS 0.1% BSA and analyzed at FACS CantoII (Software FACS Diva).

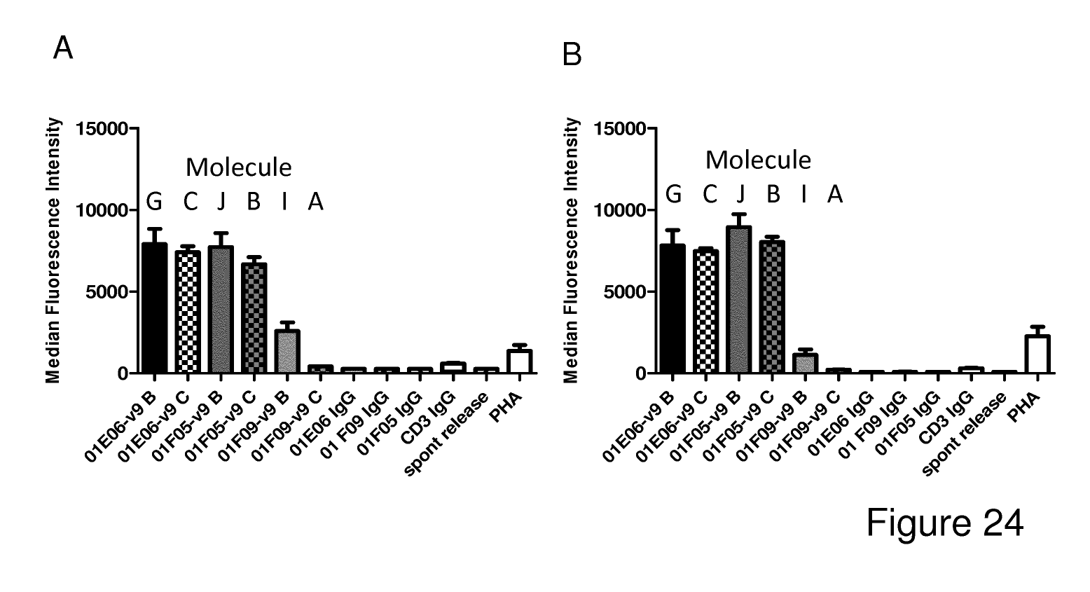

The results are shown in FIG. 24.

### Example 21

T Cell Mediated Killing of Murine Endothelial Cells (MS-1) by Mouse Splenocytes Induced by Anti-Robo 4/Anti-CD3 Bispecific Antibodies

T cell mediated killing of MS-1 mouse endothelial cells by freshly isolated murine splenocytes, induced by the anti-Robo 4 (clone 01F05)/anti-mouse CD3 (clone 2C11, described in GenBank [www.ncbi.nlm.nih.gov] accession nos. U17871.1 and U17870.1) Fab-CrossFab bispecific antibody was assessed (molecule K, SEQ ID NOs 49, 53 and 81).

Briefly, MS-1 cells were harvested with Cell Dissociation Buffer, washed and plated at a density of 30 000 cells/well using flat-bottom 96-well plates. Cells were left to adhere overnight. Spleens were isolated from C57BL/6 mice, transferred into a GentleMACS C-tube (Miltenyi Biotech #130-093-237) containing MACS buffer (PBS+0.5% BSA+2 mM EDTA) and dissociated with the GentleMACS Dissociator to obtain single-cell suspensions according to the manufacturer's instructions. The cell suspension was passed through a pre-separation filter to remove remaining undissociated tissue particles. After centrifugation at 400×g for 4 min at 4° C., ACK Lysis Buffer was added to lyse red blood cells (incubation for 5 min at room temperature). The remaining cells were washed with assay medium twice, automatically counted (ViCell) and immediately used for further assays.

For the killing assay, the respective antibody dilutions were added at the indicated concentrations (concentration range of 32 pM-500 nM, in triplicate). Murine splenocytes were added at a final E:T ratio of 10:1. A 5% solution of “rat T-Stim with ConA” (BD #354115) was used as a positive control for murine splenocyte activation. Target cell killing was assessed after 48 h and 72 h incubation at 37° C., 5% CO₂ by quantification of LDH released in cell supernatants by apoptotic/necrotic cells (LDH detection kit, Roche Applied Science, #11 644 793 001), according to the manufacturer's instructions.

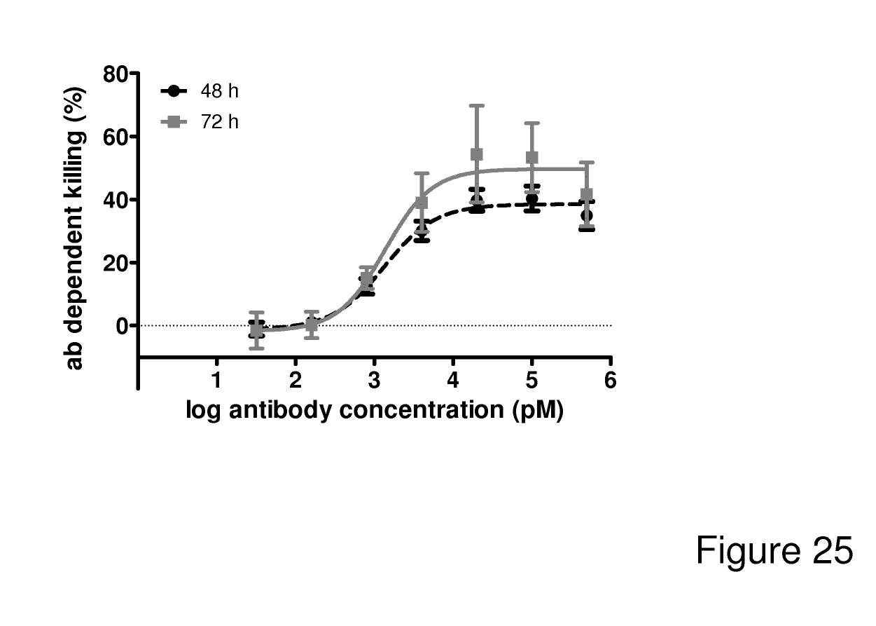

The results of the experiment are shown in FIG. 25. Maximal lysis of the target cells (=100%) was achieved by incubation of target cells with 1% Triton X-100. Minimal lysis (=0%) refers to target cells co-incubated with effector cells without bispecific construct or control IgG. EC50 values related to killing assays, calculated using GraphPadPrism5, were 1.3 nM at both incubation times (48 and 72 h).

### Example 22

In Vivo Anti-Tumor Efficacy of Anti-Robo 4/Anti-CD3 Bispecific Antibodies

Anti-tumor efficacy in N-Ras melanoma-bearing human CD3ε transgenic C57BL/6 mice (these mice express both mouse and human CD3ε on their T cells) mediated by the anti-Robo 4 (clone 01F05)/anti-mouse CD3 (clone 2C11) Fab-CrossFab bispecific antibody (molecule K, SEQ ID NOs 49, 53 and 81), or by the anti-Robo 4 (clone 01F05)/anti-human CD3 (clone V9) Fab-CrossFab bispecific antibody (molecule J, SEQ ID NOs 47, 53 and 79) was assessed.

Briefly, C57BL/6 mice were inoculated subcutaneously (s.c.) with 150,000 N-Ras melanoma cells (originally generated at Roche Glycart AG from a spontaneous melanoma tumor developing in N-Ras transgenic mice (Ackermann et al., Cancer Res 65, 4005-4011 (2005))). Eight days after tumor cell inoculation, mice received bi-daily intra-peritoneal (i.p.) injection of either vehicle, molecule K at 125 μg/kg cumulative daily dose, or molecule J at 50 μg/kg cumulative daily dose. Tumor volume was measured 3 times a week by digital caliper. Treatment was administered until 20 days after tumor cell inoculation, which corresponds to the day of study termination.

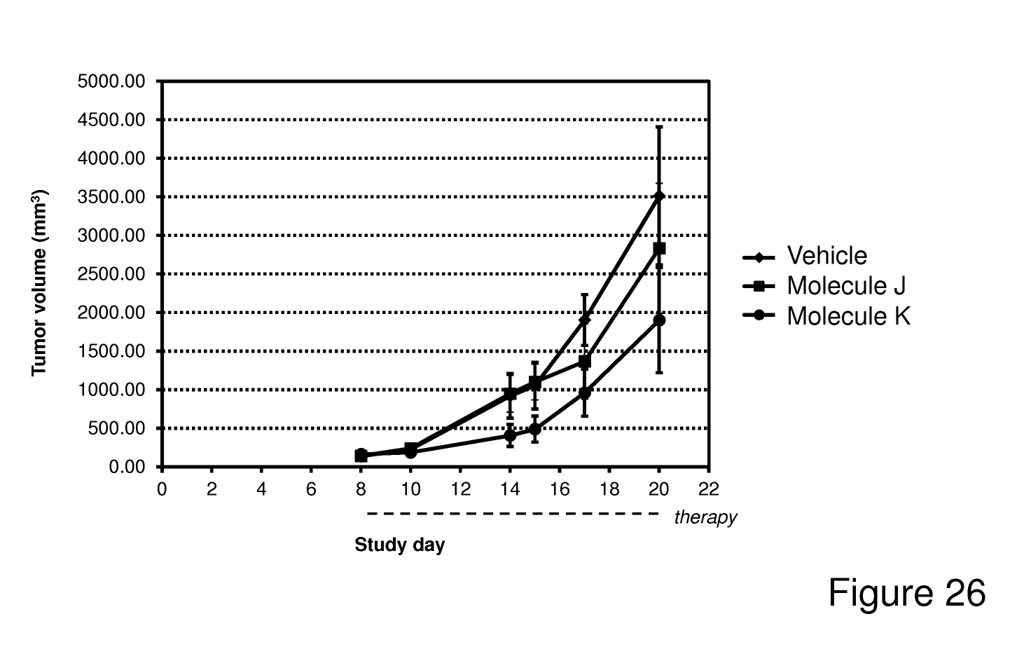

The results of the experiment are shown in FIG. 26. Results show average and SEM of tumor volume measurements in the different study groups (n=10). The dashed line below the graph indicates the therapeutic window.

### Example 23

Ex Vivo Peripheral T Cell Analysis from Tumor-Bearing Mice Treated with Anti-Robo 4/Anti-CD3 Bispecific Antibodies

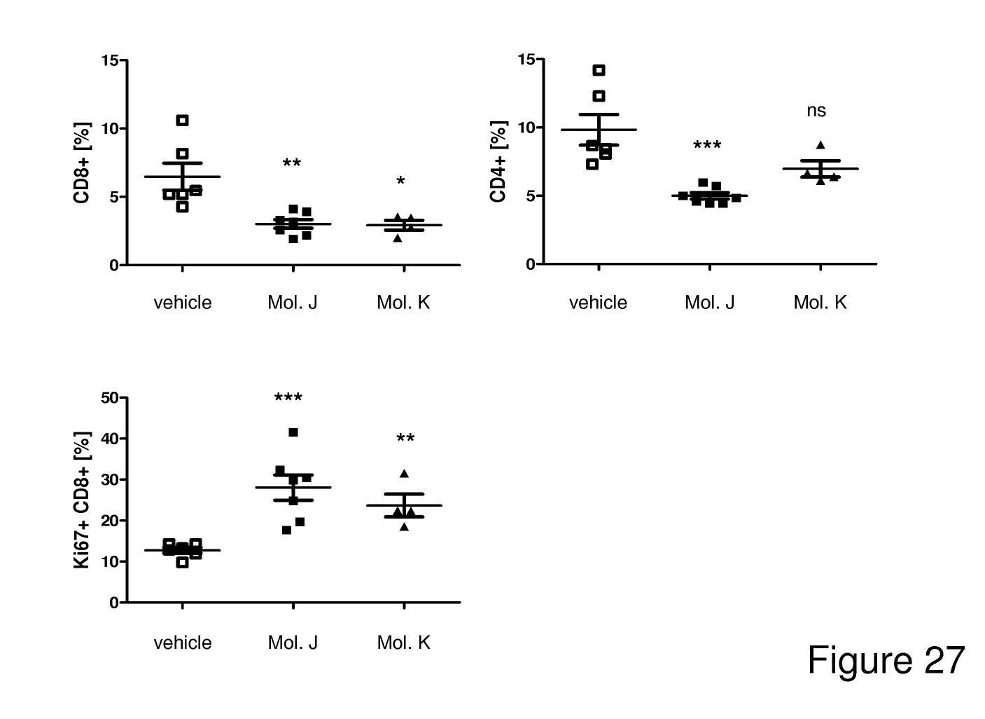

N-Ras melanoma-bearing human CD3ε transgenic C57BL/6 mice were treated as described in Example 22. Eleven days after therapy injection, mouse PBMC from all groups were analysed by ex vivo FACS analysis for different T cell surface markers and for the proliferation marker Ki67. Results are shown in FIG. 27 and they represent single values for each therapeutic group (n=6-7). The horizontal bars represent average values. For statistical analysis, a t-test was used (*p<0.05, **p<0.01, ***p<0.001).

Both therapeutic treatments mediated a significant reduction in the frequency of blood CD8⁺ T cells (upper left panel), and molecule J also mediated a significant reduction in the frequency of blood CD4⁺ T cells (upper right panel). Both treatments mediated a significant increase in the frequency of Ki67⁺ cells among CD8⁺ T cells (lower panel).

### Example 24

Quantification of CD3 Positive Cells in Tumor Tissue from Mice Treated with Anti-Robo 4/Anti-CD3 Bispecific Antibodies

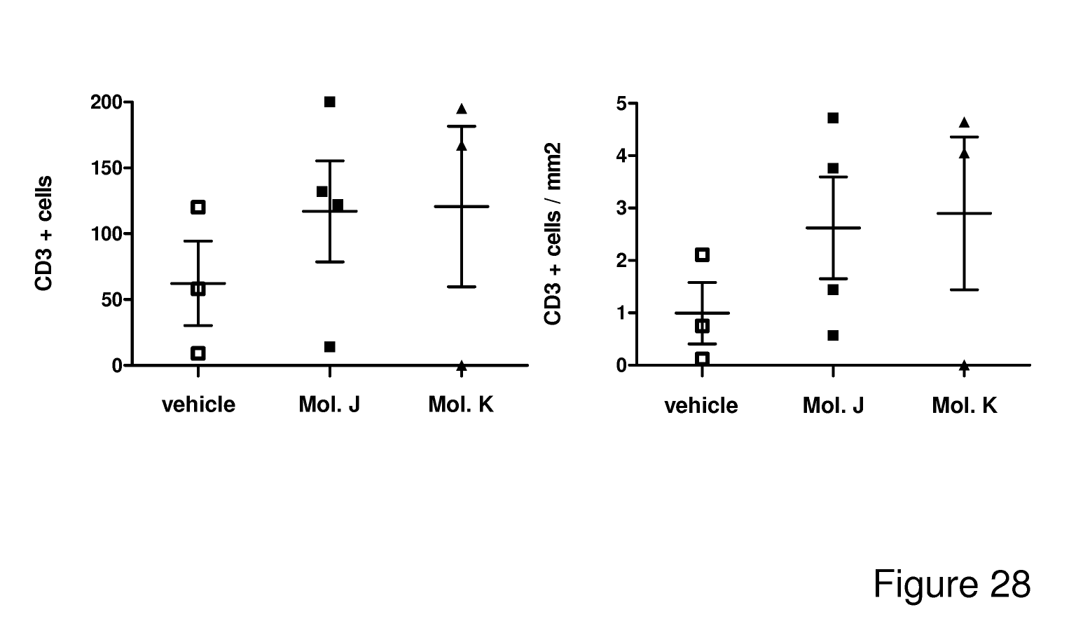

N-Ras subcutaneous tumors (see Example 22) were harvested (day 20) and fixed in 10% neutral buffered formalin overnight. Formalin paraffin embedded tissue blocks were prepared in an embedding machine (Leica Automatic Tissue Processor TP1020). 4 μm sections were cut with a microtome (Leica Rotary microtome RM2235). The staining was performed with an anti-CD3 antibody (rabbit monoclonal anti-CD3 clone SP7, Labvision #RM-9107), developed with alkaline phosphatase and counterstained with hematoxylin. The CD3 positive cells were scored manually on a whole slide scan. Results are shown in FIG. 28. Each plot represents one tissue section of one mouse. The mean and the SEM are shown.

### Example 25

Preparation of Anti-Robo 4/Anti-CD3 T Cell Bispecific (TCB) Molecules with Charge Modifications

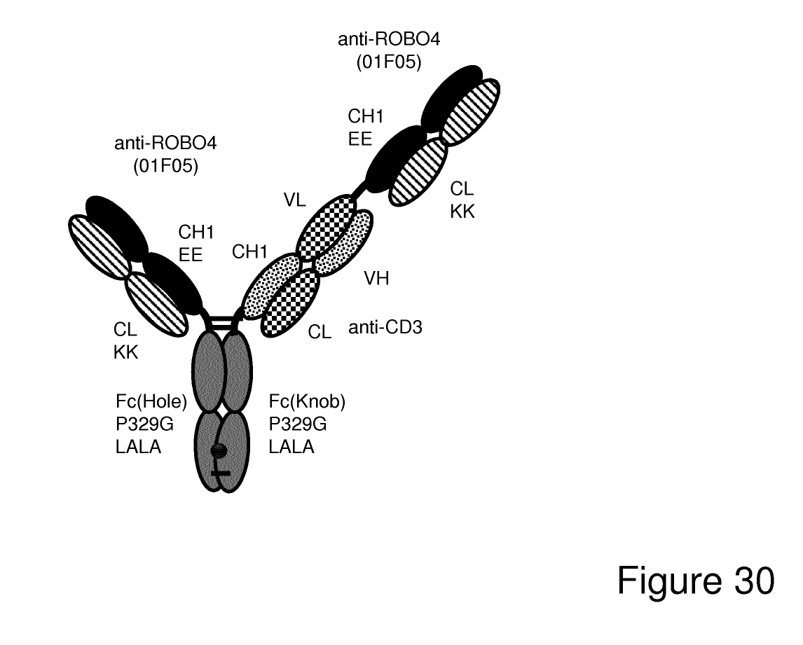

The following molecule was prepared in this example; a schematic illustration thereof is shown in FIG. 30:
M. “2+1 CrossFab-IgG, inverted” with charge modifications (VH/VL exchange in CD3 binder, charge modification in Robo 4 binders, CD3 binder of SEQ ID NOs 140 (VH) and 144 (VL), Robo 4 binders based on 01F05) (FIG. 30, SEQ ID NOs 151-154).

The variable region of heavy and light chain DNA sequences were subcloned in frame with either the constant heavy chain or the constant light chain pre-inserted into the respective recipient mammalian expression vector. Protein expression is driven by an MPSV promoter and a synthetic polyA signal sequence is present at the 3′ end of the CDS. In addition each vector contains an EBV OriP sequence.

The molecules were produced by co-transfecting HEK293-EBNA cells growing in suspension with the mammalian expression vectors using polyethylenimine (PEI). The cells were transfected with the corresponding expression vectors in a 1:2:1:1 ratio (“vector heavy chain (VH-CH1-VL-CH1-CH2-CH3)”: “vector light chain (VL-CL)”: “vector heavy chain (VH-CH1-CH2-CH3)”: “vector light chain (VH-CL)”).

For transfection HEK293 EBNA cells were cultivated in suspension serum free in Excell culture medium containing 6 mM L-glutamine and 250 mg/l G418. For the production in 600 ml tubespin flasks (max. working volume 400 mL) 600 million HEK293 EBNA cells were seeded 24 hours before transfection. For transfection cells were centrifuged for 5 min at 210×g and supernatant was replaced by 20 ml pre-warmed CD CHO medium. Expression vectors were mixed in 20 ml CD CHO medium to a final amount of 400 μg DNA. After addition of 1080 μl PEI solution (2.7 μg/ml) the mixture was vortexed for 15 s and subsequently incubated for 10 min at room temperature. Afterwards cells were mixed with the DNA/PEI solution, transferred to a 600 ml tubespin flask and incubated for 3 hours at 37° C. in an incubator with a 5% CO₂ atmosphere. After incubation, 360 ml Excell+6 mM L-glutamine+5 g/L Pepsoy+1.0 mM VPA medium was added and cells were cultivated for 24 hours. One day after transfection 7% Feed 7 was added. After 7 days cultivation supernatant was collected for purification by centrifugation for 20-30 min at 3600×g (Sigma 8K centrifuge), the solution was sterile filtered (0.22 μm filter) and sodium azide in a final concentration of 0.01% w/v was added. The solution was kept at 4° C.

The concentration of the molecules in the culture medium was determined by Protein A-HPLC. The basis of separation was binding of Fc-containing molecules to Protein A at pH 8.0 and step elution from pH 2.5. There were two mobile phases. These were Tris (10 mM)—glycine (50 mM)—NaCl (100 mM) buffers, identical except that they were adjusted to different pHs (8 and 2.5). The column body was an Upchurch 2×20 mm pre-column with an internal volume of −63 μl packed with POROS 20A. 100 μl of each sample was injected on equilibrated material with a flow rate of 0.5 ml/min. After 0.67 minutes the sample was eluted with a pH step to pH 2.5. Quantitation is done by determination of 280 nm absorbance and calculation using a standard curve with a concentration range of human IgG₁ from 16 to 166 mg/l.

The secreted protein was purified from cell culture supernatants by affinity chromatography using Protein A affinity chromatography, followed by a size exclusion chromatographic step. For affinity chromatography supernatant was loaded on a HiTrap Protein A HP column (CV=5 mL, GE Healthcare) equilibrated with 25 ml 20 mM sodium phosphate, 20 mM sodium citrate, 0.5 M NaCl, 0.01% Tween-20 pH 7.5. Unbound protein was removed by washing with at least 10 column volumes 20 mM sodium phosphate, 20 mM sodium citrate, 0.5 M NaCl, 0.01% Tween-20 pH 7.5 and target protein was eluted in 6 column volumes 20 mM sodium citrate, 0.5 M sodium chloride, 0.01% Tween-20, pH 2.5. Protein solution was neutralized by adding 1/10 of 0.5 M sodium phosphate, pH 8.0. Target protein was concentrated and filtrated prior loading on a HiLoad Superdex 200 column (GE Healthcare) equilibrated with 20 mM histidine, 140 mM sodium chloride, 0.01% Tween-20, pH 6.0.

For in-process analytics after Protein A chromatography the purity and molecular weight of the molecules in the single fractions were analyzed by SDS-PAGE in the absence of a reducing agent and staining with Coomassie (InstantBlue™, Expedeon). The NuPAGE® Pre-Cast gel system (4-12% Bis-Tris, Invitrogen) was used according to the manufacturer's instruction. The protein concentration of purified protein sample was determined by measuring the optical density (OD) at 280 nm, using the molar extinction coefficient calculated on the basis of the amino acid sequence.

Purity and molecular weight of the molecule after the final purification step were analyzed by CE-SDS analyses in the presence and absence of a reducing agent. The Caliper LabChip GXII system (Caliper Lifescience) was used according to the manufacturer's instruction.

The aggregate content of the molecule was analyzed using a TSKgel G3000 SW XL analytical size-exclusion column (Tosoh) in 25 mM K₂HPO₄, 125 mM NaCl, 200 mM L-arginine monohydrocloride, 0.02% (w/v) NaN₃, pH 6.7 running buffer at 25° C.

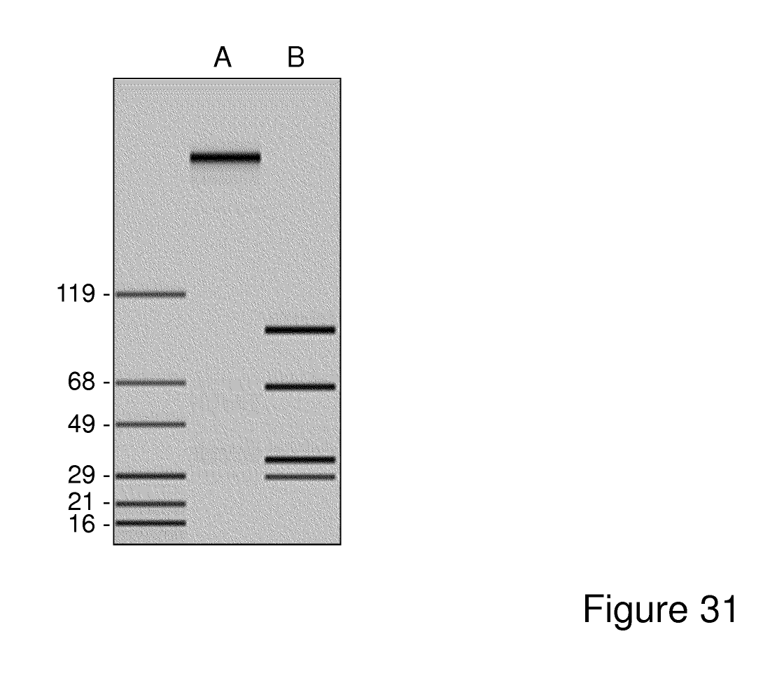

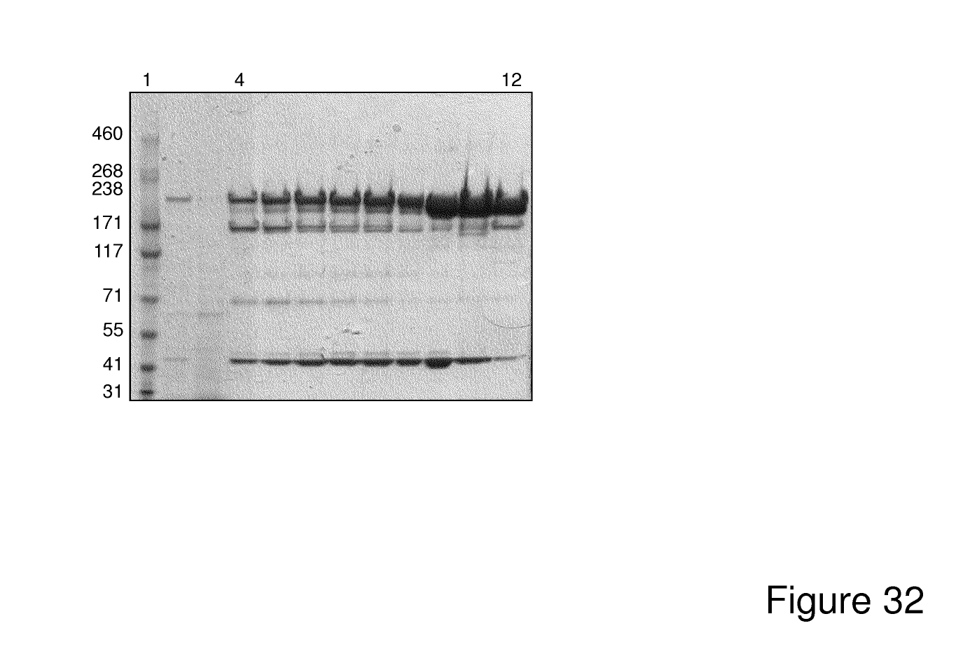

The final quality of the molecule was very good, with nearly 100% monomer content and 100% purity on CE-SDS (Table 11 and 12, FIG. 31).

TABLE 11
Summary of production and purification of anti-Robo
4/anti-CD3 TCB molecule with charge modifications.

| | Titer | Recovery | Yield | Analytical SEC |
|---|---|---|---|---|
| Molecule | [mg/l] | [%] | [mg/l] | (HMW/Monomer/LMW) [%] |
| M | 88 | 37 | 32 | 0.2/99.8/0 |

TABLE 12
CE-SDS analyses (non-reduced) of anti-Robo 4/anti-
CD3 TCB molecule with charge modifications.

| | | Size | Purity |
|---|---|---|---|
| Molecule | Peak # | [kDa] | [%] |
| M | 1 | 216 | 100 |

### Example 26

T-Cell Killing Induced by Anti-Robo 4/Anti-CD3 Bispecific Antibodies of Different Formats

T-cell mediated killing of human endothelial cells (HUVECs) and murine endothelial cells (MS-1) induced by anti-Robo 4/anti-CD3 bispecific antibodies of different bispecific antibody formats was compared: the Fab-CrossFab format (molecule J), the 2+1 CrossFab-IgG format (molecule F)—both comprising the anti-Robo 4 binder 01F05 and the anti-human CD3 binder V9—and the 2+1 CrossFab-IgG format with charge modifications (molecule M)—comprising the anti-CD3 binder of SEQ ID NOs 140 (VH) and 144 (VL). A non-binding 2+1 CrossFab-IgG format was used as control (“untargeted”, having VH and VL regions of SEQ ID NOs 155 and 156, respectively, instead of Robo 4 binding VH and VL regions).

The killing assay was performed essentially as described above, using freshly isolated human PBMCs. Briefly, HUVEC and MS-1 cells were harvested with Cell Dissociation Buffer, washed, and plated at a density of 30 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from local blood banks or of fresh blood from healthy human donors as described above. For the killing assay, the respective antibody dilutions were added at the indicated concentrations (6 pM-100 nM, in triplicate). Human PBMCs were added at a final E:T ratio of 10:1. Target cell killing was assessed after 24 and 48 h incubation at 37° C., 5% CO₂ by quantification of LDH released in cell supernatants by apoptotic/necrotic cells (LDH detection kit, Roche Applied Science, #11 644 793 001), according to the manufacturer's instructions.

Maximal lysis of the target cells (=100%) was achieved by incubation of target cells with 1% Triton X-100. Minimal lysis (=0%) refers to target cells co-incubated with effector cells without bispecific construct or control IgG.

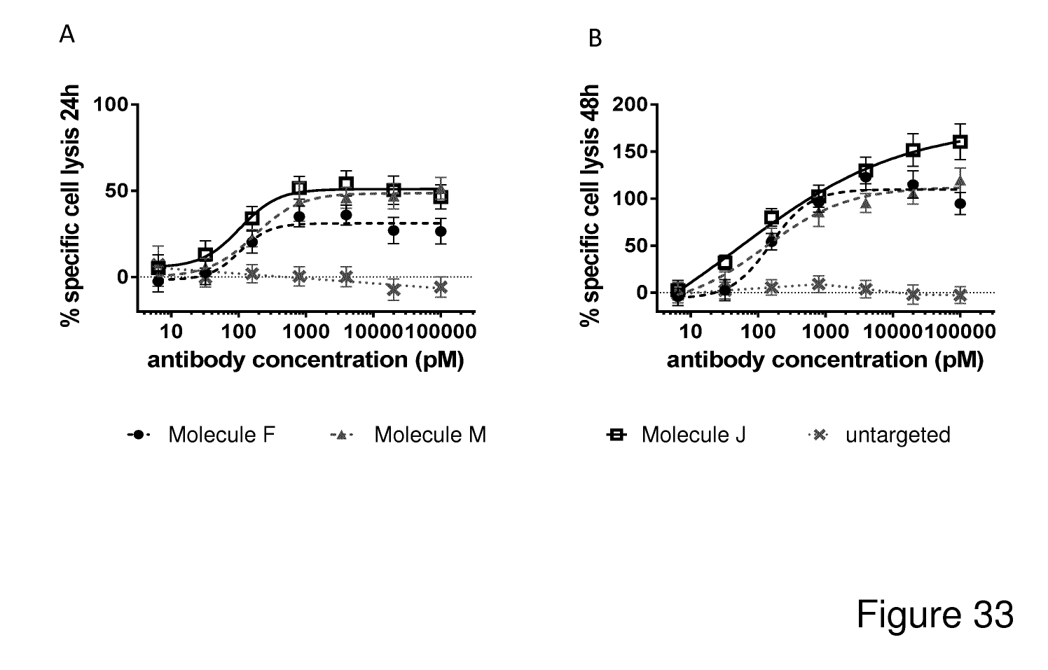

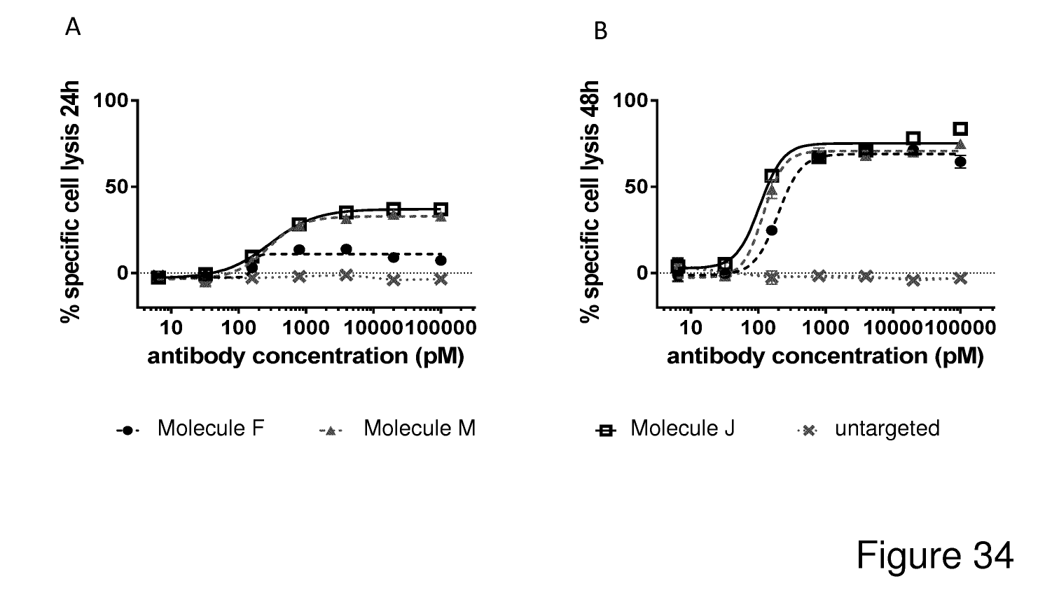

The results of the experiment are shown in FIG. 33 (HUVEC) and FIG. 34 (MS-1). The Fab-CrossFab construct (molecule J) and the 2+1 CrossFab-IgG construct with charge modifications (molecule M) are equally good in inducing T cell mediated killing of HUVEC and MS-1 cells. Molecule F is less potent after 24 h of incubation, but catches up with prolonged incubation time (48 h). EC50 values related to killing assays, calculated using GraphPadPrism6, are given in Table 13 (HUVEC) and Table 14 (MS-1).

TABLE 13
EC50 values (pM) for T-cell mediated killing
of human endothelial cells (HUVECs) induced by
anti-Robo 4/anti-CD3 bispecific antibodies.

EC50 (pM)

| Molecule | 24 h | 48 h |
|---|---|---|
| M | 173.4 | 125.0 |
| F | 103.9 | 154.4 |
| J | 105.2 | 47.2 |

TABLE 14
EC50 values (pM) for T-cell mediated killing of murine endothelial
cells (MS-1) induced by anti-Robo 4/anti-CD3 bispecific antibodies.

EC50 (pM)

| Molecule | 24 h | 48 h |
|---|---|---|
| M | 275.3 | 118.8 |
| F | ~164.0 * | 195.8 |
| J | 294.2 | 105.7 |

* ambiguous

### Example 27

CD25 and CD69 Upregulation on Human Effector Cells after T Cell-Mediated Killing of Human and Murine Endothelial Cells Induced by Anti-Robo 4/Anti-CD3 Bispecific Antibodies

Activation of CD4+ and CD8+ T cells after T-cell mediated killing of HUVECs and MS-1 cells induced by anti-Robo 4/anti-CD3 bispecific antibodies of different bispecific antibody formats (molecule J (Fab-CrossFab format), molecule F (2+1 CrossFab-IgG format)—both comprising the anti-Robo 4 binder 01F05 and the anti-human CD3 antibody V9—and molecule M (2+1 CrossFab-IgG format with charge modifications)—comprising the anti-CD3 binder of SEQ ID NOs 140 (VH) and 144 (VL)) was assessed by FACS analysis using antibodies recognizing the T cell activation markers CD25 (late activation marker) and CD69 (early activation marker). A non-binding 2+1 CrossFab-IgG format was used as control (“untargeted”, having VH and VL regions of SEQ ID NOs 155 and 156, respectively, instead of Robo 4 binding VH and VL regions).

The killing assay was performed essentially as described above (Example 26), using an E:T ratio of 10:1 and an incubation time of 48 h.

After incubation, PBMCs were transferred to a round-bottom 96-well plate, centrifuged at 350×g for 5 min and washed twice with PBS containing 0.1% BSA. Surface staining for CD8

(Biolegend #344714), CD4 (Biolegend #300532), CD69 (BD #555530) and CD25 (BD #302612) was performed according to the suppliers' indications. Cells were washed twice with 150 μl/well PBS containing 0.1% BSA and fixed for 20 min at 4° C. using 100 μl/well 1% PFA. After centrifugation, the samples were resuspended in 200 μl/well PBS 0.1% BSA and analyzed at FACS Cantoll (Software FACS Diva).

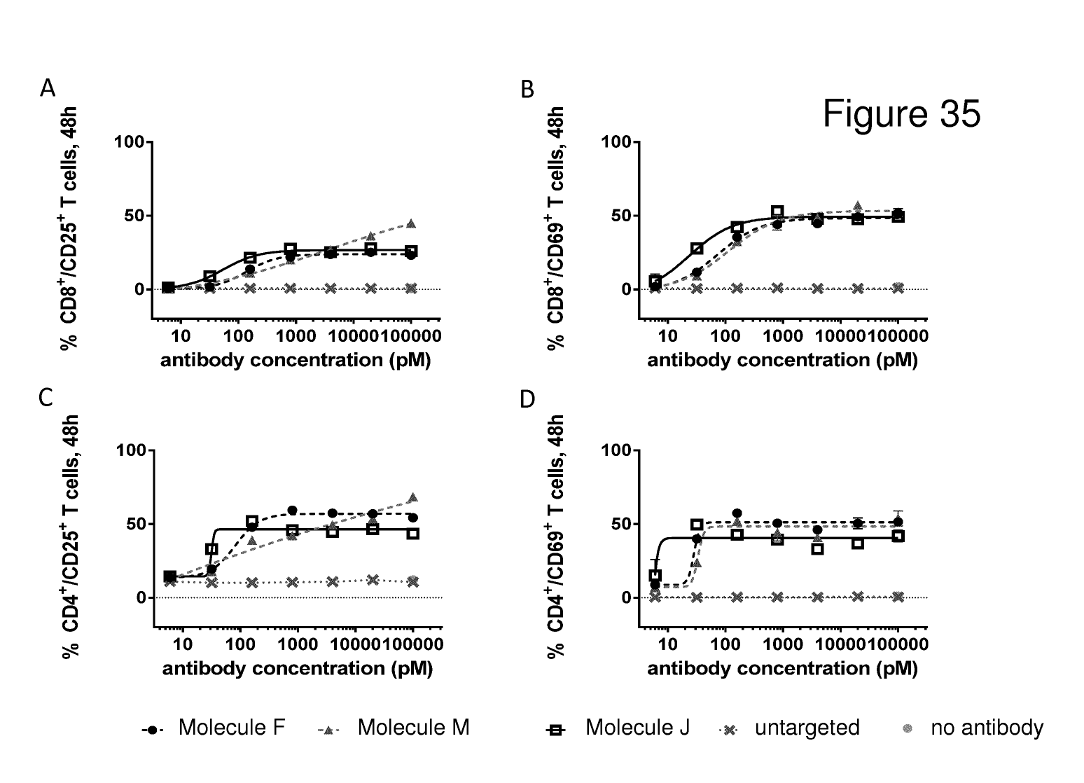

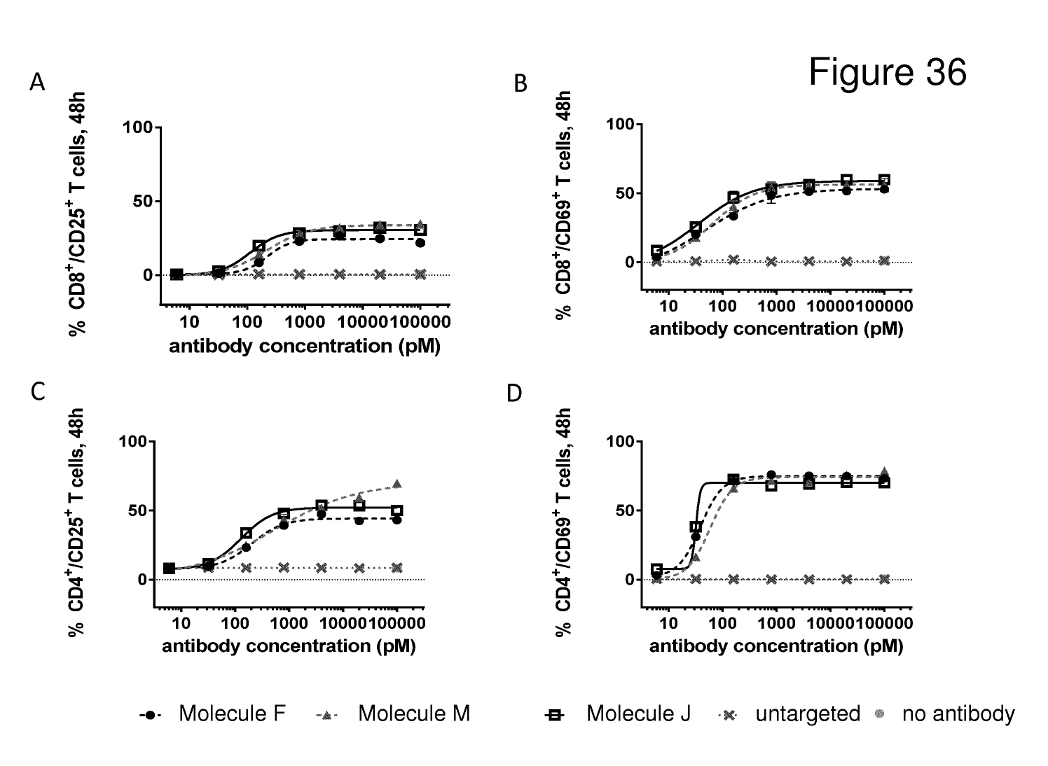

The results are shown in FIG. 35 (HUVEC) and FIG. 36 (MS-1). As for the killing activity after 48 h (see FIGS. 33B and 34B) activation of CD4+ and CD8+ T cells after T-cell mediated killing looks comparable for all anti-Robo 4/anti-CD3 bispecific antibodies with slightly stronger effect for molecule J when HUVECs are used as target cells. As expected the non-binding control molecule induced no T cell activation.

### Example 28

Cytokine Secretion by Human Effector Cells after T Cell-Mediated Killing of Human Endothelial Cells Induced by Anti-Robo 4/Anti-CD3 Bispecific Antibodies

Cytokine secretion by human PBMCs after T-cell mediated killing of HUVECs induced by the above mentioned anti-Robo 4/anti-CD3 bispecific antibodies (molecule J, molecule F and molecule M) was assessed by FACS analysis of cell supernatants after the killing assay. The killing assay was performed essentially as described above (Example 26), using an E:T ratio of 10:1 and an incubation time of 48 h.

At the end of the incubation time, the plate was centrifuged for 5 min at 350×g, the supernatants transferred in a new 96-well plate and stored at −20° C. until subsequent analysis. Granzyme B, TNFα, interferon-γ, IL-2 and IL-10 secreted into in cell supernatants were detected using the BD CBA Human Soluble Protein Flex Set, according to manufacturer's instructions on a FACS CantoII. The following kits were used: BD CBA human Granzyme B Flex Set #BD 560304; BD CBA human TNF Flex Set #BD 560112; BD CBA human IFN-γ Flex Set #BD 558269; BD CBA human IL-2 Flex Set #BD 558270; BD CBA human IL-10 Flex Set #BD 558274.

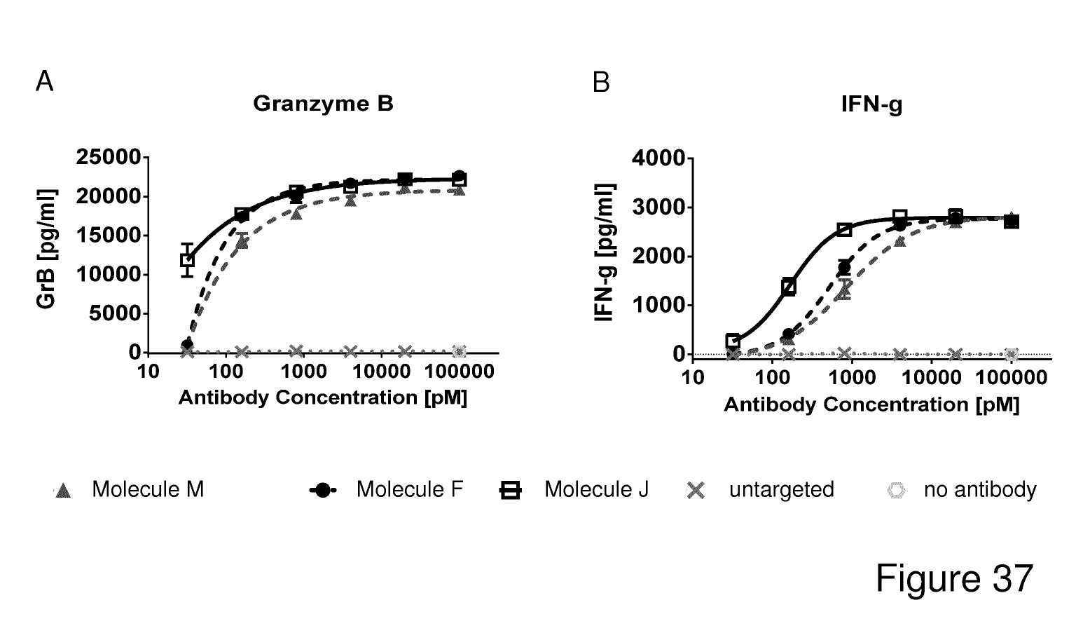

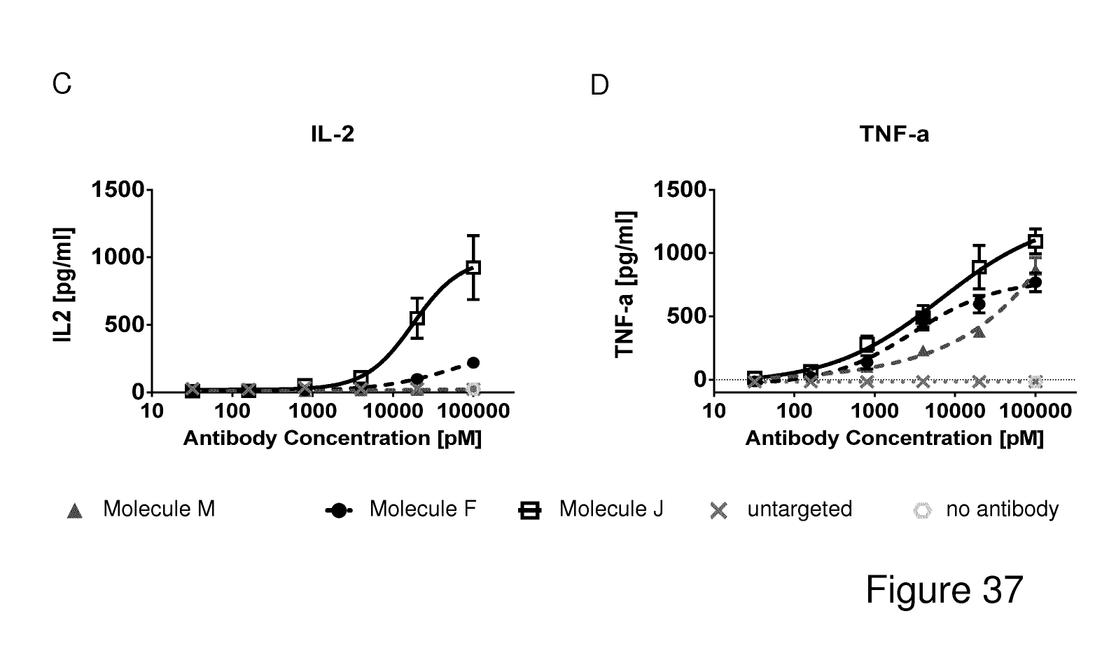

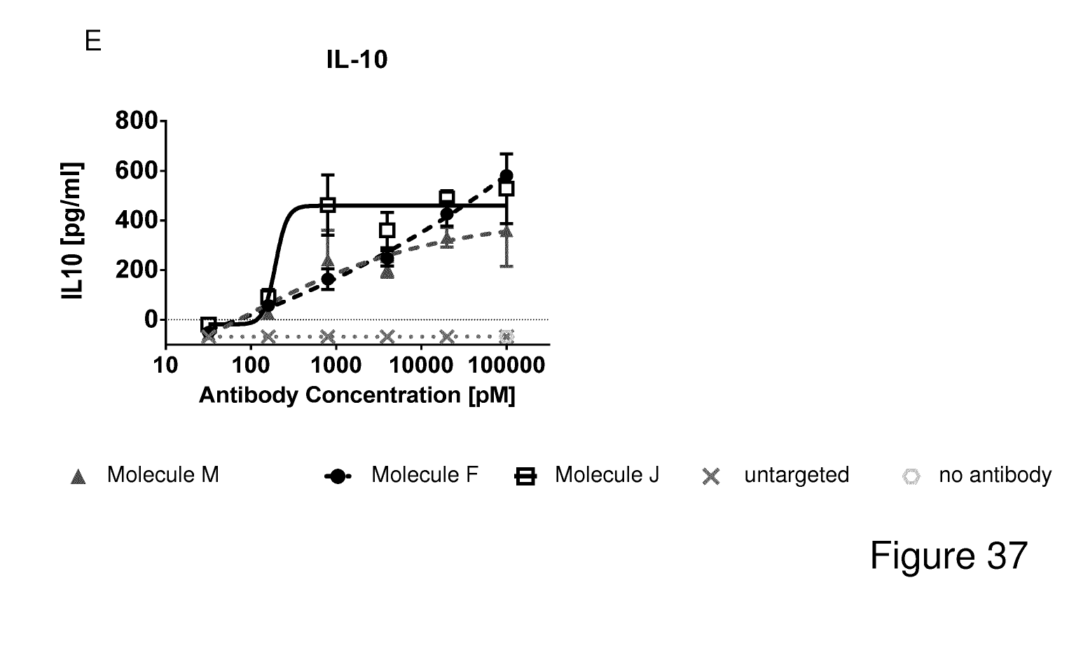

The results are shown in FIG. 37 A-E. All bispecific antibodies (except the non-binding control) induced dose dependent Granzyme B, IFNγ, TNFα and IL-10 secretion. Molecule J (Fab-CrossFab format) was the most efficacious in inducing cytokine secretion after T cell mediated killing and was the only construct that induced a considerable IL-2 release.

### Example 29

CD3 Activation on Jurkat-NFAT Reporter Cells Induced by Anti-Robo 4/Anti-CD3 Bispecific Antibodies in the Presence of Human and Mouse Endothelial Cells

The capacity of different anti-Robo 4/anti-CD3 bispecific antibodies (molecule J, molecule F and molecule M) to induce T cell cross-linking and subsequently T cell activation was assessed using co-cultures of Robo4-expressing endothelial cells and Jurkat-NFAT reporter cells (a CD3-expressing human acute lymphatic leukemia reporter cell line with a NFAT promoter, GloResponse Jurkat NFAT-RE-luc2P, Promega #CS176501). Upon simultaneous binding of anti-Robo 4/anti-CD3 bispecific antibodies to Robo4 antigen (expressed on endothelial cells) and CD3 antigen (expressed on Jurkat-NFAT reporter cells), the NFAT promoter is activated and leads to expression of active firefly luciferase. The intensity of luminescence signal (obtained upon addition of luciferase substrate) is proportional to the intensity of CD3 activation and signaling.

For the assay, human (HUVEC) and mouse (MS-1) endothelial cells were harvested and viability determined using ViCell. 20 000 cells/well were plated in a flat-bottom, white-walled 96-well-plate (#655098, greiner bio-one) and 50 μl/well of diluted antibodies or medium (for controls) was added. Subsequently, Jurkat-NFAT reporter cells were harvested and viability assessed using ViCell. Cells were resuspended at 2 mio cells/ml in cell culture medium and added to tumor cells at 0.1×10⁶ cells/well (50 μl/well) to obtain a final E:T of 5:1 and a final volume of 100 μl per well. Cells were incubated for 6 h at 37° C. in a humidified incubator. At the end of the incubation time, 100 μl/well of ONE-Glo solution (1:1 ONE-Glo and assay medium volume per well) were added to wells and incubated for 10 min at room temperature in the dark. Luminescence was detected using WALLAC Victor3 ELISA reader (PerkinElmer2030), 5 sec/well as detection time.

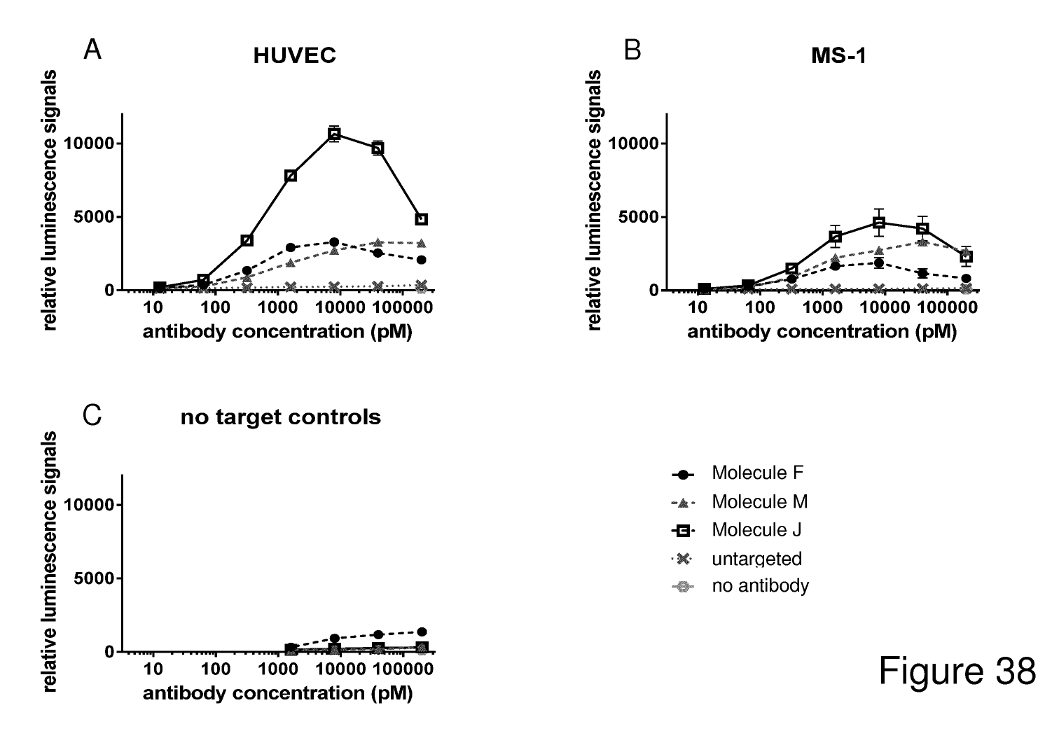

The results are shown in FIG. 38. All bispecific antibodies (except the non-binding control) induce T cell cross-linking and subsequently T cell activation. Molecule J (Fab-CrossFab) is the most efficacious of the anti-Robo 4/anti-CD3 bispecific antibodies tested.

### Example 30

Single Dose PK of Robo4 TCB in Healthy NOG Mice

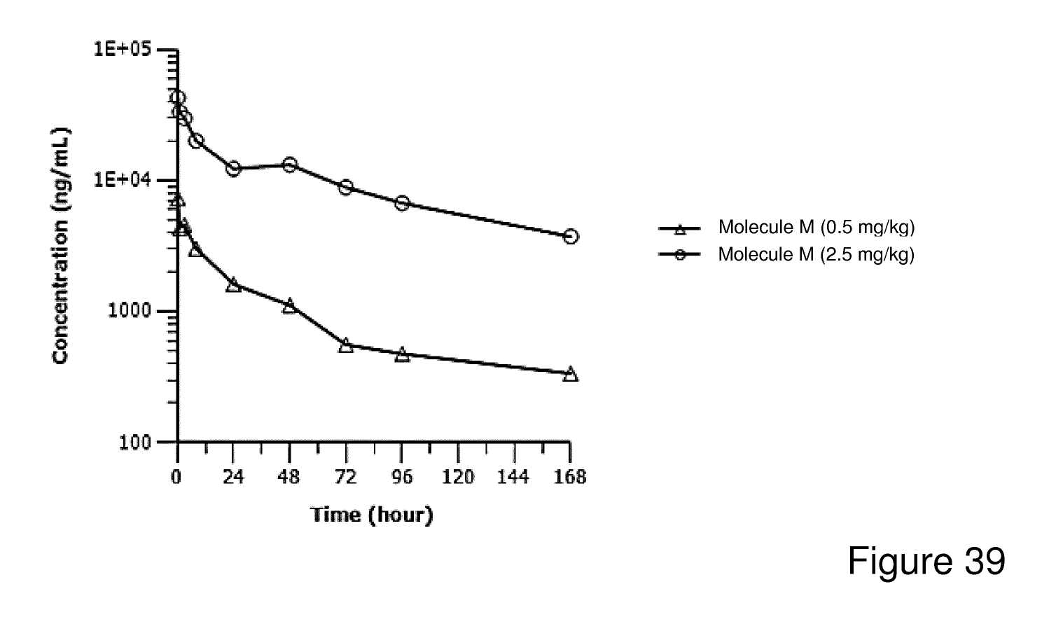

A single dose pharmacokinetic study (SDPK) was performed to evaluate exposure of molecule M in vivo (FIG. 39). An iv bolus administration of 0.5 mg/kg and of 2.5 mg/kg was administered to NOG mice and blood samples were taken at selected time points for pharmacokinetic evaluation. A generic immunoassay was used for measuring total concentrations of molecule M. The calibration range of the standard curve for molecule M was 0.78 to 50 ng/ml, where 15 ng/ml is the lower limit of quantification (LLOQ).

A biphasic decline was observed with a beta half-life of 6 days (non-compartmental analysis) and clearance of 30 mL/d/kg (2-compartmental model) at the high dose. The clearance was faster than expected as compared to a normal untargeted IgG.

Phoenix v6.2 from Pharsight Ltd was used for PK analysis, modelling and simulation.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A T cell activating bispecific antigen binding molecule comprising
(a) a first antigen binding moiety which specifically binds to a first antigen;
(b) a second antigen binding moiety which specifically binds to a second antigen;
wherein the first antigen is an activating T cell antigen and the second antigen is Robo 4, or the first antigen is Robo 4 and the second antigen is an activating T cell antigen.

2. The T cell activating bispecific antigen binding molecule according to claim 1, wherein the first and/or the second antigen binding moiety is a Fab molecule.

3. The T cell activating bispecific antigen binding molecule according to claim 1 or 2, wherein the second antigen binding moiety is a Fab molecule which specifically binds to a second antigen, and wherein the variable domains VL and VH or the constant domains CL and CH1 of the Fab light chain and the Fab heavy chain are replaced by each other.

4. The T cell activating bispecific antigen binding molecule according to any one of claims 1-3, wherein the first antigen is Robo 4 and the second antigen is an activating T cell antigen.

5. The T cell activating bispecific antigen binding molecule according to any one of claims 1-4, wherein the activating T cell antigen is CD3, particularly CD3 epsilon.

6. The T cell activating bispecific antigen binding molecule according to any one of claims 1-5, wherein the antigen binding moiety which specifically binds to the activating T cell antigen comprises a heavy chain variable region comprising the heavy chain complementarity determining region (HCDR) 1 of SEQ ID NO: 141, the HCDR 2 of SEQ ID NO: 142, the HCDR 3 of SEQ ID NO: 143, and a light chain variable region comprising the light chain complementarity determining region (LCDR) 1 of SEQ ID NO: 145, the LCDR 2 of SEQ ID NO: 146 and the LCDR 3 of SEQ ID NO: 147.

7. The T cell activating bispecific antigen binding molecule according to any one of claims 1-6, wherein the antigen binding moiety which specifically binds to the activating T cell antigen comprises a heavy chain variable region comprising an amino acid sequence that is at least about 95%, 96%, 97%, 98%, 99% or 100% identical to the amino acid sequence of SEQ ID NO: 140 and a light chain variable region comprising an amino acid sequence that is at least about 95%, 96%, 97%, 98%, 99% or 100% identical to the amino acid sequence of SEQ ID NO: 144.

8. The T cell activating bispecific antigen binding molecule according to any one of claims 1-7, wherein the antigen binding moiety which specifically binds to Robo 4 specifically binds to an epitope in the Ig-like domain 1 (position 20-119 of SEQ ID NO: 15) and/or the Ig-like domain 2 (position 20-107 of SEQ ID NO: 17) of the extracellular domain of Robo 4.

9. The T cell activating bispecific antigen binding molecule according to any one of claims 1-8, wherein the antigen binding moiety which specifically binds to Robo 4 comprises
(i) a heavy chain variable region comprising the heavy chain complementarity determining region (HCDR) 1 of SEQ ID NO: 91, the HCDR 2 of SEQ ID NO: 92 and the HCDR 3 of SEQ ID NO: 93, and a light chain variable region comprising the light chain complementarity determining region (LCDR) 1 of SEQ ID NO: 94, the LCDR 2 of SEQ ID NO: 95 and the LCDR 3 of SEQ ID NO: 96;
(ii) a heavy chain variable region comprising the heavy chain complementarity determining region (HCDR) 1 of SEQ ID NO: 103, the HCDR 2 of SEQ ID NO: 104 and the HCDR 3 of SEQ ID NO: 105, and a light chain variable region comprising the light chain complementarity determining region (LCDR) 1 of SEQ ID NO: 106, the LCDR 2 of SEQ ID NO: 107 and the LCDR 3 of SEQ ID NO: 108; or
(iii) a heavy chain variable region comprising the heavy chain complementarity determining region (HCDR) 1 of SEQ ID NO: 109, the HCDR 2 of SEQ ID NO: 110 and the HCDR 3 of SEQ ID NO: 111, and a light chain variable region comprising the light chain complementarity determining region (LCDR) 1 of SEQ ID NO: 112, the LCDR 2 of SEQ ID NO: 113 and the LCDR 3 of SEQ ID NO: 114.

10. The T cell activating bispecific antigen binding molecule according to any one of claims 1-9, wherein the antigen binding moiety which specifically binds to Robo 4 comprises
(i) a heavy chain variable region comprising an amino acid sequence that is at least about 95%, 96%, 97%, 98%, 99% or 100% identical to the amino acid sequence of SEQ ID NO: 19 and a light chain variable region comprising an amino acid sequence that is at least about 95%, 96%, 97%, 98%, 99% or 100% identical to the amino acid sequence of SEQ ID NO: 21;
(ii) a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 29; or
(iii) a heavy chain variable region comprising an amino acid sequence that is at least about 95%, 96%, 97%, 98%, 99% or 100% identical to the amino acid sequence of SEQ ID NO: 31 and a light chain variable region comprising an amino acid sequence that is at least about 95%, 96%, 97%, 98%, 99% or 100% identical to the amino acid sequence of SEQ ID NO: 33.

11. The T cell activating bispecific antigen binding molecule according to any one of claims 1-7, wherein the antigen binding moiety which specifically binds to Robo 4 specifically binds to an epitope in the fibronectin-like domain 1 (position 20-108 of SEQ ID NO: 11) and/or the fibronectin-like domain 2 (position 20-111 of SEQ ID NO: 11) of the extracellular domain of Robo 4.

12. The T cell activating bispecific antigen binding molecule according to any one of claim 1-7 or 11, wherein the antigen binding moiety which specifically binds to Robo 4 comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO: 100, the LCDR 2 of SEQ ID NO: 101 and the LCDR 3 of SEQ ID NO: 102.

13. The T cell activating bispecific antigen binding molecule according to any one of claim 1-7, 11 or 12, wherein the antigen binding moiety which specifically binds to Robo 4 comprises a heavy chain variable region comprising an amino acid sequence that is at least about 95%, 96%, 97%, 98%, 99% or 100% identical to the amino acid sequence of SEQ ID NO: 23 and a light chain variable region comprising an amino acid sequence that is at least about 95%, 96%, 97%, 98%, 99% or 100% identical to the amino acid sequence of SEQ ID NO: 25.

14. The T cell activating bispecific antigen binding molecule according to any one of claims 1-13, wherein the first antigen binding moiety under (a) is a first Fab molecule which specifically binds to a first antigen, the second antigen binding moiety under (b) is a second Fab molecule which specifically binds to a second antigen wherein the variable domains VL and VH of the Fab light chain and the Fab heavy chain are replaced by each other;
and
i) in the constant domain CL of the first Fab molecule under a) the amino acid at position 124 is substituted independently by lysine (K), arginine (R) or histidine (H) (numbering according to Kabat), and wherein in the constant domain CH1 of the first Fab molecule under a) the amino acid at position 147 or the amino acid at position 213 is substituted independently by glutamic acid (E), or aspartic acid (D) (numbering according to Kabat EU index); or
ii) in the constant domain CL of the second Fab molecule under b) the amino acid at position 124 is substituted independently by lysine (K), arginine (R) or histidine (H) (numbering according to Kabat), and wherein in the constant domain CH1 of the second Fab molecule under b) the amino acid at position 147 or the amino acid at position 213 is substituted independently by glutamic acid (E), or aspartic acid (D) (numbering according to Kabat EU index).

15. The T cell activating bispecific antigen binding molecule according to claim 14, wherein in the constant domain CL of the first Fab molecule under a) the amino acid at position 124 is substituted independently by lysine (K), arginine (R) or histidine (H) (numbering according to Kabat), and wherein in the constant domain CH1 of the first Fab molecule under a) the amino acid at position 147 or the amino acid at position 213 is substituted independently by glutamic acid (E), or aspartic acid (D) (numbering according to Kabat EU index).

16. The T cell activating bispecific antigen binding molecule according to claim 14 or 15, wherein in the constant domain CL of the first Fab molecule under a) the amino acid at position 124 is substituted independently by lysine (K), arginine (R) or histidine (H) (numbering according to Kabat), and wherein in the constant domain CH1 of the first Fab molecule under a) the amino acid at position 147 is substituted independently by glutamic acid (E), or aspartic acid (D) (numbering according to Kabat EU index).

17. The T cell activating bispecific antigen binding molecule according to any one of claims 14-16, wherein in the constant domain CL of the first Fab molecule under a) the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and wherein in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

18. The T cell activating bispecific antigen binding molecule according to any one of claims 14-17, wherein in the constant domain CL of the first Fab molecule under a) the amino acid at position 124 is substituted by lysine (K) (numbering according to Kabat) and the amino acid at position 123 is substituted by lysine (K) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

19. The T cell activating bispecific antigen binding molecule according to any one of claims 14-17, wherein in the constant domain CL of the first Fab molecule under a) the amino acid at position 124 is substituted by lysine (K) (numbering according to Kabat) and the amino acid at position 123 is substituted by lysine (K) (numbering according to Kabat), and wherein in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

20. The T cell activating bispecific antigen binding molecule according to claim 14, wherein in the constant domain CL of the second Fab molecule under b) the amino acid at position 124 is substituted independently by lysine (K), arginine (R) or histidine (H) (numbering according to Kabat), and wherein in the constant domain CH1 of the second Fab molecule under b) the amino acid at position 147 or the amino acid at position 213 is substituted independently by glutamic acid (E), or aspartic acid (D) (numbering according to Kabat EU index).

21. The T cell activating bispecific antigen binding molecule according to claim 14 or 20, wherein in the constant domain CL of the second Fab molecule under b) the amino acid at position 124 is substituted independently by lysine (K), arginine (R) or histidine (H) (numbering according to Kabat), and wherein in the constant domain CH1 of the second Fab molecule under b) the amino acid at position 147 is substituted independently by glutamic acid (E), or aspartic acid (D) (numbering according to Kabat EU index).

22. The T cell activating bispecific antigen binding molecule according to any one of claims 14, 20 and 21, wherein in the constant domain CL of the second Fab molecule under b) the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and wherein in the constant domain CH1 of the second Fab molecule under b) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

23. The T cell activating bispecific antigen binding molecule according to any one of claims 14 and 20-22, wherein in the constant domain CL of the second Fab molecule under b) the amino acid at position 124 is substituted by lysine (K) (numbering according to Kabat) and the amino acid at position 123 is substituted by lysine (K) (numbering according to Kabat), and wherein in the constant domain CH1 of the second Fab molecule under b) the amino acid at position 147 is substituted by glutamic acid (E) (numbering according to Kabat EU index) and the amino acid at position 213 is substituted by glutamic acid (E) (numbering according to Kabat EU index).

24. The T cell activating bispecific antigen binding molecule according to any one of claims 14 and 20-22, wherein in the constant domain CL of the second Fab molecule under b) the amino acid at position 124 is substituted by lysine (K) (numbering according to Kabat) and the amino acid at position 123 is substituted by lysine (K) (numbering according to Kabat), and wherein in the constant domain CH1 of the second Fab molecule under b) the amino acid at position 147 is substituted by glutamic acid (E) (numbering according to Kabat EU index) and the amino acid at position 213 is substituted by glutamic acid (E) (numbering according to Kabat EU index).

25. The T cell activating bispecific antigen binding molecule according to any one of claims 1-24, further comprising
c) a third antigen binding moiety which specifically binds to the first antigen.

26. The T cell activating bispecific antigen binding molecule according to claim 25, wherein the third antigen binding moiety is a Fab molecule.

27. The T cell activating bispecific antigen binding molecule according to claim 25 or 26, wherein the third antigen binding moiety is identical to the first antigen binding moiety.

28. The T cell activating bispecific antigen binding molecule according to any one of claims 25-27, wherein the first and the third antigen binding moiety specifically bind to a target cell antigen, and the second antigen binding moiety specifically binds to an activating T cell antigen, particularly CD3, more particularly CD3 epsilon.

29. The T cell activating bispecific antigen binding molecule according to any one of claims 1 to 28, additionally comprising
d) an Fc domain composed of a first and a second subunit capable of stable association.

30. The T cell activating bispecific antigen binding molecule according to any one of claims 1 to 29, wherein the first and the second antigen binding moiety are fused to each other, optionally via a peptide linker.

31. The T cell activating bispecific antigen binding molecule according to any one of claims 1 to 30, wherein the first and the second antigen binding moieties are Fab molecules and the second antigen binding moiety is fused at the C-terminus of the Fab heavy chain to the N-terminus of the Fab heavy chain of the first antigen binding moiety.

32. The T cell activating bispecific antigen binding molecule of any one of claims 1 to 30, wherein the first and the second antigen binding moieties are Fab molecules and the first antigen binding moiety is fused at the C-terminus of the Fab heavy chain to the N-terminus of the Fab heavy chain of the second antigen binding moiety.

33. The T cell activating bispecific antigen binding molecule of claim 31 or 32, wherein the first and the second antigen binding moieties are Fab molecules and the Fab light chain of the first antigen binding moiety and the Fab light chain of the second antigen binding moiety are fused to each other, optionally via a peptide linker.

34. The T cell activating bispecific antigen binding molecule according to claim 29, wherein the first and the second antigen binding moieties are Fab molecules and the second antigen binding moiety is fused at the C-terminus of the Fab heavy chain to the N-terminus of the first or the second subunit of the Fc domain.

35. The T cell activating bispecific antigen binding molecule according to claim 29, wherein the first and the second antigen binding moieties are Fab molecules and the first antigen binding moiety is fused at the C-terminus of the Fab heavy chain to the N-terminus of the first or the second subunit of the Fc domain.

36. The T cell activating bispecific antigen binding molecule according to claim 29, wherein the first and the second antigen binding moieties are Fab molecules and the first and the second antigen binding moiety are each fused at the C-terminus of the Fab heavy chain to the N-terminus of one of the subunits of the Fc domain.

37. The T cell activating bispecific antigen binding molecule according to any one of claim 29, 34 or 35, wherein the third antigen binding moiety is a Fab molecule and is fused at the C-terminus of the Fab heavy chain to the N-terminus of the first or second subunit of the Fc domain.

38. The T cell activating bispecific antigen binding molecule of claim 29, wherein the first, second and third antigen binding moieties are Fab molecules and the second and the third antigen binding moiety are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety.

39. The T cell activating bispecific antigen binding molecule according to claim 29, wherein the first, second and third antigen binding moieties are Fab molecules and the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety.

40. The T cell activating bispecific antigen binding molecule according to claim 39, wherein the first and the third antigen binding moiety and the Fc domain are part of an immunoglobulin molecule, particularly an IgG class immunoglobulin.

41. The T cell activating bispecific antigen binding molecule according to any one of claims 29-40, wherein the Fc domain is an IgG, specifically an IgG₁ or IgG₄, Fc domain.

42. The T cell activating bispecific antigen binding molecule according to any one of claims 29-41, wherein the Fc domain is a human Fc domain.

43. The T cell activating bispecific antigen binding molecule according to any one of claims 29-42, wherein the Fc domain comprises a modification promoting the association of the first and the second subunit of the Fc domain.

44. The T cell activating bispecific antigen binding molecule of claim 43, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

45. The T cell activating bispecific antigen binding molecule of claim 44, wherein said amino acid residue having a larger side chain volume is selected from the group consisting of arginine (R), phenylalanine (F), tyrosine (Y), and tryptophan (W), and said amino acid residue having a smaller side chain volume is selected from the group consisting of alanine (A), serine (S), threonine (T), and valine (V).

46. The T cell activating bispecific antigen binding molecule of claim 44 or 45, wherein in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V), and optionally in the second subunit of the Fc domain additionally the threonine residue at position 366 is replaced with a serine residue (T366S) and the leucine residue at position 368 is replaced with an alanine residue (L368A) (numberings according to Kabat EU index).

47. The T cell activating bispecific antigen binding molecule of any one of claims 44-46, wherein in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index).

48. The T cell activating bispecific antigen binding molecule of any one of claims 44-47, wherein the first subunit of the Fc domain comprises amino acid substitutions S354C and T366W, and the second subunit of the Fc domain comprises amino acid substitutions Y349C, T366S, L368A and Y407V (numbering according to Kabat EU index).

49. The T cell activating bispecific antigen binding molecule according to any one of claims 29-48, wherein the Fc domain exhibits reduced binding affinity to an Fc receptor and/or reduced effector function, as compared to a native IgG₁ Fc domain.

50. The T cell activating bispecific antigen binding molecule according to any one of claims 29-49, wherein the Fc domain comprises one or more amino acid substitution that reduces binding to an Fc receptor and/or effector function.

51. The T cell activating bispecific antigen binding molecule according to claim 50, wherein said one or more amino acid substitution is at one or more position selected from the group of L234, L235, and P329 (Kabat EU index numbering).

52. The T cell activating bispecific antigen binding molecule according to any one of claims 29-51, wherein each subunit of the Fc domain comprises three amino acid substitutions that reduce binding to an activating Fc receptor and/or effector function wherein said amino acid substitutions are L234A, L235A and P329G (Kabat EU index numbering).

53. The T cell activating bispecific antigen binding molecule of any one of claims 49-52, wherein the Fc receptor is an Fcγ receptor.

54. The T cell activating bispecific antigen binding molecule of any one of claims 49-53, wherein the effector function is antibody-dependent cell-mediated cytotoxicity (ADCC).

55. One or more isolated polynucleotide encoding the T cell activating bispecific antigen binding molecule of any one of claims 1 to 54.

56. One or more vector, particularly expression vector, comprising the polynucleotide(s) of claim 55.

57. A host cell comprising the polynucleotide(s) of claim 55 or the vector(s) of claim 56.

58. A method of producing a T cell activating bispecific antigen binding molecule capable of specific binding to Robo 4 and an activating T cell antigen, comprising the steps of a) culturing the host cell of claim 57 under conditions suitable for the expression of the T cell activating bispecific antigen binding molecule and b) optionally recovering the T cell activating bispecific antigen binding molecule.

59. A T cell activating bispecific antigen binding molecule produced by the method of claim 58.

60. A pharmaceutical composition comprising the T cell activating bispecific antigen binding molecule of any one of claim 1 to 54 or 59 and a pharmaceutically acceptable carrier.

61. The T cell activating bispecific antigen binding molecule of any one of claim 1 to 54 or 59 or the pharmaceutical composition of claim 60 for use as a medicament.

62. The T cell activating bispecific antigen binding molecule of any one of claim 1 to 54 or 59 or the pharmaceutical composition of claim 60 for use in the treatment of a disease in an individual in need thereof.

63. The T cell activating bispecific antigen binding molecule or the pharmaceutical composition of claim 62, wherein the disease is cancer.

64. Use of the T cell activating bispecific antigen binding molecule of any one of claim 1 to 54 or 59 for the manufacture of a medicament for the treatment of a disease in an individual in need thereof.

65. A method of treating a disease in an individual, comprising administering to said individual a therapeutically effective amount of a composition comprising the T cell activating bispecific antigen binding molecule of any one of claim 1 to 54 or 59 in a pharmaceutically acceptable form.

66. The use of claim 64 or the method of claim 65, wherein said disease is cancer.

67. A method for inducing lysis of a target cell, comprising contacting a target cell with the T cell activating bispecific antigen binding molecule of any one of claim 1-54 or 59 in the presence of a T cell.

68. The method of claim 67, wherein the target cell expresses Robo 4.

69. An antibody which specifically binds to Robo 4, wherein said antibody specifically binds to an epitope in the Ig-like domain 1 (position 20-119 of SEQ ID NO: 15) and/or the Ig-like domain 2 (position 20-107 of SEQ ID NO: 17) of the extracellular domain of Robo 4.

70. The antibody of claim 69, wherein said antibody comprises
(i) a heavy chain variable region comprising the heavy chain complementarity determining region (HCDR) 1 of SEQ ID NO: 91, the HCDR 2 of SEQ ID NO: 92 and the HCDR 3 of SEQ ID NO: 93, and a light chain variable region comprising the light chain complementarity determining region (LCDR) 1 of SEQ ID NO: 94, the LCDR 2 of SEQ ID NO: 95 and the LCDR 3 of SEQ ID NO: 96;
(ii) a heavy chain variable region comprising the heavy chain complementarity determining region (HCDR) 1 of SEQ ID NO: 103, the HCDR 2 of SEQ ID NO: 104 and the HCDR 3 of SEQ ID NO: 105, and a light chain variable region comprising the light chain complementarity determining region (LCDR) 1 of SEQ ID NO: 106, the LCDR 2 of SEQ ID NO: 107 and the LCDR 3 of SEQ ID NO: 108; or
(iii) a heavy chain variable region comprising the heavy chain complementarity determining region (HCDR) 1 of SEQ ID NO: 109, the HCDR 2 of SEQ ID NO: 110 and the HCDR 3 of SEQ ID NO: 111, and a light chain variable region comprising the light chain complementarity determining region (LCDR) 1 of SEQ ID NO: 112, the LCDR 2 of SEQ ID NO: 113 and the LCDR 3 of SEQ ID NO: 114.

71. The antibody according to claim 69 or 70, wherein said antibody comprises
(i) a heavy chain variable region comprising an amino acid sequence that is at least about 95%, 96%, 97%, 98%, 99% or 100% identical to the amino acid sequence of SEQ ID NO: 19 and a light chain variable region comprising an amino acid sequence that is at least about 95%, 96%, 97%, 98%, 99% or 100% identical to the amino acid sequence of SEQ ID NO: 21;
(ii) a heavy chain variable region comprising an amino acid sequence that is at least about 95%, 96%, 97%, 98%, 99% or 100% identical to the amino acid sequence of SEQ ID NO: 27 and a light chain variable region comprising an amino acid sequence that is at least about 95%, 96%, 97%, 98%, 99% or 100% identical to the amino acid sequence of SEQ ID NO: 29; or
(iii) a heavy chain variable region comprising an amino acid sequence that is at least about 95%, 96%, 97%, 98%, 99% or 100% identical to the amino acid sequence of SEQ ID NO: 31 and a light chain variable region comprising an amino acid sequence that is at least about 95%, 96%, 97%, 98%, 99% or 100% identical to the amino acid sequence of SEQ ID NO: 33.

72. The antibody of claim 70, wherein said antibody comprises human heavy and light chain variable region framework sequences.

73. An antibody which specifically binds to Robo 4, wherein said antibody competes with the antibody of claim 71 for binding an epitope of Robo4.

74. An antibody which specifically binds to Robo 4, wherein said antibody specifically binds to an epitope in the fibronectin-like domain 1 (position 20-108 of SEQ ID NO: 11) and/or the fibronectin-like domain 2 (position 20-111 of SEQ ID NO: 11) of the extracellular domain of Robo 4.

75. The antibody of claim 74, wherein said antibody comprises a heavy chain variable region comprising the heavy chain complementarity determining region (HCDR) 1 of SEQ ID NO: 97, the HCDR 2 of SEQ ID NO: 98 and the HCDR 3 of SEQ ID NO: 99, and a light chain variable region comprising the light chain complementarity determining region (LCDR) 1 of SEQ ID NO:
100, the LCDR 2 of SEQ ID NO: 101 and the LCDR 3 of SEQ ID NO: 102.

76. The antibody according to claim 74 or 75, wherein said antibody comprises a heavy chain variable region comprising an amino acid sequence that is at least about 95%, 96%, 97%, 98%, 99% or 100% identical to the amino acid sequence of SEQ ID NO: 23 and a light chain variable region comprising an amino acid sequence that is at least about 95%, 96%, 97%, 98%, 99% or 100% identical to the amino acid sequence of SEQ ID NO: 25.

77. The antibody of claim 75, wherein said antibody comprises human heavy and light chain variable region framework sequences.

78. An antibody which specifically binds to Robo 4, wherein said antibody competes with the antibody of claim 76 for binding an epitope of Robo4.

79. The invention as described hereinbefore.


SEQUENCE LISTING (162 sequences)

SEQ ID NO: 1 - protein, 493 aa, Artificial Sequence
  Human Robo4 ECD_PreS_Avi_His
MDMRVPAQLLGLLLLWFPGARCQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRVDLEVLFQGPGSGLNDIFEAQKIEWHEARAHHHHHH

SEQ ID NO: 2 - DNA, 1479 bp, Artificial Sequence
  Human Robo4 ECD_PreS_Avi_His
atggacatgagggtccccgctcagctcctgggcctcctgctgctctggttcccaggtgccaggtgtcaggactccccgccccagatcctagtccacccccaggaccagctgttccagggccctggccctgccaggatgagctgccaagcctcaggccagccacctcccaccatccgctggttgctgaatgggcagcccctgagcatggtgcccccagacccacaccacctcctgcctgatgggacccttctgctgctacagccccctgcccggggacatgcccacgatggccaggccctgtccacagacctgggtgtctacacatgtgaggccagcaaccggcttggcacggcagtcagcagaggcgctcggctgtctgtggctgtcctccgggaggatttccagatccagcctcgggacatggtggctgtggtgggtgagcagtttactctggaatgtgggccgccctggggccacccagagcccacagtctcatggtggaaagatgggaaacccctggccctccagcccggaaggcacacagtgtccggggggtccctgctgatggcaagagcagagaagagtgacgaagggacctacatgtgtgtggccaccaacagcgcaggacatagggagagccgcgcagcccgggtttccatccaggagccccaggactacacggagcctgtggagcttctggctgtgcgaattcagctggaaaatgtgacactgctgaacccggatcctgcagagggccccaagcctagaccggcggtgtggctcagctggaaggtcagtggccctgctgcgcctgcccaatcttacacggccttgttcaggacccagactgccccgggaggccagggagctccgtgggcagaggagctgctggccggctggcagagcgcagagcttggaggcctccactggggccaagactacgagttcaaagtgagaccatcctctggccgggctcgaggccctgacagcaacgtgctgctcctgaggctgccggaaaaagtgcccagtgccccacctcaggaagtgactctaaagcctggcaatggcactgtctttgtgagctgggtcccaccacctgctgaaaaccacaatggcatcatccgtggctaccaggtctggagcctgggcaacacatcactgccaccagccaactggactgtagttggtgagcagacccagctggaaatcgccacccatatgccaggctcctactgcgtgcaagtggctgcagtcactggtgctggagctggggagcccagtagacctgtctgcctccttttagagcaggccatggagcgagccacccaagaacccagtgagcatggtccctggaccctggagcagctgagggtcgacctggaagttctgttccaggggcccggctcaggcctgaacgacatcttcgaggcccagaagatcgagtggcacgaggctcgagctcaccaccatcaccatcac

SEQ ID NO: 3 - protein, 384 aa, Artificial Sequence
  Murine Robo4 ECD_PreS_Avi_His
MDMRVPAQLLGLLLLWFPGARCEDFQIQPRDTVAVVGESLVLECGPPWGYPKPSVSWWKDGKPLVLQPGRRTVSGDSLMVSRAEKNDSGTYMCMATNNAGQRESRAARVSIQESQDHKEHLELLAVRIQLENVTLLNPEPVKGPKPGPSVWLSWKVSGPAAPAESYTALFRTQRSPRDQGSPWTEVLLRGLQSAKLGGLHWGQDYEFKVRPSSGRARGPDSNVLLLRLPEQVPSAPPQGVTLRSGNGSVFVSWAPPPAESHNGVIRGYQVWSLGNASLPAANWTVVGEQTQLEIATRLPGSYCVQVAAVTGAGAGELSTPVCLLLEQAMEQSARDPRKHVPWTLEQLRVDLEVLFQGPGSGLNDIFEAQKIEWHEARAHHHHHH

SEQ ID NO: 4 - DNA, 1152 bp, Artificial Sequence
  Murine Robo4 ECD_PreS_Avi_His
atggacatgagggtccccgctcagctcctgggcctcctgctgctctggttcccaggtgccaggtgtgaggacttccagatccaacctcgggacacagtggccgtggtgggagagagcttggttcttgagtgtggtcctccctggggctacccaaaaccctcggtctcatggtggaaagacgggaaacccctggtcctccagccagggaggcgcacagtatctggggattccctgatggtgtcaagagcagagaagaatgactcggggacctatatgtgtatggccaccaacaatgctgggcaacgggagagccgagcagccagggtgtctatccaggaatcccaggaccacaaggaacatctagagcttctggctgttcgcattcagctggaaaatgtgaccctgctaaaccccgaacctgtaaaaggtcccaagcctgggccatccgtgtggctcagctggaaggtgagcggccctgctgcacctgctgagtcatacacagctctgttcaggactcagaggtcccccagggaccaaggatctccatggacagaggtgctgctgcgtggcttgcagagtgcaaagcttgggggtctccactggggccaagactatgaattcaaagtgagaccgtcctccggccgggctcgaggccctgacagcaatgtgttgctcctgaggctgcctgaacaggtgcccagtgccccacctcaaggagtgaccttaagatctggcaacggtagtgtctttgtgagttgggctccaccacctgctgaaagccataatggtgtcatccgtggttaccaggtctggagcctgggcaatgcctcattgcctgctgccaactggaccgtagtgggtgaacagacccagctggagatcgccacacgactgccaggctcctattgtgtgcaagtggctgcagtcactggagctggtgctggagaactcagtacccctgtctgcctccttttagagcaggccatggagcaatcagcacgagaccccaggaaacatgttccctggaccctggaacagctgagggtcgacctggaagttctgttccaggggcccggctcaggcctgaacgacatcttcgaggcccagaagatcgagtggcacgaggctcgagctcaccaccatcaccatcac

SEQ ID NO: 5 - protein, 492 aa, Artificial Sequence
  Human Robo4 ECD_AcTEV_Avi_His
MDMRVPAQLLGLLLLWFPGARCQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRVDEQLYFQGGSGLNDIFEAQKIEWHEARAHHHHHH

SEQ ID NO: 6 - DNA, 1476 bp, Artificial Sequence
  Human Robo4 ECD_AcTEV_Avi_His
atggacatgagggtccccgctcagctcctgggcctcctgctgctctggttcccaggtgccaggtgtcaggactccccgccccagatcctagtccacccccaggaccagctgttccagggccctggccctgccaggatgagctgccaagcctcaggccagccacctcccaccatccgctggttgctgaatgggcagcccctgagcatggtgcccccagacccacaccacctcctgcctgatgggacccttctgctgctacagccccctgcccggggacatgcccacgatggccaggccctgtccacagacctgggtgtctacacatgtgaggccagcaaccggcttggcacggcagtcagcagaggcgctcggctgtctgtggctgtcctccgggaggatttccagatccagcctcgggacatggtggctgtggtgggtgagcagtttactctggaatgtgggccgccctggggccacccagagcccacagtctcatggtggaaagatgggaaacccctggccctccagcccggaaggcacacagtgtccggggggtccctgctgatggcaagagcagagaagagtgacgaagggacctacatgtgtgtggccaccaacagcgcaggacatagggagagccgcgcagcccgggtttccatccaggagccccaggactacacggagcctgtggagcttctggctgtgcgaattcagctggaaaatgtgacactgctgaacccggatcctgcagagggccccaagcctagaccggcggtgtggctcagctggaaggtcagtggccctgctgcgcctgcccaatcttacacggccttgttcaggacccagactgccccgggaggccagggagctccgtgggcagaggagctgctggccggctggcagagcgcagagcttggaggcctccactggggccaagactacgagttcaaagtgagaccatcctctggccgggctcgaggccctgacagcaacgtgctgctcctgaggctgccggaaaaagtgcccagtgccccacctcaggaagtgactctaaagcctggcaatggcactgtctttgtgagctgggtcccaccacctgctgaaaaccacaatggcatcatccgtggctaccaggtctggagcctgggcaacacatcactgccaccagccaactggactgtagttggtgagcagacccagctggaaatcgccacccatatgccaggctcctactgcgtgcaagtggctgcagtcactggtgctggagctggggagcccagtagacctgtctgcctccttttagagcaggccatggagcgagccacccaagaacccagtgagcatggtccctggaccctggagcagctgagggtcgacgaacagttatattttcagggcggctcaggcctgaacgacatcttcgaggcccagaagatcgagtggcacgaggctcgagctcaccaccatcaccatcac

SEQ ID NO: 7 - protein, 889 aa, Artificial Sequence
  Human Robo1 ECD_PreS_Avi_His
MDMRVPAQLLGLLLLWFPGARCSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQVGSEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLVDLEVLFQGPGSGLNDIFEAQKIEWHEARAHHHHHH

SEQ ID NO: 8 - DNA, 2667 bp, Artificial Sequence
  Human Robo1 ECD_PreS_Avi_His
atggacatgagggtccccgctcagctcctgggcctcctgctgctctggttcccaggtgccaggtgttcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagttgggtctgaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcatggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgtcgacctggaagttctgttccaggggcccggctcaggcctgaacgacatcttcgaggcccagaagatcgagtggcacgaggctcgagctcaccaccatcaccatcac

SEQ ID NO: 9 - protein, 490 aa, Artificial Sequence
  Cynomolgus Robo4 ECD_AcTEV_Avi_His
MGWSCIILFLVATATGVHSQDSPPQILVHPQDQLFQGPGPARMSCRASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQLTLECGPPWGHPEPTVSWWKDGKPLVLQPGRYTVSGGSLLMARAEKSDAGAYMCVAANSAGHRESRAARVSIQEPQDYTEPVELLAVRIQLENVTLLNPDPAKGPKPGPAVWLSWKVSGPAAPAQSYTALFRTQTAPGDQGAPWTEELLAGWQSAELGGLHWGQDYEFKVRPSSGRARGPDSNVLLLRLPEKVPSAPPQEVTLKPGNGSVLVSWVPPSAENHNGTIRGYQVWSLGNTSLPPANWTVVGEQTQLEIATRMPGSYCVQVAAVTGAGAGEPSSPVCLLLEQAMERATREPSEHGPWTLEQLRVDLEVLFQGPGSGLNDIFEAQKIEWHEARAHHHHHH

SEQ ID NO: 10 - DNA, 1470 bp, Artificial Sequence
  Cynomolgus Robo4 ECD_AcTEV_Avi_His
atgggatggagctgtatcatcctcttcttggtagcaacagctaccggtgtgcattcccaggactccccgccccagatcctagtccacccgcaagaccagctgttccagggccctggccctgccaggatgagctgccgagcctcgggccagccacctcccaccatccgctggctgctgaatgggcagcccctgagcatggtacccccagacccacaccacctccttcctgatgggacccttctgctgctgcagccccctgcccggggacatgcccacgatggccaggccctgtccacagaccttggtgtctacacatgtgaggccagcaaccggctgggcacagcagtcagcagaggcgctcggctctctgtggctgtcctccgggaggatttccagatccagcctcgggacatggtagctgtggtgggtgagcagttaactctggaatgtgggccgccctggggccacccagagcccacagtctcatggtggaaagatgggaaacccctggtcctccagcccggaaggtacacggtgtccggggggtccctgctgatggcaagagcagagaagagtgacgcaggggcctacatgtgtgtggccgccaacagcgcaggacacagggagagccgcgcagcccgggtgtccatccaggagccccaggactacacagagcctgtggagcttttggctgtgcgaattcagctggaaaatgtgacactgctgaacccggaccccgcaaagggccccaagcctggaccggctgtgtggctcagctggaaggtgagcggccctgctgcacctgcccaatcttacacggccttgttcaggacccagactgccccgggagaccagggagctccatggacagaggagctgctggctggctggcagagcgcagagcttggaggcctccactggggccaagactatgagttcaaagtgagaccatcctccggccgggctcgaggccctgacagcaacgtgctgctcctgaggctgccggaaaaagtgcccagtgccccaccccaggaggtgaccctaaaacctggcaatggcagtgtccttgtgagctgggtcccaccatctgctgaaaaccacaatggcaccatccgtggctaccaggtctggagcctgggcaacacgtcactgcccccagccaactggactgtggttggtgagcagacccagctggaaatcgccacccgcatgccaggctcctactgtgtgcaagtggctgcagtcactggtgctggagctggggaacccagtagccctgtctgcctccttttagagcaggccatggagcgagccacccgagaacccagtgagcatggtccctggaccctggagcagctgagggtcgacctggaagttctgttccaggggcccggctcaggcctgaacgacatcttcgaggcccagaagatcgagtggcacgaggctcgagctcaccaccatcaccatcac

SEQ ID NO: 11 - protein, 370 aa, Artificial Sequence
  Human Robo4 FN-like domain 1_Fc knob_Avi
MGWSCIILFLVATATGVHSPDPAEGPKPRPAVWLSWKVSGPAAPAQSYTALFRTQTAPGGQGAPWAEELLAGWQSAELGGLHWGQDYEFKVRPSSGRARGPDSNVLLLRLVDGGSPTPPTPGGGSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 12 - DNA, 1110 bp, Artificial Sequence
  Human Robo4 FN-like domain 1_Fc knob_Avi
atgggatggagctgtatcatcctcttcttggtagcaacagctaccggtgtgcattccccggatcctgcagagggccccaagcctagaccggcggtgtggctcagctggaaggtcagtggccctgctgcgcctgcccaatcttacacggccttgttcaggacccagactgccccgggaggccagggagctccgtgggcagaggagctgctggccggctggcagagcgcagagcttggaggcctccactggggccaagactacgagttcaaagtgagaccatcctctggccgggctcgaggccctgacagcaacgtgctgctcctgaggctggtcgacggtggtagtccgacacctccgacacccgggggtggttctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 13 - protein, 371 aa, Artificial Sequence
  Human Robo4 FN-like domain 2_Fc knob_Avi
MGWSCIILFLVATATGVHSPSAPPQEVTLKPGNGTVFVSWVPPPAENHNGIIRGYQVWSLGNTSLPPANWTVVGEQTQLEIATHMPGSYCVQVAAVTGAGAGEPSRPVCLLVDGGSPTPPTPGGGSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 14 - DNA, 1113 bp, Artificial Sequence
  Human Robo4 FN-like domain 2_Fc knob_Avi
atgggatggagctgtatcatcctcttcttggtagcaacagctaccggtgtgcattcccccagtgccccacctcaggaagtgactctaaagcctggcaatggcactgtctttgtgagctgggtcccaccacctgctgaaaaccacaatggcatcatccgtggctaccaggtctggagcctgggcaacacatcactgccaccagccaactggactgtagttggtgagcagacccagctggaaatcgccacccatatgccaggctcctactgcgtgcaagtggctgcagtcactggtgctggagctggggagcccagtagacctgtctgcctccttgtcgacggtggtagtccgacacctccgacacccgggggtggttctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 15 - protein, 379 aa, Artificial Sequence
  Human Robo4 Ig-like domain 1_Fc knob_Avi
MGWSCIILFLVATATGVHSPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVDGGSPTPPTPGGGSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 16 - DNA, 1137 bp, Artificial Sequence
  Human Robo4 Ig-like domain 1_Fc knob_Avi
atgggatggagctgtatcatcctcttcttggtagcaacagctaccggtgtgcattccccccagatcctagtccacccccaggaccagctgttccagggccctggccctgccaggatgagctgccaagcctcaggccagccacctcccaccatccgctggttgctgaatgggcagcccctgagcatggtgcccccagacccacaccacctcctgcctgatgggacccttctgctgctacagccccctgcccggggacatgcccacgatggccaggccctgtccacagacctgggtgtctacacatgtgaggccagcaaccggcttggcacggcagtcagcagaggcgctcggctgtctgtcgacggtggtagtccgacacctccgacacccgggggtggttctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 17 - protein, 367 aa, Artificial Sequence
  Human Robo4 Ig-like domain 2_Fc knob_Avi
MGWSCIILFLVATATGVHSEDFQIQPRDMVAVVGEQFTLECGPPWGHPEPTVSWWKDGKPLALQPGRHTVSGGSLLMARAEKSDEGTYMCVATNSAGHRESRAARVSVDGGSPTPPTPGGGSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 18 - DNA, 1101 bp, Artificial Sequence
  Human Robo4 Ig-like domain 2_Fc knob_Avi
atgggatggagctgtatcatcctcttcttggtagcaacagctaccggtgtgcattccgaggatttccagatccagcctcgggacatggtggctgtggtgggtgagcagtttactctggaatgtgggccgccctggggccacccagagcccacagtctcatggtggaaagatgggaaacccctggccctccagcccggaaggcacacagtgtccggggggtccctgctgatggcaagagcagagaagagtgacgaagggacctacatgtgtgtggccaccaacagcgcaggacatagggagagccgcgcagcccgggtttccgtcgacggtggtagtccgacacctccgacacccgggggtggttctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 19 - protein, 119 aa, Cricetulus migratorius
EVQLVESGGGLSQPGNSLKLSCEASGFTFRNYDMSWVRQAPGKGLEWVAYISSGGGPIYYVDAVKGRFTISRDNAKNLLFLQMNNLRSEDTAVYYCARDLADIGVFDYWGQGTMVTVSS

SEQ ID NO: 20 - DNA, 357 bp, Cricetulus migratorius
gaggtgcagctggtggagtctgggggaggcttatcacagcctggaaattccctgaaactctcctgtgaggcctccggattcaccttcagaaattatgacatgagctgggtccgccaggctccagggaagggactggagtgggtcgcatacattagtagtggcggtggcccaatctattatgtcgatgctgtgaagggccggttcaccatctccagagacaacgccaagaacttactgttcctacaaatgaacaatctcaggtctgaggacacagccgtgtattactgtgcaagagatttggcggatataggagtttttgattattggggccaaggaaccatggtcaccgtctcctca

SEQ ID NO: 21 - protein, 106 aa, Cricetulus migratorius
DFKMTQSPDILSPSLGESVTITCQSSQNIYSNLAWYQQKPGEHPKLLIYTASILADGIPSRFTGSGSGTQFSLKISSLQPDDVANYYCQQYVYYRTFGPGTKLEIK

SEQ ID NO: 22 - DNA, 318 bp, Cricetulus migratorius
gattttaagatgactcagtctccagacatcctatcaccatcactgggggaaagtgtcactatcacatgccagtcaagtcagaatatttacagtaatttagcatggtatcagcagaaaccaggggaacatcctaagctcctgatctatactgcaagcatcttggcagatggaatcccttcaaggttcactggcagtggatctggaacacagttttctctcaagatcagcagcctgcagcctgacgatgtggcaaattattactgtcaacagtacgtttactatcggacgttcggacctggcaccaagctggaaatcaaa

SEQ ID NO: 23 - protein, 123 aa, Cricetulus migratorius
QVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSS

SEQ ID NO: 24 - DNA, 369 bp, Cricetulus migratorius
caggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctca

SEQ ID NO: 25 - protein, 111 aa, Cricetulus migratorius
QLVLTQSPSASASLGASVKLTCTLSSQHSSYGITWLQQHPDKAPKYVMYLKSDGSHTKGADIPDRFSGSSSGVHRYLSISNVQPEDEAIYFCVTYDSTHVFGSGTQLTVLG

SEQ ID NO: 26 - DNA, 333 bp, Cricetulus migratorius
caacttgttctgactcagtcaccctctgcctctgcctctctgggagcctcagtcaaactcacctgcaccttgagtagtcagcacagcagttatggcattacttggctccagcaacatccagacaaggctcctaagtatgtgatgtatcttaagagtgatggaagccataccaagggagctgatatcccggatcgcttctctggctccagttctggagttcatcgctacttaagcatctccaacgtgcagcctgaggatgaagcaatctatttctgtgttacatatgatagcactcatgtttttggcagcggaacccagctcaccgtcctaggt

SEQ ID NO: 27 - protein, 119 aa, Cricetulus migratorius
QIQLQESGPGLVKPSQSLSLTCSVTGYSISSGSLWTWIRQFPGNNLEWMGYISYAGGIDYNPSLTSRISITRDTSRNQFFLQLESVTTQDTATYYCATPGGYPFHFDYWGQGTMVTVSS

SEQ ID NO: 28 - DNA, 357 bp, Cricetulus migratorius
cagatccagctgcaggagtcaggacctggcctggtgaagccctcacagtcactgtccctcacttgctcagtcactggctactccatcagcagtggttccttgtggacatggatcaggcagttcccagggaataacctggagtggatgggatacataagttatgctggtggcattgactataatccttccctcacgagccgaatctccatcaccagagacacatccaggaaccagttcttcctacagttggagtctgtgaccactcaggacacagccacatattactgtgcaactccgggcggatatccgtttcactttgattactggggccaaggaaccatggtcaccgtctcctca

SEQ ID NO: 29 - protein, 116 aa, Cricetulus migratorius
QPVLTQSPSASASLGASVKLTCTLNSQFSTYNIGWYQQHRDKPPKYVMFVKGDGGHSKADGIPDRFSGSSSGADRYLTISNIQAEDEAIYFCGADYNNAGQYGCVFGSGTHFTVLG

SEQ ID NO: 30 - DNA, 348 bp, Cricetulus migratorius
caacctgtgctgactcagtcaccctctgcctctgcctccctgggagcctcagtcaaactcacctgtaccctgaatagtcaatttagcacctataatattggttggtatcaacaacatcgagacaaacctccgaagtatgtgatgtttgttaagggtgatggaggccacagcaaggcagatgggatccctgatcgcttctctggctccagttctggggccgaccgctatttaaccatctccaacatccaggctgaagatgaggctatctatttctgtggtgcagattataacaatgctggacaatatgggtgtgtttttggcagcggaacccacttcaccgtcctaggt

SEQ ID NO: 31 - protein, 117 aa, Artificial Sequence
  7G2 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGTGIFDYWGQGTLVTVSS

SEQ ID NO: 32 - DNA, 351 bp, Artificial Sequence
  7G2 VH
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaagggggtactgggatttttgactactggggccaaggaaccctggtcaccgtctcgagt

SEQ ID NO: 33 - protein, 108 aa, Artificial Sequence
  7G2 VL
EIVLTQSPGTLSLSPGERATLSCRASQSVSSSYLAWYQQKPGQAPRLLIYGASSRATGIPDRFSGSGSGTDFTLTISRLEPEDFAVYYCQQGQLPPRTFGQGTKVEIK

SEQ ID NO: 34 - DNA, 324 bp, Artificial Sequence
  7G2 VL
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagggtcagttgcctccccgtacgttcggccaggggaccaaagtggaaatcaaa

SEQ ID NO: 35 - protein, 671 aa, Artificial Sequence
  V9 VL-CH1 - 01E06 VH-CH1 - Fc knob (P329G LALA)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLSQPGNSLKLSCEASGFTFRNYDMSWVRQAPGKGLEWVAYISSGGGPIYYVDAVKGRFTISRDNAKNLLFLQMNNLRSEDTAVYYCARDLADIGVFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 2013 bp, Artificial Sequence
  V9 VL-CH1 - 01E06 VH-CH1 - Fc knob (P329G LALA)
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcagctggtggagtctgggggaggcttatcacagcctggaaattccctgaaactctcctgtgaggcctccggattcaccttcagaaattatgacatgagctgggtccgccaggctccagggaagggactggagtgggtcgcatacattagtagtggcggtggcccaatctattatgtcgatgctgtgaagggccggttcaccatctccagagacaacgccaagaacttactgttcctacaaatgaacaatctcaggtctgaggacacagccgtgtattactgtgcaagagatttggcggatataggagtttttgattattggggccaaggaaccatggtcaccgtctcctcagctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - protein, 445 aa, Artificial Sequence
  01E06 VH-CH1 - V9 VL-CH1
EVQLVESGGGLSQPGNSLKLSCEASGFTFRNYDMSWVRQAPGKGLEWVAYISSGGGPIYYVDAVKGRFTISRDNAKNLLFLQMNNLRSEDTAVYYCARDLADIGVFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - DNA, 1335 bp, Artificial Sequence
  01E06 VH-CH1 - V9 VL-CH1
gaggtgcagctggtggagtctgggggaggcttatcacagcctggaaattccctgaaactctcctgtgaggcctccggattcaccttcagaaattatgacatgagctgggtccgccaggctccagggaagggactggagtgggtcgcatacattagtagtggcggtggcccaatctattatgtcgatgctgtgaagggccggttcaccatctccagagacaacgccaagaacttactgttcctacaaatgaacaatctcaggtctgaggacacagccgtgtattactgtgcaagagatttggcggatataggagtttttgattattggggccaaggaaccatggtcaccgtctcctcagctagcaccaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggtggatccgacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgc

SEQ ID NO: 39 - protein, 213 aa, Artificial Sequence
  01E06 VL-CL
DFKMTQSPDILSPSLGESVTITCQSSQNIYSNLAWYQQKPGEHPKLLIYTASILADGIPSRFTGSGSGTQFSLKISSLQPDDVANYYCQQYVYYRTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 639 bp, Artificial Sequence
  01E06 VL-CL
gattttaagatgactcagtctccagacatcctatcaccatcactgggggaaagtgtcactatcacatgccagtcaagtcagaatatttacagtaatttagcatggtatcagcagaaaccaggggaacatcctaagctcctgatctatactgcaagcatcttggcagatggaatcccttcaaggttcactggcagtggatctggaacacagttttctctcaagatcagcagcctgcagcctgacgatgtggcaaattattactgtcaacagtacgtttactatcggacgttcggacctggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 41 - protein, 675 aa, Artificial Sequence
  V9 VL-CH1 - 01F05 VH-CH1 Fc knob (P329G LALA)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 2025 bp, Artificial Sequence
  V9 VL-CH1 - 01F05 VH-CH1 Fc knob (P329G LALA)
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctcaggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - protein, 675 aa, Artificial Sequence
  2C11 VL-CH1 - 01F05 VH-CH1-Fc knob (P329G LALA)
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 2025 bp, Artificial Sequence
  2C11 VL-CH1 - 01F05 VH-CH1-Fc knob (P329G LALA)
gacatccagatgacccagagccccagcagcctgcctgccagcctgggcgacagagtgaccatcaactgccaggccagccaggacatcagcaactacctgaactggtatcagcagaagcctggcaaggcccccaagctgctgatctactacaccaacaagctggccgacggcgtgcccagcagattcagcggcagcggctccggcagagacagcagcttcaccatctccagcctggaaagcgaggacatcggcagctactactgccagcagtactacaactacccctggaccttcggccctggcaccaagctggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctcaggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 45 - protein, 453 aa, Artificial Sequence
  01F05 VH-CH1-Fc hole (P329G LALA)
QVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - DNA, 1359 bp, Artificial Sequence
  01F05 VH-CH1-Fc hole (P329G LALA)
caggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - protein, 449 aa, Artificial Sequence
  01F05 VH-CH1 - V9 VL-CH1
QVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - DNA, 1347 bp, Artificial Sequence
  01F05 VH-CH1 - V9 VL-CH1
caggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggtggatccgacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgc

SEQ ID NO: 49 - protein, 449 aa, Artificial Sequence
  2C11 VL-CH1 - 01F05 VH-CH1
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 50 - DNA, 1347 bp, Artificial Sequence
  2C11 VL-CH1 - 01F05 VH-CH1
gacatccagatgacccagagccccagcagcctgcctgccagcctgggcgacagagtgaccatcaactgccaggccagccaggacatcagcaactacctgaactggtatcagcagaagcctggcaaggcccccaagctgctgatctactacaccaacaagctggccgacggcgtgcccagcagattcagcggcagcggctccggcagagacagcagcttcaccatctccagcctggaaagcgaggacatcggcagctactactgccagcagtactacaactacccctggaccttcggccctggcaccaagctggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctcaggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgat

SEQ ID NO: 51 - protein, 686 aa, Artificial Sequence
  (01F05 VH-CH1)2 - V9 VL-CH1
QVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 52 - DNA, 2058 bp, Artificial Sequence
  (01F05 VH-CH1)2 - V9 VL-CH1
caggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacggcggaggagggtccggaggcggtggctcccaggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacggcggaggagggtccggcggcggtggatccgacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgc

SEQ ID NO: 53 - protein, 216 aa, Artificial Sequence
  01F05 VL-CL
QLVLTQSPSASASLGASVKLTCTLSSQHSSYGITWLQQHPDKAPKYVMYLKSDGSHTKGADIPDRFSGSSSGVHRYLSISNVQPEDEAIYFCVTYDSTHVFGSGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - DNA, 648 bp, Artificial Sequence
  01F05 VL-CL
caacttgttctgactcagtcaccctctgcctctgcctctctgggagcctcagtcaaactcacctgcaccttgagtagtcagcacagcagttatggcattacttggctccagcaacatccagacaaggctcctaagtatgtgatgtatcttaagagtgatggaagccataccaagggagctgatatcccggatcgcttctctggctccagttctggagttcatcgctacttaagcatctccaacgtgcagcctgaggatgaagcaatctatttctgtgttacatatgatagcactcatgtttttggcagcggaacccagctcaccgtcctaggtcaacccaaggctgcccccagcgtgaccctgttcccccccagcagcgaggaactgcaggccaacaaggccaccctggtctgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggagaaaaccgtggcccccaccgagtgcagc

SEQ ID NO: 55 - protein, 671 aa, Artificial Sequence
  V9 VL-CH1 - 01F09 VH-CH1 Fc knob (P329G LALA)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQIQLQESGPGLVKPSQSLSLTCSVTGYSISSGSLWTWIRQFPGNNLEWMGYISYAGGIDYNPSLTSRISITRDTSRNQFFLQLESVTTQDTATYYCATPGGYPFHFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 2013 bp, Artificial Sequence
  V9 VL-CH1 - 01F09 VH-CH1 Fc knob (P329G LALA)
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctcagatccagctgcaggagtcaggacctggcctggtgaagccctcacagtcactgtccctcacttgctcagtcactggctactccatcagcagtggttccttgtggacatggatcaggcagttcccagggaataacctggagtggatgggatacataagttatgctggtggcattgactataatccttccctcacgagccgaatctccatcaccagagacacatccaggaaccagttcttcctacagttggagtctgtgaccactcaggacacagccacatattactgtgcaactccgggcggatatccgtttcactttgattactggggccaaggaaccatggtcaccgtctcctcagctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 57 - protein, 445 aa, Artificial Sequence
  01F09 VH-CH1 - V9 VL-CH1
QIQLQESGPGLVKPSQSLSLTCSVTGYSISSGSLWTWIRQFPGNNLEWMGYISYAGGIDYNPSLTSRISITRDTSRNQFFLQLESVTTQDTATYYCATPGGYPFHFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 58 - DNA, 1335 bp, Artificial Sequence
  01F09 VH-CH1 - V9 VL-CH1
cagatccagctgcaggagtcaggacctggcctggtgaagccctcacagtcactgtccctcacttgctcagtcactggctactccatcagcagtggttccttgtggacatggatcaggcagttcccagggaataacctggagtggatgggatacataagttatgctggtggcattgactataatccttccctcacgagccgaatctccatcaccagagacacatccaggaaccagttcttcctacagttggagtctgtgaccactcaggacacagccacatattactgtgcaactccgggcggatatccgtttcactttgattactggggccaaggaaccatggtcaccgtctcctcagctagcaccaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggtggatccgacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgc

SEQ ID NO: 59 - protein, 221 aa, Artificial Sequence
  01F09 VL-CL
QPVLTQSPSASASLGASVKLTCTLNSQFSTYNIGWYQQHRDKPPKYVMFVKGDGGHSKADGIPDRFSGSSSGADRYLTISNIQAEDEAIYFCGADYNNAGQYGCVFGSGTHFTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 60 - DNA, 663 bp, Artificial Sequence
  01F09 VL-CL
caacctgtgctgactcagtcaccctctgcctctgcctccctgggagcctcagtcaaactcacctgtaccctgaatagtcaatttagcacctataatattggttggtatcaacaacatcgagacaaacctccgaagtatgtgatgtttgttaagggtgatggaggccacagcaaggcagatgggatccctgatcgcttctctggctccagttctggggccgaccgctatttaaccatctccaacatccaggctgaagatgaggctatctatttctgtggtgcagattataacaatgctggacaatatgggtgtgtttttggcagcggaacccacttcaccgtcctaggtcaacccaaggctgcccccagcgtgaccctgttcccccccagcagcgaggaactgcaggccaacaaggccaccctggtctgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggagaaaaccgtggcccccaccgagtgcagc

SEQ ID NO: 61 - protein, 669 aa, Artificial Sequence
  V9 VL-CH1 - 7G2 VH CH1 Fc knob (P329G LALA)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGTGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 2007 bp, Artificial Sequence
  V9 VL-CH1 - 7G2 VH CH1 Fc knob (P329G LALA)
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaagggggtactgggatttttgactactggggccaaggaaccctggtcaccgtctcgagtgctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 63 - protein, 443 aa, Artificial Sequence
  7G2 VH-CH1 - V9 VL-CH1
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGTGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - DNA, 1329 bp, Artificial Sequence
  7G2 VH-CH1 - V9 VL-CH1
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaagggggtactgggatttttgactactggggccaaggaaccctggtcaccgtctcgagtgctagcaccaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggtggatccgacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgc

SEQ ID NO: 65 - protein, 215 aa, Artificial Sequence
  7G2 VL-CL
EIVLTQSPGTLSLSPGERATLSCRASQSVSSSYLAWYQQKPGQAPRLLIYGASSRATGIPDRFSGSGSGTDFTLTISRLEPEDFAVYYCQQGQLPPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 645 bp, Artificial Sequence
  7G2 VL-CL
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagggtcagttgcctccccgtacgttcggccaggggaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 67 - protein, 667 aa, Artificial Sequence
  V9 VL-CH1 - DP47GS VH-CH1-Fc hole (P329G LALA)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - DNA, 2001 bp, Artificial Sequence
  V9 VL-CH1 - DP47GS VH-CH1-Fc hole (P329G LALA)
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgcctctaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 69 - protein, 667 aa, Artificial Sequence
  2C11 VL-CH1 - DP47 VH-CH1 Fc hole (P329G LALA)
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - DNA, 2001 bp, Artificial Sequence
  2C11 VL-CH1 - DP47 VH-CH1 Fc hole (P329G LALA)
gacatccagatgacccagagccccagcagcctgcctgccagcctgggcgacagagtgaccatcaactgccaggccagccaggacatcagcaactacctgaactggtatcagcagaagcctggcaaggcccccaagctgctgatctactacaccaacaagctggccgacggcgtgcccagcagattcagcggcagcggctccggcagagacagcagcttcaccatctccagcctggaaagcgaggacatcggcagctactactgccagcagtactacaactacccctggaccttcggccctggcaccaagctggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgccagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 71 - protein, 445 aa, Artificial Sequence
  DP47GS VH-CH1-Fc knob (P329G LALA)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1335 bp, Artificial Sequence
  DP47GS VH-CH1-Fc knob (P329G LALA)
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 73 - protein, 670 aa, Artificial Sequence
  V9 VL-CH1 - (DP47GS VH-CH1)2
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 74 - DNA, 2010 bp, Artificial Sequence
  V9 VL-CH1 - (DP47GS VH-CH1)2
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtcgtgaccgtgcctagcagctctctgggcacccagacctacatctgcaacgtgaaccacaagcccagcaacaccaaggtggacaagaaggtggaacccaagagctgcggcggaggcggatctggcgggggaggatctgaagtgcagctgctggaatctggcggcggactggtgcagcctggcggatctctgagactgagctgtgccgccagcggcttcaccttcagcagctacgccatgagctgggtgcgccaggcccctggaaaaggcctggaatgggtgtccgccatctctggctctggcggcagcacctactacgccgatagcgtgaagggccggttcaccatcagccgggacaacagcaagaacaccctgtacctgcagatgaacagcctgcgggccgaggacaccgccgtgtactattgtgccaagggctccggcttcgactactggggccagggcacactcgtgacagtctcgagtgctagcaccaagggccccagcgtgttccctctggcccctagcagcaagtctaccagcggaggaacagccgccctgggctgcctcgtgaaggactactttcccgagcctgtgaccgtgtcctggaacagcggagccctgacaagcggcgtgcacacctttccagccgtgctgcagagcagcggcctgtactctctgtccagcgtcgtgacagtgcccagctctagcctgggaacacagacatatatctgtaatgtgaatcacaaaccctctaataccaaagtggataagaaagtggaacctaagtcctgcgacggcggagggggctccggaggcggcggaagcgaggtgcagctgctggaaagtgggggaggcctggtgcagccagggggaagcctgagactgtcttgtgccgcttccggctttacctttagctcttacgccatgtcttgggtgcggcaggctccaggcaagggactggaatgggtgtcagctatcagcggcagcggcggctccacatattacgccgactctgtgaagggcagattcacaatctcccgcgacaactccaagaatactctgtacctgcagatgaattccctgcgcgccgaagatacagctgtgtattactgcgccaagggcagcggctttgattattggggacagggaaccctcgtgacagtctcgagtgctagcacaaaaggaccttccgtgtttcccctggctcccagctccaagagcacatccggcggaacagctgctctgggatgtctcgtgaaagattattttcctgaacccgtgactgtgtcttggaattctggcgccctgacctccggggtgcacacattccctgctgtgctgcagtcctccggcctgtatagcctgtcctccgtcgtgactgtgccatccagcagcctggggactcagacttacatctgcaatgtgaatcataagccttccaacacaaaagtggacaaaaaagtggaacccaaaagttgcgac

SEQ ID NO: 75 - protein, 670 aa, Artificial Sequence
  2C11 VL-CH1 - (DP47GS VH-CH1)2
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 76 - DNA, 2010 bp, Artificial Sequence
  2C11 VL-CH1 - (DP47GS VH-CH1)2
gacatccagatgacccagagccccagcagcctgcctgccagcctgggcgacagagtgaccatcaactgccaggccagccaggacatcagcaactacctgaactggtatcagcagaagcctggcaaggcccccaagctgctgatctactacaccaacaagctggccgacggcgtgcccagcagattcagcggcagcggctccggcagagacagcagcttcaccatctccagcctggaaagcgaggacatcggcagctactactgccagcagtactacaactacccctggaccttcggccctggcaccaagctggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggaggagggtccgaggtgcaattgttggagtctgggggaggcttggtacagcctggggggtccctgagactctcctgtgcagcctctggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaggtggagcccaagagctgcgacggcggaggagggtccggaggcggtggctccgaggtgcaattgttggagtctgggggaggcttggtacagcctggggggtccctgagactctcctgtgcagcctctggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggataagaaagttgagcccaaatcttgtgac

SEQ ID NO: 77 - protein, 215 aa, Artificial Sequence
  DP47GS VL-CL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 645 bp, Artificial Sequence
  DP47GS VL-CL
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtatggtagctcaccgctgacgttcggccaggggaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 79 - protein, 229 aa, Artificial Sequence
  V9 VH-CL
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 687 bp, Artificial Sequence
  V9 VH-CL
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 81 - protein, 223 aa, Artificial Sequence
  2C11 VH-CL
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 669 bp, Artificial Sequence
  2C11 VH-CL
gaggtgcagctggtggaaagcggcggaggcctggtgcagcccggcaagagcctgaagctgagctgcgaggccagcggcttcaccttcagcggctacggcatgcactgggtgagacaggcccctggcagaggactggaaagcgtggcctacatcaccagcagcagcatcaacattaagtacgccgacgccgtgaagggccggttcaccgtgtccagggataacgccaagaacctgctgttcctgcagatgaacatcctgaagtccgaggacaccgctatgtattactgcgccagattcgactgggacaagaactactggggccagggcaccatggtcacagtgtctagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 83 - protein, 227 aa, Artificial Sequence
  Fc hole (P329G LALA)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - DNA, 681 bp, Artificial Sequence
  Fc hole (P329G LALA)
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 85 - protein, 122 aa, Artificial Sequence [sequence omitted]

SEQ ID NO: 86 - DNA, 366 bp, Artificial Sequence
  V9 VH
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagc

SEQ ID NO: 87 - protein, 107 aa, Artificial Sequence [sequence omitted]

SEQ ID NO: 88 - DNA, 321 bp, Artificial Sequence
  V9 VL
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaag

SEQ ID NO: 89 - protein, 249 aa, Artificial Sequence
  Fc hole
MDMRVPAQLLGLLLLWFPGARCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - DNA, 747 bp, Artificial Sequence
  Fc hole
atggacatgagggtccccgctcagctcctgggcctcctgctgctctggttcccaggtgccaggtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 91 - protein, 8 aa, Cricetulus migratorius
GFTFRNYD

SEQ ID NO: 92 - protein, 8 aa, Cricetulus migratorius
ISSGGGPI

SEQ ID NO: 93 - protein, 12 aa, Cricetulus migratorius
DLADIGVFDYWG

SEQ ID NO: 94 - protein, 6 aa, Cricetulus migratorius
QNIYSN

SEQ ID NO: 95 - protein, 4 aa, Cricetulus migratorius
YTAS

SEQ ID NO: 96 - protein, 12 aa, Cricetulus migratorius
QQYVYYRTFGPG

SEQ ID NO: 97 - protein, 7 aa, Cricetulus migratorius
VYTFTYG

SEQ ID NO: 98 - protein, 8 aa, Cricetulus migratorius
IDPDSGNS

SEQ ID NO: 99 - protein, 17 aa, Cricetulus migratorius
SMRYSGYRDYALDLWGQ

SEQ ID NO: 100 - protein, 7 aa, Cricetulus migratorius
SQHSSYG

SEQ ID NO: 101 - protein, 8 aa, Cricetulus migratorius
LKSDGSHT

SEQ ID NO: 102 - protein, 12 aa, Cricetulus migratorius
VTYDSTHVFGSG

SEQ ID NO: 103 - protein, 7 aa, Cricetulus migratorius
GYSISSG

SEQ ID NO: 104 - protein, 12 aa, Cricetulus migratorius
NPSLTSRISITR

SEQ ID NO: 105 - protein, 12 aa, Cricetulus migratorius
PGGYPFHFDYWG

SEQ ID NO: 106 - protein, 7 aa, Cricetulus migratorius
SQFSTYN

SEQ ID NO: 107 - protein, 11 aa, Cricetulus migratorius
VMFVKGDGGHS

SEQ ID NO: 108 - protein, 12 aa, Cricetulus migratorius
GADYNNAGQYGC

SEQ ID NO: 109 - protein, 8 aa, Artificial Sequence
  7G2 HCDR1
GFTFSSYA

SEQ ID NO: 110 - protein, 9 aa, Artificial Sequence
  7G2 HCDR2
AISGSGGST

SEQ ID NO: 111 - protein, 12 aa, Artificial Sequence
  7G2 HCDR3
GGTGIFDYWGQG

SEQ ID NO: 112 - protein, 7 aa, Artificial Sequence
  7G2 LCDR1
QSVSSSY

SEQ ID NO: 113 - protein, 4 aa, Artificial Sequence
  7G2 LCDR2
GASS

SEQ ID NO: 114 - protein, 13 aa, Artificial Sequence
  7G2 LCDR3
QQGQLPPRTFGQG

SEQ ID NO: 115 - DNA, 1611 bp, Artificial Sequence
  library template DP47-3 library; complete Fab coding region comprising PelB leader sequence + Vk3_20 kappa V-domain + CL constant domain for light chain and PelB + VH3_23 V-domain + CH1 constant domain for heavy chain
atgaaatacctattgcctacggcagccgctggattgttattactcgcggcccagccggccatggccgaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtatggtagctcaccgctgacgttcggccaggggaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggagccgcagaacaaaaactcatctcagaagaggatctgaatggagccgcagactacaaggacgacgacgacaagggtgccgcataataaggcgcgccaattctatttcaaggagacagtcatatgaaatacctgctgccgaccgctgctgctggtctgctgctcctcgctgcccagccggcgatggccgaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaccgtttccgtattttgactactggggccaaggaaccctggtcaccgtctcgagtgctagcaccaaaggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaagtggacaagaaagttgagcccaaatcttgtgacgcggccgcaagcactagtgcccatcaccatcaccatcacgccgcggcatag

SEQ ID NO: 116 - DNA, 26 bp, Artificial Sequence
  primer LMB3
caggaaacagctatgaccatgattac

SEQ ID NO: 117 - DNA, 63 bp, Artificial Sequence
  primer LibL1b_new
  misc_feature (26)..(27): n is a, c, g, or t
  misc_feature (32)..(33): n is a, c, g, or t
  misc_feature (35)..(36): n is a, c, g, or t
  misc_feature (38)..(39): n is a, c, g, or t
cactttggtcccctggccgaacgtmnngggmnnmnnmnnaccctgctgacagtaatacactgc

SEQ ID NO: 118 - DNA, 28 bp, Artificial Sequence
  primer MS63
tttcgcacagtaatatacggccgtgtcc

SEQ ID NO: 119 - DNA, 25 bp, Artificial Sequence
  primer MS64
acgttcggccaggggaccaaagtgg

SEQ ID NO: 120 - DNA, 60 bp, Artificial Sequence
  primer Lib2H
  misc_feature (23)..(24): n is a, c, g, or t
  misc_feature (26)..(27): n is a, c, g, or t
  misc_feature (29)..(30): n is a, c, g, or t
  misc_feature (32)..(33): n is a, c, g, or t
  misc_feature (35)..(36): n is a, c, g, or t
ggccgtatattactgtgcgaaannknnknnknnknnktttgactactggggccaaggaac

SEQ ID NO: 121 - DNA, 30 bp, Artificial Sequence
  primer fdseqlong
gacgttagtaaatgaattttctgtatgagg

SEQ ID NO: 122 - protein, 5 aa, Artificial Sequence
  V9 HCDR1
GYTMN

SEQ ID NO: 123 - protein, 17 aa, Artificial Sequence
  V9 HCDR2
LINPYKGVSTYNQKFKD

SEQ ID NO: 124 - protein, 13 aa, Artificial Sequence
  V9 HCDR3
SGYYGDSDWYFDV

SEQ ID NO: 125 - protein, 11 aa, Artificial Sequence
  V9 LCDR1
RASQDIRNYLN

SEQ ID NO: 126 - protein, 7 aa, Artificial Sequence
  V9 LCDR2
YTSRLES

SEQ ID NO: 127 - protein, 9 aa, Artificial Sequence
  V9 LCDR3
QQGNTLPWT

SEQ ID NO: 128 - protein, 5 aa, Artificial Sequence
  anti-CD3 HCDR1
TYAMN

SEQ ID NO: 129 - protein, 19 aa, Artificial Sequence
  anti-CD3 HCDR2
RIRSKYNNYATYYADSVKD

SEQ ID NO: 130 - protein, 14 aa, Artificial Sequence
  anti-CD3 HCDR3
HGNFGNSYVSWFAY

SEQ ID NO: 131 - protein, 14 aa, Artificial Sequence
  anti-CD3 LCDR1
RSSTGAVTTSNYAN

SEQ ID NO: 132 - protein, 7 aa, Artificial Sequence
  anti-CD3 LCDR2
GTNKRAP

SEQ ID NO: 133 - protein, 9 aa, Artificial Sequence
  anti-CD3 LCDR3
ALWYSNLWV

SEQ ID NO: 134 - protein, 125 aa, Artificial Sequence
  anti-CD3 VH
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 135 - protein, 109 aa, Artificial Sequence
  anti-CD3 VL
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 136 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 137 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 138 - protein, 1007 aa, Homo sapiens
MGSGGDSLLGGRGSLPLLLLLIMGGMAQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPEVIATCGVALWLLLLGTAVCIHRRRRARVHLGPGLYRYTSEDAILKHRMDHSDSQWLADTWRSTSGSRDLSSSSSLSSRLGADARDPLDCRRSLLSWDSRSPGVPLLPDTSTFYGSLIAELPSSTPARPSPQVPAVRRLPPQLAQLSSPCSSSDSLCSRRGLSSPRLSLAPAEAWKAKKKQELQHANSSPLLRGSHSLELRACELGNRGSKNLSQSPGAVPQALVAWRALGPKLLSSSNELVTRHLPPAPLFPHETPPTQSQQTQPPVAPQAPSSILLPAAPIPILSPCSPPSPQASSLSGPSPASSRLSSSSLSSLGEDQDSVLTPEEVALCLELSEGEETPRNSVSPMPRAPSPPTTYGYISVPTASEFTDMGRTGGGVGPKGGVLLCPPRPCLTPTPSEGSLANGWGSASEDNAASARASLVSSSDGSFLADAHFARALAVAVDSFGFGLEPREADCVFIDASSPPSPRDEIFLTPNLSLPLWEWRPDWLEDMEVSHTQRLGRGMPPWPPDSQISSQRSQLHCRMPKAGASPVDYS

SEQ ID NO: 139 - protein, 1015 aa, Mus musculus
MGQGEEPRAAMGSGGTGLLGTEWPLPLLLLFIMGGEALDSPPQILVHPQDQLLQGSGPAKMRCRSSGQPPPTIRWLLNGQPLSMATPDLHYLLPDGTLLLHRPSVQGRPQDDQNILSAILGVYTCEASNRLGTAVSRGARLSVAVLQEDFQIQPRDTVAVVGESLVLECGPPWGYPKPSVSWWKDGKPLVLQPGRRTVSGDSLMVSRAEKNDSGTYMCMATNNAGQRESRAARVSIQESQDHKEHLELLAVRIQLENVTLLNPEPVKGPKPGPSVWLSWKVSGPAAPAESYTALFRTQRSPRDQGSPWTEVLLRGLQSAKLGGLHWGQDYEFKVRPSSGRARGPDSNVLLLRLPEQVPSAPPQGVTLRSGNGSVFVSWAPPPAESHNGVIRGYQVWSLGNASLPAANWTVVGEQTQLEIATRLPGSYCVQVAAVTGAGAGELSTPVCLLLEQAMEQSARDPRKHVPWTLEQLRATLRRPEVIASSAVLLWLLLLGITVCIYRRRKAGVHLGPGLYRYTSEDAILKHRMDHSDSPWLADTWRSTSGSRDLSSSSSLSSRLGLDPRDPLEGRRSLISWDPRSPGVPLLPDTSTFYGSLIAEQPSSPPVRPSPKTPAARRFPSKLAGTSSPWASSDSLCSRRGLCSPRMSLTPTEAWKAKKKQELHQANSSPLLRGSHPMEIWAWELGSRASKNLSQSPGEAPRAVVSWRAVGPQLHRNSSELASRPLPPTPLSLRGASSHDPQSQCVEKLQAPSSDPLPAAPLSVLNSSRPSSPQASFLSCPSPSSSNLSSSSLSSLEEEEDQDSVLTPEEVALCLELSDGEETPTNSVSPMPRAPSPPTTYGYISIPTCSGLADMGRAGGGVGSEVGNLLYPPRPCPTPTPSEGSLANGWGSASEDNVPSARASLVSSSDGSFLADTHFARALAVAVDSFGLSLDPREADCVFTDASSPPSPRGDLSLTRSFSLPLWEWRPDWLEDAEISHTQRLGRGLPPWPPDSRASSQRSWLTGAVPKAGDSS

SEQ ID NO: 140 - protein, 125 aa, Artificial Sequence
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 141 - protein, 5 aa, Artificial Sequence
  CD3 HCDR1
TYAMN

SEQ ID NO: 142 - protein, 19 aa, Artificial Sequence
  CD3 HCDR2
RIRSKYNNYATYYADSVKG

SEQ ID NO: 143 - protein, 14 aa, Artificial Sequence
  CD3 HCDR3
HGNFGNSYVSWFAY

SEQ ID NO: 144 - protein, 109 aa, Artificial Sequence
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 145 - protein, 14 aa, Artificial Sequence
  CD3 LCDR1
GSSTGAVTTSNYAN

SEQ ID NO: 146 - protein, 7 aa, Artificial Sequence
  CD3 LCDR2
GTNKRAP

SEQ ID NO: 147 - protein, 9 aa, Artificial Sequence
  CD3 LCDR3
ALWYSNLWV

SEQ ID NO: 148 - protein, 10 aa, Artificial Sequence
  linker
GGGGSGGGGS

SEQ ID NO: 149 - protein, 11 aa, Artificial Sequence
  linker
DGGGGSGGGGS

SEQ ID NO: 150 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 151 - protein, 451 aa, Artificial Sequence
  Robo4 VH-CH1(EE)-Fc(hole, P329G LALA)
QVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 152 - protein, 676 aa, Artificial Sequence
  Robo4 VH-CH1(EE)-CD3 VL-CH1-Fc(knob, P329G LALA)
QVQLKQSGAELVKPGASVKISCKTSVYTFTYGYMHWVEQKPGQGLEWIGRIDPDSGNSMYNQKFQGRATLTRDKSSSTVYMELRSLTSEDSAVYYCARSMRYSGYRDYALDLWGQGTQVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 153 - protein, 232 aa, Artificial Sequence
  CD3 VH-CL
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 216 aa, Artificial Sequence
  Robo4 VL-CL(KK)
QLVLTQSPSASASLGASVKLTCTLSSQHSSYGITWLQQHPDKAPKYVMYLKSDGSHTKGADIPDRFSGSSSGVHRYLSISNVQPEDEAIYFCVTYDSTHVFGSGTQLTVLGQPKAAPSVTLFPPSSKKLQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 155 - protein, 115 aa, Artificial Sequence
  DP47 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 156 - protein, 108 aa, Artificial Sequence
  DP47 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 157 - DNA, 375 bp, Artificial Sequence
  VH CD3
gaggtgcagctgctggaatctggcggcggactggtgcagcctggcggatctctgagactgagctgtgccgccagcggcttcaccttcagcacctacgccatgaactgggtgcgccaggcccctggcaaaggcctggaatgggtgtcccggatcagaagcaagtacaacaactacgccacctactacgccgacagcgtgaagggccggttcaccatcagccgggacgacagcaagaacaccctgtacctgcagatgaacagcctgcgggccgaggacaccgccgtgtactattgtgtgcggcacggcaacttcggcaacagctatgtgtcttggtttgcctactggggccagggcaccctcgtgaccgtgtcatct

SEQ ID NO: 158 - DNA, 327 bp, Artificial Sequence
  VL CD3
caggccgtcgtgacccaggaacccagcctgacagtgtctcctggcggcaccgtgaccctgacatgtggcagttctacaggcgccgtgaccaccagcaactacgccaactgggtgcaggaaaagcccggccaggccttcagaggactgatcggcggcaccaacaagagagcccctggcacccctgccagattcagcggatctctgctgggaggaaaggccgccctgacactgtctggcgcccagccagaagatgaggccgagtactactgcgccctgtggtacagcaacctgtgggtgttcggcggaggcaccaagctgacagtccta

SEQ ID NO: 159 - DNA, 648 bp, Artificial Sequence
  Robo4 VL-CL(KK)
caacttgttctgactcagtcaccctctgcctctgcctctctgggagcctcagtcaaactcacctgcaccttgagtagtcagcacagcagttatggcattacttggctccagcaacatccagacaaggctcctaagtatgtgatgtatcttaagagtgatggaagccataccaagggagctgatatcccggatcgcttctctggctccagttctggagttcatcgctacttaagcatctccaacgtgcagcctgaggatgaagcaatctatttctgtgttacatatgatagcactcatgtttttggcagcggaacccagctcaccgtcctaggtcaacccaaggctgcccccagcgtgaccctgttcccccccagcagcaagaaactgcaggccaacaaggccaccctggtctgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggagaaaaccgtggcccccaccgagtgcagc

SEQ ID NO: 160 - DNA, 1353 bp, Artificial Sequence
  Robo4 VH-CH1(EE)-Fc(hole, P329G LALA)
caggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggcccctccgtgttccccctggcccccagcagcaagagcaccagcggcggcacagccgctctgggctgcctggtcgaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacgagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccg

SEQ ID NO: 161 - DNA, 2028 bp, Artificial Sequence
  Robo4 VH-CH1(EE)-CD3 VL-CH1-Fc(knob, P329G LALA)
caggtccagctgaagcagtctggggctgagctggtgaagcctggagcctcagtgaagatatcctgcaagacttcagtctacaccttcacttatggttatatgcactgggttgagcagaagcctgggcagggtctggagtggattggaagaattgatcctgatagtggtaatagtatgtacaatcagaagttccagggcagggccacactgactagagacaaatcctccagcacagtctacatggagctcagaagtctgacatctgaggactctgctgtatattactgtgcaagatcgatgcgatatagcggatatagggactatgctctggatttgtggggtcaagggacccaagtcactgtctcctcagctagcaccaagggcccctccgtgtttcctctggccccttccagcaagtccacctctggcggaactgccgctctgggctgcctggtggaagattacttccccgagcccgtgaccgtgtcctggaattctggcgctctgacctccggcgtgcacacctttccagctgtgctgcagtcctccggcctgtactccctgtcctccgtcgtgacagtgccctccagctctctgggcacccagacctacatctgcaacgtgaaccacaagccctccaacaccaaggtggacgagaaggtggaacccaagtcctgcgacggtggcggaggttccggaggcggaggatcccaggctgtcgtgacccaggaaccctccctgacagtgtctcctggcggcaccgtgaccctgacctgtggatcttctaccggcgctgtgaccacctccaactacgccaattgggtgcaggaaaagcccggccaggccttcagaggactgatcggcggcaccaacaagagagcccctggcacccctgccagattctccggttctctgctgggcggcaaggctgccctgactctgtctggtgctcagcctgaggacgaggccgagtactactgcgccctgtggtactccaacctgtgggtgttcggcggaggcaccaagctgaccgtgctgtccagcgcttccaccaagggacccagtgtgttccccctggcccccagctccaagtctacatccggtggcacagctgccctgggatgtctcgtgaaggactactttcctgagcctgtgacagtgtcttggaacagcggagccctgaccagcggagtgcacacattccctgcagtgctgcagagcagcggcctgtatagcctgagcagcgtcgtgaccgtgccttcctctagcctgggaacacagacatatatctgtaatgtgaatcataagcccagtaataccaaagtggataagaaagtggaacctaagagctgcgataagacccacacctgtcccccctgccctgctcctgaagctgctggtggccctagcgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcctagagaggaacagtacaactccacctaccgggtggtgtccgtgctgacagtgctgcaccaggactggctgaacggcaaagagtacaagtgcaaggtgtccaacaaggccctgggcgctcccatcgaaaagaccatctccaaggccaagggccagccccgggaaccccaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccg

SEQ ID NO: 162 - DNA, 696 bp, Artificial Sequence
  CD3 VH-CL
gaagtgcagctgctggaatccggcggaggactggtgcagcctggcggatctctgagactgtcttgtgccgcctccggcttcaccttctccacctacgccatgaactgggtgcgacaggctcctggcaagggcctggaatgggtgtcccggatcagatccaagtacaacaactacgccacctactacgccgactccgtgaagggccggttcaccatctctcgggacgactccaagaacaccctgtacctgcagatgaactccctgcgggccgaggacaccgccgtgtactattgtgtgcggcacggcaacttcggcaactcctatgtgtcttggtttgcctactggggccagggcaccctcgtgaccgtgtcatctgctagcgtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaacagccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgc